(19) United States
(12) Patent Application Publication
(10) Pub. No.: US 2012/0310140 A1
(43) Pub. Date: Dec. 6, 2012

(54) DIRECTED DELIVERY OF AGENTS TO NEURAL ANATOMY

(75) Inventors: Jeffery Kramer, San Francisco, CA (US); Mir A. Imran, Los Altos, CA (US)

(73) Assignee: SPINAL MODULATION, INC., Menlo Park, CA (US)

(21) Appl. No.: 13/309,429

(22) Filed: Dec. 1, 2011

Related U.S. Application Data
(60) Provisional application No. 61/418,721, filed on Dec. 1, 2010.

(51) Int. Cl. A61M 37/00; A61N 1/30; A61N 1/36; A61M 5/00

(52) U.S. Cl. 604/20; 604/93.01; 607/3

(57) ABSTRACT

The present invention is directed generally to systems, devices and methods for direct delivery of agents, e.g., pharmaceutical agents, to target spinal and neuronal anatomies, e.g., the dorsal root ganglia (DRG), for the treatment of various disorders, particularly pain and pain related disorders, such as chronic itch, sensory disorders, multiple sclerosis, post-herpetic neuralgia and the like. The system, devices and methods of the invention encompass the agents to be delivered to the target anatomy alone or in combination with electrical stimulation. The delivery device and systems and methods as disclosed herein place the distal end of the delivery element, which comprises at least one agent delivery structure, and optionally at least one electrode, in close proximity, or in contact with or next to the target spinal anatomy, e.g., DRG. A variety of agents can be delivered using the device, including sodium channel blockers, biologics, neuroinflammatory modulators, toxins etc., to selectively neuromodulate the neurons. Agent delivery and/or electrical stimulation can be automated and/or can be controlled automatically or by a pre-determined program, or by a patient control pump (PCA).

### CROSS REFERENCE TO RELATED APPLICATIONS

This application claims priority under 35 U.S.C. 119(e) of U.S. Provisional Patent Application Ser. No. 61/418,721 filed on Dec. 1, 2010, the contents of which are incorporated herein by reference in their entirety.

### FIELD OF THE INVENTION

The present invention is directed to methods, devices and systems for neurostimulation of target neural anatomies, particularly the dorsal root and dorsal root ganglion. Such methods, devices and systems include agent delivery alone or in combination with electrical stimulation for the treatment of various conditions, particularly pain and pain-related disorders.

### BACKGROUND OF THE INVENTION

Pain affects more Americans than heart disease, diabetes and cancer combined. In fact, about 50 million Americans suffer from chronic pain and spend about $100 billion for treatments per year. Unfortunately, many of the strongest available analgesics have serious side-effects including addiction, dependence and increased risk of heart attack and stroke. Moreover, many chronic pain conditions cannot be effectively treated with existing medications. Considering the revenue of drugs like CELEBREX® ($2.8 billion in 2004; G.D.Searle & Co., Skokie, Ill., United States of America) and VIOXX® ($1.4 billion in 2004, Merck & Co., Inc., Whitehouse Station, N.J., United States of America), a safe and effective treatment for chronic pain would significantly benefit human health. Accordingly, there is an unmet need for effective pain treatments. The present invention aims to meet at least some of these objectives.

### SUMMARY OF THE INVENTION

The present invention is directed generally to systems, devices and methods for direct delivery of agents, e.g., drugs, directly to the spinal anatomy of humans, particularly to at least one region or a combination of regions selected from the dorsal root (DR) and/or dorsal root ganglia (DRG) and/or dorsal root entry zone (DREZ) and/or intrathecal space. Such delivery can be used to treat a variety of conditions, including, for example, the treatment of pain and pain related disorders, including but not limited to neuropathic pain, chronic itch, puritis, sensory disorders, multiple sclerosis, post-herpetic neuralgia and the like.

In some embodiments, the system and devices as disclosed herein can be used to deliver at least one agent alone to the target spinal anatomy, or alternatively in combination with electrical stimulation. In some embodiments, the delivery of an agent to the target anatomy using the devices as disclosed herein is in a temporal pattern which is coordinated with a temporal pattern of electrical stimulation of the target anatomy, such that an agent is delivered to the target spinal anatomy in combination with electrical stimulation. In some embodiments, the delivery of the agent can be simultaneous with the electrical stimulation, or alternatively the delivery of the agent may be before or after the electrical stimulation of the target spinal anatomy.

The present invention has numerous advantages over existing methods and devices for the treatment of pain. In particular, one advantage is that selected target spinal anatomies undergo pathological changes, referred to as “neuronal plasticity” in certain pain pathologies, e.g., during inflammatory pain and neuronal injury. If the changes occur in the peripheral nervous system, this is referred to as peripheral sensitization. For example, without wishing to be bound by theory, nociceptors have a characteristic thresholds or sensitivity that distinguish them from other sensory nerve fibers. Depending on the nociceptor type, the can be excited by intense noxious heat, intense pressure or irritant chemicals, but not innocuous stimuli such as warming or light touch. In particular, alternations in pain pathways lead to hypersensitivity, such that pain outlives its usefulness as an acute warning system and instead becomes chronic and debilitating. This may be seen, at some level, as an extension of the normal healing process, whereby tissue or nerve damage elicits hyperactivity to promote guarding of the injured area. For example, sunburn produces temporary sensitization of the affected area. As a result normally innocuous stimuli, such as light touch or warmth, are perceived as painful (a phenomenon referred to as allodynia), or normally painful stimuli elicit pain of greater intensity (referred to as hyperalgesia). At its extreme, the sensitization does not resolve. Indeed, individuals who suffer from arthritis, postherpetic neuralgia (after a bout of shingles), or bone cancer experience intense and often unremitting pain that is not only physiologically and psychologically debilitating, but may also hamper recovery. Chronic pain may even persist long after an acute injury. Accordingly, in many pain instances, e.g., inflammatory pain or nerve injury, the expression of certain receptors and ion channels can be upregulated and downregulated in dorsal root ganglion cells (DRG), the cell body of the of primary sensory neurons, which can decrease the threshold of activation of these nociceptor neurons, resulting in an increased pain sensation in the subject by a stimuli which would not normall cause pain. As an example, under sustained peripheral inflammation, pro-longed c-fiber activation alters the pattern of gene transcription from the DRG and the dorsal horn neurons. Additionally, some components of the inflammatory soup (e.g., protons, ATP, serotonin, lipids) alter the excitability of neurons directly by interacting with ion-channels on the sensory neuron cell surface. For example, NGF activates TrkA on neurons, bradykinin activates BK₂ receptor, serotonin activates 5-HT₃ receptors, ATP activates P2X₃ receptor, protons (H⁺) activate ASIC3/VR1 receptors, lipids activate the PGE₂, CB1 and VR1 receptors, and heat activate the VR1/VRL-1 receptors, belonging to the TRPV family of ion channels, thus sensitizing (e.g., lowing the threshold of activation) or exciting the terminals of the nociceptors. Accordingly, a subject suffering from inflammatory pain could be treated with a specific pharmacological agent at the target spinal anatomy, e.g., DRG to ameliorate the effects of the inflammatory mediators on the activation of ion channels and other receptors in the DRG.

Accordingly, an agent delivered with the devices and systems as disclosed herein for the treatment of clinical pain would not have an effect on normal patients not experiencing chronic pain where such pathological changes have not occurred. Another advantage of the devices, systems and methods as disclosed herein is that it allows for specific and localized delivery of agents to specific target spinal anatomies, such as but not limited to the DRG. Accordingly, lower doses can be used and therefore avoids any off-target and/or systemic side affects associated with the delivered agent. Another advantage of the present system allows for the combination of electrical stimulation to be used in combination with delivering the agent to the target spinal anatomy. For example, the combination of electrical stimulation and concurrent agent delivery is useful for activating channels present in specific cells present at the target spinal antatomy, e.g., DRG, and thus allowing entry of the agents into specific cells of interest, which increases the specificity and selectivity of the agents delivered. Additionally, the electrical stimulation can be used to activate certain agents, e.g., from a pro-drug to a biologically active agent at the target spinal anatomy location.

Any agent can be delivered using the device and system as disclosed herein, including but not limited to ion channel agonists and antagonists, sodium channel blockers, biologics, neuroinflammatory modulators, toxins etc., to selectively neuromodulate or inhibit the electrical impulses from the neurons. In some embodiments to selectively destroy the neurons. For example, in some embodiments, a toxin which binds to and is specific for a specific neuronal cell-type can be used to selectively ablate certain pain-transmitting neuronal types, e.g., neurons firing ectopically or spontaneously as a result of innocuous stimuli.

In particular, in some embodiments, the agent delivered to the target spinal anatomy using the devices, systems and methods as disclosed herein is selected based on the particular pain indication to be treated. For example and without wishing to be limited to theory, inflammatory mediators such as, but not limited to Prostaglandin E2 (PGE2) increase the excitability of DRG neurons in part by reducing the extent of membrane depolarization needed to activate TTX-R Na+ channels. Accordingly, sensory neurons have increased spontaneous firing and repetitive spiking, resulting in increased intense pain sensation by the subject. Additionally, other pro-inflammatory agents, such as bradykinin and capsaicin increase activation of the Vanilloid Receptor [VR1]) and increase the effect the TTX-R Na+ channel. Embodiments of the present invention advantageously utilize aspects of the pain pathway and neurochemistry to modify electrophysiological excitability of the DRG neurons where electrical stimulation is coupled with pharmacological agents (electrical stimulation alone or in combination with a pharmacological agent) to optimize the efficacy of the stimulation system.

Other aspects of the present invention relate to a combination of agent delivery and/or electrical stimulation to the target anatomy, which can be automated and/or can be “on demand” for example, controlled by a patient controlled analgesia (PCA) pump. In particular, the present invention is different from the neurostimulation methods and systems as disclosed in International Application WO2006/029257, and U.S. Application US 2008/0167698 (which are incorporated herein in their entirety by reference), in that the delivery device as disclosed herein allows for controlled and precise delivery of one or more pharmacological agents to a target anatomy, which can be specifically tailored to a particular delivery regimine by the physician and/or patient.

Additionally, the present application provides for coordinated delivery of one or more pharmacological agents with electrical stimulation so that the delivery of the agent and/or electrical stimulation can be timed with resepect to each other, e.g., temporally regulated (e.g., an agent is delivered (e.g. “on”) or not delivered (e.g., “off”)) according to a particular electrical stimulation patterning or treatment regimine and/or can be delivered “on demand” by the patient using a patient controlled analgesia (PCA) pump.

Additionally, the delivery device as disclosed herein allows the delivery of one or more agents in a coordinated manner, or in concert with the electrical stimulation, for example, where the coordinated delivery allows the pharmacological agent to act synergistically with the electrical stimulation, such that the efficacy of the agent is enhanced by the electrical stimulation. For instance, but not wishing to be bound by theory, an agent to be delivered using the device as disclosed herein selected to be delivered based on its ability for its activity or efficacy to be enhanced by the electrical stimulation. Such electrical-stimulation induced enhancement of the agent can be by a variety of mechanisms, e.g., the agent becomes activated on electrical stimulation, or the target receptors or ion channels that the agent modulates becomes activated or open by the electrical stimulation such that the agent only acts on activated receptors and/or open ion channels etc, or migration of an agent to particular cell subtype on electrical stimulation etc. Furthermore, in some embodiments of the present delivery device as disclosed herein, the electrodes and the agent delivery structure, such as an outlet port, are close together (in some embodiments, the electrodes are interdispursed between outlet ports) such that the electrical stimulation can activate the agent being delivered by the device, therefore enabling a better control of electrical stimulation with agent delivery such that the electrical stimulation and delivery of the agent function synergistically to reduce the pain sensation in the subject.

Accordingly, in some embodiments, the devices, methods and systems as disclosed herein provide improvements over existing systems in that they allow delivery of selected agents which are enhanced by electrical stimulation. Some additional advantages of the delivery device and methods as disclosed herein include but are not limited to the temporal pattern of the delivery of the agent alone, or in conjunction with a coordinated temporal electrical stimulation, such that the stimulation parameters specifically activate the agent.

Additional advantages of the delivery device and methods as disclosed herein include but are not limited to the delivery of the agent by the device in a delivery agent, e.g., a vector or carrier particle, such that the delivered agent remains in the location it was delivered for a period of time for effective therapeutic effect (e.g., reduction in pain sensation by the subject).

In some embodiments, the electrical stimulation can be used to deliver the agent to the target spinal anatomy. For example, in some embodiments, the present invention can be adapted so that the electrical stimulation is used for electrophoritic (also referred to as “iontophoretic flux” or “iontrophoretic”) agent delivery, where an electrically conducting wire in the delivery lumen 140 can be used to charge the agent (e.g., either a positive or negative charge) within the lumen, and as the charge is greater than the charge in the subjects body, the charged agent is driven out of the lumen and through the outlet ports 40 and into close proximity of the target site, such as the DRG.

Accordingly, the present invention relates to the combination neurostimulation and pharmacological agent delivery element, where the inventors have discovered that a pre-determined temporal pattern of neurostimulation and agent delivery surprisingly results in a greater efficacy of the agent and reduced pain sensation in the subject as compared to either delivery of the agent alone, or the electrical stimulation alone, thus being able to obtain the desired stimulation or modulation level.

Other aspects of the disclosure relate to methods for treating chronic pain. For example, in one embodiment, the disclosure relates to a method for treating chronic nerve pain in a subject, e.g., a mammal such as a human. In some embodiments, in accordance with the method, the area of pain in the subject is identified, and the spinal level within the mammal that is associated with the chronic pain is determined. A delivery device as disclosed herein is provided for introducing an agent at the location of the DRG associated with the chronic pain.

Other aspects of the present invention relate to a method for targeted treatment of pain and pain related disorders and/or conditions with minimal deleterious side effects, such as undesired side effects as a result of off-target non-specific effects of an agent as well as undesired motor responses or stimulation of unaffected body regions. In some embodiments, the system and devices as disclosed herein achive minimal deleterious side effects by directly delivering the agent to the target anatomy in combination with selectively neuromodulating the target anatomy, e.g., the DRG to modulate or decrease pain and a pain related disorder or condition, while minimizing or excluding undesired side effects by avoiding non-specific or systemic administration of a pain agent or analgesic, or generalized neuromodulation of other anatomies. In most embodiments, delivery of the agent to the target anatomy can be alone or in combination with neurostimulation, such as electrical stimulation, however it may be appreciated that neurostimulation may include a variety of forms of altering or modulating nerve activity by at least one agent and optionally, delivering electrical stimulation directly to the target anatomy. For illustrative purposes, descriptions herein will be provided in terms of agent delivery to the DRG in combination with electrical stimulation, with exemplary stimulation parameters as well as temporal patterning of the agent delivery with the electrical stimulation, however, it may be appreciated that such descriptions are not so limited and may include a combination or variety of agent delivery methods, e.g., continuous, on-demand, intermittent based on a predefined temporal pattern of delivery to the DRG, and in combination with electrical stimulation, using a variety of different parameters, such as intermittent and in a temporal regulated pattern so the electrical stimulation of the DRG works synergistically with the delivery of the agent to the DRG.

In particular, the combining of direct delivery of an agent to the DRG with electrical stimulation of the DRG as disclosed herein provides provide several advantages. For example, the delivered agent and electrical stimulation can function synergistically to decrease pain sensation in a subject, and/or enhance the therapeutic effect of the agent and the electrical stimulation as compared to their use alone. Alternatively, in some embodiments, the electrical stimulation increases the selectivity of an agent to target DRG cell bodies. Alternatively, in some embodiments, the electrical stimulation enables targeted activation of an agent delivered to the DRG. In another embodiment, the electrical stimulation causes differential enhancement of an agent to delivered target DRG cell bodies.

Typically, the agent-neurostimulatory systems and delivery devices as disclosed herein are used to neuromodulate portions of neural tissue of the pairs of nerves along the spinal cord which are known as spinal nerves. The spinal nerves include both dorsal and ventral roots that integrate near the intravertebral foramen to create a mixed nerve which is part of the peripheral nervous system. At least one dorsal root ganglion (DRG) is disposed along each dorsal root prior to the point of mixing. Thus, the neural tissue of the central nervous system is considered to include the dorsal root ganglions and exclude the portion of the nervous system beyond the dorsal root ganglions, such as the mixed nerves of the peripheral nervous system. Typically, the agent-neurostimulatory systems and delivery devices as disclosed herein are used to neuromodulate one or more spinal anatomy, for example, but not limited to one or more dorsal root ganglia, dorsal roots, dorsal root entry zones, or portions thereof, while minimizing or excluding undesired stimulation of other tissues, such as surrounding or nearby tissues, ventral root and portions of the anatomy associated with body regions which are not targeted for treatment. However, it may be appreciated that stimulation of other tissues are contemplated. In some embodiments, it is also envisioned that a system or device can neuromodulate different neural anatomies in the same subject, for example, for illustration purposes only but by no way a limitation, the device or system may be configured and positioned in a subject so that an agent and electrical stimulation is delivered to a spinal anatomy such as DRG, and can also be delivered to a different spinal anatomy of the subject, such as dorsal root. Or, the device or system may be configured and positioned in a subject so that an agent and electrical stimulation is delivered to a spinal anatomy such as DRG, and can also be delivered to a different spinal anatomy of the subject, such as a spinal cord. Or, the device or system may be configured and positioned in a subject so that an agent and electrical stimulation is delivered to a spinal anatomy such as DRG, and can also be delivered to a different neural anatomy of the subject, such as a sympathetic ganglion or peripheral nerve. Accordingly, any combination of different neural anatomies can be targeted for agent delivery and electrical stimulation by the methods, systems and devices as disclosed herein. It is also encompassed that any combination of different neural anatomies at different spinal cord levels can be targeted in a subject by the devices and systems as disclosed herein.

Accordingly, as the devices, systems and methods to treat various disorders as disclosed herein enable both an agent and in some embodiments, electrical stimulation, to be delivered at a specific dose and specific stimulation energy levels to a defined anatomical location, e.g., in proximity to the dorsal root, in particular, the dorsal root ganglion (DRG), the devices, systems and methods have numerous advantages including reduced side effects associated with systematic delivery agents, and/or adverse side effects from spinal cord electrical stimulation (SCS). Additionally, as the localized delivery of the agent to the DRG can be coordinated with the specific electrical stimulation of the DRG, it provides a superior level of control and specificity of agent efficacy and/or the electrical simulation effect which is not easily achievable with other systems.

Accordingly, the present invention relates generally to devices, systems and methods for direct delivery of agents, e.g., analgesics and pain medicine to the DRG. Herein, in one embodiment, the device for direct delivery of agents to a target neural anatomy, e.g., DRG is referred to as delivery device (DD) 10, which comprises a agent release module 20 connected to a delivery element 30 for transporting the agents from the agent release module, where they are stored and released in a controlled manner, to the delivery site at the anatomical target spinal anatomy location, which can be any of, but not limited to one or more dorsal root ganglia, dorsal roots, dorsal root entry zones and other spinal anatomies. In some embodiments, the delivery element 30 is configured as a catheter comprising a lumen for delivery of at least one agent to at least one target neural anatomy. In alternative embodiments, the delivery element 30 is configured as a lead comprising at least one electrode which is connected to a pulse generator for electrical stimulation of at least one target spinal anatomy.

Accordingly, the agent release module of the delivery device is placed in the subject's body in an anatomically convenient location, such as in the back or buttocks, and the agent or drug formulation is transported along fluidly connected agent delivery elements such that the agent or the drug formulation is released at least one target spinal anatomy, e.g., a DRG delivery site. A target delivery site is in close proximity to at least one target spinal anatomy, e.g., a DRG, and in some embodiments the released drug formulation functions on the cell bodies in the DRG to modulate the pain response.

In some embodiments, the delivery device is further configured for combining the delivery of a drug formulation in close proximity to the DRG with electrical stimulation of the DRG. In such embodiments, the agent release module further comprises a pulse generator and a battery and is connected to leads which comprise electrodes near its distal end which are positioned in close proximity to the DRG, allowing for a combined electrical stimulation and agent delivery either simultaneously (e.g., at the same time) or in a pre-determined temporal pattern of electrical stimulation and agent delivery.

In some embodiments, an agent or drug formulation is stored within an agent release module (e.g., contained in a reservoir or impregnated within a matrix within the agent release module). The drug formulation comprises an amount of drug sufficient for treatment and is stable at body temperatures (i.e., no unacceptable degradation) for the entire pre-selected treatment period. The agent delivery devices store the drug formulation safely (e.g., without dose dumping), provide sufficient protection from bodily processes to prevent unacceptable degradation of the formulation, and release the drug formulation in a controlled fashion at a therapeutically effective rate to treat pain

One object of the invention is to provide a method for convenient, long-term management of pain.

One advantage of the invention is that the delivery devices, systems and methods described herein provide effective management of pain by administration of an agent, e.g., a drug formulation, directly to the DRG, providing adequate pain relief and a reduction in adverse side effects relative to systematic or delivery of agents to other locations. Another advantage is that the present invention relates to a combined used of electric stimulation of the target anatomy, e.g., DRG concurrently with, or in a temporal pattern with direct delivery of an agent to the DRG. This provides several advantages, which include a synergistic effect of the electrical stimulation to increase the efficacy of the agent and vice versa (e.g., synergistic analgesia), such that the therapeutic effect of the agent and the electrical stimulation are each enhanced when they are used together, as compared to their use alone, or an increase of the selectivity of an agent (e.g., agent targeting) to target anatomy, e.g., DRG cell bodies in the presence of electrical stimulation, or an increased targeted activation of an agent (e.g., compound activation) delivered to the target anatomy, e.g., DRG in the presence of electrical stimulation. Another advantage of concurrent or temporal delivery of agents and electrical stimulation is that e-fields can result in differential enhancement of an agent (e.g., cell specific target enhancement) delivered to the target anatomy, e.g., DRG cell bodies.

Given the adverse effects of many pain drugs, e.g., opioid analgesics, one of the advantages of the delivery device as disclosed herein is lower doses which still provide considerable benefit to those desiring pain relief, particularly in relatively long term (e.g., 1-4 months) pain situations. Furthermore, the delivery devices can also be more cost-effective, and thus may make pain management available to a broader population. Such target specific delivery can also reduce escalating tolerance, dependence and incomplete effectiveness due to localized concentrations of the effective agent delivered at a concentration sufficient to achieve the desired effect at the target spinal anatomy, e.g., DRG.

Another advantage of the invention is that the invention can be used to deliver relatively small quantities of pain drugs to a subject accurately and precisely and thus safely delivering such agents and pain drugs despite the extreme potency of these agents. Thus, the invention allows for the convenient use of a variety of different pain drugs for treatment of pain ranging in severity from mild to severe.

Another surprising advantage of the systems and devices as disclosed herein relates to the combined use of electrical stimulation of the DRG in combination with direct delivery of agents to the DRG, which enables tailoring the pain treatment to the patients needs, as well as providing sufficiently effective therapy over a relatively long duration of therapy.

One notable advantage of the agent-neurostimulatory systems as disclosed herein avoids the need for placement of external needles and/or catheters in the subject, which might provide sites susceptible to infection. In addition, use of delivery devices in a subject increases the patient compliance with a prescribed therapeutic regimen, substantially decreases or completely avoids the risk of abuse of the agent by the patient or others in contact with the patient, and affords greater mobility and easier outpatient management.

Another advantage of the agent-neurostimulatory systems and delivery devices as disclosed herein is that a selective agent, or combination of agents can be delivered directly to the DRG with such accuracy and precision and at such low quantities of agent is required, and allows long-term use of such agents to treat pain. Additionally, the agent-neurostimulatory systems as disclosed herein also allows for effective pain management by the subject via the patient programmer 60 allowing treatment of breakthrough pain episodes, as well as tailoring the delivery of the agent with electrical stimulation of the DRG for tailoring the pain treatment in real-time to meet the needs of the subject for pain relief at that particular time period.

Another advantage of the agent-neurostimulatory systems as disclosed herein allows the delivery of agents which marked potency, e.g., delivery of agents such as opioids, Na+ channel blockers, etc., in small amounts and volumes directly to the DRG, avoiding undesired side-effects of systemic administration or risks of subject addiction and the like.

Yet another advantage is that the invention provides for precise delivery of an agent to the DRG, thus allowing delivery of lower doses and/or for delivery of precisely metered doses of a particular agent at a consistent delivery volume rates (e.g., on the order of microliters to milliliters per hour) which can be controlled precisely and maintained for pre-determined periods of time.

Micro-electrode and stimulation system embodiments of the present invention may be placed in close proximity to a single nerve root ganglion (e.g., DRG) utilizing method as disclosed herein. In some embodiments, the distal end of the delivery element, which comprises the agent delivery structure, such as outlet ports, (and optionally the electrodes) are placed in close proximity, or in contact with the dorsal root ganglion epinurium, or just below the surface of the dorsal root ganglion epinurium. In some embodiments, the distal end of the delivery element does not penetrate or is not implanted into the DRG (e.g, see embodiments shown in FIGS. 3, 5, 12, 13, 22 and 26).

The methods as described herein provide numerous advantages, including but not limited to: low risk percutaneous access route similar to other procedures, direct delivery of localized quantities of pharmacological agents at the specific target spinal anatomy, e.g., DRG or nerve root when using an embodiment of the device having electrodes, and electrode placement that enables preferential, selective nerve fiber stimulation along with pharmacological agent delivery.

One aspect of the present invention relates to a neuromodulation system comprising (a) a delivery element having a distal end and at least one outlet port disposed near the distal end, wherein the distal end is configured for positioning at least one of the at least one outlet ports near a dorsal root ganglion; (b) an agent release module connectible with the delivery element, the agent release module having an agent release mechanism; and (c) an agent releaseable from the agent release mechanism so as to be delivered from the at least one outlet port according to a controlled release pattern to at least assist in neuromodulating the dorsal root ganglion. In some embodiments, the agent is chargeable and the agent release mechanism includes a mechanism for charging the agent so that the agent is delivered by iontophoretic flux according to the controlled release pattern.

In some embodiments of all aspects of the invention as disclosed herein, an agent which is delivered can be, for example, but is not limited to, one or more or a combination of: lidocaine, epinephrine, fentanyl, fentanyl hydrochloride, ketamine, dexamethasone, hydrocortisone, peptides, proteins, Angiotension II antagonist, Antriopeptins, Bradykinin, Tissue Plasminogen activator, Neuropeptide Y, Nerve growth factor (NGF), Neurotension, Somatostatin, octreotide, Immunomodulating peptides and proteins, Bursin, Colony stimulating factor, Cyclosporine, Enkephalins, Interferon, Muramyl dipeptide, Thymopoietin, TNF, growth factors, Epidermal growth factor (EGF), Insulin-like growth factors I & II (IGF-I & II), Inter-leukin-2 (T-cell growth factor) (Il-2), Nerve growth factor (NGF), Platelet-derived growth factor (PDGF), Transforming growth factor (TGF) (Type I or δ) (TGF), Cartilage-derived growth factor, Colony-stimulating factors (CSFs), Endothelial-cell growth factors (ECGFs), Erythropoietin, Eye-derived growth factors (EDGF), Fibroblast-derived growth factor (FDGF), Fibroblast growth factors (FGFs), Glial growth factor (GGF), Osteosarcoma-derived growth factor (ODGF), Thymosin, or Transforming growth factor (Type II or β)(TGF). In some embodiments, an agent delivered is selected from one or more or a combination of: opioids, COX inhibitors, PGE2 inhibitors, Na+ channel inhibitors.

In some embodiments of all aspects of the invention as disclosed herein, an agent which is delivered can be, for example, an agonist or antagonist of a receptor or ion channel expressed by a dorsal root ganglion, for example, an agonist or antagonist of a receptor or ion channel which is upregulated in a dorsal root ganglion in response to nerve injury, inflammation, neuropathic pain, and/or nociceptive pain. In some embodiments, an ion channel expressed by the dorsal root ganglion is selected from any one of, or a combination of: voltage gated sodium channels (VGSC), voltage gated Calcium Channels (VGCC), voltage gated potassium channel (VGPC), acid-sensing ion channels (ASICs). In some embodiments, a voltage-gated sodium channel (VGSC) includes TTX-resistant (TTX-R) voltage gated sodium channels, such as, but not limited to, Naᵥ1.8 and Naᵥ1.9. In some embodiments, a voltage-gated sodium channel (VGSC) is a TTX-sensitive (TTX-S) voltage gated sodium channel, for example, but not limited to, Brain III (Naᵥ1.3). In some embodiments, a receptor is selected from any one of, or a combination of, ATP receptor, NMDA receptors, EP4 recetors, metrix metalloproteins (MMPs), TRP receptors, neurtensin receptors.

In some embodiments of all aspects of the invention as disclosed herein, a delivery element further comprises at least one electrode which is capable of delivering electrical energy, for example, to provide electrical energy to assists in creating the iontophoretic flux of the agent, amoung other effects of the electrical stimulation, such as activating or opening specific ion channels and/or receptors on the soma of the sensory neurons. In some embodiments, least one electrode in close proximity to the at least one agent delivery structure, e.g., an agent outlet port, and in some embodiments, the electrodes can be intermittent between one or more agent delivery structures.

In some embodiments, an agent release module further comprises a pulse generator which provides the electrical energy in a manner which impacts the effect of the agent on at least a portion of the dorsal root ganglion. In some embodiments, the electrical energy is provided once the agent has targeted at least a portion of the dorsal root ganglion. In some embodiments, the electrical energy is provided in a manner that targets at least one particular type of cell within the dorsal root ganglion, for example the cell body of a sensory neuron, e.g., but not limited to the soma of a c-fiber sensory neuron.

In some embodiments, the controlled release pattern of the electrical release pattern and/or agent release is determined to impact an effect of the electrical energy on at least a portion of the dorsal root ganglion, or alternatively, where the agent and/or the controlled release pattern is determined to enhance the ability of the electrical energy to excite or inhibit a primary sensory neuron in the dorsal root ganglion. In some embodiments, the agent and/or the controlled release pattern is determined to cause a change in the open probability of at least one sodium channel.

In some embodiments, the agent release mechanism delivers the agent to assist in neuromodulating the dorsal root ganglion over time. In some embodiments, the agent release mechanism comprises a matrix impregnated with the agent so that the matrix releases the agent over time according to the controlled release pattern, for example, an erodible material matrix.

In some embodiments, the agent is delivered in conjunction with a carrier particle, for example, but not limited to one or more or any combination of: a macromolecule complex, nanocapsule, microsphere, bead or lipid-based system, micelle, mixed micelle, liposome or lipid:oligonucleotide complex of uncharacterized structure, dendrimer, virosome, nanocrystal, quantum dot, nanoshell or nanorod. In further embodiments, an agent can also be conjugated or associated with a targeting molecule which targets the dorsal root ganglion, for example, but not limited to, a targeting molecule which has a specific affinity for a cell surface marker expressed on at least one cell within the dorsal root ganglion, for example, expressed on at least one cell body of a c-fiber.

In some embodiments, the agent can be delivered in conjunction with a gellable material which retains the agent near the dorsal root ganglion after delivery, for example, a gellable material which gells upon delivery (e.g., release from the agent delivery structure).

In some embodiments of all aspects of the invention as disclosed herein, the positioning the distal end of the delivery element comprises positioning at least one of the at least one outlet port on or in contact with the dorsal root ganglion epinurium. In some embodiments, the delivery element is not implanted or does not penetrate into the dorsal root ganglion.

Another aspect of the present invention relates to an intrathecal agent delivery system comprising: (a) a delivery element having a distal end and at least one outlet port disposed near the distal end, wherein the delivery element is configured for advancement within an intrathecal space along a spinal cord and then along a dorsal root to position at least one of the at least one outlet ports near an associated dorsal root ganglion; (b) an agent release module connectible with the delivery element, the agent release module having an agent release mechanism; and (c) an agent releaseable from the agent release mechanism so as to be delivered from the at least one outlet port to at least assist in neuromodulating the dorsal root ganglion.

In some embodiments, an intrathecal delivery system comprises a delivery element which includes a stylet, wherein the stylet has a curved distal end configured to assist in guiding the delivery element along a root sleeve angulation of the dorsal root during advancement. In some embodiments, the intrathecal delivery system can be used to deliver an agent to the DRG, and in some embodiments, the agent comprises a targeting molecule which targets the agent to the dorsal root ganglion, as disclosed herein, where a targeting molecule has a specific affinity for a cell surface marker expressed on at least one cell within the dorsal root ganglion, such as but not limited to a c-fiber cell body.

In some embodiments of the intrathecal delivery system, an agent delivered is selected from any or a combination of a benzodiazepine, clonazepam, morphine, baclofen and/or ziconotide. In some embodiments, the agent comprises a genomic agent or biologic. In some embodiments, an agent delivered by the intrathecal delivery system is activatable by electrical stimulation. In alterantive embodiments, an agent delivered by the intrathecal delivery system enhances the ability of electrical stimulation to excite or inhibit a primary sensory neuron in the dorsal root ganglion, or alternatively, can enhance the ability of electrical stimulation to target at least one specific cell within the dorsal root ganglion.

In some embodiments of all aspect of the present invention, an agent release module includes electronic circuitry capable of generating stimulation energy for delivery of the agent to the delivery element. In such embodiments, an electronic circuitry includes memory programmable with an electrical stimulation parameter set and an agent delivery parameter set, for example, where the set parameters cause the agent and the stimulation energy to be delivered in a predetermined coordinated manner.

Another aspect of the present invention relates to an agent delivery system comprising: (a) a delivery element having a distal end, at least one agent delivery structure disposed near the distal end and at least one electrode disposed near the distal end, wherein the distal end is configured for positioning at least one of at least one agent delivery structures and at least one of the at least one electrodes near a dorsal root ganglion; (b) a pulse generator connectable with the delivery element, wherein the pulse generator includes memory programmable with an electrical stimulation parameter set that controls delivery of electrical energy from the at least one electrode in a predetermined manner dependent on the delivery of an agent from the at least one of the at least one agent delivery structures.

In some embodiments of all aspect of the present invention, an agent delivery structure comprises an agent-eluting coating or an agent-eluting structure, for example where the agent delivery structure comprises an agent outlet port. In some embodiments, an agent delivery system as disclosed herein comprises a pulse generator which comprises an agent release mechanism which releases agent from the at least one agent outlet port. In some embodiments, a pulse generator includes memory programmable with an agent delivery parameter set that controls delivery of the agent from the agent release mechanism. In some embodiments, the delivery of the electrical energy is controlled to impact the effect of the agent on at least a portion of the dorsal root ganglion, and can be optionally timed to maximize the effect of the agent on the at least a portion of the dorsal root ganglion. In some embodiments, the delivery of the electrical energy is controlled based on an impact the delivery agent has on the effect of the electrical energy on at least a portion of the dorsal root ganglion. In some embodiments, the delivery of the electrical energy is reduced during delivery of the agent.

Another aspect of the present invention relates to a neuromodulation system comprising: (a) an agent delivery system including a delivery element having a distal end, at least one agent delivery structure disposed near the distal end and at least one electrode disposed near the distal end, wherein the distal end is configured for positioning at least one of the at least one agent delivery structure and at least one of the at least one electrodes near a dorsal root ganglion; (b) an agent releaseable from the at least one agent delivery structure, wherein electrical energy provided by the at least one electrode assists in neuromodulating the dorsal root ganglion by activating a cell body within the dorsal root ganglion so that the cell body is preferentially targeted by the agent.

In some embodiments, activating the cell body comprises depolarizing the cell body, for example, but not limited to, a cell body selected based on its size and/or membrane properties.

In some embodiments of all aspects of the invention as disclosed herein, an agent can be a toxin, for example, for selectively ablating a particular neuronal subtype or non-neuronal subtype. In some embodiments, toxin agents can be associated with targeting molecules to increase the selectivity and specificity to targeting a particular neuronal subtype, e.g., c-fibers and the like.

Another aspect of the present invention relates to a neuromodulation system comprising: (a) an agent delivery system including a delivery element having a distal end, at least one agent delivery structure disposed near the distal end and at least one electrode disposed near the distal end, wherein the distal end is configured for positioning at least one of the agent delivery structures and at least one of the one electrodes near a dorsal root ganglion; (b) an agent releaseable from the at least one agent delivery structure, wherein electrical energy provided by the at least one electrode selectively activates the agent in a first cell type within the dorsal root ganglion while not activating the agent in a second cell type within the dorsal root ganglion.

In some embodiments of all aspects of the invention as disclosed herein, an agent can be a pro-drug. In some embodiments of all aspects of the invention as disclosed herein, an agent can be selected from one or any combination of agents, for example, but not limited to opioids, COX inhibitors, PGE2 inhibitors, Na+ channel inhibitors. In some embodiments, agent can be an agonist or antagonist of a receptor or ion channel which is upregulated in a dorsal root ganglion in response to nerve injury, inflammation, neuropathic pain, and/or nociceptive pain.

Another aspect of the present invention relates to a method for administering a pharmacological agent to a target spinal anatomy of a subject, the method comprising: (a) positioning a distal end of a delivery element in proximity to the subjects target spinal anatomy, wherein the delivery element comprises at least one outlet port near the distal end, at least one lumen having a distal end and a proximal end, and wherein the lumen proximal end is connected to a first reservoir, and wherein the lumen distal end is connected to the at least one outlet port; and (b) delivering at least one pharmacological agent to the target spinal anatomy from the at least one outlet port, wherein the pharmacological agent is administered in a controlled manner to the target spinal anatomy. In some embodiments, the pharmacological agent is in a composition comprising a delivery agent, e.g., for example, a delivery vehicle such as a nanoparticle, vector, gel, or the like as disclosed herein to facilitate the delivery of the agent at the target spinal anatomy.

In some embodiments, the target spinal anatomy is at least one dorsal root ganglion (DRG), within the intrathecal space and/or within the epidural space. In some embodiments, the positioning the distal end of the delivery element comprises advancing the delivery element within an intrathecal space of the subject. In some embodiments, the positioning the distal end of the delivery element comprises advancing the distal end of the delivery element within an epidural space of the subject. In some embodiments, the positioning of the distal end of the delivery element comprises placing the outlet ports and/or the electrodes in close proximity, or in contact with the dorsal root ganglion epinurium, (but where the distal end of the delivery element is not implanted into, or penetrating the DRG) (e.g. see embodiments shown in herein in FIGS. 3, 5, 12, 13, 22, 26). In some embodiments, the distal end of the delivery element is positioned so that at least one of the outlet ports is adjacent to a portion of a dorsal root.

In many aspects of the embodiments as disclosed herein, the pharmacological agent modulates a pain sensation in the subject, for example, a human subject. In some embodiments, at least one outlet port includes any one of: a void, opening, hole or side wall aperture in the tube wall of the shaft, or in alternative embodiments, at least one outlet port includes a permeable portion of the delivery element. In some embodiments, the permeable portion extends around a circumference of a portion of the delivery element.

In some embodiments, the delivery device further comprises a tensile element.

In many aspects of the embodiments as disclosed herein, the delivery element can further comprise at least one electrode disposed near the distal end of the delivery element, and can be used in a method to provide electrical stimulation energy to the at least one electrode so to stimulate at least a portion of the target spinal anatomy. In some embodiments, the electrical stimulation can be used to charge an agent, to allow, for example iontophoretic flux of the agent out of the outlet ports at the target spinal anatomy. In some embodiments, at least one agent is delivered at the same time as the occurrence of the electrical stimulation energy. In alternative embodiments, at least one agent is delivered intermittently with providing electrical stimulation. In some embodiments, the electrical stimulation can be used to activate specific neuronal cell types and/or non-neuronal cells, e.g., glial cells or satellite cells and/or astrocytes so that it will enhance the efficacy of the pharmacological agent. For example, but not being limited to, the electrical stimulation can open ion channels, activate receptors present on specific neuronal cell types in the DRG, allowing the pharmacological agent to modulate said ion channels or receptors. In some embodiments, the electrical stimulation energy is generated by a pulse generator, for example, a pulse generator controlled by a controller. In some embodiments, a controller can additionally control the output of agent from the reservoir, thus control the output of the agent from the outlet port in the delivery element.

In some embodiments, the controller can control the generation of stimulation energy and/or the output of agent from the reservoir using a preset program, e.g., a program regimen determined by the physician and/or the patient, such that the release of the agent from the outlet ports is in a controlled manner, and can in some embodiments, be temporally regulated in a coordinated manner with the electrical stimulation. In some embodiments, a controller can controls the signal generator and/or output of agent from the reservoir and thus its release form the outlet ports of the delivery element using at least one of a plurality of predetermined programs selected by the physician. In an alternative embodiments, a controller can control the signal generator and/or output of agent from the reservoir, and thus release of the agent from the outlet ports on the delivery element in an “on demand” manner, as determined by the subject, for example, when the subject is experiencing breakthrough pain.

In some embodiments, the output from the reservoir is controlled by a controller, for example, where a controller controls the output of agent from the reservoir, and thus its release from the outlet port of the delivery agent using a preset program, or alternatively, “on demand” by the subject.

In all aspects of the embodiments as disclosed herein, an agent can be an agonist or antagonist of a receptor or ion channel expressed by a dorsal root ganglion, for example, an agonist or antagonist of a receptor or ion channel which is upregulated in a dorsal root ganglion in response to nerve injury, inflammation, neuropathic pain, and/or nociceptive pain. In some embodiments, an ion channel expressed by the dorsal root ganglion is selected from the group consisting of: voltage gated sodium channels (VGSC), voltage gated Calcium Channels (VGCC), voltage gated potassium channel (VGPC), acid-sensing ion channels (ASICs). In some embodiments, a voltage-gated sodium channel includes TTX-resistant voltage gated sodium channels, such as, but not limited to, Nav1.8 and Nav1.9. In some embodiments, a voltage-gated sodium channel is a TTX-sensitive voltage gated sodium channel, such as, but not limited to, Brain III (Nav1.3). In some embodiments, a receptor is selected from one or any combination of an ATP receptor, a NMDA receptor, a EP4 receptor, a matrix metalloproteins (MMPs), a TRP receptor, a neurtensin receptor, VR1 and the like.

Another aspect of the present invention relates to a system for delivering at least one agent to a target spinal anatomy in a subject, such as the DRG, DR, DREZ, comprising: (a) a delivery element with a distal and proximal end, and at least one outlet port near the distal end; and at least one lumen disposed within the delivery element, having a distal end and a proximal end, wherein the lumen proximal end is connected to a first reservoir, and wherein the lumen distal end is connected to the at least one outlet port; (b) a reservoir comprising an agent; and (c) a controller to control the output of the agent from the reservoir, and thus controlling the release of the agent from the outlet port of the delivery element.

In some embodiments, each delivery element can comprise at least one, or at least two lumens, for delivery of multiple agents to the target spinal anatomy. In such an embodiment, the proximal end of the second lumen can be connected to a second reservoir, and the distal end is connected to a second outlet port on the delivery element. In some embodiments, the delivery agent comprises at least one electrode disposed near the end of the delivery element, and where the controller can control output of electrical stimulation to the target spinal anatomy via the at least one electrode. In some embodiments, the electrode is located between (e.g., interspersed) between one or more outlet ports on the delivery element. In some embodiments, a controller can control the output of the pharmacological agent and/or electrical stimulation in a controlled manner to treat pain in a subject.

Additional objects and advantages of the disclosure will be set forth in part in the description which follows, and/or can be learned by practice of the disclosure. The objects and advantages of the disclosure will be realized and attained by means of the elements and combinations particularly pointed out in the appended claims.

### BRIEF DESCRIPTION OF THE DRAWINGS

The accompanying drawings illustrate an embodiment of the invention and depict the above-mentioned and other features of this invention and the manner of attaining them.

FIG. 1 shows an illustration of an embodiment of an agent-neurostimulation system 1000 comprising a delivery device 10, a patient programmer 60 and a clinical programmer 65.

FIG. 2 is a prospective view illustration of various embodiments of the agent release module 20 showing at least two delivery elements 30 connected to the outputs 120 of the agent release module.

FIG. 3 is a schematic illustration showing an embodiment of the placement of the distal ends of the delivery elements and the associated agent outlet ports 40 and the electrodes 50 within a subject's anatomy.

FIG. 4 shows an antegrade approach to a target DRG wherein the delivery element is positioned along a nerve root sleeve angulation so that at least one of the outlet ports 40 and electrodes 50 are positioned within a clinically effective distance to the target anatomy, such as the target DRG.

FIG. 5 illustrates a cross-sectional view of an individual spinal level showing an embodiment of the distal end of a delivery element of the agent-neurostimulatory system positioned near a target DRG. Also shown is an example area of the agent release and e-field of electrical stimulation 180.

FIG. 6 is a schematic illustration showing how the delivery element connects to the agent release module 20.

FIG. 7A-7B is a cross-sectional view to illustrate various embodiments of the delivery element, with FIG. 7A showing a lumen 145 for transporting the agent, and at least four conductor cables 150. FIG. 7B shows an embodiment of the delivery element comprising a solid, multi-lumen shaft having a deliverylumen 145 and conductor cables 150, among other features.

FIGS. 8A-8D illustrate an embodiment of an agent delivery element comprising a lead and components of a delivery system for use in placing the delivery element within the subject's anatomy. FIG. 8A shows an embodiment of the lead having a plurality of electrodes 50, FIG. 8B shows an embodiment of the sheath 30, FIG. 8C shows an embodiment of a stylet 140. FIG. 8D shows the combination of the sheath, stylet and lead during delivery.

FIGS. 9A-9C show illustrations of various embodiments of the delivery element 30 comprising a lead having at least one electrode 50. FIG. 9A shows an illustration of an agent delivery lumen 140 which is fluidly connected to at least one outlet port 40. Disposed also in the element is a conductor cable 150 connected to the at least one electrode 50. FIG. 9B shows a variation of the embodiment of FIG. 9A, where disposed within the element is a plurality of lumens (140(i), 140(ii)) each connected to at least one outlet port 40(i), 40(i′), 40(ii) and 40(ii′), and a plurality of conductor cable 150 each connected to an electrode 50. FIG. 9C shows a variation of the embodiment of FIG. 9B, where disposed within the element is a plurality of lumens (140(i), 140(ii)) each connected to at least two outlet ports 40(i), 40(i′), 40(ii) and 40(ii′), and a plurality of conductor cables 150 each connected to an electrode 50.

FIGS. 10A-10C show illustrations of various embodiments of a delivery element 30. FIG. 10A shows an illustration of an agent delivery lumen 140 which is fluidly connected to at least one (two are shown) outlet port 40 in the element. FIG. 10B shows a variation of the embodiment of FIG. 10A, showing a plurality of outlet ports 40 connected to the lumen 140. FIG. 10C shows a variation of the embodiment of FIG. 10A, showing a plurality of lumens (140(i), 140(ii)) each connected to at least one outlet port 40(i), 40(i′), 40(ii) and 40(ii′).

FIG. 11 shows an illustration of an embodiment of the agent release module 20.

FIG. 12 shows an illustration of one embodiment of the delivery element 30 advanced within the epidural space so that several outlet ports 40 are positioned in close proximity to the dorsal root ganglia (DRG). In this embodiment, the delivery element 30 is advanced along the spinal cord S within the epidural space E to a desired spinal level and advanced at least partially through a foramen, between the pedicles PD. VR=ventral root, DR=dorsal root, E=epidural space, S=spinal cord, VB=vertebral body.

FIG. 13 shows an illustration of one embodiment of the position of a gel 200 delivery vehicle delivered to the epidural space E adjacent to the target DRG.

FIG. 14 shows an illustration of one embodiment of a delivery element 30 having electrodes 50 and an agent-eluting coating 250 covering its distal end.

FIGS. 15A-15B show an illustration of embodiments of the delivery element 30 having an agent-eluting structure 260 disposed on the surface of the distal end of a delivery element 30, where the structure 260 comprises circumferential stripes or strips 262 that extend around the shaft of the delivery element 30. FIG. 15A shows an embodiment of the delivery element 30 comprises a catheter and the strips 262 are spaced apart along the distal end of the delivery element 30. FIG. 15B shows an embodiment of the delivery element 30 which comprises a lead having electrodes 50, with the structures 260 as circumferential stripes or strips 262 that are disposed between the electrodes. Thus, the agent is eluted near the electrodes 50, such as for use in combination with electrical stimulation.

FIG. 16 shows an illustration of an embodiment of the delivery element 30 having agent-eluting structures 260 disposed as longitudinal stripes or strips along specific portions of the delivery element 30.

FIG. 17 shows an illustration of an embodiment of the delivery element 30 having agent-eluting structures 260 disposed as dots longitudinally and circumferentially around the delivery agent 30.

FIG. 18 shows an illustration of an embodiment of the delivery element 30 having an agent-eluting structure 260 extending along a portion of the distal end of the delivery agent 30, wherein the structure 260 extends at least partially around the shaft of the delivery element 30 and includes an opening for at least one outlet port 40.

FIG. 19A-19B shows an illustration of embodiments of the delivery element 30 having agent-eluting structures 260 as protrusions such as flexible hair-like protrusions 264. FIG. 19A shows an illustration of an embodiment of a delivery element 30 comprising a catheter having protrusions 264 extending radially outwardly from the shaft of the delivery element 30. FIG. 19B shows an illustration of an embodiment of a delivery element 30 comprising a lead having at least one electrode 50, at least one outlet port 40 and at least one protrusion 264.

FIG. 20 shows an illustration of an embodiment of the placement of a sheet 300 positioned adjacent the DRG, wrapping partially around the DRG, where the sheet 300 is positioned within the epidural space E at least partially within a foramen between the pedicles PD.

FIG. 21 shows an illustration of an embodiment of the placement of a tube 350 positioned within a foramen, between the pedicles PD, so that the tube 350 extends around the DRG. Since the tube 350 is positioned within the epidural space E, the tube 350 extends along the surface of the dura layer D which surrounds both the DRG and the nearby ventral root VR.

FIG. 22 shows an illustration of an embodiment of the position of the delivery device 30 placed intrathecally or into the subarachnoid or intrathecal space. In this embodiment, the delivery element 30 is inserted into the intrathecal space and advanced in an antegrade direction within the intrathecal space along the spinal cord S, where the delivery element 30 comprises a catheter having at least one outlet port 40, and is advanced through the patient anatomy so that at least one of the outlet ports 40 is within a clinically effective distance to the DRG.

FIG. 23 shows an embodiment of e-fields radiating from the electrodes 50 at the distal end of the delivery element. The electrodes 50 are positioned either side of two outlet ports 40 allowing both a combination of electrical stimulation and agent delivery to the DRG, either concurrently, or in a temporal pattern of electrical stimulation and agent delivery.

FIG. 24A-24C provides schematic illustrations of treatment to a target DRG. FIG. 24A shows an embodiment of electrical stimulation 402 only of the DRG. FIG. 24B shows an embodiment of agent delivery 400 only of the DRG. The combination of electrical stimulation 402 and agent delivery 400 to the DRG can be concurrently, or in a pre-defined temporal pattern of electrical stimulation 402 and agent delivery 400.

FIG. 25A-25B shows various embodiments using the agent-neurostimulation system. FIG. 25A shows distribution of an agent 400, e.g., a toxin, administered around a DRG cell. FIG. 25B shows that when the DRG is activated, for example, using neurostimulation of the DRG from the electrodes, the DRG cell becomes activated, allowing agent binding and/or entry 402 into the cell, and where the agent is a toxin, results in selective molecular neuroablation of the activated cell.

FIG. 26A-26B shows another embodiment of using the agent-neurostimulation system. FIG. 26A shows delivery of an agent 400, e.g., a prodrug to cells within the DRG using the DRG delivery device. FIG. 26B shows activation of the prodrug agent 400 by the electrical stimulation 402 to render the agent active, resulting in no activation (e.g., no effect) or activation or selective cell ablation of specific cell subtypes in the DRG on electrical stimulation (A), whereas other cell subtypes are activated by the active agent (B), resulting in modulation of the cell activity and/or cell death.

FIG. 27A-27B shows another embodiment of using the agent-neuromodulation of the system. FIG. 27A shows an agent 400 is delivered and has specificity or selectivity for some cell types (e.g., cell A) in the DRG and not other cell types cells (e.g., cell B). An agent can be selective for one cell-type by having a higher binding affinity for that cell type, and/or bind to cell-surface receptors on that cell-type, or be ligand for a channel and/or receptor on that particular cell type. FIG. 27B shows when electrical stimulation 402 is applied to all the DRGs, the cells that are sensitive to the agent 400 (e.g., where the agent is selective to that cell type) are activated and have altered activity as compared to the cells subjected to electrical stimulation 402 in the absence of the agent, or cells which are not sensitive to the agent 400.

FIGS. 28A-28E shows an embodiment of altered input and output electrical excitement dynamics using the agent-neuromodulation system.

### DETAILED DESCRIPTION OF THE INVENTION

The present invention generally relates to devices, systems and methods for delivering an agent to various levels of the spinal anatomy, particularly to various dorsal roots (DR), more particularly to various dorsal root ganglia (DRG), in a subject. For example, one aspect relates to a device for direct delivery of an agent, e.g., a drug formulation to at least target spinal anatomy, for example, to at least one DRG, where the agent is stored in an agent release module and is transported via an agent delivery element to the target anatomy, e.g., to at least one target DRG. In another aspect, the present invention relates to a device for direct delivery of an agent, e.g., a drug formulation to at least target spinal anatomy, for example, via the intrathecal space and/or the epidural space.

In some embodiments, the device, method and system can be further configured to enable direct and specific electrical stimulation, e.g., neurostimulation of the target anatomy, e.g., DRG, in combination with delivery of the agent to the DRG.

In some embodiments, electrical stimulation of the DRG is in a temporal pattern which is coordinated with a temporal pattern of delivery of the agent to the DRG. In some embodiments, the device allows delivery of an agent to a spinal nerve ganglion which is a dorsal root ganglion (DRG), while in alternative embodiments, the device enables delivery of an agent to a nerve root ganglion in the sympathetic nervous system, e.g., delivery of an agent to a sympathetic chain ganglion. The following examples will illustrate embodiments of specific temporal patterns of delivery of agents to the DRG alone, or in combination with temporal patterns of electrical stimulation of the DRG, however, the invention is not limited to such embodiments. Also described are a delivery device for delivering agents to the DRG, and where the delivery device is configured to enable electrical stimulation of the DRG in combination (e.g., concurrently) or intermittently, e.g., substantially simultaneously, before or after, delivery of an agent to the DRG. It may be appreciated that other elements, such as different agent release modules, and pulse generators may be used alternatively or in addition to the modules of the delivery device for delivery of agents to the DRG, alone, or in combination with electrical stimulation of a DRG at one or more various spinal cord levels.

The devices, systems and methods of the present invention allow for targeted delivery of an agent to at least one spinal anatomy, such as, but not limited to a DRG, and enables targeted treatment of such desired spinal anatomies. Accordingly, such targeted delivery of agents alone or in combination with electrical stimulation provides targeted treatment which minimizes deleterious side effects, such as undesired motor responses or undesired stimulation of unaffected body regions. This is achieved by directly delivering an agent to the DRG and, in some embodiments, neuromodulating a target anatomy associated with the condition while minimizing or excluding undesired neuromodulation of other anatomies. For example, this may include stimulating the dorsal root ganglia, dorsal roots, dorsal root entry zones, or portions thereof while minimizing or excluding undesired stimulation of other tissues, such as surrounding or nearby tissues, portions of the ventral root and portions of the anatomy associated with body regions which are not targeted for treatment. Such stimulation is typically achieved with the agent delivery device as disclosed herein which has been adapted to include at least one lead having at least one electrode thereon. The distal end of the delivery device is advanced through the patient anatomy so that the delivery element, comprising at least one agent delivery structure, such as an outlet port, for agent release and at least one electrode, is positioned on, near or about the target DRG. In some embodiments, the lead and electrode(s) are sized and configured so that the electrode(s) are able to minimize or exclude undesired stimulation of other anatomies. In other embodiments, the stimulation signal or other aspects are configured so as to minimize or exclude undesired stimulation of other anatomies. In addition, it may be appreciated that stimulation of other tissues are also contemplated.

Embodiments of the present invention provide novel stimulation systems and methods that enable direct and specific neurostimulation techniques. For example, there is provided a method of delivering an agent to the DRG and simultaneously stimulating a nerve root ganglion, comprising placing an electrode of a delivery element in close proximity, or near to, the target spinal anatomy, e.g., a nerve root ganglion or DRG or spinal cord and delivering an agent and also activating the electrode to stimulate the nerve root ganglion. As discussed in greater detail below, the nerve root ganglion may be a dorsal root ganglion in some embodiments while in other embodiments, the nerve root ganglion may be a nerve root ganglion in the sympathetic nervous system or other ganglion, e.g., sympathetic chain ganglion.

Another aspect of the present invention provides an agent delivery device to deliver agents to the intrathecal space near the target DRG, combined with an electrical stimulation systems and methods of use. For example, provided herein is a method of delivering an agent to the intrathecal space near the target DRG and simultaneously stimulating the target DRG with the use of another delivery device positioned within the epidural space. Thus, an agent is delivered intrathecally in conjunction with also activating the electrode placed epidurally.

### A DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “pain” as used herein, unless specifically noted otherwise, is meant to encompass pain of any duration and frequency, including, but not limited to, acute pain, chronic pain, intermittent pain, and the like. Causes of pain may be identifiable or unidentifiable. Where identifiable, the origin of pain may be, for example, of malignant, non-malignant, infectious, non-infectious, or autoimmune origin. Of particular interest is the management of pain associated with disorders, diseases, or conditions that require long-term therapy, e.g., chronic and/or persistent diseases or conditions for which therapy involves treatment over a period of several days (e.g., about 3 days to 10 days), to several weeks (e.g., about 2 weeks or 4 weeks to 6 weeks), to several months or years, up to including the remaining lifetime of the subject. Subjects who are not presently suffering from a disease or condition, but who are susceptible to such may also benefit from prophylactic pain management using the devices and methods of the invention, e.g., prior to traumatic surgery. Pain amenable to therapy according to the invention may involve prolonged episodes of pain alternating with pain-free intervals, or substantially unremitting pain that varies in severity. Pain includes all types of clinical pain, including but not limited to, nociceptive pain, pathological pain, neuropathic pain, somatic pain, cutaneous pain, chronic pain syndrome, referred pain, radicular pain, breakthrough pain or incidence pain, phantom limb pain, intractable pain and idiopathic pain, as defined in Hawthorn and Redmond, Pain: causes and managements, (Blackwell Science, Ed).

The term “breakthrough pain” is also referred to as incident pain, refers to short periods of sharper more intense pain that “breaks through” a background or constant discomfort from pain. Breakthrough pain can be caused by movement, pressure or treatment interventions.

The term “nociceptive pain” refers to pain produced from an identifiable cause.

The term “pathological pain” refers to pain felt due to activity in the nociceptive pathway which may be due to an identifiable cause or due to a disruption in the normal sensory mechanisms. Pathological pain is usually disproportionate to the causative factors and can be inappropriate and can outlast the original trauma, due to neuronal plasticity, including peripheral sensitization and/or central sensitization.

The term “idiopathic pain” refers to pain from an unknown origin or has not apparent underlying cause, or pain which is excessive in comparison to the underlying cause. Idiopathic pain is not nociceptive, neuropathic or even psychogenic. Idiopathic pain may be made worse by psychological distress, and is more common in people who already have a pain disorder such as TMJ and fibromyalgia. Idiopathic pain, like psychogenic pain, is often more difficult to treat than nociceptive or neuropathic pain. A person who has back pain with no apparent cause may be diagnosed as having idiopathic back pain.

The term “chronic pain” refers to long-lasting pain or pain disproportionate to the cause. Chronic pain may be pathological pain associated with changes in the central or peripheral nervous system or may be due to a constant stimulus.

The term “chronic pain syndrome” refers to a syndrome induced by long-term pain where pain and responses to pain are not well correlated with the underlying condition. In some embodiments, subjects with chronic pain may experience changes in personality, behavior and changes to functional ability.

The term “referred pain” refers to pain felt in a different area to the source of the pain. Referred pain is commonly referred from organs and deep tissue to muscles and skin.

The term “pain-related disorder” as used herein refers to any disease, condition or malady where the subject is experiencing pain.

The term “delivery site” as used herein is meant to refer to an area of the body to which the agent or drug is delivered. A delivery site can be in close proximity to the target spinal anatomy, which means that the delivery site is in a close enough proximity or location to deliver the agent and/or electrical stimulation to the target spinal anatomy, and includes but is not limited to dorsal root ganglia (DRGs), dorsal roots, dorsal root entry zones, or portions thereof.

The term “implantation” or “implant” or “implanted” are used interchangeably herein, and refer to the penetration or insertion of an element into a tissue, e.g., penetration of the distal end of the delivery element into neuronal tissue. In some aspects, implantation can also refer to inserting the device or pumps into cavities in the body.

The term “proximity to” as used herein refers to placing an element next to, or in a position near to, or in contact with another element or anatomical structure or tissue.

The term “drug delivery device” or “agent delivery device” includes the implantable agent release module and means, e.g., delivery elements to transport the agent or drug formulations to the desired target area or target anatomy in the subject.

The term “drug release module” also referred herein as a “controlled drug pump device” or “agent release module” refers to any device suitable for placing subcutaneously or in desired location in a subject for the storage and controlled release of an agent or drug formulation for pain management according to the method of the invention. The agent or drug or other desired substance contained in the pump is released in a controlled manner (e.g., rate, timing of release), which is controlled by or determined by the device itself, which in turn can be controlled by the subject user, or the clinician according to a predetermined program or treatment protocol. In some embodiments, the release of the agent or drug or other substance can be an osmotic pump where the agent is released according to the environmental use, e.g., where diffusion and osmotic concentrations control the release of the agent or drug from the pump in a controlled manner. The term “drug release module” or “agent release module” also encompasses any device with any mechanism of action including diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

The term “controlled drug pump device” is meant to encompass any device wherein the release (e.g., rate, timing of release) of a drug or other desired substance contained therein is controlled by or determined by the device itself and not the environment of use.

The term “patterned” or “temporal” as used in the context of drug delivery and/or electronic stimulation refers to the delivery of the agent and/or electrical stimulation in a pattern, generally a substantially regular pattern, over a pre-selected period of time (e.g., other than a period associated with, for example a bolus injection). The term “patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

By “substantially continuous” as used in, for example, the context of “substantially continuous subcutaneous infusion” or “substantially continuous delivery” is meant to refer to delivery of an agent or drug in a manner that is substantially uninterrupted for a pre-selected period of drug delivery (other than a period associated with, for example, a bolus injection). Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

The term “systemic delivery” is meant to encompass all parenteral routes of delivery which permit drug to enter into the systemic circulation, e.g., intravenous, intra-arterial, intramuscular, subcutaneous, intra-adipose tissue, intra-lymphatic, etc.

The term “block” or “blockade” or “blocking” are used interchangeably herein and refers to inhibition, disruption, prevention or inhibition of the conduction or propagation of action potentials and nerve impulse transmission along the axons of the target nerves, either partially or completely. The terms “block” or “blockade” or “blocking” also refer to the blockage of electrical signals along non-neuronal cell types, e.g., glial cells and astrocytes and the like, as well as refers to inhibition of intracellular signaling from cell surface markers and increases in soma size.

The term “nerve ablation” or “nerve lesioning” as used herein refers to the destruction of one or more axons of a target nerve so as to result in a nerve blockade in which conduction or propagation of action potentials in the target nerve is attenuated or abolished, either reversibly or permanently, as evidenced by the attenuation or abolition of sensation normally mediated by the nerve or weakness or paralysis of the body tissue innervated by the target nerve lasting more than a week, more than two weeks, or more than a month.

As used herein, the term “neurodegenerative disease or disorder” includes any disease disorder or condition that affects neuronal homeostasis, e.g., results in the degeneration or loss of neuronal cells. Neurodegenerative diseases include conditions in which the development of the neurons, i.e., motor or brain neurons, is abnormal, as well as conditions in which result in loss of normal neuron function. Examples of such neurodegenerative disorders include Alzheimer's disease and other tauopathies such as frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-park-insonism-amytrophy complex, Pick's disease, or Pick's disease-like dementia, corticobasal degeneration, frontal temporal dementia, Parkinson's disease, Huntington's disease, amyotrophic lateral sclerosis (ALS), multiple sclerosis, Friedreich's ataxia, Lewybody disease, spinal muscular atrophy, and parkinsonism linked to chromosome 17.

As used herein, the term “inflammation” refers to any cellular processes that lead to the activation of caspase-1, or caspase-5, the production of cytokines IL-1 and IL-8, and/or the related downstream cellular events resulting from the actions of the cytokines thus produced, for example, fever, fluid accumulation, swelling, abscess formation, and cell death. As used herein, the term “inflammation” refers to both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

As used herein, the term “inflammation” includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation include, but are not limited to, diffuse inflammation, focal inflammation, croupous inflammation, interstitial inflammation, obliterative inflammation, parenchymatous inflammation, reactive inflammation, specific inflammation, toxic inflammation and traumatic inflammation.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “drug” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

As use herein, the term “ion-channel” refers to a transmembrane pore that presents a hydrophilic channel for specific ions to cross a lipid bilayer down their electrochemical gradients. There are over 300 types of ion-channels in a living cell (Gabashvili, et al., “Ion-channel gene expression in the inner ear”, J. Assoc. Res. Otolaryngol. 8 (3): 305-28 (2007). The ion-channels are classified upon their ion specificity, biological function, regulation or molecular structure, and nature of their gating. Examples of ion-channels are voltage gated ion-channels, Gap junction ion-channels, ligand-gated ion-channels, ATP-gated ion-channels, heat-activated ion-channels, intracellular ion-channels, ion-channels gated by intracellular ligands such as cyclic nucleotide-gated channels or calcium-activated ion-channels. As used herein the term “gated ion-channel” is defined as an ion-channel the passage of ions through which is dependent on the presence of an analyte. As used herein, the term “voltage gated ion-channel” as used herein refers to an ion-channel where the passage of ions through which is dependent on the presence of voltage activation of the channel, where activation above a certain threshold level for the ion channel allows ions to pass through the ion channel. As used herein “ion channel” also encompasses transporters which transportions and other charged molecules across a membrane, including but not limited to Na⁺/K⁺ channels, Na⁺/Ca²⁺ and other ion transporters.

As used herein, the term “ion-channel modulator” refers to a compound that modulates at least one activity of an ion-channel, for example, but not limited to, and agent which increases or decreases the opening frequency and/or duration of the ion-channel, or an agent which increases and/or reduces the sensitivity of opening and/or closing of the ion-channel from the normal threshold of activation (opening) or deactivation (closing). In some embodiments, an ion-channel modulator is an agent which alters the selectivity of the ion channel to allow or prevent different ions from entering the ion channel, as well as an agent which alters (increases or decreases) the activation of the ion-channel by different receptor activation. The term “ion-channel modulator” as used herein is intended to include agents that interact with the channel pore itself, or that may act as an allosteric modulator of the channel by interacting with a site on the channel complex. The term “ion-channel modulator” as used herein is also intended to include agents that modulate activity of an ion-channel indirectly. By “indirectly,” as used in reference to modulator interactions with ion-channel, means the ion-channel modulator does not directly interact with the ion-channel itself, i.e., ion-channel modulator interacts with the ion-channel via an intermediary. Accordingly, the term “indirectly” also encompasses the situations wherein the ion-channel modulator requires another molecule in order to bind or interact with the ion-channel.

As used herein, the term “modulate” refers to a change or alternation in at least one biological activity of the ion-channel or receptor, e.g., cell-surface receptor. Modulation may be an increase or decrease the ion-channel or receptor activity, change the binding characteristics, or any other change in the biological, functional, or immunological properties of the ion-channel or receptor. Modulation can include, for example, a decreased or increased threshold of activation, an increased or decreased sensitivity to activation or deactivation, an increased or decreased selectivity for particular ions and/or ligands (e.g., endogenous or exogenous ligands or biologics) of the ion channel and/or receptor, respectively. Modulation can also include an alteration in the mode of action of the ion channel or the receptor, e.g., for example, an agent can modulate an ion channel to efflux ions rather than influx ions into the cell, or alternatively, alter the ion which is transmitted through the ion channel. In some embodiments of the aspects described herein, the ion-channel modulator modulates the passage of ions through the ion-channel.

The term “analgesic” as used herein refers to any member of the diverse group of drugs used to relieve pain. Analgesic drugs include, but are not limited to, the nonsteroidal anti-inflammatory drugs (NSAIDs) such as the salicylates, narcotic drugs such as morphine, and synthetic drugs with narcotic properties such as tramadol. Other classes of drugs not normally considered analgesics are used to treat neuropathic pain syndromes; these include tricyclic antidepressants and anticonvulsants. Without wishing to be bound by theory, NSAIDs including aspirin, naproxen, and ibuprofen not only relieve pain but also reduce fever and inflammation. Narcotic analgesics, including opiates and opioids, including Oxycodone, (also known as brand names DAZIDOX™, ETH-OXYDOSET™, ENDOCODONE™, OXYIR™, OXYCONTINT™, OXYFAST™, PERCOLONE™, ROXICODONE™) and a hydrocodone/paracetamol (or acetaminophen) mix (also known as brand names VICODIN™, ANEXSIA™, ANOLOR DH5™, BANCAP HC™, ZYDONE™, DOLACET™, LORCET™, LORTAB™, AND NORCO™), largely work through specific opioid receptors in the peripheral and central nervous system and alter the perception of pain (nociception). Analgesics can be used in combination, and can also be used in combination with vasoconstrictor drugs such as pseudoephedrine for sinus-related preparations, or with antihistamine drugs for allergy sufferers.

The term “agonist” as used herein refers to any agent or entity capable of increasing the expression and/or the biological activity of a protein, polypeptide portion thereof, or polynucleotide which is the target of the agonist agent. Thus, an agonist can operate to increase the transcription, translation, post-transcriptional or post-translational processing or otherwise activate the activity of the protein, polypeptide or polynucleotide in any way, such as functioning as a ligand to activate a receptor or via other forms of direct or indirect action. In some embodiments, an agonist refers to an agent which increases the biological activity of the target protein by a statistically significant amount as compared to in the absence of the agonist. In some embodiments, an agonist refers to an agent which increases the biological activity of the target protein by a clinically statistically significant amount as compared to in the absence of the agonist, such that the effect of the agonist on the target protein produces a clinically measurable change in outcome. In some embodiments, the term “agonist” as can refer to an agent which increases the biological activity of the target protein by about at least about 5%, e.g., an agonist to a target ion channel increases the activity of the ion channel expressed on the DRG by at least 5% or more than 5%. By way of example only, an agonist which activates an ion channel, e.g., a sodium channel can be any entity or agent which functions as a co-factor or ligand which opens a sodium channel or decreases the threshold of activation of a sodium channel, or promotes beta-subunit association with the sodium channel, or alternatively any agent which interacts with the sodium channel to increase its opening or decrease its threshold of activation (if the sodium channel is a voltage gated sodium channel). An agonist can be, for example a nucleic acid, peptide, or any other suitable chemical compound or molecule or any combination of these. Additionally, it will be understood that in indirectly activating the activity of a protein, polypeptide of polynucleotide, an agonist may affect the activity of the cellular molecules which may in turn act as regulators or the protein, polypeptide or polynucleotide itself. Similarly, an agonist may affect the activity of molecules which are themselves subject to the regulation or modulation by the protein, polypeptide of polynucleotide. An agonist is also referred to herein as an “activating agent”.

The term “antagonist” as used herein refers to any agent or entity capable of inhibiting the expression and/or the biological activity of a protein, polypeptide portion thereof, or polynucleotide. Thus, the antagonist may operate to prevent transcription, translation, post-transcriptional or post-translational processing or otherwise inhibit the activity of the protein, polypeptide or polynucleotide in any way, such as functioning as a ligand to activate a receptor or via other forms of direct or indirect action. In some embodiments, an antagonist refers to an agent which decreases the biological activity of the target protein by a statistically significant amount as compared to in the absence of the antagonist. In some embodiments, an antagonist refers to an agent which decreases the biological activity of the target protein by a clinically statistically significant amount as compared to in the absence of the antagonist, such that the effect of the antagonist on the target protein produces a clinically measurable change in outcome. In some embodiments, an “antagonist” refers to an agent that can decrease the biological activity of the target protein by at least about 5%, e.g., an antagonist to a target ion channel decreases the activity of the ion channel expressed on the DRG by at least 5% or greater than 5%. By way of example only, an antagonist which inhibits a sodium channel, for example a sodium channel blocker can be any entity or agent which functions as a to competitively block the channel pore of the sodium channel, or alternatively any agent which is a non-competitive inhibitor of a sodium channel which interacts at a region of a sodium channel which is not the pore) to inhibit channel opening or increase the threshold of activation (e.g., if the sodium channel is a voltage gated sodium channel). An antagonist may for example, be any agent, such as but not limited to a nucleic acid, peptide, or any other suitable chemical compound or molecule or any combination of these. Additionally, it will be understood that in indirectly impairing the activity of a protein, polypeptide of polynucleotide, the antagonist may affect the activity of the cellular molecules which may in turn act as regulators or the protein, polypeptide or polynucleotide itself. Similarly, the antagonist may affect the activity of molecules which are themselves subject to the regulation or modulation by the protein, polypeptide of polynucleotide.

The term “treating”, as used herein, refers to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

The term “pain management or treatment” is used here to generally describe regression, suppression, or mitigation of pain so as to make the subject more comfortable as determined by subjective criteria, objective criteria, or both. In general, pain is assessed subjectively by patient report, with the health professional taking into consideration the patient's age, cultural background, environment, and other psychological background factors known to alter a person's subjective reaction to pain.

The term “therapeutically effective amount” as used herein refers an amount of an agent, or a rate of delivery of an agent, effective to facilitate a desired therapeutic effect or benefit or desired clinical result upon treatment, e.g., a measurable decrease in the sensation of pain experienced by the subject. The precise desired therapeutic effect (e.g., the degree of pain relief, and source of the pain relieved, etc.) will vary according to the condition to be treated, the agent and/or drug formulation to be administered, as well as the effect in combination with electrical stimulation and a variety of other factors that are appreciated by those of ordinary skill in the art. In general, the method of the invention involves the suppression or mitigation of pain in a subject suffering from pain that may be associated with any of a variety of identifiable or unidentifiable etiologies. The phrase “therapeutically-effective amount” as used herein means that amount of a compound, material, or composition comprising an agent, e.g., an ion-channel modulator which is effective for producing some desired therapeutic effect in at least a sub-population of cells in an animal at a reasonable benefit/risk ratio applicable to any medical treatment. For example, an amount of an ion-channel modulator administered to a subject that is sufficient to produce a clinically meaningful or statistically significant measurable decrease in the pain experienced by the subject. A therapeutically effective amount will vary, as recognized by those skilled in the art, depending on the specific disease treated, the excipient selected, and the possibility of combination therapy, e.g., effect of the delivery of the agent in combination with electrical stimulation of the DRG.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents. Furthermore, therapeutically effective amounts will vary, as recognized by those skilled in the art, depending on the specific disease treated, the route of administration, the excipient selected, and the possibility of combination therapy.

The term “pharmaceutically acceptable excipient”, as used herein, refers to carriers and vehicles that are compatible with the active ingredient (for example, a compound of the invention) of a pharmaceutical composition of the invention (and preferably capable of stabilizing it) and not deleterious to the subject to be treated. For example, solubilizing agents that form specific, more soluble complexes with the compounds of the invention can be utilized as pharmaceutical excipients for delivery of the compounds. Suitable carriers and vehicles are known to those of extraordinary skill in the art. The term “excipient” as used herein will encompass all such carriers, adjuvants, diluents, solvents, or other inactive additives. Suitable pharmaceutically acceptable excipients include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical compositions of the invention can also be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like, which do not deleteriously react with the active compounds of the invention.

Thus, as used herein, the term “pharmaceutically acceptable salt,” is a salt formed from an acid and a basic group of a compound of the invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate salts.

The term “pharmaceutically acceptable salt” also refers to a salt prepared from a compound of the invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. Other pharmaceutically acceptable salts are described in the Handbook of Pharmaceutical Salts. Properties, Selection, and Use (P. Heinrich Stahl and C. Wermuth, Eds., Verlag Helvetica Chica Acta, Zurich, Switzerland (2002)).

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The term “subject” is used interchangeably herein with “patient” and refers to a vertebrate, preferably a mammal, more preferably a primate, still more preferably a human. Mammals include, without limitation, humans, primates, wild animals, feral animals, farm animals, sports animals, and pets. Mammals include, without limitation, humans, primates, wild animals, rodents, feral animals, farm animals, sports animals, and pets. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. The terms, “patient” and “subject” are used interchangeably herein. A subject can be male or female. A subject can be one who has been previously diagnosed with or identified as suffering from a condition, such as pain or having a pain-related disorder. A subject can also one who is currently being treated for pain, e.g., chronic pain or a pain-related disorder. In addition, the methods and compositions described herein can be used to treat domesticated animals and/or pets.

As used herein, a “prodrug” refers to compounds that can be converted via some chemical or physiological process (e.g., enzymatic processes and metabolic hydrolysis) to either S-α-methyl-hydrocinnamic acid or R-α-methyl-hydrocinnamic acid. A prodrug may be inactive when administered to a subject, i.e. an ester, but is converted in vivo to an active compound (e.g. S-α-methyl-hydrocinnamic acid or R-α-methyl-hydrocinnamic acid), for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. ll,:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenyloin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989).

In some embodiments of the aspects described herein, the method further comprising diagnosing a subject with pain or a pain-related disorder before treatment with the systems, devices and methods as described herein. Methods of diagnosing pain, such as chronic pain, neuropathic and inflammatory pain are well known in the art.

In some embodiments, the method further comprising selecting a subject identified to have pain, such as chronic pain, before treatment with the systems, devices and methods as described herein.

The terms “decrease”, “reduce”, “reduction” or “inhibit” are all used herein generally to mean a decrease by a statistically significant amount. However, for avoidance of doubt, “reduced”, “reduction” or “decrease” or “inhibit” means a decrease by at least 5%, or by at least 10% as compared to a reference level, for example a decrease by at least about 5% or about 10%, or about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increase”, or “enhance” or “activate” are all used herein to generally mean an increase by a statistically significant amount. However, for the avoidance of any doubt, the terms “increased”, “increase” or “enhance” or “activate” means an increase of at least about 5%, or least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) as compared to the other value. The term refers to statistical evidence that there is a difference. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and the include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean±1%.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.).

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### B. AGENT DELIVERY SYSTEM EMBODIMENTS

In some embodiments of the invention, the delivery system delivers an agent or drug formulation to one or more target spinal anatomies, e.g., a dorsal root (DR) or dorsal root ganglion (DRG) of the subject.

1. Delivery Elements Connectable with Agent Delivery Module

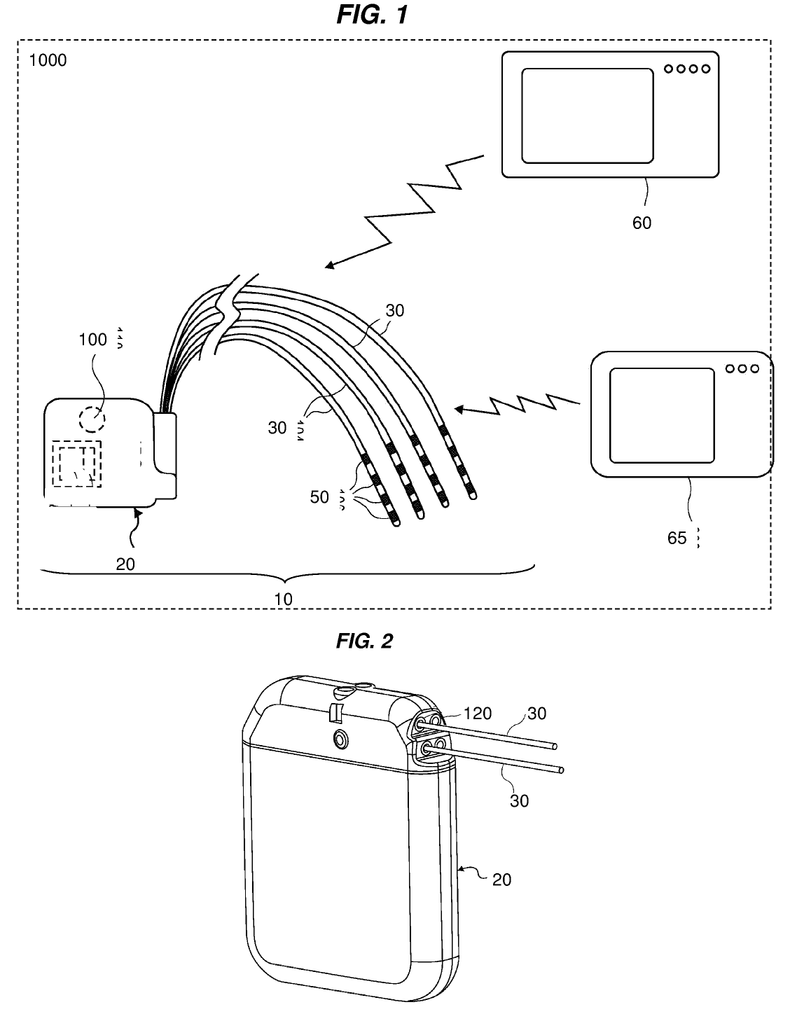

FIG. 1 illustrates an example embodiment of neuromodulation system 1000 comprising an agent delivery system 10, a clinical programmer 65 and a patient programmer 60. In this embodiment, the agent delivery system 10 comprises two main components: 1) an agent release module 20 which stores and releases an agent, e.g., a drug formulation which is to be delivered to the target anatomical site of agent delivery, e.g., the DRG, and 2) at least one delivery element 30 connected to thereto, where the delivery element includes at least one delivery lumen which delivers an agent, e.g. a drug formulation from the agent release module 20 to the target anatomical site of agent delivery, e.g., the DRG. As indicated by zig-zag arrows, the clinical programmer 65 and/or the patient programmer 60 wirelessly communicate with the agent delivery module 20 to provide agent delivery program information, receive data, and/or perform various other functions as will be described further herein.

In this embodiment, the delivery elements 30 deliver both an agent and electrical stimulation to the target anatomical site. Thus, in this embodiment, each delivery element 30 includes at least one electrode 50 and at least one outlet port 40 for delivery of the drug or agent there through. In such instances wherein a delivery element 30 includes at least one electrode 50 the delivery element is referred to as a lead. It is encompassed that the delivery elements 30 may alternatively not include electrodes, wherein the delivery elements are for delivery of an agent independent of electrical stimulation. Such delivery elements are referred to as catheters, herein. It may be appreciated that the agent delivery system 10 may include leads, catheters or leads and catheters.

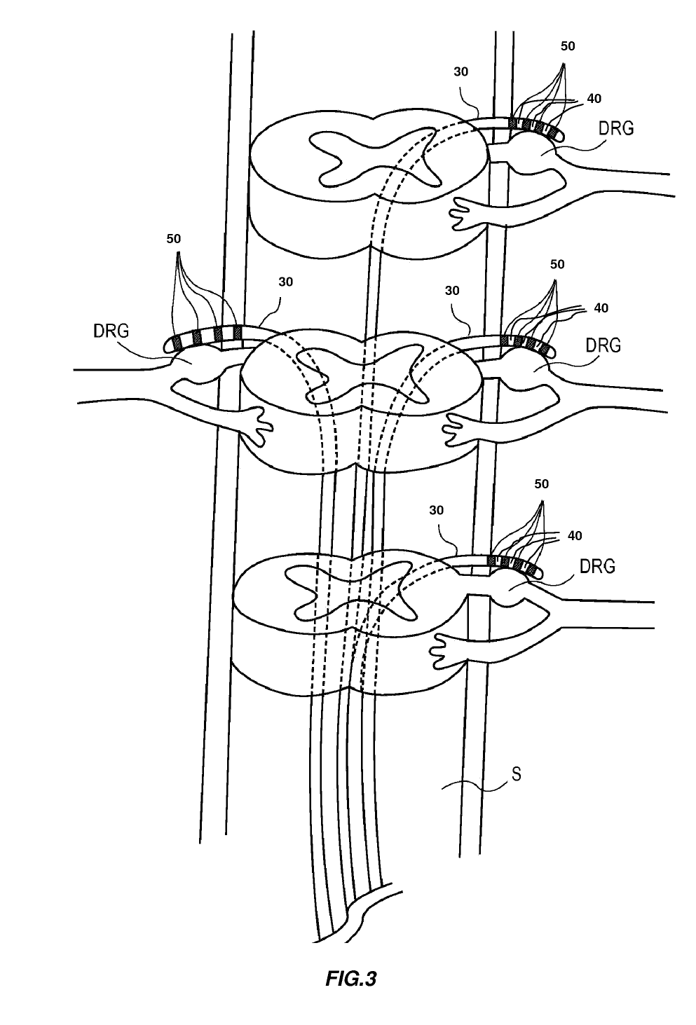

FIG. 3 illustrates example placement of delivery elements 30 of the delivery system 10 of FIG. 1. The delivery elements 30 are shown positioned along portions of the central nervous system. Typically, the delivery system is used to neuromodulate portions of neural tissue of the central nervous system, wherein the central nervous system includes the spinal cord and the pairs of nerves along the spinal cord which are known as spinal nerves. The spinal nerves include both dorsal and ventral roots which fuse in the intravertebral foramen to create a mixed nerve which is part of the peripheral nervous system. At least one dorsal root ganglion (DRG) is disposed along each dorsal root prior to the point of mixing. Thus, the neural tissue of the central nervous system is considered to include the dorsal root ganglions and exclude the portion of the nervous system beyond the dorsal root ganglions, such as the mixed nerves of the peripheral nervous system. In some embodiments, the systems and devices of the present invention are used to neuromodulate one or more dorsal root ganglia, dorsal roots, dorsal root entry zones, or portions thereof. FIG. 3 illustrates the delivery elements 30 of the delivery system 10 of FIG. 1 positioned so that the distal end of each of the delivery elements is near a DRG. In particular, each distal end is positioned so that at least one electrode thereon and at least one agent delivery port is within a distance of the target DRG so as to allow neuromodulation of the target DRG, more particularly selective neuromodulation of the DRG

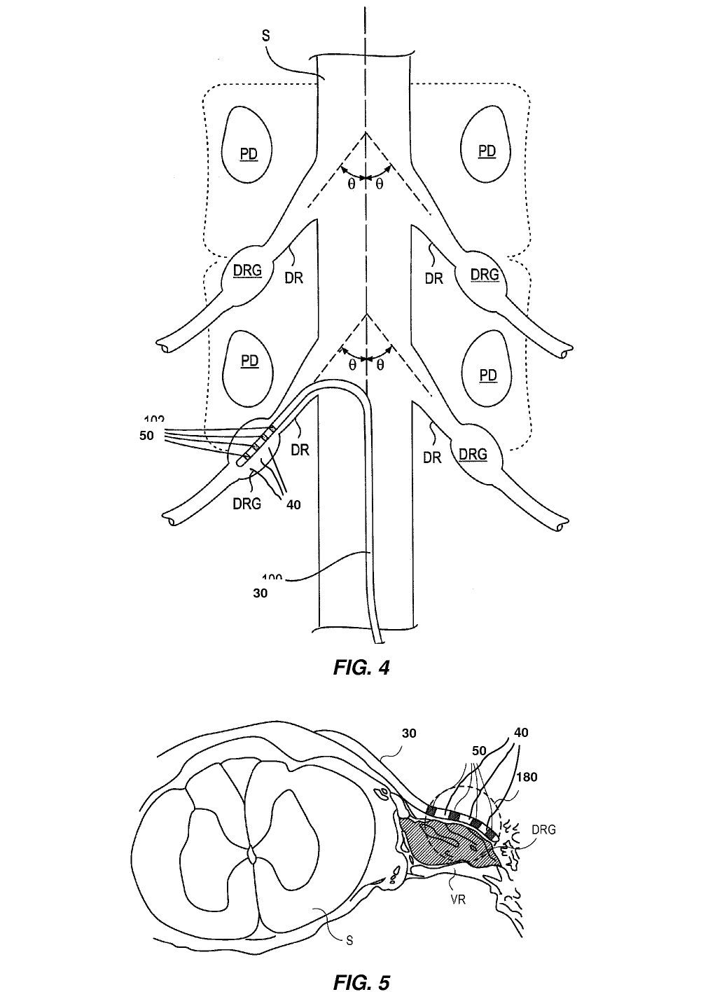

Accessing these areas is challenging, particularly from an antegrade epidural approach. FIG. 4 schematically illustrates portions of the anatomy of FIG. 3 including anatomical placement of the pedicles PD. As shown, each DRG is disposed along a dorsal root DR and typically resides at least partially between the pedicles PD or within a foramen. Each dorsal root DR exits the spinal cord S at an angle θ. This angle θ is considered the nerve root sleeve angulation and varies slightly by patient and by location along the spinal column. The average nerve root angulation in the lumbar spine is significantly less than 90 degrees and typically less than 45 degrees. Therefore, accessing this anatomy from an antegrade approach involves making a sharp turn through, along or near the nerve root sleeve angulation. It may be appreciated that such a turn may follow the nerve root sleeve angulation precisely or may follow various curves in the vicinity of the nerve root sleeve angulation.

FIG. 4 illustrates an embodiment of a delivery element 30 of FIG. 1 inserted epidurally and advanced in an antegrade direction within the epidural space along the spinal cord S. The delivery element 30 having at least one electrode 50 thereon, is advanced through the patient anatomy so that at least one of the electrodes 50 is positioned on a target DRG. Likewise, the delivery element 30 is positioned so that at least one of the outlet ports is positioned within a clinically effective distance to the target anatomy, such as the target DRG. Such advancement of the lead 100 toward the target DRG in this manner involves making a sharp turn along the angle θ. A turn of this severity is achieved with the use of a variety of delivery tools and design features of the delivery elements 30 which will be described in more detail herein. In addition, the spatial relationship between the nerve roots, DRGs and surrounding structures are significantly influenced by degenerative changes, particularly in the lumbar spine. Thus, patients may have nerve root angulations which differ from the normal anatomy, such as having even smaller angulations necessitating even tighter turns. The present invention also accommodates these anatomies.

The devices, systems and methods of the present invention allow for targeted treatment of the desired anatomies. Such targeted treatment minimizes deleterious side effects, such as undesired motor responses or undesired stimulation or neuromodulation of unaffected body regions. This is achieved by directly neuromodulating a target anatomy associated with the condition while minimizing or excluding undesired neuromodulation of other anatomies. For example, this may include stimulating the dorsal root ganglia, dorsal roots, dorsal root entry zones, or portions thereof while minimizing or excluding undesired stimulation of other tissues, such as surrounding or nearby tissues, portions of the ventral root and portions of the anatomy associated with body regions which are not targeted for treatment. In addition, it may be appreciated that stimulation of other tissues are also contemplated.

FIG. 5 illustrates an example cross-sectional view of an individual spinal level showing a delivery element 30 of FIG. 1 positioned on, near or about a target DRG. The delivery element 30 is advanced along the spinal cord S within the epidural space to the appropriate spinal level wherein the delivery element 30 is advanced laterally toward the target DRG. In some instances, the delivery element 30 is advanced through or partially through a foramen. At least one, some or all of the electrodes 50 and agent delivery outlet ports 40 are positioned on, about or in proximity to the DRG. In preferred embodiments, the delivery element 30 is positioned so that the electrodes 50 and outlet ports 40 are disposed along a surface of the DRG opposite to the ventral root VR, as illustrated in FIG. 5. It may be appreciated that the surface of the DRG opposite the ventral root VR may be diametrically opposed to portions of the ventral root VR but is not so limited. Such a surface may reside along a variety of areas of the DRG which are separated from the ventral root VR by a distance.

As mentioned, the delivery elements 30 of FIG. 1 are configured to include electrodes for intermittent (e.g., temporally patterned) or simultaneous electrical stimulation as well as delivery of the agent and/or drug formulation to the target site. Such configuration may include a variety of design features, including agent delivery parameters, electrical signal parameters, which are able to minimize undesired delivery or stimulation of other anatomies. FIG. 5 shows an example area of agent release and e-field of electrical stimulation 180 as indicated by dashed line. The area 180 extends within the DRG but does not extent to the ventral root VR. Thus, such placement of the delivery element 30 may assist in reducing any possible stimulation of the ventral root VR due to distance. However, it may be appreciated that the electrodes 50 and agent outlet ports 40 may be positioned in a variety of locations in relation to the DRG and may selectively stimulate the DRG due to factors other than or in addition to distance, such as due to stimulation profile shape, stimulation signal parameters, agent selection, agent concentration, dosing schedule, to name a few. It may also be appreciated that the target DRG may be approached by other methods, such as a retrograde epidural approach. Likewise, the DRG may be approached from outside of the spinal column wherein the delivery element 30 is advanced from a peripheral direction toward the spinal column, optionally passes through or partially through a foramen and is positioned so that at least some of the electrodes 106 are positioned on, about or in proximity to the DRG.

It may be appreciated that the delivery elements 30 can be used for selective electrical stimulation or neuromodulation in a number of different configurations. Example configurations include unilateral (on or in one root ganglion on a level), bi-lateral (on or in two root ganglion on the same level), unilevel (one or more root ganglion on the same level) or multi-level (at least one root ganglion is stimulated on each of two or more levels) or combinations of the above including stimulation of a portion of the sympathetic nervous system and one or more dorsal root ganglia associated with the neural activity or transmission of that portion of the sympathetic nervous system. Likewise, example configurations include combinations of the above including stimulation of a portion of the spinal cord and one or more dorsal root ganglia associated with the neural activity. As such, embodiments of the present invention may be used to create a wide variety of stimulation control schemes, individually or overlapping, to create and provide zones of treatment.

In some embodiments, the delivery device and systems as disclosed herein are based on improved versions of the neurostimulation devices as disclosed in International Application WO2010/083308, and WO2006/029257, and US Patent Applications US2010/0137938 and US2008/0167698, each of which are incorporated herein in its entirety by reference.

In alternate embodiments, the identified DRG or numerous DRGs are identified and are selected for placement of the distal end of the shaft of the delivery element 30, such that the plurality of sidewall apertures, e.g., outlet ports 40 for agent delivery allows delivery of an agent proximal to the DRG. In such an embodiment, placement of the device may be achieved through methods as disclosed in U.S. patent applications 2010/0137938, 2010/0249875, US2008/0167698 and International Application, WO2010/083308, WO2008/070807, WO2006/029257, which are incorporated herein in their entirety by reference.

a. Delivery Elements

As mentioned previously, the delivery elements 30 are connected by their proximal ends to the agent release module 20, such as depicted in FIG. 1. In this embodiment, the agent delivery device 10 includes four delivery elements 30, however, it may be appreciated that any number of delivery elements 30 may be used including one, two, three, four, five, six, seven, eight, about 8-10, about 10-20, about 20-30, about 30-50, or about 50 or more, or about 58 or more. In some embodiments, the delivery element 30 includes at least one agent delivery structure, such as an agent outlet 40, near its distal end. As mentioned previously, delivery elements 30 having at least one electrode 50 is considered a lead. Delivery elements 30 without at least one electrode are considered a catheter.

1) Leads

As stated above, delivery elements 30 which are considered leads include at least one electrode 50, typically near its distal end. It may be appreciated that each lead can comprise any number of electrodes 50, including one, two, three, four five, six, seven, eight, nine, ten, eleven, twelve, thirteen, fourteen, fifteen, sixteen or more. Typically, each electrode can be configured as off, anode or cathode. In some embodiments, only one lead is providing stimulation energy at any given time. In other embodiments, more than one lead is providing stimulation energy at any given time, or stimulation by the leads is staggered or overlapping. Likewise, it may be appreciated that at any given time only one electrode per lead is providing stimulation energy, or more than one electrode per lead is providing stimulation energy wherein the more than one electrodes are providing the stimulation energy simultaneously, staggered or overlapping. In some embodiments, even though each electrode is independently configurable, at any given time the software ensures only one lead is stimulating at any time. In other embodiments, more than one lead is stimulating at any time, or stimulation by the leads is staggered or overlapping.

In some embodiments, each lead includes at least one outlet port 40. The outlet port(s) 40 typically are located near the distal end of the lead and may be located near one or more electrodes 50. In some embodiments, the lead includes at least 2 or a least 3 outlet ports 40, and at least 3 or at least 4 electrodes 50 positioned between the outlet ports 40, as shown in FIG. 1.

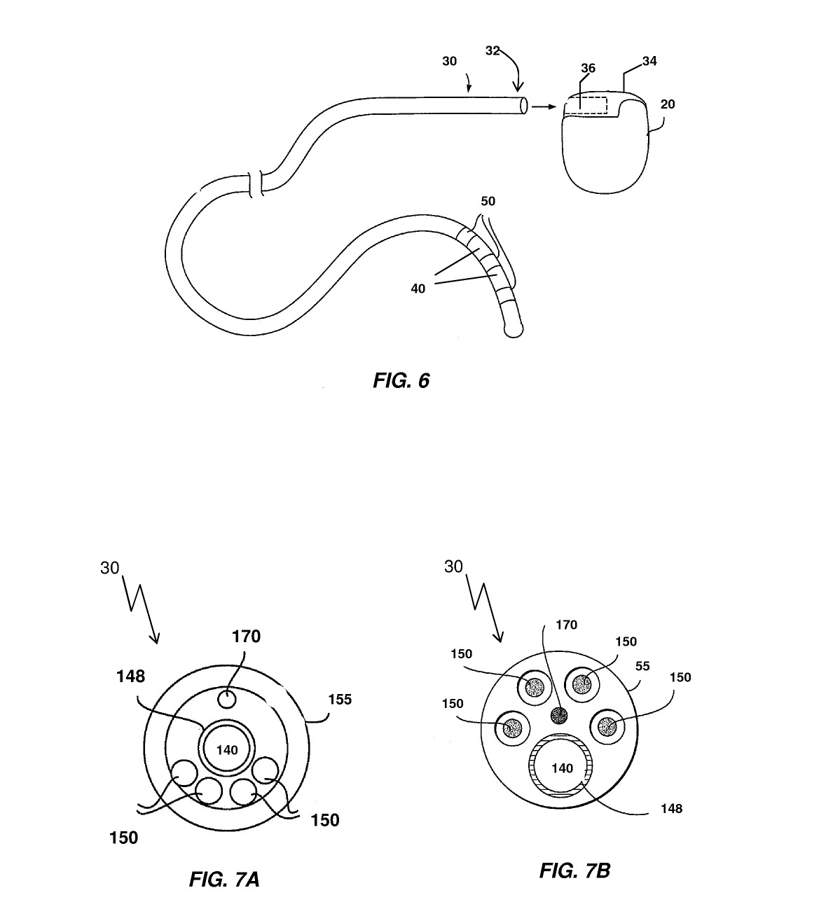

FIG. 6 illustrates an agent release module 20 and a single delivery element 30 having a plurality of electrodes 50 and a plurality of outlet ports 40. The delivery element 30 has a proximal end 32 which is connectable with the agent release module 20, particularly insertable into a header 34 having lead receptacle 36 or lead connection assembly.

FIG. 7A shows a cross-sectional view of an embodiment of the delivery element 30, such as illustrated in FIG. 6. The delivery element 30 includes a shaft 55 having a plurality of components extending therethrough. In this embodiment, the components include a tube 148 having an agent delivery lumen 140 therethrough. The components also include a plurality of conductor cables 150, each connecting with an electrode near the distal end of the delivery element 30. In this embodiment, the element 30 has four electrodes 50; therefore, there are four conductor cables 150 shown. In addition, the delivery element 30 includes a tensile element 170 which provides tensile strength to the lead. FIG. 7B shows an alternative embodiment of the delivery element 30. In this embodiment, the shaft 55 comprises a multi-lumen extruded tube and each of the components extend through dedicated lumens. For example, each conductor cable 150, the tensile element 170 and the tube 148 extend through separate dedicated lumens.

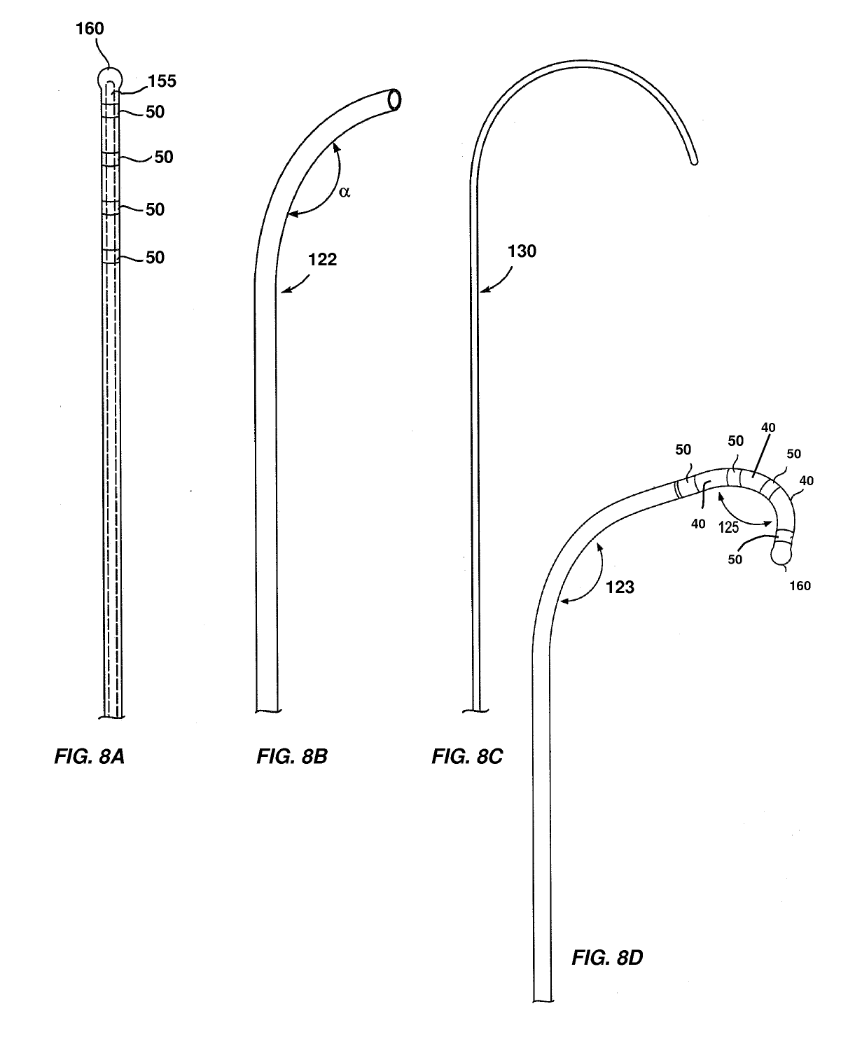

Referring to FIGS. 8A-8C, an embodiment of a delivery element 30 (FIG. 8A) and delivery system including a sheath 122 (FIG. 8B) and stylet 130 (FIG. 8C) are shown. The delivery system is used for placing the delivery element within the subject's anatomy. In this embodiment, the distal end of the delivery element 30 has a closed-end distal tip 160. The distal tip 160 may have a variety of shapes including a rounded shape, such as a ball shape (shown) or tear drop shape, and a cone shape, to name a few. These shapes provide an atraumatic tip for the delivery element as well as serving other purposes. The delivery element 30 also includes stylet lumen 155 (which in some embodiments also functions as an agent delivery lumen) which extends toward the closed-end distal tip 160.

FIG. 8B illustrates an embodiment of the sheath 122 having a distal end 128 which is pre-curved to have an angle α, wherein the angle α is in the range of approximately 80 to 165 degrees. The sheath 122 is sized and configured to be advanced over the shaft of the delivery element 30 until a portion of its distal end abuts the distal tip 160 of the delivery element 30. Thus, the ball shaped tip 160 of this embodiment also prevents the sheath 122 from extending thereover. Passage of the sheath 122 over the delivery element 30 causes the element 30 to bend in accordance with the precurvature of the sheath 122. Thus, the sheath 122 assists in steering the delivery element 30 along the spinal column S and toward a target DRG, such as in a lateral direction. It may be appreciated that the angle α may optionally be smaller, such as less than 80 degrees, forming a U-shape or tighter bend.

Referring to FIG. 8C, an embodiment of a stylet 130 is illustrated having a distal end which is pre-curved. In some embodiments, the distal end has a radius of curvature is in the range of approximately 0.1 to 0.5 inches. The stylet 130 is sized and configured to be advanced within the stylet lumen 155 of the delivery element 30. Typically the stylet 130 extends therethrough so that its distal end aligns with the distal end of the delivery element 30. Passage of the stylet 130 through the delivery element 30 causes the delivery element 30 to bend in accordance with the precurvature of the stylet 130. Typically, the stylet 130 has a smaller radius of curvature, or a tighter bend, than the sheath 122. Therefore, as shown in FIG. 8D, when the stylet 130 is disposed within the delivery element 30, extension of the delivery element 30 and stylet 130 through the sheath 122 bends or directs the delivery element 30 through a first curvature 123. Further extension of the delivery element 30 and stylet 130 beyond the distal end of the sheath 122 allows the delivery element 30 to bend further along a second curvature 125. When approaching a target DRG, the second curvature allows the laterally directed delivery element 30 to now curve around toward the target DRG, such as along the nerve root angulation. This two step curvature allows the delivery element 30 to be successfully positioned so that at least one of the electrodes 50 and agent delivery outlet ports 40 is on, near or about the target DRG, particularly by making a sharp turn along the angle θ. In addition, the electrodes 50 and/or delivery ports 40 are spaced to assist in making such a sharp turn.

It may be appreciated some embodiments of the delivery element 30 and the delivery devices as disclosed herein are based on the neurostimulation devices as disclosed in U.S. patent application Ser. No. 12/687,737, entitled “Stimulation Leads, Delivery Systems and Methods of Use” and incorporated herein in its entirety by reference, therefore sharing similar features and delivery methods as described therein.

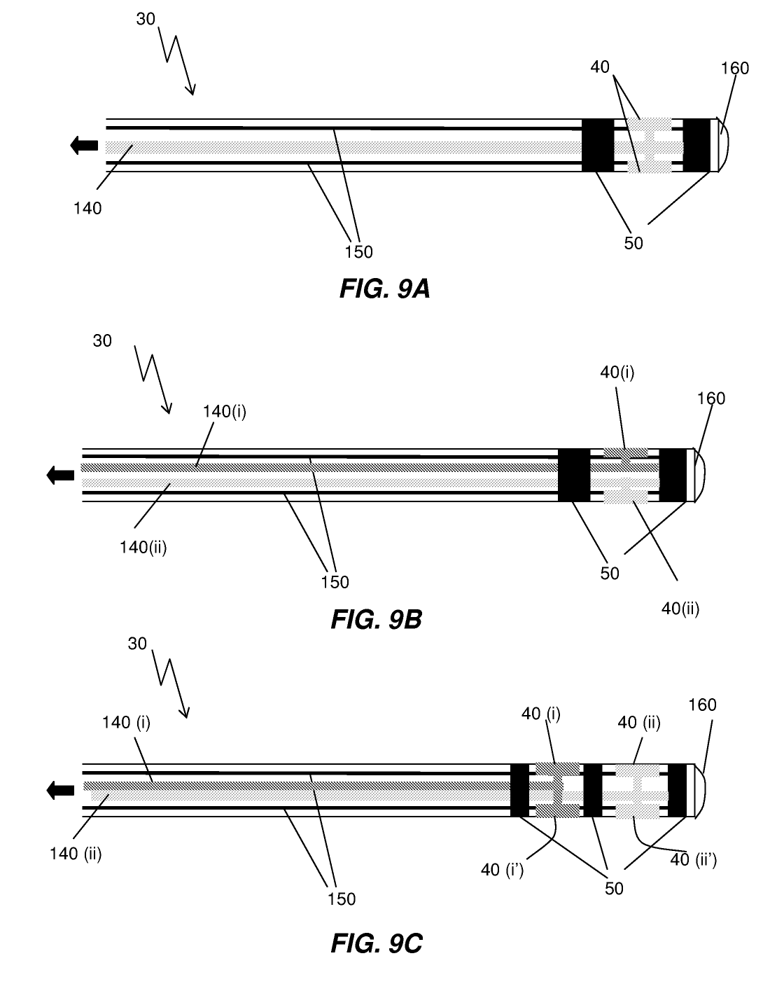

FIGS. 9A-9C illustrate various embodiments of delivery elements 30 having at least one electrode 50 and at least one agent delivery outlet port 40. Referring to FIG. 9A, the delivery element 30 includes one delivery lumen 140 therein, which has its proximal end in fluid connection with the reservoir 70 of the agent release module 20, and extends towards a distal tip 160. The diameter of the delivery lumen can be of any diameter, for example, 0.1 mm, 0.2 mm, 0.5 mm, 1 mm, 2 mm, 5 mm, or more than 5 mm, and any integer between 0.1 mm and 5 mm in diameter. In some embodiments, the distal end of the delivery element 30 has a closed-end distal tip 160. In such embodiments, the distal tip 160 may have a variety of shapes including a rounded shape, a ball shape, a tear-drop shape, or a cone shape, to name a few. These shapes provide an atraumatic tip for the delivery element 30 as well as serving other purposes. When the distal tip 160 has a closed end, the delivery lumen 140 connects with at least one outlet port 40 located in the wall of the element 30. It may be appreciated that in some embodiments the delivery element 30 has an open-end distal tip 160. FIG. 9A illustrates a closed end distal tip 160 and a single delivery lumen 140 extending toward the distal tip 160, connecting with two agent delivery outlet ports 40, each disposed between a pair of electrodes 50. The delivery element 30 further includes a conductor cable 150 extending from the distal end of the delivery element 30 to each electrode 50.

FIG. 9B illustrates another embodiment of the delivery element 30. In this embodiment, the delivery element 30 comprising a plurality of agent delivery lumens 140, each fluidly connected with an outlet port 40. This allows for delivery of more than one different agent to the DRG via each delivery lumen, or alternatively, delivery of the same agent but at a variety of different doses via each delivery lumen. In this embodiment, a first delivery lumen 140(i) is connected to a first outlet port 40(i) and a second delivery lumen 140(ii) is connected with a second outlet port 40(ii). In this embodiment, both the first outlet port 40(i) and the second outlet port 40(ii) are disposed between the pair of electrodes 50, each facing an opposite direction. It may be appreciated that any number of outlet ports 40 may be present, including one, two, three, four, five, six, seven, eight, etc., and the outlet ports 40 may be arranged in any configuration in relation to each other and to the electrodes 50.

FIG. 9C illustrates another embodiment of a delivery element 30 having a plurality of delivery lumens 140(i), 140(ii) each in fluid connection with at least one reservoir 70 at their proximal end and at least one outlet port in the element wall near their distal end. In this embodiment, a first delivery lumen 140(i) is in fluid connection with two outlet ports 40(i), 40(i′) and a second delivery lumen 140(ii) is in fluid connection with two outlet ports 40(ii), 40 (ii′). Each pair of each outlet ports, such as the pair of outlet ports 40(i), 40(i′), is disposed between different pairs of electrodes 50. Again, the plurality of delivery lumens allows for delivery of more than one different agent to the DRG via each delivery lumen, or alternatively, delivery of the same agent but at a variety of different doses via each delivery lumen. It is also appreciated that the delivery element 30 can comprise any number of delivery lumens, including one, two, three, four five, six, seven, eight, nine, ten, eleven, twelve, thirteen, fourteen, fifteen, sixteen or more. Each delivery lumen can be used for delivering the same agent (e.g., the proximal end of each is connected to the same reservoir) to the target anatomy, e.g., the DRG, or a different agent (e.g., the proximal end of each lumen is connected to a different reservoir) to the target anatomy, e.g., the DRG. In some embodiments, each delivery lumen disposed within the delivery element are independently configurable. For example, in some embodiments, software can ensure an agent is delivered from specific lumens, at specific rates, at particular times. Thus, agent release can be staggered or overlapping from different delivery lumens.

In regards to the combination neurostimulation and pharmacological agent delivery element, the distal tip of the delivery element 30 comprising the electrodes 50 and agent outlet ports 40 can be placed in any location near the target spinal anatomy, e.g., the DRG, to obtain the desired stimulation or modulation level. Additionally, the distal tip of the delivery element 30 comprising the electrodes 50 and agent outlet ports 40 can be placed so that modulation or stimulation energy patterns are selective to the target tissue, such as will remain within or dissipate only within the targeted neural tissue.

2) Catheters

In some embodiments, the agent or drug formulation is transported to the target spinal anatomy delivery site, such as a DRG, dorsal root, dorsal root entry zones etc, from the reservoir 70 (or agent holding chamber) in the agent release module 20 via an agent delivery lumen within a delivery element 30, which is in fluid communication with the reservoir 70. An agent delivery element 30 is generally a substantially hollow elongate member or shaft having a first end (or “proximal” end) associated with the agent release module of the delivery device, and a second end (or “distal” end) for delivery of the agent or drug formulation to a desired target delivery site. In some embodiments, the proximal (e.g., first) end of the agent delivery element is in fluid communication or attached to the agent release module 20 so that the lumen of the agent delivery element is in communication with the agent reservoir in the agent release module, so that a drug formulation contained in the reservoir can move into the agent delivery lumen, and out of an output port which is positioned near the desired target anatomy delivery site. It may be appreciated that such a delivery element may be termed a delivery catheter.

The agent delivery lumen is to have a diameter compatible with providing leak-proof delivery of an agent, e.g., a drug formulation from the agent release module. Where the agent release module dispenses an agent, e.g., a drug formulation by convection (as in, e.g., osmotic drug delivery systems), the size of the drug delivery lumen leading from the reservoir can be designed as described by Theeuwes (1975) J. Pharm. Sci. 64:1987-91.

The body of the agent delivery element 30 can be of any of a variety of dimensions and geometries (e.g., curved, substantially straight, tapered, etc.), that can be selected according to their suitability for the flexibility and to withstand physical forces for delivery of the agent to the DRG. The distal end of the agent delivery element can provide a distinct opening at an output port for delivery of an agent, or as a series of openings or outlet ports which are positioned near the target anatomy delivery site, such as the DRG.

In some embodiments, portions of the agent delivery element can comprise additional materials or agents (e.g., coatings on the external or internal catheter body surface(s)) to facilitate agent delivery and/or to provide other desirable characteristics to the agent delivery element. For example, portions of the agent delivery element inner and/or outer walls can be coated with silver or otherwise coated or treated with antimicrobial agents, thus further reducing the risk of infection at the site of agent release module implantation and DRG agent delivery.

In one embodiment, an agent delivery lumen is primed with an agent, e.g., drug formulation, e.g., is substantially pre-filled with the agent prior to implantation into the subject. Priming of the agent delivery lumen reduces delivery start-up time, i.e., time related to movement of the agent from the agent delivery module to the distal end of the agent delivery element. This feature is particularly advantageous in the present invention where the agent release module of the agent delivery device releases an agent at relatively low flow rates.

In any of the forgoing embodiments, the delivery element may have a coating for preventing or lessening infection or immune response in the adjacent tissue. One can use a variety of coatings, for example, but not limited to silver or silver-based coatings.

In some embodiments, it may be desirable to discourage tissue ingrowth into the sidewall apertures, and therefore a suitable coating may be applied to at least a portion of the distal end of the tube for deterring tissue ingrowth. Alternatively, the material selected for the flexible tubing may have an inherent characteristic of deterring tissue ingrowth. Such materials or coatings may include coatings having hyaluronidase inhibitors, coatings having hyaluronidase enzymatic proteolytic chemistry, or coatings having a dilute papain enzymatic action.

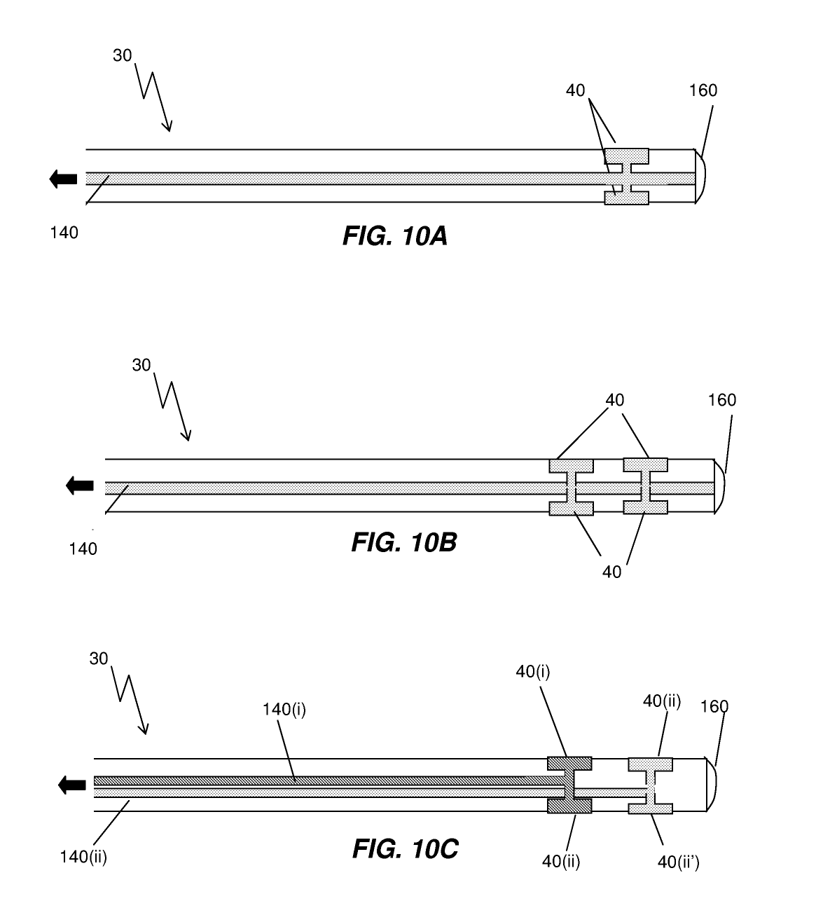

FIGS. 10A-10C illustrate various embodiments of delivery elements 30 having at least one agent delivery outlet port 40. Referring to FIG. 10A, the delivery element 30 includes at least one delivery lumen 140 therein, which has its proximal end in fluid connection with the reservoir 70 of the agent release module 20, and extends towards a distal tip 160. The diameter of the delivery lumen can be of any diameter, for example, 0.1 mm, 0.2 mm, 0.5 mm, 1 mm, 2 mm, 5 mm, or more than 5 mm, and any integer between 0.1 mm and 5 mm in diameter. In some embodiments, the distal end of the delivery element 30 has a closed-end distal tip 160. In such embodiments, the distal tip 160 may have a variety of shapes including a rounded shape, a ball shape, a tear-drop shape, or a cone shape, to name a few. These shapes provide an atraumatic tip for the delivery element 30 as well as serving other purposes. When the distal tip 160 has a closed end, the delivery lumen 140 connects with at least one outlet port 40 located in the wall of the element 30. It may be appreciated that in some embodiments the delivery element 30 has an open-end distal tip 160. In such embodiments, the delivery lumen 140 may connect to the open-end distal tip 160 wherein the distal-tip 160 acts as a agent outlet port 40.

FIG. 10B illustrates one embodiment of the delivery element 30 comprising a plurality of agent outlet ports 40, each fluidly connected to a delivery lumen 140. In this embodiment, one delivery lumen is connected to four outlet ports 40 in the wall of the delivery element. It may be appreciated that any number of outlet ports 40 may be present, including one, two, three, four, five, six, seven, eight, etc.

Alternatively, as shown in FIG. 10C, a delivery element 30 can comprise a plurality of delivery lumens 140(i), 140(ii) each in fluid connection with at least one reservoir 70 at their proximal end and at least one outlet port in the element wall near their distal end. In this embodiment, a first delivery lumen 140(i) is in fluid communication with two outlet ports 40(i), 40(i′) and a second delivery lumen 140(ii) is in fluid communication with two outlet ports 40(ii), 40(ii′). Such an embodiment allows for delivery of more than one different agent to the DRG via each delivery lumen, or alternatively, delivery of the same agent but at a variety of different doses via each delivery lumen. It is also appreciated that the delivery element 30 can comprise any number of delivery lumens, including one, two, three, four five, six, seven, eight, nine, ten, eleven, twelve, thirteen, fourteen, fifteen, sixteen or more. Each delivery lumen can be used for delivering the same agent (e.g., the proximal end of each is connected to the same reservoir) to the target anatomy, e.g., the DRG, or a different agent (e.g., the proximal end of each lumen is connected to a different reservoir) to the target anatomy, e.g., the DRG. In some embodiments, each delivery lumen disposed within the delivery element are independently configurable. For example, in some embodiments, software can ensure an agent is delivered from specific lumens, at specific rates, at particular times. Thus, agent release can be staggered or overlapping from different delivery lumens.

In regards to the combination neurostimulation and pharmacological agent delivery element, the distal tip of the delivery element 30 comprising the electrodes 50 and agent outlet ports 40 can be placed in any location near the target spinal anatomy, e.g., the DRG, to obtain the desired stimulation or modulation level. Additionally, the distal tip of the delivery element 30 comprising the electrodes 50 and agent outlet ports 40 can be placed so that modulation or stimulation energy patterns generated by the electrode will remain within or dissipate only within the targeted neural tissue.

b. Agent Delivery Module

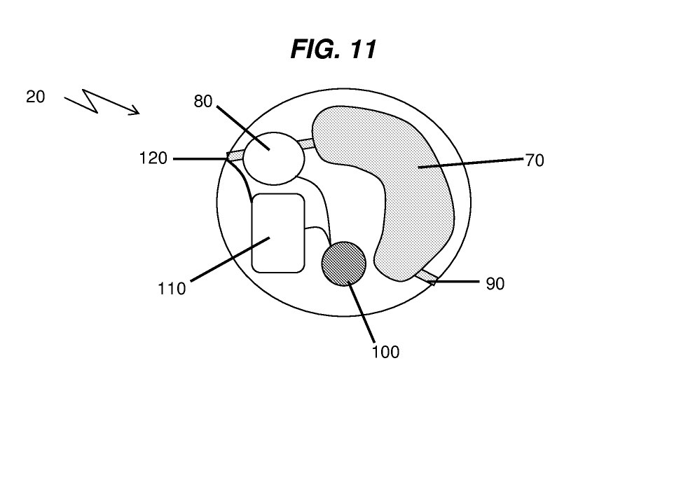

FIG. 11 shows a simplified schematic representation of a specific illustrative embodiment of an agent release module 20 of the delivery device 10 of FIG. 1. In particular, FIG. 11 illustrates the components within this embodiment of the agent release module 20 comprising a agent holding device or drug formulation or agent storage well 70, in fluid connection with a pump 80 for controlled release of the agent from the reservoir to an output 120 of the agent release module 20. In some embodiments, the storage well 70 includes a reservoir, or can in alternative embodiments be a permeable matrix capable of functioning as a support for an agent which releases the agent in a predefined controlled manner.

In some embodiments, the agent release module 20 also comprises a pulse generator 110 and a power supply 100, e.g., a battery, such as a rechargeable or non-rechargeable battery, so the agent delivery device can operate independently of external power sources. It may be appreciated that alternatively, the power supply may be located outside of the housing of the agent release module 20, such as within an external device which supplies power to the agent release module, such as via inductive coupling, RF or photoactivation. The power supply 100 can be used to power the various other components of the agent release module 10, including the agent pump and the pulse generator 110. Accordingly, the power supply 100 can be used to generate electrical stimulation pulses. As such, the power supply 100 can be coupled to the pulse generator 110. Example pulse generators 110 for use in the agent release module 20 are disclosed in U.S. patent applications 2010/0137938, 2010/0249875, US2008/0167698 and International Application, WO2010/083308, WO2008/070807, WO2006/029257, which are all incorporated herein in their entirety by reference. In some embodiments a power supply can also be coupled to controller and a switch device, as well as memory (not shown) in the agent release module.

In some embodiments, the agent release module 10 may also comprise a voltage regulator (not shown) which can be used to alter the voltage of the electrical pulse, e.g., step up or step down a voltage provided by the power supply 100 to produce one or more predetermined voltages useful for powering such components of the agent release module 10. Additional electronic circuitry, such as capacitors, resistors, transistors, and the like, can be used to generate stimulation pulses.

In some embodiments, a pulse generator 110 is coupled to electrodes 50 of the lead(s) via a switch device. A pulse generator 110 can be a single- or multi-channel pulse generator, and can be capable of delivering a single stimulation pulse or multiple stimulation pulses at a given time via a single electrode combination or multiple stimulation pulses at a given time via multiple electrode combinations. In some embodiments, a pulse generator 110 and a switch device can be configured to deliver electrical stimulation pulses to multiple channels on a time-interleaved basis, in which case the switch device time division multiplexes the output of pulse generator 110 across different electrode combinations at different times to deliver multiple programs or channels of stimulation energy to the patient.

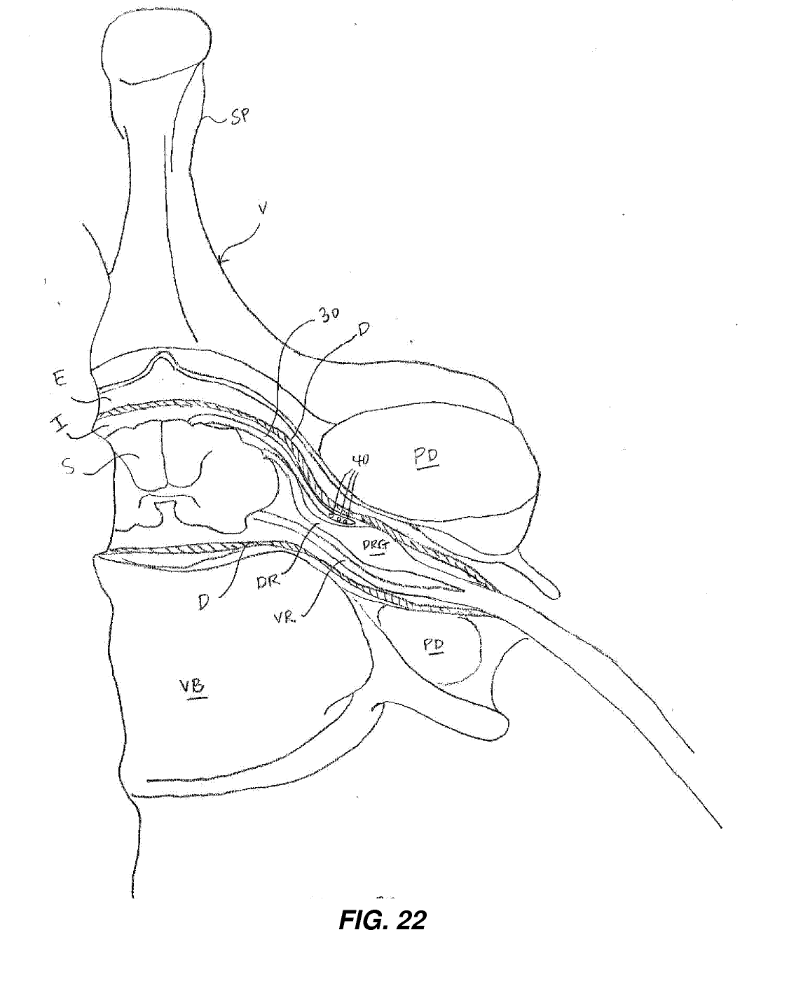

As mentioned previously, in some embodiments, the at least one external programming device comprises a clinical programmer 65 and/or a patient programmer 60. The clinical programmer 65 is used to program the agent release (e.g., controls the agent pump 80) and/or the electrical stimulation information from the pulse generator 110, as determined by the clinician or investigator. The electrical stimulation information includes signal parameters such as voltage, current, pulse width, repetition rate, and burst rates. FIG. 22 illustrates an example of possible parameters of both agent delivery and electrical stimulation signal which may be varied. Using embodiments of the present invention, the amplitude, current, pulse width and repetition rate (also referred to as frequency) which provide the optimal therapeutic result can be determined. It may be appreciated that a constant current with a constant amplitude may be used.

The patient programmer 60 allows the patient to adjust the agent delivery and stimulation settings of the agent release module 20 within limits preset by the clinician. The patient programmer 60 also allows the patient to turn the agent delivery off or increase agent delivery or dose, and turn on or off the electrical stimulation, if necessary. The clinical and patient programmers 65, 60 are portable, hand-held devices that can be plugged into a power outlet or powered by an internal battery. The battery is typically rechargeable using a power supply and a power outlet. In some embodiments, the programmers 65, 60 contain an internal magnet to initiate communication with the agent release module 20. The patient programmer 65 is designed to be easy to use and establishes two-way communication with the agent release module 20 to control the agent delivery to the DRG and/or electrical stimulation. Together the delivery device 10, clinical programmer 65 and patient programmer 60 form an agent-neurostimulatory system 1000, which operates to provide personalized treatment for each patient, as will be described in more detail herein.

Referring back to FIG. 11, the controller (not shown) can control the pulse generator 110 to generate stimulation pulses, and control the switch device to couple the stimulation energy to selected electrodes. More specifically, the controller can also control the pulse generator 110 and the switch device to deliver electrical stimulation energy in accordance with parameters specified by one or more stimulation parameter sets stored within a memory. Exemplary programmable electrical stimulation parameters that can be specified include the pulse amplitude, pulse width, and pulse rate (also known as repetition rate or frequency) for a stimulation waveform (also known as a stimulation signal). Additionally, the controller can control the switch device to select different electrode configurations for delivery of stimulation energy from the pulse generator 110. In other words, additional programmable electrical stimulation parameters that can be specified include which electrodes 50 of which lead(s) are to be used for delivering stimulation energy and the polarities of the selected electrodes 50. Each electrode 50 can be connected as an anode (having a positive polarity), a cathode (having a negative polarity), or a neutral electrode (in which case the electrode is not used for delivering stimulation energy, i.e., is inactive). A set of electrical stimulation parameters can be referred to as a stimulation parameter set since they define the stimulation therapy to be delivered to a patient. One stimulation parameter set may be useful for treating a condition in one location of the body of the patient, while a second stimulation parameter set may be useful for treating a condition in a second location. It may be appreciated that each of the electrodes on an individual lead may provide a signal having the same signal parameters or one or more electrodes on the lead may provide a signal having differing signal parameters. Likewise, an individual electrode may provide a signal having differing signal parameters over time.

A controller can include a microprocessor, a microcontroller, a digital signal processor (DSP), an application specific integrated circuit (ASIC), a field-programmable gate array (FPGA), a state machine, or similar discrete and/or integrated logic circuitry. A switch device can include a switch array, switch matrix, multiplexer, and/or any other type of switching device suitable to selectively couple stimulation energy to selected electrodes. A memory can include RAM, ROM, NVRAM, EEPROM or flash memory, but is not limited thereto. Various agent release programs and/or electrical stimulation parameter sets can be stored in the memory, examples of which are discussed herein.

Once a desired agent delivery rate and regimen and/or electrical stimulation parameter set is determined, the agent release module can be programmed with the optimal parameters of the set. Thus, when agent delivery and electrical stimulation is desired, the appropriate agent pump 80 controlling the agent delivery and the appropriate electrode(s) 50 on the lead(s) are activated, to affect the nerve tissue with the determined neuromodulatory delivery.

The proximal end of the at least one delivery element 30 is connected to the agent release module 20 and is fluidly connected to a source of the drug formulation or agent, e.g., the reservoir 70. The agent release module 20 comprises at least one reservoir, each of which is configured to be fluidly connected to at least one delivery element. Each reservoir 70 also comprises in input port, which allows one-way fluid flow into the associated reservoir for adding a fluid agent into the reservoir. The input port for each reservoir can be connected to a septum which is percutaneously accessible and can be used for periodically and repeatedly refilling at least one reservoir with fluid agent through an externally introduced cannula. The access site of the input port may not be visible after healing of the incisions, and may only be detectable by touch, ultrasound, or other medical imaging technique. A cut-away of the agent release module is shown in FIG. 11. FIG. 2 provides a perspective view of an exemplary agent release module 20.

In some embodiments, the reservoir may be affixed to the reservoir end of the catheter and then implanted, septum side out, at the access site. The reservoir of the delivery device may periodically and repeatedly filled with an agent for alleviating the chronic nerve pain.

In order to charge the agent release module 20 with a fluid agent, the reservoir or agent holding chamber 70 can be connected to an external cannula, via the reservoir inlet port 90 so the agent is added to the reservoir. In one embodiment, the inlet port 90 can be connected to syringe filled with an agent using a hypodermic needle. In another embodiment, the cannula may be removably attached to a source of fluid agent. In some embodiments, the simple construction and operation of the delivery device as disclosed herein also advantageously avoids the need for moving parts that might malfunction.

After the agent release module 20 has been charged with a fluid agent, it may be desirable to flush the reservoir with a saline solution. Without removing the tip of the cannula from the reservoir, the source of fluid agent can be removed from the cannula, and a source of saline (not shown) may be affixed. Fluid flow pressure may be applied to the saline to flush any remaining agent in the reservoir.

1) Agent Delivery Module Size

In some embodiments, the agent release module 20 has a volume not exceeding approximately 32 cc, and a thickness not exceeding approximately 1.2 cm or a weight not exceeding approximately 30 g. It may be appreciated that in other embodiments, an agent release module 20 has a volume not exceeding approximately, 0.2, 5, 10, 15, 20, 30, 40, 50, 60 or 70 cc. In some embodiments, an agent release module 20 can have a variety of shapes, including an oval, circular (as shown in FIG. 11), rounded square or rounded rectangular shape, as shown in FIG. 1. In some embodiments, an agent release module 20 has a height of approximately 61 mm, a width of approximately 48 mm and a thickness of approximately 11 mm. In some embodiments, the reservoir has a volume from about 100 μl to 100 ml, for example, at least about 10 μl, or about 10 μl, or about 100 μl, or about 200 μl, or about 300 μl, or about 400 μl, or about 500 μl, or about 600 μl, or about 700 μl, or about 1000 μl, or about 2 ml, or about 3 ml, or about 4 ml, or about 5 ml, or about 10 ml, or about 15 ml, or about 20 ml, or about 25 ml, or about 30 ml, or about 40 ml, or about 50 ml, or about 60 ml, or about 70 ml, or more than about 70 ml.

2) Agent Delivery Module Materials

In some embodiments, an agent release module comprises a reservoir which is capable of carrying an agent in such quantities and concentration as therapeutically required, and must provide sufficient protection to the agent formulation from attack by body processes for the duration of implantation and delivery. Accordingly, in some embodiments, the exterior of an agent release module is made of a material that has properties to diminish the risk of leakage, cracking, breakage, or distortion so as to prevent expelling of its contents in an uncontrolled manner under stresses it would be subjected to during use, e.g., due to physical forces exerted upon the agent release module as a result of movement by the subject or physical forces associated with pressure generated within the reservoir associated with delivery of the agent to the DRG. In alternative embodiments, an agent release module includes other means for holding or containing an agent which must also be of such material as to avoid unintended reactions with the active agent formulation, and is preferably biocompatible (e.g., where the agent release module is implanted, it is substantially non-reactive with respect to a subject's body or body fluids).

Example suitable materials for the reservoir or agent holding means for use in the agent release module of the delivery device are disclosed herein. For example, a reservoir material may comprise a non-reactive polymer or a biocompatible metal or alloy. Suitable polymers include, but are not necessarily limited to, acrylonitrile polymers such as acrylonitrile-butadiene-styrene polymer, and the like; halogenated polymers such as polytetrafluoroethylene, polyurethane, polychlorotrifluoroethylene, copolymer tetrafiuoroethylene and hexafluoropropylene; polyethylene vinylacetate (EVA), polyimide; polysulfone; polycarbonate; polyethylene; polypropylene; polyvinylchloride-acrylic copolymer; polycarbonate-acrylonitrile-butadiene-styrene; polystyrene; cellulosic polymers; and the like. Further exemplary polymers are described in The Handbook of Common Polymers, Scott and Roff, CRC Press, Cleveland Rubber Co., Cleveland, Ohio.

Metallic materials suitable for use in a reservoir of the agent release module include stainless steel, titanium, platinum, tantalum, gold and their alloys; gold-plated ferrous alloys; platinum-plated titanium, stainless steel, tantalum, gold and their alloys as well as other ferrous alloys; cobalt-chromium alloys; and titanium nitride-coated stainless steel, titanium, platinum, tantalum, gold, and their alloys.

Exemplary materials for use in polymeric matrices include, but are not necessarily limited to, biocompatible polymers, including biostable polymers and biodegradable polymers. Exemplary biostable polymers include, but are not necessarily limited to silicone, polyurethane, polyether urethane, polyether urethane urea, polyamide, polyacetal, polyester, poly ethylene-chlorotrifluoroethylene, polytetrafluoroethylene (PTFE or “TEFLONT™”), styrene butadiene rubber, polyethylene, polypropylene, polyphenylene oxide-polystyrene, poly-a-chloro-p-xylene, polymethylpentene, polysulfone and other related biostable polymers. Exemplary biodegradable polymers include, but are not necessarily limited to, polyanhydrides, cyclodestrans, polylactic-glycolic acid, polyorthoesters, n-vinyl alcohol, polyethylene oxide/polyethylene terephthalate, polyglycolic acid, polylactic acid and other related bioabsorbable polymers.

In some embodiments the agent, e.g., a drug formulation, is stored in a reservoir comprising metal or a metal alloy. In particular, in some embodiments the reservoir is comprised of titanium or a titanium alloy having greater than 60%, often greater than 85%, titanium. Titanium is preferred for size-critical applications, for high payload capability and for long duration applications and for those applications where the formulation is sensitive to body chemistry at the implantation site or where the body is sensitive to the formulation. Typically, the agent release module is designed for storage with an agent at room temperature or higher.

3) Controlled Agent Release

Agent delivery devices suitable for use with the present invention can take advantage of any of a variety of controlled agent release devices. In general, the agent release devices suitable for use a variety of embodiments of the present invention comprise an agent reservoir for retaining a drug formulation or alternatively some substrate or matrix which can hold agent (e.g., polymer, binding solid, etc.). Controlled agent release devices suitable for use in the present invention generally can provide for delivery of the agent from the device at a selected or otherwise patterned amount and/or rate to a selected site in the subject.

Any of a variety of agent release modules can be used in the delivery device of the present invention to accomplish delivery of an agent, e.g., a drug formulation to the DRG. In general, the agent release module is connectable with an agent delivery element 30, such as a catheter or lead, where the implantation site of the agent release module 20 is distant from the target DRG delivery site.

In some embodiments, an agent release module 20 suitable for use with the present invention can take advantage of any of a variety of controlled agent release devices. In general, a agent release module suitable for use in the invention comprise a agent reservoir for retaining an agent, e.g., drug formulation or alternatively some substrate or matrix which can hold the agent or agent (e.g., polymer, binding solid, etc.). Controlled agent release devices suitable for use in the present invention generally can provide for delivery of the agent from the agent release module at a selected or otherwise patterned amount and/or rate to the DRG target site in the subject.

In some embodiments, the agent release module is an implantable device based on diffusive, erodible and/or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some embodiments, the pump works by mechanisms such as but not limited to (i) active pumping, (ii) passive pumping (e.g., diffusion), and/or (iii) electrophoritic drug delivery. In some embodiments, where electrophoritic drug delivery is desired, an electrically conducting wire is inserted into the delivery lumen 140 and where the conducting wire is used to charge the agent (e.g., either a positive or negative charge) within the lumen, and as the charge is greater than the charge in the subjects body, the charged agent is driven out of the lumen and through the outlet ports 40 and into close proximity of the target site, such as the DRG. Agents suitable for delivery using such electrophoritic drug delivery, also referred to in the art as iontophoretic flux or “iontrophoretic delivery” include, without limitation, lidocaine, Epinephrine, fentanyl, fentanyl hydrochloride, ketamine, dexamethasone, hydrocortisone, as well as peptides including but not limited to peptides and proteins such as Angiotension II antagonist, Antriopeptins, Bradykinin, and Tissue Plasminogen activator, Neuropeptide Y, and Nerve growth factor (NGF), Neurotension, Somatostatin and its analogs such as octreotide. Immunomodulating peptides and proteins such as Bursin, Colony stimulating factor, Cyclosporine, Enkephalins, Interferon, Muramyl dipeptide, Thymopoietin, and TNF, and other growth factors such as Epidermal growth factor (EGF), Insulin-like growth factors I & II (IGF-I & II), Inter-leukin-2 (T-cell growth factor) (Il-2), Nerve growth factor (NGF), Platelet-derived growth factor (PDGF), Transforming growth factor (TGF) (Type I or δ) (TGF), Cartilage-derived growth factor, Colony-stimulating factors (CSFs), Endothelial-cell growth factors (ECGFs), Erythropoietin, Eye-derived growth factors (EDGF), Fibroblast-derived growth factor (FDGF), Fibroblast growth factors (FGFs), Glial growth factor (GGF), Osteosarcoma-derived growth factor (ODGF), Thymosin, and Transforming growth factor (Type II or β)(TGF), as disclosed in U.S. Pat. Nos. 5,494,679 and 6,730,667, which are incorporated herein in their entirety by reference.

Release of agent from the agent release module is typically a controlled release of the agent, and can be accomplished in any of a variety of ways, e.g., by incorporation of an agent into a polymer that provides for substantially controlled diffusion of the agent from within the polymer, incorporation of an agent in a biodegradable polymer, providing for delivery of an agent from an osmotically-driven device, etc. In some embodiments, an agent can be delivered through the agent delivery element (e.g., agent delivery lumen) to the target DRG delivery site as a result of capillary action, for example, as a result of pressure generated from the agent release module, by diffusion, by electrodiffusion or by electroosmosis through the device and/or the element. Likewise, examples of stimuli that may be used to bring about release include pH, enzymes, light, magnetic fields, temperature, ultrasonics, osmosis and more recently electronic control of MEMS and NEMS.

In some embodiments, where the delivery device is configured to comprise a lead for electrical stimulation of the DRG, an agent can be delivered through the agent delivery element, e.g., agent delivery lumen via iontophoresis, as disclosed in Dixit et al., Current Drug Delivery, 2007; 4; 1-10, which is incorporated herein in its entirety by reference.

In some embodiments, an agent release module suitable for use in the delivery device as disclosed herein can be based on any of a variety of modes of operation. For example, an agent release module can be based upon a diffusive system, a convective system, or an erodible system (e.g., an erosion-based system). For example, an agent release module can be an osmotic pump, an electroosmotic pump, a vapor pressure pump, or osmotic bursting matrix, e.g., where an agent is incorporated into a polymer and the polymer provides for release of agent, e.g., a drug formulation concomitant with degradation of an agent-impregnated polymeric material (e.g., a biodegradable, drug-impregnated polymeric material). In other embodiments, an agent release module can be based upon an electrodiffusion system, an electrolytic pump, an effervescent pump, a piezoelectric pump, a hydrolytic system, etc is used and controls the release of the agent into the connected delivery lumen for delivery to the DRG.

An agent release module useful in the agent delivery device as disclosed herein can include a mechanical and/or electromechanical infusion pump. In some embodiments, the delivery device as disclosed herein comprises an agent release module which includes any of a variety of refillable, non-exchangeable pump systems. In some instances, pumps and other convective systems are preferred due to their generally more consistent, controlled release over time. In some instances, osmotic pumps are particularly preferred due to their combined advantages of more consistent controlled release and relatively small size.

In one embodiment, an agent release module useful in the agent delivery device as disclosed herein is an osmotically-driven device. Osmotically-driven agent release systems are those that can provide for release of an agent or drug in a range of rates of from about 0.01 μg/hr to about 200 μg/hr, and which can be delivered at a volume rate of from about 0.01 μl/day to about 100 μl/day (i.e., from about 0.0004 μl/hr to about 4 μl/hr), preferably from about 0.04 μl/day to about 10 μl/day, generally from about 0.2 μl/day to about 5 μl/day, typically from about 0.5 μl/day to about 1 μl/day.

Additional details of combination neurostimulation and delivery of agents to the DRG using the pulse generator 110 and the agent release module 20 typically use a combined pump 80 and reservoir 70 with the pulse generator 110, however, it is to be appreciated that the pump 80 for moving the agent from the reservoir 70 out of agent release module 20 to the DRG and the pulse generator 110 connected to the electrodes 40 may be two separate components that operate in a coordinated fashion. Pumps and reservoirs may be any of those suited for controlled delivery of the particular pharmacological agent being delivered. Suitable pumps include any device adapted for whole implantation in a subject, and suitable for delivering the formulations for pain management or other pharmacological agents described herein. In general, the pump and reservoir provide for movement of agent from the reservoir (defined by a housing of the pump or a separate vessel in communication with the pump) by action of an operatively connected pump, e.g., osmotic pumps, vapor pressure pumps, electrolytic pumps, electrochemical pumps, effervescent pumps, piezoelectric pumps, or electromechanical pump systems.

The present disclosure also provides methods of using a delivery device as disclosed herein for providing long-term relief from various conditions, including chronic pain in a subject, e.g., a human subject. The long-term treatment, such as pain relief, may be provided by periodically and repeatedly refilling the reservoirs in the agent release module with a suitable drug formulation or agent. The delivery device can remain fully enclosed within a patient's body during the entire term of use, which may range from about 1 day to about the end of the patient's lifespan. A desirable term of use for the delivery device as disclosed herein can range from about one week to about 50 years, with a further suitable example ranging from about 1 year to about 25 years.

c. DRG as a Target

The dorsal root ganglion (DRG) is a spinal neural structure that partly contains the primary sensory neurons. The primary sensory neurons are fairly unique in that they are bipolar, or quasiunipolar, cells. Each sensory neuron comprises a cell body (soma) and two axons, one carrying sensory information from the periphery to the soma and one carrying information from the soma to the spinal cord. The soma itself is located within the DRG and the axons extend therefrom, such as through the dorsal root into the spinal cord and the sensory fiber axon to the peripheral target, e.g., skin.

Without wishing to be bound by theory, in chronic pain conditions, neurons in the dorsal root ganglion that are specific for the transduction of pain are hypersensitized, as a result of changes in membrane physiology (induced by in the receptor and ion channel expression amoung other things), sensitivity and activation at the central nerve terminal and peripheral nerve terminal (referred to central sensitization and peripheral sensitization, respectively). The result of this hypersensitization is that the neuron responds to typical nociceptive or non-nociceptive inputs in an exaggerated way, thus resulting in a larger perception of pain than would normally be expected for a given input. This response is called hyperalgesia. Contributing to the increased excitability of pain neurons in the DRG is the increased expression of various sodium channels (NaV) subtypes as well as other ion channels in the primary sensory neurons.

Sodium channels (NaV) are integral membrane proteins involved in the transport of sodium ion across the semi-permeable membrane in neurons. These channels form a “family” of channels, with there being several different subtypes of sodium channels. Sodium channels, in essence, provide basic excitability to neurons. They allow the transduction of sodium ions from the extracellular space to the intracellular space thus producing membrane depolarization and action potentials. Sodium channels are a critical element in the transduction of nerve signals and impulses. Sodium channels have been implicated in the development and maintenance of chronic pain. Since sodium channels are a primary driver of neuronal membrane excitability, the increased expression as well as alterations in the channel kinetics can significantly contribute to the pathophysiological alterations in cellular function and, ultimately, in the contribution to chronic pain conditions.

Accordingly, local anesthetics predominantly function by blocking sodium channels, in turn producing the ability to effectively block the transduction of pain. These anesthetic agents are currently used in a variety of ways including local infiltration, epidural anesthesia, regional anesthesia and also as diagnostic nerve blocks. Acute blockade of sodium channels can be used for diagnostic procedures, but the chronic delivery of sodium channel blockers in the treatment of chronic pain is limited due to inefficiency and potential side effects.

Accordingly, the present invention is advantageous in allowing direct delivery of agents, e.g., analgesic agents directly to the target spinal anatomy, e.g., the DRG to specifically direct their action in a localized fashion, thus eliminating undesired non-specific effects and increasing efficiency. The direct delivery of agents can be used to target the soma (e.g., cell body) of the primary sensory neurons in the DRG, as this is primarily the location of the pathophysiological changes which occur during nociceptive and neuropathic pain syndromes. In some embodiments, the delivery of agents to the target spinal anatomy, e.g., DRG can act on the cell body membrane and integral membranes, cell nucleus and intranuclear structures, ribosomes, mitochondria, t-junction, as well as peripheral and central axons emanating from the biplolar cell.

In some embodiments, the present invention can be used to delivery agents to non-neuronal cells in proximity to the target spinal anatomy, e.g., glial cells (e.g., satalite cells) and astrocytes and other non-neuronal supporting cells, and/or inflammatory cells within the proximity of the DRG or cell bodies of the sensory neurons.

When combined agent delivery and neurostimulation are desired, a delivery element 30 (which comprises the lumen) also comprises a lead. The lead includes at least one electrode 50 and where the at least one electrode 50 is in place on, in or adjacent the desired spinal anatomy, e.g., a nerve root ganglion (DRG), the activating step proceeds by coupling a programmable electrical signal to the electrode. In one embodiment, the amount of stimulation energy provided into the target anatomy, e.g., a nerve ganglion is sufficient to selectively stimulate target anatomy, e.g., the DRG. In such an embodiment, the stimulation energy provided only stimulates neural tissue within the targeted DRG and stimulation energy beyond the DRG is below a level sufficient to stimulate, modulate or influence nearby neural tissue.

In an example where the electrode is implanted into a target tissue which is a dorsal root ganglion (DRG), the stimulation level may be selected as one that preferentially activates myelinated, large diameter fibers and/or soma (such as Aβ and Aα fibers) over unmyelinated, small diameter fibers (such as c-fibers). In additional embodiments, the stimulation energy used to activate an electrode to stimulate neural tissue remains at an energy level below the level to used ablate, lesion or otherwise damage the neural tissue. For example, during a radiofrequency percutaneous partial rhizotomy, an electrode is placed into a dorsal root ganglia and activated until a thermolesion is formed (i.e., at an electrode tip temperature of about 67° C.) resulting in a partial and temporary sensory loss in the corresponding dermatome. In one embodiment, the stimulation energy levels applied to a DRG remain below the energy levels used during thermal ablation, RF ablation or other rhizotomy procedures.

Tissue stimulation is mediated when current flow through the tissue reaches a threshold, which causes cells experiencing this current flow to depolarize. Current is generated when a voltage is supplied, for example, between two electrodes with specific surface area. The current density in the immediate vicinity of the stimulating electrode is an important parameter. For example, a current of 1 mA flowing through a 1 mm² area electrode has the same current density in its vicinity as 10 mA of current flowing through a 10 mm² area electrode that is 1 mA/mm². In this example, cells close to the electrode surface experience the same stimulation density. The difference is that the larger electrode can stimulate a larger volume of cells and the smaller electrode can stimulate a smaller volume of cells in proportion to their surface area.

In many instances, the preferred effect is to stimulate or reversibly block nervous tissue. Use of the term “block” or “blockade” in this application means disruption, modulation, and inhibition of nerve impulse transmission. Abnormal regulation can result in an excitation of the pathways or a loss of inhibition of the pathways, with the net result being an increased perception or response. Therapeutic measures can be directed towards either blocking the transmission of signals or stimulating inhibitory feedback. Electrical stimulation permits such stimulation of the target neural structures and, equally importantly, prevents the total destruction of the nervous system. Additionally, electrical stimulation parameters can be adjusted so that benefits are maximized and side effects are minimized

In some embodiments, the neuromodulation system 1000 includes a pulse generator 110 that provides stimulation energy in programmable patterns adapted for direct stimulation of neural tissue using small area, high impedance microelectrodes. The level of stimulation provided is selected to preferentially stimulate the Aβ and Aα fibers over the c-fibers. Stimulation energy levels used by embodiments of the present invention utilize lower stimulation energy levels than conventional non-direct, non-specific stimulations systems because the electrode 50 is advantageously placed on, in or about a dorsal root ganglion (DRG). Without wishing to be bound by theory, one advantage of stimulating the faster transmitting Aβ and Aα fibers by the electrical stimulation methods of the present invention may release opioids at the junction of the dorsal root and the spinal cord from the stimulated fibers. This release raises the response threshold at that junction (elevated junction threshold). The slower and later arriving c-fiber action potential signals remains below the elevated junction threshold and goes undetected.

Accordingly, some embodiments of the present invention provide selective electrical stimulation of the spinal cord, peripheral nervous system and/or one or more dorsal root ganglia. As used herein in one embodiment, selective electrical stimulation means that the stimulation substantially only neuromodulates or neurostimulates a nerve root ganglion. In one embodiment, selective stimulation of a dorsal root ganglion leaves the motor nerves unstimulated or unmodulated. In addition, in other embodiments, selective stimulation can also mean that within the nerve sheath, the A-myelinated fibers are preferentially stimulated or neuromodulated as compared to the c-unmyelinated fibers. As such, embodiments of the present invention advantageously utilize the fact that A-fibers carry neural impulses more rapidly (almost twice as fast) as c-fibers. Some embodiments of the present invention are adapted to provide stimulation levels intended to preferentially stimulate A-fibers over c-fibers.

In some embodiments, the pulse generator 110 provides stimulation energy at a level below a threshold for Aβ fiber recruitment. In other embodiments, the pulse generator provides stimulation energy at a level below a threshold for Aβ fiber cell body recruitment. In other embodiments, the pulse generator provides stimulation energy at a level above a threshold for Aδ fiber cell body recruitment. In still other embodiments, the pulse generator provides stimulation energy at a level above a threshold for C fiber cell body recruitment. In some embodiments, the pulse generator provides stimulation energy at a level above a threshold for small myelenated fiber cell body recruitment. And, in some embodiments, the pulse generator provides stimulation energy at a level above a threshold for unmyelenated fiber cell body recruitment.

In some embodiments, the electrical stimulation signal has a current amplitude of less than or equal to approximately 10 mA. In some embodiments, the electrical stimulation signal has a current amplitude of between 10-100 mA, or between about 100-200 mA, or between about 200-300 mA, or between about 300-500 mA, or between about 500-800 mA, or between about 800-1000 mA, or more than 1000 mA. In some instances the at least one of the at least one electrodes has an average electrode surface area of less than or equal to approximately 6 mm². Optionally, the average electrode surface area is less than or equal to approximately 4 mm².

In some embodiments, the electrical stimulation signal has a stimulation signal having a current amplitude which is less than 100 μA. Typically, the target spinal neural tissue comprises a dorsal root ganglion.

In some embodiments, the pulse generator 110 provides the stimulation signal which has an energy of less than approximately 100 nJ per pulse. In some embodiments, the stimulation signal has an energy of less than approximately 50 nJ per pulse. Alternatively, the stimulation signal can have an energy of between about 12-24 nJ, or less than approximately 10 nJ per pulse. Typically, the at least a portion of the target dorsal root comprises a dorsal root ganglion.

Likewise, in some embodiments, the at least one signal parameter includes pulse width and the pulse width is less than 500 μs. In some embodiments, a pulse generator 110 provides the stimulation signal having a current amplitude which is adjustable in increments of 50 μA or less.

Due to variability in patient anatomy, pain profiles, pain perception and lead placement, to name a few, signal parameter settings will likely vary from patient to patient and from lead to lead within the same patient. Signal parameters include voltage, current amplitude, pulse width and repetition rate, pulse waveform shape, to name a few. In some embodiments of the stimulation system of the present invention, the voltage provided is in the range of approximately 0-7 volts. In some embodiments, the current amplitude provided is less than approximately 4 mA, particularly in the range of approximately 0.5-2 mA, more particularly in the range of approximately 0.5-1.0 mA, 0.1-1.0 mA, or 0.01-1.0 mA. Further, in some embodiments, the pulse width provided is less than approximately 2000 μs, particularly less than approximately 1000 μs, more particularly less than approximately 500 μs, or more particularly 10-120 μs. And, in some embodiments, the repetition rate is in the range of approximately 2-120 Hz, up to 200 Hz or up to 30,000 Hz, or more than 30,000 Hz.

Typically, stimulation parameters are adjusted until satisfactory clinical results are reached. Thus, there is an envelope of stimulation parameter value combinations between the threshold for DRG stimulation and ventral root stimulation for any given lead positioned in proximity to any given DRG per patient. The specific combinations or possible combinations that could be used to successfully treat the patient are typically determined perioperatively and/or postoperatively and depend on a variety of factors, such as the placement of the electrodes and the type and severity of the pain experienced by the subject. One factor is lead placement. The closer the desired electrodes are to the DRG the lower the energy required to stimulate the DRG. Other factors include electrode selection, the anatomy of the patient, the pain profiles that are being treated and the psychological perception of pain by the patient, to name a few. Over time, the parameter values for any given lead to treat the patient may change due to changes in lead placement, changes in impedance or other physical or psychological changes. In any case, the envelope of parameter values is exceedingly lower than those of conventional stimulation systems which require energy delivery of at least an order of magnitude higher to treat the patient's pain condition.

Given the lower ranges of parameter values, the granularity of control is also smaller in comparison to conventional stimulation systems. For example, current in a conventional stimulation system is typically adjustable in increments of 0.1 mA. In some embodiments of the present invention, this increment is larger than the entire range of current amplitude values that may be used to treat the patient. Thus, smaller increments are needed to cycle through the signal parameter values to determine the appropriate combination of values to treat the condition. In some embodiments, the system of the present invention provides control of current amplitude at a resolution of approximately 25 μA, particularly when using a current amplitude under, for example, 2 mA, however it may be appreciated that smaller increments may be used such as approximately 10 μA, 5 μA or 1 μA. In other embodiments, control of current amplitude is provided at a resolution of approximately 50 μA, particularly when using a current amplitude of, for example, 2 mA or greater. It may be appreciated that such a change in resolution may occur at other levels, such as 1 mA. Similarly, voltage in a conventional stimulation system is typically adjustable in increments of 100 mV. In contrast, some embodiments of the present invention provide control of voltage at a resolution of 50 mV. Likewise, some embodiments of the present invention provide control of pulse width at a resolution of 10 μs. Thus, it may be appreciated that the present invention provides a high granularity of control of stimulation parameters due to the low ranges of parameter values.

It may be appreciated that in some instances even lower levels of energy may be used to successfully treat a patient using the stimulation system of the present invention. The closer a lead is positioned to a target DRG, the lower the level of energy that may be needed to selectively stimulate the target DRG. Thus, signal parameter values may be lower than those stated herein with correspondingly higher granularity of control.

Such reductions in energy allows a reduction in electrode size, among other benefits. In some embodiments, the average electrode surface area is approximately 1-6 mm², particularly approximately 2-4 mm², more particularly 3.93 mm² whereas conventional spinal cord stimulators typically have a much larger average electrode surface area, such as 7.5 mm² for some leads or 12.7 mm² for traditional paddle leads. Likewise, in some embodiments an average electrode length is 1.25 mm whereas conventional spinal cord stimulators typically have an average electrode length of 3 mm. Such reduced electrode sizing allows more intimate positioning of the electrodes in the vicinity of the DRG and allows for the pulse generator 110 in the agent release module 20 having different control and performance parameters for providing direct and selective stimulation of a targeted neural tissue, particularly the DRG. In addition, in some embodiments the overall dimensions of one or more electrodes and the spacing of the electrodes is selected to match or nearly match the overall dimensions or size of the stimulation target.

Effective treatment of a condition may be achieved by directly stimulating a target anatomy associated with the condition while minimizing or excluding undesired stimulation of other anatomies. When such a condition is limited to or primarily affects a single dermatome, the present invention allows for stimulation of a single dermatome or regions within a dermatome (also referred to as subdermatomal stimulation).

In one aspect of the present invention, a method of treating a condition associated with a spinal neural tissue is provided, wherein the treatment is applied substantially within a single dermatome. In some embodiments, the method comprises positioning a lead having at least one electrode so that at least one of the at least one electrodes is in proximity to the spinal neural tissue within an epidural space, and energizing the at least one of the at least one electrodes so as to stimulate the spinal neural tissue causing a treatment effect within the single dermatome while maintaining body regions outside of the single dermatome substantially unaffected. In some embodiments, energizing the at least one electrode comprises energizing the at least one of the at least one electrode so as to stimulate the spinal neural tissue causing a treatment affect within a particular body region within the single dermatome while maintaining body regions outside of the particular body region substantially unaffected. Typically, the spinal neural tissue comprises a dorsal root ganglion and the treatment effect comprises paresthesia. In some embodiments, the particular body region comprises a foot.

In another aspect of the present invention, a method of treating a condition of a patient is provided, wherein the condition is associated with a portion of a dorsal root ganglion and is not substantially associated with other portions of the dorsal root ganglion. In some embodiments, the method comprises positioning a lead having at least one electrode so that at least one of the at least one electrode resides in proximity to the portion of a dorsal root ganglion, and providing a stimulating signal to the at least one of the at least one electrode so as to stimulate the portion of the dorsal root ganglion in a manner that affects the condition while not substantially stimulating the other portions. In some embodiments, the condition comprises pain. In such embodiments, affecting the condition may comprise alleviating the pain without causing a perceptible motor response.

In some embodiments, the condition is sensed by a patient at a location within a dermatome, and the other portions of the dorsal root ganglion are associated with other locations within the dermatome. In some embodiments, a stimulating signal may have a current amplitude of less than or equal to approximately 4 mA. Optionally, a stimulating signal may have current amplitude of less than or equal 1 mA. Typically, positioning the lead comprises advancing the lead using an epidural approach but is not limited to such a method.

In another aspect of the present invention, a method of providing subdermatomal stimulation is provided comprising positioning a lead having at least one electrode so that at least one of the at least one electrode resides near a dorsal root ganglion within a dermatome, and providing a stimulating signal to the at least one of the at least one electrode so as to stimulate the dorsal root ganglion in a manner which affects a condition in a subdermatomal region of the dermatome.

In another aspect of the present invention, a system is provided for stimulating a portion of a dorsal root ganglion, wherein the portion of the dorsal root ganglion is associated with a particular region within a dermatome. In some embodiments, the system comprises a lead having at least one electrode, wherein the lead is configured to be positioned so that at least one of the at least one electrode is able to stimulate the portion of the dorsal root ganglion, and a pulse generator connectable with the lead, wherein the generator provides a stimulation signal to the at least one of the at least one electrode which stimulates the portion of the dorsal root ganglion to cause an effect within the particular region of the dermatome.

In some embodiments, the combination of the at least one of the at least one electrode and the stimulation signal creates an electric field having a shape which allows for stimulation of the portion of the dorsal root ganglion while substantially excluding other portions of the dorsal root ganglion. In some embodiments, the at least one of the at least one electrode comprises two electrodes spaced 0.250 inches apart from approximate center to center of each electrode. In some embodiments, stimulation signal has a current amplitude of less than or equal to approximately 4 mA. Optionally, the stimulating signal may have a current amplitude of less than or equal 1 mA. In some embodiments, the stimulation signal has an energy of less than approximately 100 nJ per pulse

In some embodiments, the pulse generator 110 provides stimulation energy at a level which is capable of modulating glial cell function within the dorsal root ganglion. For example, in some embodiments, the pulse generator provides stimulation energy at a level which is capable of modulating satellite cell function within the dorsal root ganglion. In other embodiments, the pulse generator provides stimulation energy at a level which is capable of modulating Schwann cell function within the dorsal root ganglion.

In some instances, the pulse generator provides stimulation energy at a level which is capable of causing at least one blood vessel associated with the dorsal root ganglion to release an agent or send a cell signal which affects a neuron or glial cell within the dorsal root ganglion.

A signal of “stimulation on” indicates any of a wide variety of stimulation patterns and degrees of stimulation. The “stimulation on” signal may be an oscillating electrical signal may be applied continuously or intermittently. Furthermore, if an electrode is implanted directly into or adjacent to more than one ganglion, the oscillating electrical signal may be applied to one electrode and not the other and vice versa. One can adjust the stimulating poles, the pulse width, the amplitude, as well as the frequency of stimulation and other controllable electrical and signally factors to achieve a desired modulation or stimulation outcome.

The application of the oscillating electrical signal stimulates the area of the nerve chain where the electrode 115 is placed. This stimulation may either increase or decrease nerve activity. The frequency of this oscillating electrical signal is then adjusted until the symptoms manifest by physiological disorder being treated has been demonstrably alleviated. This step may be performed using patient feedback, sensors or other physiological parameter or indication. Once identified, this frequency is then considered the ideal frequency. Once the ideal frequency has been determined, the oscillating electrical signal is maintained at this ideal frequency by storing that frequency in the controller.

In one specific example, the oscillating electrical signal is operated at a voltage between about 0.5 V to about 20 V or more. More preferably, the oscillating electrical signal is operated at a voltage between about 1 V to about 30 V or even 40 V. For micro stimulation, it is preferable to stimulate within the range of 1 V to about 20 V, the range being dependent on factors such as the surface area of the electrode. Preferably, the electric signal source is operated at a frequency range between about 10 Hz to about 10,000 Hz. More preferably, the electric signal source is operated at a frequency range between about 30 Hz to about 500 Hz. Preferably, the pulse width of the oscillating electrical signal is between about 25 microseconds to about 500 microseconds. More preferably, the pulse width of the oscillating electrical signal is between about 50 microseconds to about 300 microseconds.

The application of the oscillating electrical signal may be provided in a number of different ways including, but not limited to: (1) a monopolar stimulation electrode and a large area non-stimulating electrode return electrode; (2) several monopolar stimulating electrodes and a single large area non-stimulating return electrode; (3) a pair of closely spaced bi-polar electrodes; and (4) several pairs of closely spaced bi-polar electrodes. Other configurations are possible. For example, the stimulation electrode(s) of the present invention may be used in conjunction with another non-stimulating electrode—the return electrode—or a portion of the stimulation system may be adapted and/or configured to provide the functionality of a return electrode. Portions of the stimulation system that may be adapted and/or configured to provide the functionality of the return electrode include, without limitation, the battery casing or the pulse generator casing.

It will be appreciated that embodiments of the present invention can stimulate specific dermatome distributions to probe which electrode or group of electrodes or combination of electrodes (including agent coated or delivery electrodes) is best positioned or correlates most closely to one or more specific areas of pain. As such, a stimulation system according to an embodiment of the present invention may be “fine tuned” to a specific area of coverage or type of pain. The results obtained from such testing can be used to one or more stimulation or treatment regimes (i.e., series of stimulations in the presence of or in combination with a therapeutic agent from a coated electrode) for a particular patent for a particular type of pain. These pain treatment regimes may be programmed into a suitable electronic controller or computer controller system (described below) to store the treatment program, control and monitor the system components execution of the stimulation regime as the desired therapeutic regime is executed.

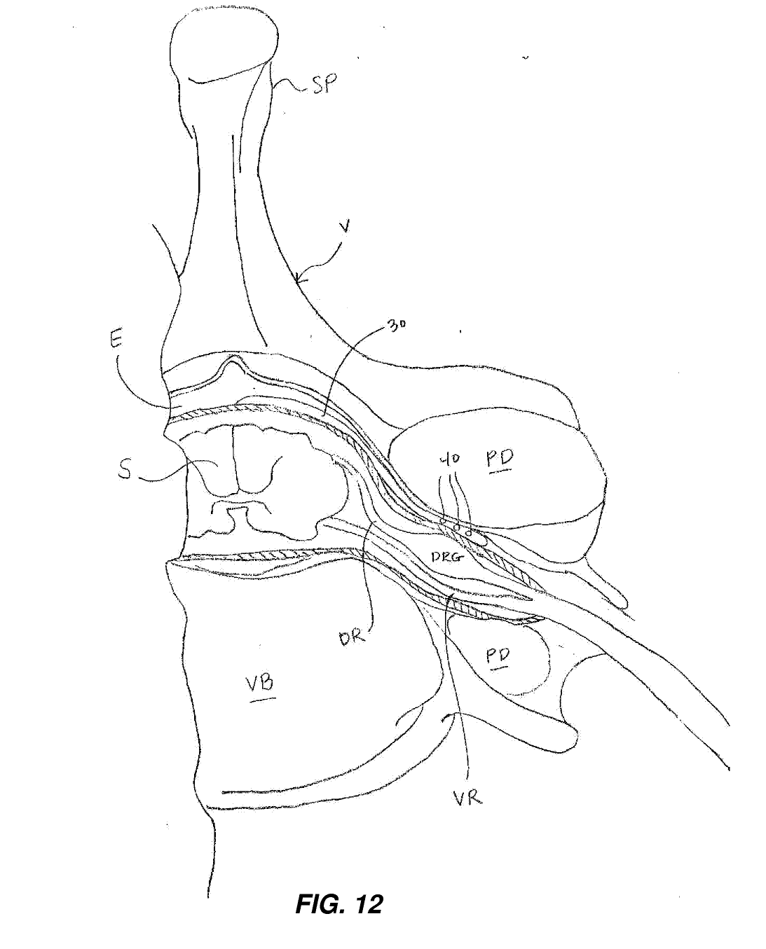

Synergy of electrical and pharmacological modulation may also be obtained using a number of other available pharmacological blockers or other therapeutic agents using a variety of administration routes in combination with specific, directed stimulation of a nerve root ganglion, a dorsal root ganglia, the spinal cord or the peripheral nervous system. Pharmacological blockers include, for example, Na+ channel blockers, Ca++ channel blockers, NMDA receptor blockers and opioid analgesics. As illustrated in FIGS. 12A-16, a combined stimulation and agent delivery electrode results in several effects, including but not limited to, (i) synergistic action of the agent and electrical stimulation, (ii) an increase in the selectivity of an agent to target DRG cell bodies, (iii) targeted activation of an agent delivered to the DRG and (iv) differential enhancement of an agent to delivered target DRG cell bodies. For example, for (iv) because the activation potential of the c-fiber has been lowered, the larger diameter A-fiber is preferentially stimulated or the response of the A-fiber remains above the threshold of activation.

Embodiments of the present invention also provide numerous advantageous combinational therapies. For example, a pharmacological agent may be provided that acts within or influences reactions within the dorsal root ganglia in such a way that the amount of stimulation provided by electrode 50 may be reduced and yet still achieve a clinically significant effect. Alternatively, a pharmacological agent may be provided that acts within or influences reactions within the dorsal root ganglia in such a way that the efficacy of a stimulation provided is increased as compared to the same stimulation provided in the absence of the pharmacological agent. In one specific embodiment, the pharmacological agent is a channel blocker that, after introduction, the c-fiber receptors are effectively blocked such that a higher level of stimulation may be used that may be used in the presence of the channel blocking agent. In some embodiments, the agent may be released prior to stimulation. In other embodiments, the agent may be released during or after stimulation, or in combinations thereof. For example, there may be provided a treatment therapy where the agent is introduced alone, stimulation is provided alone, stimulation is provided in the presence of the agent, or provided at a time interval after the introduction of the agent in such a way that the agent has been given sufficient time to introduce a desired pharmacological effect in advance of the applied stimulation pattern.

Embodiments of the stimulation systems and methods of the present invention enable fine tuning of C-fiber and Aβ-fiber thresholds using the DRG delivered agents coupled with electrical stimulation. Representative pharmacological agents include, but are not limited to: Na+ channel inhibitors, Phenyloin, Carbamazepine, Lidocaine GDNF, Opiates, Vicodin, Ultram, and Morphine.

2. Agent Delivery Vehicles and Methods

The agent is deliverable to the target tissue, e.g. the DRG, by itself or via an agent or agent delivery vehicle or method. Example agent delivery vehicles and methods include nanoparticles, micelles, dendrimers, liposomes, mists, microdroplets, aerosols, atomizations, gels, artifical DNA nanostructures and biologic vectors, to name a few. At least some of these will be described herein.

In some embodiments, the agent delivery vehicle comprises a biodegradable polymer which requires no follow up surgical removal once the agent supply is depleted. In some embodiments, aliphatic polyesters such as poly (lactic acid), poly (glycolic acid), poly (lactide-co-glycolide), poly (decalactone), poly ε-caprolactone are used. Various other polymers like triblock polymer systems composed of poly(D,L-lactide)-block-poly(ethylene glycol)-block-poly(DL-lactide), blends of low molecular weight poly(D,L-lactide) and poly(ε-caprolactone) may also be used. These polymers are mainly used for the injectable in situ formulations. The feasibility of lactide/glycolide polymers as excipients for the controlled release of bioactive agents is well proven. These materials have been subjected to extensive animal and human trials without evidence of any harmful side effects. When properly prepared under GMP conditions from purified monomers, the polymers exhibit no evidence of inflammatory response or other adverse effects upon implantation.

FIG. 12 illustrates example delivery of an agent or agent containing delivery vehicle with the use of a delivery element 30. The delivery element 30 is shown advanced along the spinal cord S within the epidural space E to the appropriate spinal level and advanced at least partially through a foramen, between the pedicles PD. In this example, the delivery element 30 comprises a catheter having outlet ports 40. The delivery element 30 is positioned so that the outlet ports 40 are near or in proximity to the target DRG. The agent or agent containing delivery vehicle is advanced through one or more of the outlet ports 40 into the epidural space. It may be appreciated that in some embodiments the agent and/or agent containing delivery vehicle permeates, penetrates or pervades the dura layer D and the epinurium of the DRG so as to be delivered to within the DRG. It may also be appreciated that the agent may be delivered to the epidural space near the DRG for other purposes, such as to affect neurostimulation, as will be discussed in later sections. It may also be appreciated that the delivery element 30 may approach the target DRG from outside of the spinal column, such as with an extraforminal approach, wherein the delivery element 30 is advanced into the foramen toward the spinal cord S.

a. Nanoparticles

In some embodiments, an agent delivered to the target spinal anatomy, e.g., DRG can be delivered in a carrier particle, wherein the carrier particle comprises an agent. Carrier particles as disclosed herein include any carrier particle for transporting an agent according to the methods as disclosed herein. In some embodiments, carrier particles include colloidal dispersion systems, which include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure. In some embodiments, a carrier particle is a liposome, a dendrimers, a nanocrystal, a quantum dot, a nanoshell or a nanorod, or similar structures.

In some embodiments, a carrier particle as a delivery tool to deliver a desired agent to the target spinal anatomy include, for example but are not limited to, a micro-lipid particle or nano-lipid particle, e.g., liposomes, spheres, micelles, or nanoparticles. In some embodiments the carrier particles are unilammar, (meaning the carrier particles comprise more than one layer or are multi-layered). In some embodiments, carrier particles include colloidal dispersion systems, which include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure. Other carrier particles are cellular uptake or membrane-disruption moieties, for example polyamines, e.g. spermidine or spermine groups, or polylysines; lipids and lipophilic groups; polymyxin or polymyxin-derived peptides; octapeptin; membrane pore-forming peptides; ionophores; protamine; aminoglycosides; polyenes; and the like. Other potentially useful functional groups include intercalating agents; radical generators; alkylating agents; detectable labels; chelators; or the like.

The term “carrier particle” as used herein refers to any entity with the capacity to associate with and carry (or transport) an agent in the body. As discussed herein in some embodiments, a carrier particle can carry both an insoluble agent and a soluble agent simultaneously. In alternative embodiments, a carrier particle can carry an insoluble agent or a soluble agent. Carrier particles can be a lipid particle, such as but not limited to a liposome or a protein or peptide carrier particle. Carrier particles include but are not limited to liposomal or polymeric nanoparticles such as liposomes, proteins, and non-protein polymers. Carrier particles can be selected according to (i) their ability to transport the agent of choice and (ii) the ability to associate with the islet-targeting molecule as disclosed herein.

The term “nanoparticle” as used herein refers to a microscopic particle whose size is measured in nanometers. A carrier particle here can be a nanoparticle.

In some embodiments, as disclosed herein, the carrier particle can be a polymer. Soluble non-protein polymers useful as carrier particles, include, but are not limited to polyvinylpyrrolidone, pyran copolymer, polyhydroxypropylrnethacrylamidephenol, polyhydroxyethylaspartamidephenol, or polyethyleneoxidepolylysine substituted with palitoyl residues. Furthermore, the therapeutic agents can be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels. The therapeutic agents can also be affixed to rigid polymers and other structures such as fullerenes or Buckeyballs.

In such embodiments virtually any agent or drug can be encapsulated in the carriers via lyophilization and reconstitution with an agent suspended in aqueous solution. For example, as disclosed herein, use of the amphiliphic poly (D,L-lactide-co-glycolide)-block-poly(ethylene glycol) (PLGA-b-PEG-COOH) co-polymer allows for spontaneous self-assembly into nanoparticles in aqueous solution. Accordingly, if the aqueous solution comprise an agent to be delivered to a particular cell-type within the target spinal anatomy, e.g., DRG, DR or DREZ a targeting molecule, where an agent can be automatically be encapsulated in the carrier particle nanoparticle on spontaneous self-assembly. Such amphiliphic poly (D,L-lactide-co-glycolide)-block-poly(ethylene glycol) (PLGA-b-PEG-COOH) co-polymers which self-assemble are advantage as it simplifies optimization and large-scale production of carrier-particles enaspulating an agent of interest.

Accordingly, in some embodiments, a polymer carrier particle is a co-polymer, for example, but not limited to a PLGA-PEG co-polymer, for example, but not limited to [PLGA-b-PEG-COOH]n. In some embodiments, where a block co-polymer is [PLGA-b-PEG-COOH]n, there can be various blend composition of PLGA to PEG, for example different ratios such as (75:25, 50:50 etc, and vice versa), and can in some embodiments, be or include other biodegradable polymers such as polycaprolactone, polylactic acid and polyglycolide.

The term “polymer” as used herein, refers to a linear chain of two or more identical or non-identical subunits joined by covalent bonds. A peptide is an example of a polymer that can be composed of identical or non-identical amino acid subunits that are joined by peptide linkages. A co-polymer is a linking of different non-identical subunits in a repeated unit form.

In some embodiments, a co-polymer useful in the compositions and methods as disclosed herein is a synthetic biocompatible and biodegradable copolymer, for example, such as but not limited to any one or a combination of the following: polylactides, polyglycolides, polycaprolactones, polyanhydrides, poly(glycerol sebacate), polyamides, polyurethanes, polyesteramides, polyorthoesters, polydioxanones, polyacetals, polyketals, polyorthocarbonates, polydihydropyrans, polyphosphazenes, polyhydroxybutyrates, polyhydroxyvalerates, polyalkylene oxalates, polyalkylene succinates, poly(malic acid), poly(acrylic acid), polyvinylpyrrolidone, polyhydroxycellulose, polymethyl methacrylate.

In some embodiments, a co-polymer useful as a carrier particle for delivering an agent as disclosed herein is a synthetic biocompatible and non-degradable copolymer, for example, such as but not limited to any one or a combination of the following: polyethylene glycol, polypropylene glycol, pluronic (Poloxamers 407, 188, 127, 68), poly(ethylenimine), polybutylene, polyethylene terephthalate (PET), polyvinyl chloride, polystyrene, polyamides, nylon, polycarbonates, polysulfides, polysulfones, polyacrylonitrile, polyvinylacetate, cellulose acetate butyrate, nitrocellulose.

In some embodiments, a co-polymer useful in the compositions and methods as disclosed herein is a natural biodegradable polymer, for example, such as but not limited to any one or a combination of the following: chitin, chitosan, elastin, gelatin, collagen, silk, alginate, cellulose, poly-nucleic acids, poly(amino acids), hyaluronan, heparin, agarose, pullulan.

In some embodiments, a copolymer useful in the compositions and methods as disclosed herein is can be a combination of biodegradable/biocompatible/natural polymers.

In some embodiments, a nanoparticle can comprise a first layer which can comprise agents that facilitate cryoprotection, long half-life in circulation, or both (PEG, hyaluronan, others). A carrier particle comprises at least one insoluble agent and/or at least one soluble agent. In some embodiments, the carrier particle can also be conjugated to an agent for specifically targeting the carrier particle to a particular spinal anatomy location, e.g., the DRG, or to a particular cell type in the DRG, e.g., cell bodies of c-fibers. Accordingly, a carrier particle can comprise a targeting molecule which can binds to (or has specific affinity for) to a cell surface marker expressed on a particular cell type, for example, but not limited to C-fiber cell bodies in the DRG. Such a targeting molecule which binds to (e.g., has specific affinity for) a cell surface marker expressed on a target cell, e.g., a c-fiber cell body in the DRG can be, for example, but not limited to, a peptide, an antibody or aptamer, or modified versions thereof.

In another embodiment, the carrier particle is a cyclodextrin-based nanoparticle. Polycation formulated nanoparticles have been used for agent delivery into the brain and are useful for delivery of any agent, such as but not limited to siRNA. A unique cyclodextrin-based nanoparticle technology has been developed for targeted gene delivery in vivo. This delivery system consists of two components. The first component is a biologically non-toxic cyclodextrin-containing polycation (CDP). CDPs self-assemble with siRNA to form colloidal particles about 50 nm in diameter and protects si/shRNA against degradation in body fluids. Moreover, the CDP has been engineered to contain imidazole groups at their termini to assist in the intracellular trafficking and release of the nucleic acid. CDP also enables assembly with the second component. The second component is an adamantane-terminated polyethylene glycol (PEG) modifier for stabilizing the particles in order to minimize interactions with plasma and to increase the attachment to the cell surface targeting markers on target neuronal cells, e.g., DRG cells). Thus, the advantages of this delivery system are: 1) the CDP protects the siRNA from degradation therefore chemical modification of the nucleic acid is unnecessary, 2) the colloidal particles do not aggregate and have extended life in biological fluids because of the surface decoration with PEG that occurs via inclusion complex formation between the terminal adamantane and the cyclodextrins, 3) cell type-specific targeted delivery is possible because some of the PEG chains can contain at least one or more targeting molecule, 4) it does not induce an immune response, and 5) in vivo delivery does not produce an interferon response even when a siRNA is used that contains a motif known to be immunostimulatory when delivered in vivo with lipids.

The glycosaminoglycan carrier particles disclosed in U.S. Patent Appl. No. 20040241248 and the glycoprotein carrier particles in WO 06/017195, which are incorporated herein in their entirety by reference, are useful in the methods of the present invention. Similar naturally occurring polymer-type carriers are also useful in the methods of the present invention.

In some embodiments, a carrier particle can be coated with a second layer containing a targeting molecule. In particular, in some embodiments, a carrier particle is selected for its ability to be modifiable by attachment of at least targeting molecule which can bind to (or has specificity to) a specific target cell, e.g., a specific type of neuronal cell or DRG cell, e.g., a c-fiber cell body in the DRG. Carrier particles can be selected according to (i) their ability to transport the agent of choice and/or (ii) their ability to associate with a targeting moiety as disclosed herein. In some embodiments, a carrier particle can comprise at least one, or at least about 2, or at least about 3, or between about 4-5, or between about 5-10, or between about 10-20, or between about 20-50, or between about 50-100, or between about 100-200, or between about 200-500 or more than 500, or any integer between 1-500 or more targeting molecules per carrier particle. It is assumed that multiple targeting molecules per carrier particle will increase the efficiency of targeting the carrier particle to a target location or particular target cells. In some embodiments, the carrier particles can comprise more than one different target molecules, thus enabling the carrier particle (comprising the agent) to be targeted to more than one target cell-type. One of ordinary skill in the art should determine the maximum about of targeting molecules without interfering with the ability of the effect of an agent attached on the outside of a carrier particle, or the ability of the carrier particle to release the agent at the site of the targeted neuronal cell.

A targeting molecule can be linked to the carrier particle, e.g., nanoparticle or other entity via any suitable means, see for example U.S. Pat. Nos. 4,625,014, 5,057,301 and 5,514,363, which are incorporated herein in their entirety by reference. Additional methods are e.g. described by Hermanson (1996, Bioconjugate Techniques, Academic Press), in U.S. Pat. No. 6,180,084 and U.S. Pat. No. 6,264,914 which are incorporated herein in their entirety by reference and include e.g. methods used to link haptens to carriers proteins as routinely used in applied immunology (see Harlow and Lane, 1988, “Antibodies: A laboratory manual”, Cold Spring Harbor Laboratory Press, Cold Spring Harbor, N.Y.). It is recognized that, in some cases, an islet-targeting molecule or carrier particle can lose efficacy or functionality upon conjugation depending, e.g., on the conjugation procedure or the chemical group utilized therein. However, given the large variety of methods for conjugation the skilled person is able to find a conjugation method that does not or least affects the efficacy or functionality of the entities to be conjugated.

In another embodiment, two or more agents can be delivered by carrier particle, for example a lipid particle or polymeric nanoparticles. In such embodiments, one agent can be an insoluble (i.e. hydrophobic or lipohilic) agent and the other agent a soluble (i.e. hydrophilic) agent. An insoluble (or hydrophobic/lipophilic) agent can be added to the lipid particle during formation of the lipid particle and can associate with the lipid portion of the lipid particle. The soluble agent (i.e. hydrophilic agent) is associated with the lipid particle by being added in the aqueous solution during the rehydration of the lyophilized lipid particle, and therefore encapsulated in the carrier particle. An exemplary embodiment of two agent delivery can include a soluble agent, such as a nucleic acid, e.g., RNAi, modRNA etc., and/or another soluble agent, which is encapsulated or entrapped in the aqueous interior of a carrier particle liposome, and where an insoluble (hydrophobic) agent and poorly soluble in aqueous solution is associated with the lipid portion of the liposome carrier particle. As used herein, “poorly soluble in aqueous solution” refers to a composition that is less that 10% soluble in water.

In one aspect of the method, a targeting molecule: carrier particle complex can be detectably labeled, for example it can comprise a carrier particle such as a liposome or polymeric nanoparticle is detectably labeled with a label selected from the group including a radioactive label, a fluorescent label, a non-fluorescent label, a dye, or a compound which enhances magnetic resonance imaging (MRI). In one embodiment, the liposome product is detected by acoustic reflectivity. The label may be attached to the exterior of the liposome or may be encapsulated in the interior of the liposome.

b. Micelles and Dendrimers

In some embodiments, the carrier particles for use in the present invention can be a micro-lipid particle or nano-lipid particle, e.g., spheres, micelles, or dendrimers. In some embodiments the carrier particles are unilammar, (meaning the carrier particles comprise more than one layer or are multi-layered).

A micelle is an aggregate of surfactant molecules dispersed in a liquid colloid. A typical micelle in aqueous solution forms an aggregate with the hydrophilic “head” regions in contact with surrounding solvent, sequestering the hydrophobic single tail regions in the micelle centre. This type of micelle is known as a normal phase micelle (oil-in-water micelle). Inverse micelles have the headgroups at the centre with the tails extending out (water-in-oil micelle).

The term “micelle” as used herein refers to an arrangement of surfactant molecules (surfactants comprise a non-polar, lipophilic “tail” and a polar, hydrophilic “head”). As the term is used herein, a micelle has the arrangement in aqueous solution in which the non-polar tails face inward and the polar heads face outward. Micelles are typically colloid particles formed by an aggregation of small molecules and are usually microscopic particles suspended in some sort of liquid medium, e.g., water, and are between one nanometer and one micrometer in size. A typical micelle in aqueous solution forms an aggregate with the hydrophilic “head” regions in contact with surrounding solvent, sequestering the hydrophobic tail regions in the micelle center. This type of micelle is known as a normal phase micelle (oil-in-water micelle). Inverse micelles have the headgroups at the centre with the tails extending out (water-in-oil micelle).

Micelles are typically smaller in diameter and circumference than liposomes as disclosed herein, and are disclosed in U.S. Pat. Nos. 7,763,271, 7,674,478, 5,510,103, 5,925,720 and U.S. Application 2011/0142884, which are incorporated herein in their entirety by reference. A micelle can be a colloidal aggregate of amphipathic molecules containing both hydrophilic and hydrophobic moieties. In polar media, such as water, the hydrophobic part of the amphiphile forming the micelle tends to locate away from the polar portion, while the polar portion of the molecule also known as the head group tends to locate at the polar micelle water (solvent) interface. On the other hand, micelles may also be formed in non-polar media, such as non-polar organic solvents, e.g., hexane, whereby the amphiphilic cluster around the small water droplets is in the center of the system. In non-polar media, the hydrophobic moieties are exposed to the non-polar media, while the hydrophilic portion tends to locate away from the solvent and towards the water droplets. Such an assembly is sometimes referred to as a reversed micelle. These two aforementioned systems represent water-in-oil and oil-in-water, respectively, types of systems.

The process of forming micellae is known as micellisation. A micelle can be produced where a suspension of an agent, antibody, antibody fragment, integrin ligand or integrin ligand fragment or variant thereof can be encapsulated in micelles to form liposomes by conventional methods (U.S. Pat. No. 5,043,164, U.S. Pat. No. 4,957,735, I5 U.S. Pat. No. 4,925,661; Connor and Huang, (1985) J. Cell Biol. 101: 581; Lasic D. D. (1992) Nature 355: 279; Novel Drug Delivery (eds. Prescott and Nimmo, Wiley, New York, 1989); Reddy et al. (1992) J. Immunol. 148:1585), which are incorporated herein in their entirety by reference.

Micelles are approximately spherical in shape. Other phases, including shapes such as ellipsoids, cylinders, and bilayers are also possible, depending on the conditions and the composition of the system, as the shape and size of a micelle is a function of the molecular geometry of its surfactant molecules and solution conditions such as surfactant concentration, temperature, pH, and ionic strength. For example, small micelles in dilute solution at approximately the critical micelle concentration (CMC) are generally believed to be spherical. However, under other conditions, they may be in the shape of distorted spheres, disks, rods, lamellae, and the like.

For example, U.S. Pat. No. 5,929,177 to Kataoka, et al. describes a polymeric molecule which is usable as, inter alia, a drug delivery carrier. The micelle can be formed from a block copolymer having functional groups on both of its ends and which comprises hydrophilic/hydrophobic segments. The polymer functional groups on the ends of the block copolymer include amino, carboxyl and mercapto groups on the α-terminal and hydroxyl, carboxyl group, aldehyde group and vinyl group on the .omega.-terminal. The hydrophilic segment comprises polyethylene oxide, while the hydrophobic segment is derived from lactide, lactone or (meth)acrylic acid ester.

In some embodiments, a carrier particle used to deliver the agent is a dendrimer. Dendrimers are precisely defined, synthetic nanomaterials that are approximately 5-10 nanometres in diameter. They are made up of layers of polymer surrounding a central core. In particular, dendrimers are branched macromolecules are constructed around a simple core unit. They have a high degree of molecular uniformity, narrow molecular weight distribution, specific size and shape characteristics, and a highly-functionalized terminal surface. The manufacturing process is a series of repetitive steps starting with a central initiator core. Each subsequent growth step represents a new “generation” of polymer with a larger molecular diameter, twice the number of reactive surface sites, and approximately double the molecular weight of the preceding generation.

Dendrimers attracted as nano carriers due to their size, possible encapsulation of drugs in the core of the dendrimer, and chemical conjugation of drugs, solubilizing groups (including polyoxyethylene glycol), and ligands to the surface of dendrimers making them ideal nanocarriers for drug delivery. In some embodiments, the surface of a dendrimer contains many different sites to which drugs or agents may be attached and also attachment sites for materials such as polyethylene glycol (PEG) which can be used to modify the way the dendrimer interacts with the body. PEG can be attached to the dendrimer to ‘disguise’ it and prevent the body's defense mechanisms from detecting it, thereby slowing the process of breakdown. This allows the delivery system to circulate in the body for an extended time period, maximizing the opportunities for the drug to reach the relevant sites.

Dendrimers for use as carriers of agents to be delivered to the target spinal anatomies as disclosed herein are disclosed in U.S. Pat. Nos. 7,316,845; 7,390,407; 7,405,042; 7,320,963; 7,354,969; 7,384,626; 7,425,582; 7,459,146; and 7,432,239, which are all incorporated herein in their entirety by reference.

c. Liposomes

In some embodiments a carrier particle is a liposome which is used to capture and deliver an agent to the targeted spinal anatomy using the methods and devices herein. Liposomes are microscopic spheres having an aqueous core surrounded by one or more outer layers made up of lipids arranged in a bilayer configuration (see, generally, Chonn et al., Current Op. Biotech. 1995, 6, 698-708). Liposomes are non-toxic, non-hemolytic and non-immunogenic even upon repeated injections; they are biocompatible and biodegradable. Lipid based, ligand coated nanocarriers can store their payload in the hydrophobic shell or the hydrophilic interior depending on the nature of the drug/contrast agent being carried.

Liposomes are completely closed lipid bilayer membranes containing an entrapped aqueous volume. Liposomes may be unilamellar vesicles possessing a single membrane bilayer or multilameller vesicles, onion-like structures characterized by multiple membrane bilayers, each separated from the next by an aqueous layer. In one preferred embodiment, the liposomes of the present invention are unilamellar vesicles. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

The liposome particles may be of any suitable structure, such as unilamellar or plurilamellar, so long as the agent is contained therein. Positively charged lipids such as N—[I-(2,3dioleoyloxi)propyl]-N,N,N-trimethyl-anunoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757 which are incorporated herein by reference. Other non-toxic lipid based vehicle components may likewise be utilized to facilitate uptake of the agent carried (e.g., encapsulated or on the outside of the carrier particle) by the pancreatic islet endothelial cell.

Liposomes useful in the methods and compositions as disclosed herein can be produced from combinations of lipid materials well known and routinely utilized in the art to produce liposomes. Lipids can include relatively rigid varieties, such as sphingomyelin, or fluid types, such as phospholipids having unsaturated acyl chains. “Phospholipid” refers to any one phospholipid or combination of phospholipids capable of forming liposomes. Phosphatidylcholines (PC), including those obtained from egg, soy beans or other plant sources or those that are partially or wholly synthetic, or of variable lipid chain length and unsaturation are suitable for use in the present invention.

Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in this invention. All of these phospholipids are commercially available. Further, phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in formulations. Other suitable phospholipids include phosphatidylethanolamines, phosphatidylinositols, sphingomyelins, and phosphatidic acids containing lauric, myristic, stearoyl, and palmitic acid chains. For the purpose of stabilizing the lipid membrane, it is preferred to add an additional lipid component, such as cholesterol. Preferred lipids for producing liposomes according to the invention include phosphatidylethanolamine (PE) and phosphatidylcholine (PC) in further combination with cholesterol (CH). According to one embodiment of the invention, a combination of lipids and cholesterol for producing the liposomes of the invention comprise a PE:PC:Chol molar ratio of 3:1:1. Further, incorporation of polyethylene glycol (PEG) containing phospholipids is also contemplated by the present invention.

Liposomes useful in the methods and compositions as disclosed herein can be obtained by any method known to the skilled artisan. For example, the liposome preparation of the present invention can be produced by reverse phase evaporation (REV) method (see U.S. Pat. No. 4,235,871), infusion procedures, or detergent dilution. A review of these and other methods for producing liposomes can be found in the text Liposomes, Marc Ostro, ed., Marcel Dekker, Inc., New York, 1983, Chapter 1. See also Szoka Jr. et al., (1980, Ann. Rev. Biophys. Bioeng., 9:467). A method for forming ULVs is described in Cullis et al., PCT Publication No. 87/00238, Jan. 16, 1986, entitled “Extrusion Technique for Producing Unilamellar Vesicles”. Multilamellar liposomes (MLV) can be prepared by the lipid-film method, wherein the lipids are dissolved in a chloroform-methanol solution (3:1, vol/vol), evaporated to dryness under reduced pressure and hydrated by a swelling solution. Then, the solution is subjected to extensive agitation and incubation, e.g., 2 hour, e.g., at 37° C. After incubation, unilamellar liposomes (ULV) are obtained by extrusion. The extrusion step modifies liposomes by reducing the size of the liposomes to a preferred average diameter.

In some embodiments, liposomes of the desired size can be selected using techniques such as filtration or other size selection techniques. While the size-selected liposomes of the invention should have an average diameter of less than about 300 nm, it is preferred that they are selected to have an average diameter of less than about 200 nm with an average diameter of less than about 100 nm being particularly preferred. When the liposome of the present invention is a unilamellar liposome, it preferably is selected to have an average diameter of less than about 200 nm. The most preferred unilamellar liposomes of the invention have an average diameter of less than about 100 nm. It is understood, however, that multivesicular liposomes of the invention derived from smaller unilamellar liposomes will generally be larger and can have an average diameter of about less than 1000 nm. Preferred multivesicular liposomes of the invention have an average diameter of less than about 800 nm, and less than about 500 nm while most preferred multivesicular liposomes of the invention have an average diameter of less than about 300 nm.

In some embodiments, the outer surface of the liposomes can be modified with a long-circulating agent, e.g., PEG, e.g., hyaluronic acid (HA). The modification of the liposomes with a hydrophilic polymer as the long-circulating agent is known to enable to prolong the half-life of the liposomes in the blood. Examples of the hydrophilic polymer include polyethylene glycol, polymethylethylene glycol, polyhydroxypropylene glycol, polypropylene glycol, polymethylpropylene glycol and polyhydroxypropylene oxide. In one embodiment, a hydrophilic polymer is polyethylene glycol (PEG). Glycosaminoglycans, e.g., hyaluronic acid, can also be used as long-circulating agents.

The liposomes can be modified with a cryoprotectant, e.g., a sugar, such as trehalose, sucrose, mannose or glucose, e.g., HA. In some embodiments, a liposome is coated with HA. HA acts as both a long-circulating agent and a cryoprotectant. The liposome is modified by attachment of the targeting moiety. In another embodiment, a targeting molecule, can be covalently attached to HA, which is bound to the liposome surface. Alternatively, a carrier particle is a micelle. Alternatively, the micelle is modified with a cryoprotectant, e.g., HA, PEG.

A method for coating the liposomes or other polymeric nanoparticles with a targeting molecule are disclosed in U.S. Provisional Application No. 60/794,361 filed Apr. 24, 2006, and International Patent Application: PCT/US07/10075 filed Apr. 24, 2007 with are incorporated in their entirety herein by reference.

In one embodiment, the agents can be delivered in carrier particles which are immunoliposomes for targeting to particular cell types within the target spinal anatomy, where a targeting molecule is associated with a carrier particle and the carrier particle comprises at least one agent.

In one embodiment, liposomes may be stored in a lyophilized condition prior to encapsulation of drug or agent, or prior to the attachment of at least one a targeting molecule.

Any suitable lipid: pharmaceutical agent ratio that is efficacious is contemplated by the present invention. In some embodiments, the lipid: pharmaceutical agent molar ratios include about 2:1 to about 30:1, about 5:1 to about 100:1, about 10:1 to about 40:1, about 15:1 to about 25:1.

In some embodiments, the loading efficiency of therapeutic or pharmaceutical agent is a percent encapsulated pharmaceutical agent of about 50%, about 60%, about 70% or greater. In one embodiment, the loading efficiency for a soluble agent is a range from 50-100%. In some embodiments, the loading efficiency of an insoluble agent to be associated with the lipid portion of the lipid particle, (i.e. a pharmaceutical agent poorly soluble in aqueous solution), is a percent loaded pharmaceutical agent of about 50%, about 60%, about 70%, about 80%, about 90%, about 100%. In one embodiment, the loading efficiency for a hydrophobic agent in the lipid layer is a range from 80-100%.

In some embodiments, a liposome can comprise multiple layers that assembled in a step-wise fashion, where each layer can comprise at least one agent to be delivered to the target spinal anatomy. In one embodiment, the first step is the preparation of empty nano-scale liposomes. Liposomes may be prepared by any method known to the skilled artisan. The second step is the addition of an agent to the first layer. The first layer is added to the liposome by covalent modification. In some embodiments, the first layer comprises hyaluronic acid, or other cryoprotectant glucosaminoglycan. A liposome composition may also be lyophilized and reconstituted at any time after the addition of the first layer. The third step is to add a second surface modification. The second layer is added by covalent attachment to the first layer. The second layer comprises at least one targeting molecule. Further layers may add to the liposome and these layers may include additional agents and/or a targeting molecule. Alternatively, the second layer may include a heterogeneous mix of a targeting molecule as well as agents. The liposome composition can be lyophilized after addition of the final targeting layer. An agent of interest to be delivered to the target spinal anatomy can be encapsulated by the liposome by rehydration of the liposome with an aqueous solution containing the agent. In one embodiment, agents that are poorly soluble in aqueous solutions or agents that are hydrophobic may be added to the composition during preparation of the liposomes in step one.

The term “stabilized liposome” as used herein refers to a liposome that comprises a cryoprotectant and/or a long-circulating agent.

The terms “encapsulation” and “entrapped,” as used herein, refer to the incorporation of an agent in a lipid particle. An agent can be present in the aqueous interior of the lipid particle, for example a hydrophilic agent. In one embodiment, a portion of the encapsulated agent takes the form of a precipitated salt in the interior of the liposome. The agent may also self precipitate in the interior of the liposome. In alternative embodiments, an agent can be incorporated into the lipid phase of a carrier particle, for example a hydrophobic and/or lipophilic agent.

The term “lipid particle” refers to lipid vesicles such as liposomes or micelles.

The term “hydrophilic” as used herein refers to a molecule or portion of a molecule that is typically charge-polarized and capable of hydrogen bonding, enabling it to dissolve more readily in water than in oil or other hydrophobic solvents. Hydrophilic molecules are also known as polar molecules and are molecules that readily absorb moisture, are hygroscopic, and have strong polar groups that readily interact with water. A “hydrophilic” polymer as the term is used herein, has a solubility in water of at least 100 mg/ml at 25° C.

The term “soluble agent” or “hydrophilic agent” and “hydrophilic agent” are used interchangeably herein, refers to any organic or inorganic compound or substance having biological or pharmacological activity and adapted or used for a therapeutic purpose having a water solubility greater than 10 mg/ml.

The term “hydrophobic” as used herein refers molecules tend to be non-polar and prefer other neutral molecules and non-polar solvents. Hydrophobic molecules in water often cluster together. Water on hydrophobic surfaces will exhibit a high contact angle. Examples of hydrophobic molecules include the alkanes, oils, fats, and greasy substances in general. Hydrophobic materials are used for oil removal from water, the management of oil spills, and chemical separation processes to remove non-polar from polar compounds. Hydrophobic molecules are also known as non-polar molecules. Hydrophobic molecules do not readily absorb water or are adversely affected by water, e.g., as a hydrophobic colloid. A “hydrophobic” polymer as the term is used herein has a solubility in water less than 10 mg/ml at 25° C., preferably less than 5 mg/ml, less than 1 mg/ml or lower.

The term “lipophilic” as used herein is used to refer to a molecule having an affinity for lipid molecules or fat molecules, pertaining to or characterized by lipophilia. Lipophilic or fat-liking molecules refers to molecules with an ability to dissolve in fats, oils, lipids, and non-polar solvents, for example such as hexane or toluene. Lipophilic substances tend to dissolve in other lipophilic substances, while hydrophilic (water-loving) substances tend to dissolve in water and other hydrophilic substances. Lipophilicity, hydrophobic and non-polarity (the latter as used to describe intermolecular interactions and not the separation of charge in dipoles) all essentially describe the same molecular attribute; the terms are often used interchangeably

The term “insoluble agent” or “hydrophobic agent” or “hydrophobic drug” are used interchangeably herein, refers to any organic or inorganic compound or substance having biological or pharmacological activity and adapted or used for a therapeutic purpose having a water solubility of less than 10 mg/ml. Typically an insoluble agent is an agent which is water insoluble, poorly water soluble, or poorly soluble in such as those agents having poor solubility in water at or below normal physiological temperatures, that is having at least less than 10 mg/ml, such as about <5 mg/ml at physiological pH (6.5-7.4), or about <1 mg/ml, or about <0.1 mg/ml.

The term “aqueous solution” as used herein includes water without additives, or aqueous solutions containing additives or excipients such as pH buffers, components for tonicity adjustment, antioxidants, preservatives, drug stabilizers, etc., as commonly used in the preparation of pharmaceutical formulations.

d. Virosomes

In some embodiments, an agent to be delivered using the devices, systems and methods as disclosed herein is encapsulated in a virosome. Virosomes are a carrier particle comprising lipid bilayers containing viral glycoproteins derived from enveloped viruses. Virosomes (or virosome-like-particles, considering that the exact size and shape of the particles) are generally produced by extraction of membrane proteins and lipids from enveloped viruses with a detergent, followed by removal of this detergent from the extracted lipids and viral membrane proteins, in fact reconstituting or reforming the characteristic lipid bilayers (envelopes) that surround the viral core or nucleocapsid.

The term “virosome” defines a specific form of virus-like particles (YLPs). Virosomes are semi-synthetic complexes derived from viral particles and produced by an in vitro procedure. They are essentially reconstituted viral coats, while the viral nucleocapsid is replaced by a compound of choice. Virosomes retain their fusogenic activity and thus deliver the incorporated compound (antigens, agents, genes) inside the target cell. They can be used for vaccines, agent delivery, or gene transfer.

Virus-like particles (VLPs) are particle structures that are in size and shape reminiscent of or even indistinguishable from their parental virus but are lacking the capability to infect and replicate in host cells. VLPs are multimeric structures composed of viral proteins (authentic or modified variants of it). In addition, VLPs may or may not contain nucleic acids, lipids, and include lipid membrane structures or not. Two typical but very distinct examples for VLPs derived from a single Virus (HBV) are HBs and HBc particles.

Virosomes are unilamellar phospholipid bilayer vesicles incorporating virus derived proteins to allow the virosomes to fuse with target cells. Virosomes are not able to replicate but are pure fusion-active vesicles. In contrast to liposomes, virosomes contain functional viral envelope glycoproteins, for example, influenza virus hemagglutinin (HA) and neuraminidase (NA) intercalated in the phospholipid bilayer membrane. Virosomes typically have a mean diameter of 150 nm, and without being limited to theory, virosomes represent reconstituted empty influenza virus envelopes, devoid of the nucleocapsid including the genetic material of the source virus.

The unique properties of virosomes partially relate to the presence of biologically active influenza HA in their membrane. This viral protein not only confers structural stability and homogeneity to virosome-based formulations, but it significantly contributes to the immunological properties of virosomes, which are clearly distinct from other liposomal and proteoliposomal carrier systems.

Virosome can be produced by solubilization of viral membranes by short-chain phospholipids and purification of the viral membrane components, followed by removal of the short-chain phospholipids. Short-chain phospholipids contain acyl chains with less than twelve carbon atoms each. In one embodiment a short-chain phospholipid is a phosphatidylcholine, e.g., 1,2-diheptanoyl-sn-phosphatidylcholine (DHPC) or 1,2-dicaproyl-sn-phosphatidylcholine (DCPC). A short-chain phospholipid can be produced synthetically or semi-synthetically. Virosomes can also be prepared by the classical detergent-dialysis method using various different compositions of naturally occurring (i.e. medium-chain to long-chain) phospholipids (J. Biochemistry and Molecular Biology, Vol. 35, No. 5 2002, pp 459-464. For example, phospholipids used by Kim Hong Sung et al. were egg PC, having primarily C16 and C18 fatty acyl chains, and dioleoyl-PE, having two C18:1 fatty acyl chains).

Virosomes for use as carriers of agents to be delivered to the target spinal anatomies as disclosed herein are disclosed in International Applications, WO1992/19267; WO1998/52603, U.S. Pat. Nos. 7,901,902; 5,565,203 and U.S. Applications: 2009/0202622; US2009/0087453, and 2006/0228376, which are incorporated herein in their entirety by reference. Additionally, commercially available virosomes can be used, e.g., such as ready-made virosomes (EPAXAL™ or Inflexal™). In some embodiments, the virosomes comprise viral glycoproteins from viruses with high trophism for neuronal cells, e.g., from viruses which have high affinity for and specifically transfect neuronal cells with high affinity and efficicy, e.g., adenovirus particles, herpes simplex virus particles and the like.

e. Mists, Microdroplets, Aerosals, Atomizations

In some embodiments, an agent delivered to the target spinal anatomy by the methods, systems and devices as disclosed herein can be administered in the form of an aerosol or by nebulization, e.g., in the form of a mist, microdroplet, aerosols and atomizations. For use as aerosols, an agent can be present in a solution or suspension and can be connected to a pressurized aerosol present in the device, and can be delivered with a suitable propellant, for example, air, hydrocarbon propellants like propane, butane, or isobutane with conventional adjuvants. An agent can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. When the active ingredients are adapted to be administered, either together or individually, via nebulizer(s) they can be in the form of a nebulized aqueous suspension or solution, with or without a suitable pH or tonicity adjustment, either as a unit dose or multidose device.

Any suitable gas can be used to apply pressure during the nebulization, with preferred gases to date being those which are chemically inert to the agent being delivered. Exemplary gases including, but are not limited to, air, nitrogen, argon or helium.

In some embodiments, an agent can also be administered as an aerosol in the form of a dry powder. For use as a dry powder, a pressure resistant canister or container is filled with a product such as a pharmaceutical composition dissolved in a liquefied propellant or micronized particles suspended in a liquefied propellant so that the correct dosage of the composition is delivered to the patient.

Dry powder aerosols are generally produced with mean diameters primarily in the range of <5 μm. As the diameter of particles exceeds 3 μm, there is increasingly less phagocytosis by macrophages. The powder compositions can be administered via an aerosol dispenser or encased in a breakable capsule which can punctured to blow the powder out in a steady stream along the catheter to the target spinal cord location. The compositions can include propellants, surfactants, and co-solvents and may be filled into aerosol containers that are closed by a suitable metering valve.

Aerosols are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S, and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

In some embodiments, the agents delivered by the devices, systems and methods as disclosed herein are in the form of microdroplets. Microdroplets, originally called monolayer vesicles, consist of spheres of organic liquid phase agent approximately 500 Angstroms in diameter and range from 200 Angstroms up to at least one micron (10,000 Angstroms) in diameter and are covered with a monolayer of a suitable phospholipid. Microdroplets are distinguished from liposomes (multilamellar-) and unilamellar phospholipid vesicles, which consist of a spherical lipid bilayer with an aqueous phase inside.

Microdroplets can be used to deliver any water-insoluble/oil-soluble agent compound or agent. The organic liquid phase may be the drug or agent itself. The advantages of the microdroplets include a relatively slow release of the agent substance to the tissues and allow for a targeted delivery with lowered metabolic degradation, first pass effects, and low toxic side-effects in the liver and other organs.

Microparticles for use can be phospholipid stabilized aqueous suspension of submicron sized particles of the agent (see U.S. Pat. Nos. 5,091,187; 5,091,188 and 5,246,707) and microdroplets that are phospholipid stabilized oil in water emulsion by dissolving the agent in a suitable bio-compatible hydrophobic carrier (see U.S. Pat. Nos. 4,622,219 and 4,725,442), which are incorporated herein in their entirety by reference. Microparticles can be produced using the device as disclosed in U.S. Pat. Nos. 6,576,264; 5,624,608; and 6,974,593 which are incorporated herein in their entirety by reference. Microdroplets can form a mist which is delivered to a target spinal anatomy by the methods and devices as disclosed herein.

f. Gels

In some embodiments, the agent is comprised of a gel. A gel is a substantially dilute cross-linked system which resembles a solid in steady state. By weight, gels are mostly liquid, yet they behave like solids due to a three-dimensional cross-linked network within the liquid. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel).

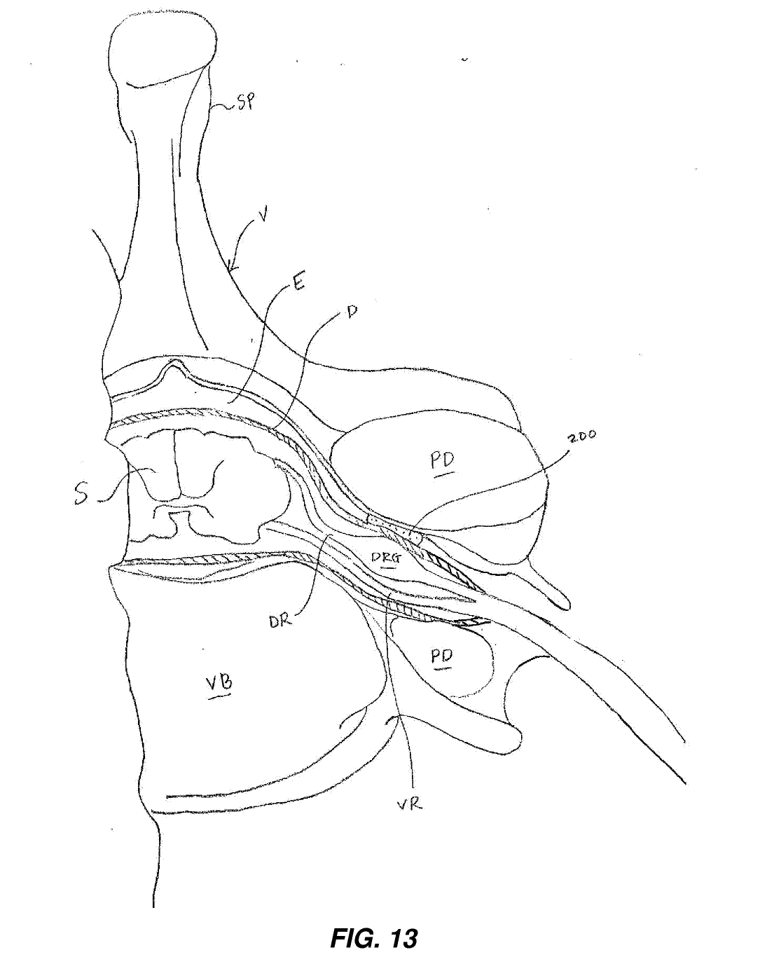

The agent may be delivered to the target tissue site, such as on, near, about or adjacent to the DRG, in a gel form or in a liquid form that gels at the target site. FIG. 13 illustrates a gel 200 delivered to the epidural space E adjacent to the target DRG. In this example, the gel 200 is delivered by the methods illustrated in FIG. 12. Gelling of the agent may be achieved by a variety of techniques, including light activation, electrical activation, temperature activation, and pH activation, to name a few.

Typically, light is delivered by the device through which the agent is delivered, such as the delivery element. In some embodiments, light is delivered by a separate device. Likewise, electrical energy may be delivered by the device through which the agent is delivered or through a separate device, such as a needle. Temperature activation may be achieved by a change in temperature provided by the natural environment. For example, the agent may be held at a particular temperature and delivery to the target site transitions the temperature of the agent to or toward the natural temperature of the target tissue thereby gelling the agent. Or, temperature activation may be achieved by directly heating or cooling the target site, such as applied by the delivery element. Likewise, pH activation may be achieved by a change in pH provided by the natural environment. For example, the agent may have a particular pH and delivery to or toward the target site transitions the pH of the agent to the natural pH of the target tissue thereby gelling the agent. Or, pH activation may be achieved by directly changing the pH at the target site, such as applied by the delivery element.

Once the gel is delivered to the target tissue site, the network structure maintains the gel at the target site while the agent is delivered, such as in a controlled release manner. Typically, the network structure is biodegradable over time.

1) Biogels

In some embodiments, the gel comprises a biogel gels in vivo and releases protein agents slowly over a sustained period of time. In some instances, the biogel is designed using biocompatible components, sodium carboxymethylcellulose and polyethyleneimine, that electrostatically link to form a gel on exposure to physiological conditions. Typically, the gel is porous enough to release the agent in a slow and controlled manner over a period of up to 15 days, while preventing biological materials from entering. This slow delivery of protein agents enhances their therapeutic benefits.

2) Nanofiber Hydrogel Scaffold

In some embodiments, the agent comprises a nanofiber hydrogel scaffold. Such a gel is comprised of small, woven protein fragments which can successfully carry and release proteins of different sizes. The rate of release can be controlled by changing the density of the gel, allowing for continuous agent delivery over a specific period of time. The proteins are released from the gel over hours, days or even months and the gel itself is eventually broken down into harmless amino acids. Such peptide hydrogels are ideally suited for agent delivery as they are pure, easy to design and use, non-toxic, non-immunogenic, bio-absorbable, and can be locally applied to a particular tissue. In addition, proteins carried by the gel emerge unscathed after delivery, with no adverse affect on their function.

3) Injectable In Situ Forming Gel

In some embodiments, the agent delivered to the target tissue forms a gel in situ. The gel is then able to provide controlled delivery of the agent to the target tissue over time. Since the agent is injectable, the agent can be stored in an agent delivery module and delivered to the target tissue with the use of delivery elements such as described above. The agent does not form a gel until it has been injected from the delivery element to the target tissue area.

In some embodiments, the agent comprises chitosan. Chitosan is a biocompatible pH dependent cationic polymer obtained by alkaline deacetylation of chitin, a natural component of shrimp and crab shell. Chitosan remains dissolved in aqueous solutions up to a pH of 6.2. Neutralization of chitosan aqueous solution to a pH exceeding 6.2 leads to the formation of a hydrated gel like precipitate. The pH gelling cationic polysaccharides solution are transformed into thermally sensitive pH dependent gel forming aqueous solutions without any chemical modification or cross linking by addition of polyol salts bearing a single anionic head such as glycerol, sorbitol, fructose or glucose phosphate salts to chitosan aqueous solution. This transformation causes the chitosan to be biodegradable and thermosensitive. The formulation is in the SOL form at room temperature, in which living cells and therapeutic proteins can be incorporated. This formulation, when injected in vivo, turns into gel implants in situ.

In other embodiments, the agent comprises an in situ cross linked system where the polymers form cross linked networks by means of free radical reactions that may occur by means of light (photopolymerizable systems) or heat (thermosetting systems). Photopolymerizable systems when introduced to the desired site via injection are photocured in situ with the use of fiber optic cables (such as within the delivery element) and then release the agent for prolonged period of time. The photo-reactions provide rapid polymerization rates at physiological temperature. Furthermore, the systems are easily placed in complex shaped volumes leading to an implant formation. In some embodiments, the photopolymerizable, biodegradable hydrogel is comprised of a macromer (PEG-oligoglycolyl-acrylate), a photosensitive initiator (eosin dye) and is used with a light source (UV or visible light). When exposed to light, the system undergoes photopolymerization to form a network. These systems can be used to release water soluble agents and enzymes at a controlled rate. Argon laser can also be used as a light source.

In other embodiments, the agent is in the sol form when initially constituted, but upon heating, it sets into its final shape. This sol-gel transition is known as curing. Curing mainly involves the formation of covalent cross links between polymer chains to form a macromolecular network. In some embodiments, the agent comprises biodegradable copolymers of DL-lactide or L-lactide with ε-caprolactone implant and slow release agent delivery. The agent is liquid outside the body and is capable of being injected through a needle or delivery element 30 and once inside the body it gels. In in situ precipitating polymeric systems, the polymer precipitation from solution may lead to gel formation in situ and this precipitation can be induced by change in temperature (thermosensitive systems), solvent removal or by change in pH.

In some embodiments, the agent comprises sucrose acetate isobutyrate (SAIB) which is a non crystalline, viscous compound that gets dissolved in some of the organic solvents such as dimethylsulphoxide. SAIB, a sucrose molecule esterified with two acetic acid and six isobutyric acid moieties, is a highly lipophilic, water insoluble sugar and exists as a very viscous liquid. SAIB forms a low viscosity solution when dissolved in organic solvents such as ethanol, NMP, triacetin, and propylene carbonate, which is mixed with active ingredient prior to administration. Once administered, the solvent diffuses out leading to the formation of depot for controlled delivery of active ingredient. The concentration of SAIB, type of solvent, and additives used affect release rate of agent from depot formed in situ.

g. Artificial DNA Nanostructure

In some embodiments, the agent comprises an artificial DNA nanostructure. An artificial DNA nanostructure is DNA that is used as a structural material rather than as a carrier of genetic information. DNA nanotechnology makes use of the fact that, due to the specificity of Watson-Crick base pairing, only portions of the strands which are complementary to each other will bind to each other to form duplex DNA. DNA nanotechnology attempts to rationally design sets of DNA strands so that desired portions of each strand will assemble in the correct positions for some desired target structure, a process called nucleic acid design.

It may be appreciated that the principles of DNA nanotechnology apply equally well to other nucleic acids such as RNA and PNA and may be used in a similar fashion as an agent as described herein.

h. Biologic Vectors

In some embodiments, an agent which is delivered to the target spinal anatomies, e.g., the DRG using the devices, methods and systems as disclosed herein is present in a biological vector. Technologies for the administration of agents in a vector which comprises a nucleic acid encoding a protein agent are well known in the art.

A variety of methods of using a biological vector for delivery of agents such as proteins and/or nucleic acids can be used for the delivering an agent to the target spinal anatomy cells, e.g., DRG cells, in a subject using the devices, systems and methods as disclosed herein and include without limitation, cellular transfection, gene therapy, direct administration with a delivery vehicle or pharmaceutically acceptable carrier, indirect delivery by providing recombinant cells comprising a nucleic acid encoding a polypeptide agent, lipofection, electroporation, biolistics, chromosome-mediated gene transfer, microcell-mediated gene transfer, nuclear transfer, and the like.

A wide variety of gene transfer/gene therapy vectors and constructs are known in the art. These vectors are readily adapted for use in the devices, systems and methods of the present invention. By the appropriate manipulation using recombinant DNA/molecular biology techniques to insert an operatively linked nucleic acid encoding a protein agent or a functional fragment, or a functional variant or derivative thereof into the selected expression/delivery vector, many equivalent vectors for the practice of the methods described herein can be generated. A vector containing a nucleic acid molecule of the invention linked to expression control elements and capable of replicating inside the cells is prepared. Alternatively the vector can be replication deficient and can require helper cells for replication and use in gene therapy.

Vectors, recombinant viruses, and other expression systems can comprise any nucleic acid which can infect, transfect, transiently or permanently transduce a neuronal cell or neuronal support cell, e.g., glia, astrocytes and the like. In one aspect, a vector can be a naked nucleic acid, or a nucleic acid complexed with protein or lipid. In one aspect, a vector can comprise viral or bacterial nucleic acids and/or proteins, and/or membranes (e.g., a cell membrane, a viral lipid envelope, etc.). In one aspect, expression systems can be replicons (e.g., RNA replicons, bacteriophages) to which fragments of DNA may be attached and become replicated. In one aspect, expression systems also include, but are not limited to RNA, autonomous self-replicating circular or linear DNA or RNA (e.g., plasmids, viruses, and the like, see, e.g., U.S. Pat. No. 5,217,879), and include both the expression and non-expression plasmids.

In one aspect, a vector can be an expression vector including both (or either) extra-chromosomal circular and/or linear nucleic acid (DNA or RNA) that has been incorporated into the host chromosome(s). In one aspect, where a vector is being maintained by a host cell, the vector may either be stably replicated by the cells during mitosis as an autonomous structure, or is incorporated within the host's genome.

In one aspect, an expression system can be commercially available, publicly available on an unrestricted basis, or can be constructed from available plasmids in accord with published procedures. Plasmids that can be used to practice this invention are well known in the art.

Another approach is introducing a gene or nucleic acid sequence into cells by such methods as electroporation, lipofection, calcium phosphate mediated transfection, or viral infection. U.S. Pat. No. 5,676,954 (which is herein incorporated by reference) reports on the injection of genetic material such as naked DNA, complexed with cationic liposome carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are all herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein all incorporated by reference) provide methods for delivering DNA-cationic lipid complexes to mammals. Accordingly, in some embodiments, such cationic lipid complexes or nanoparticles as disclosed herein can be used to deliver a nucleic acid encoding a protein agent of interest to the target spinal anatomy, e.g., DRG in a subject.

In some embodiments, the electrical stimulation portion of the device can be used to introduce naked DNA, e.g., a nucleic acid encoding an agent of interest into neuronal cells at the target spinal anatomy location (e.g., DRG) by electroporation, adapting the parameters for electroporation using the device of the present invention, based on prior parameters set forth in Wong and Neumann, Biochem. Biophys. Res. Commun. 107:584-87 (1982)) and biolistics (e.g., a gene gun; Johnston and Tang, Methods Cell Biol. 43 Pt A:353-65 (1994); Fynan et al., Proc. Natl. Acad. Sci. USA 90:11478-82 (1993).

In certain embodiments, a gene or nucleic acid sequence encoding a protein agent can also be introduced into the target spinal anatomy cells, e.g., DRG cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

In another aspect, constructs encoding the agent can be inserted into the genome of a host cell by e.g., a vector. A nucleic acid sequence can be inserted into a vector, e.g., viral vector by a variety of procedures. In general, the sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

In alternative aspects, a vector used to make or practice the invention can be chosen from any number of suitable vectors, including cosmids, YACs (Yeast Artificial Chromosomes), megaYACS, BACs (Bacterial Artificial Chromosomes), PACs (P1 Artificial Chromosome), MACs (Mammalian Artificial Chromosomes), a whole chromosome, or a small whole genome. The vector also can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook. Particular bacterial vectors which can be used include the commercially available plasmids comprising genetic elements of the well known cloning vector pBR322 (ATCC 37017), pKK223-3 (Pharmacia Fine Chemicals, Uppsala, Sweden), GEMI (Promega Biotec, Madison, Wis., USA) pQE70, pQE60, pQE-9 (Qiagen), pD10, psiX174 pBluescript 11 KS, pNII8A, pN1-116a. pN1118A, pNI-146A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

In some embodiments, a nucleic acid encoding a protein agent is administered to the target spinal anatomy, e.g., DRG cells present in a vector. In some embodiments, the concentration of virus or vector particle comprising a nucleic acid encoding an agent of interest is formulated at a titer of about at least 10¹⁰, 10¹¹, 10¹², 10¹³, 10¹⁴, 10¹⁵, 10¹⁶, or 10¹⁷ physical particles per milliliter. In one aspect, a nucleic acid encoding an agent of interest is administered in about 10, 20, 30, 40, 50, 60, 70, 80, 90, 100, 110, 120, 130, 140 or 150 or more microliter (μl) injections.

In alternative embodiments, it may be appropriate to administer multiple applications to the target spinal neurons, e.g., DRG to ensure sufficient exposure of target neurons to the nucleic acid encoding the agent of interest. In some embodiments, multiple applications of the expression construct may also be required to achieve the desired effect.

Doses and dosage regimens can be determined by a variety of range-finding techniques. For example, in alternative embodiments, about 10⁶, 10², 10⁸, 10⁹, 10¹⁰, 10¹¹, 10¹², 10¹³, 10¹⁴, 10¹⁵, 10¹⁶ or 10¹⁷ viral (e.g., Adenovirus) particles are delivered to the individual (e.g., a human patient) in one or multiple doses. In some embodiments, about 2×10⁷, or about 2×10⁶, or about 2×10⁵, particles are delivered to the individual (e.g., a human patient) in one or multiple doses.

In other embodiments, the volume of a vector composition encoding a protein agent can be administration to the target spinal neurons, e.g., DRG can be from about 0.1 μl to 1.0 μl to about 10 μl or to about 100 μl or more than 100 μl. Alternatively, dosage ranges from about 0.5 ng or 1.0 ng to about 10 μg, 100 μg to 1000 μg of a nucleic acid encoding an agent of interest is administered (either the amount in an expression construct, or as in one embodiment, naked DNA is injected). Any necessary variations in dosages and routes of administration can be determined.

Viral vector systems which can be utilized to express an agent include, but are not limited to, (a) adenovirus vectors including serotype type 5, e.g., Ad5; (b) retrovirus vectors; (c) adeno-associated virus vectors (AAV), including serotypes AAV5; (d) herpes simplex virus vectors (HSV); (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. In one embodiment, the vector is an adenovirus or an adeno-associated virus, or a Baculovirus. Replication-defective viruses can also be advantageous as well as viruses engineered to bind to or enter neurons. In particular, adenovirus type 5 (Ad-5) as well as viral vectors with enhancements in cell binding and cell entry properties like AdF2K, Adf.11D, and Ad.RGD have demonstrated tropism for DRG cells.

In some embodiments, a vector encoding an agent may or may not be incorporated into the target cells genome. The constructs may include viral sequences for transfection, if desired. Alternatively, the construct may be incorporated into vectors capable of episomal replication, e.g. EPV and EBV vectors.

Constructs for the recombinant expression of an agent which increases the level of the agent generally comprise regulatory elements, e.g., promoters, enhancers, etc., to ensure the expression of the construct in target cells. Other specifics for vectors and constructs are described in further detail below. In some embodiments, the nucleic acid encoding an agent is operatively linked to the regulatory element.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. In some embodiments, it can be advantageous to direct expression of a protein agent in a tissue- or cell-specific manner, e.g., in neuronal cells, or in dorsal root ganglion cells (DRGs). In some embodiments, a neuron specific promoter can be used, for example, but not limited to an enolase promoter or the elongation factor la promoter, neuro filament (NF) gene promoter, Tujl gene promoter, which have demonstrated effective gene expression in spiral ganglion cells, or other neuron-specific promoter known in the art.

In some embodiments, the heterologous promoter allows controlled expression of the agent to be expressed, such as for example, and agent or stress inducible promoter, such as a Tet-inducible system and the like. For example, cells can be engineered to express an endogenous gene encoding the agent under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al, which are incorporated herein in their entirety by reference.

Any viral vectors that contain nucleic acid sequences encoding the agent are encompassed for use herein. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding an agent can be cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993). Any lentiviruses belonging to the retrovirus family can be used for infecting both dividing and non-dividing cells, see e.g., Lewis et al. (1992) EMBO J. 3053-3058.

Viruses from lentivirus groups from “primate” and/or “non-primate” can be used; e.g., any primate lentivirus can be used, including the human immunodeficiency virus (HIV), the causative agent of human acquired immunodeficiency syndrome (AIDS), and the simian immunodeficiency virus (SIV); or a non-primate lentiviral group member, e.g., including “slow viruses” such as a visna/maedi virus (VMV), as well as the related caprine arthritis-encephalitis virus (CAEV), equine infectious anemia virus (EIAV) and/or a feline immunodeficiency virus (FIV) or a bovine immunodeficiency virus (BIV). Details on the genomic structure of some lentiviruses may be found in the art; e.g., details on HIV and EIAV may be found from the NCBI Genbank database, e.g., Genome Accession Nos. AF033819 (HIV) and AF033820 (EIAV). In alternative embodiments, the lentiviral vector of the invention is an HIV-based lentiviral vector or an EIAV-based lentiviral vector.

In alternative embodiments, lentiviral vectors can be pseudotyped lentiviral vectors. In one aspect, pseudotyping incorporates in at least a part of, or substituting a part of, or replacing all of, an env gene of a viral genome with a heterologous env gene, for example an env gene from another virus. Pseudotyping examples may be found in e.g., WO 99/61639, WO 98/05759, WO 98/05754, WO 97/17457, WO 96/09400, WO 91/00047 and Mebatsion et al. (1997) Cell 90:841-847. In alternative embodiments, the lentiviral vector of the invention is pseudotyped with VSV.G. In an alternative embodiment, the lentiviral vector of the invention is pseudotyped with Rabies.G.

Lentiviral vectors used to practice this invention may be codon optimized for enhanced safety purposes. Codon optimization has previously been described in e.g., WO 99/41397. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms. Codon optimization has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimization also overcomes the Rev/RRE requirement for export, rendering optimized sequences Rev independent. Codon optimization also reduces homologous recombination between different constructs within the vector system (for example between the regions of overlap in the gag-pol and env open reading frames). The overall effect of codon optimization is therefore a notable increase in viral titer and improved safety. The strategy for codon optimized gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses. In another embodiment, lentiviral vectors are used, such as the HIV based vectors described in U.S. Pat. Nos. 6,143,520; 5,665,557; and 5,981,276, which are herein incorporated in their entirety by reference.

Other viral vectors can be used and include, adenoviruses, adeno-associated viruses, vaccinia viruses, papovaviruses, lentiviruses and retroviruses of avian, murine and human origin. Adenoviruses are other viral vectors that can be used in gene therapy. Adenoviruses are especially attractive vehicles for adenovirus-based delivery to the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Another preferred viral vector is a pox virus such as a vaccinia virus, for example an attenuated vaccinia such as Modified Virus Ankara (MVA) or NYVAC, an avipox such as fowl pox or canary pox. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995).

Use of Adeno-associated virus (AAV) vectors is also contemplated (Walsh et al., Proc. Soc. Exp. Biol. Med. 204:289-300 (1993); U.S. Pat. No. 5,436,146). Recombinant adeno-associated viral vector (AAV) are described in U.S. Pat. App. Pub. No. 2002/0194630, and U.S. Pat. No. 6,943,153; lentiviral gene therapy vector are described by e.g., Dull et al. (1998) J. Virol. 72:8463-8471; or a viral vector particle, e.g., a modified retrovirus having a modified proviral RNA genome, as described, e.g., in U.S. Pat. App. Pub. No. 2003/0003582; and retroviral or a lentiviral vector as described in U.S. Pat. Nos. 7,198,950; 7,160,727; 7,122,18; 6,555,107 can be used. Recombinant adeno-associated virus (rAAV) vectors are applicable to a wide range of host cells including many different human and non-human cell lines or tissues. Because AAV is non-pathogenic and does not ellicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

The use of AAV serotypes other than AAV-2 (Davidson et al (2000), PNAS 97(7)3428-32; Passini et al (2003), J. Virol 77(12):7034-40) has demonstrated different cell tropisms and increased transduction capabilities. With respect to brain cancers, the development of novel injection techniques into the brain, specifically convection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector. In particular, AAV5 and AAV2 serotype, as well as the adenovirus subtype Ad-5 has demonstrated to be highly efficient at transducing neuronal cells. Accordingly, convention enhanced delivery (CED), which is continuous injection under positive pressure can augment the biological vector delivery mediated transfer of an agent to the target spinal anatomy by the devices as disclosed herein.

3. Agent-Eluting Coating or Structure on Delivery Element

In some embodiments, the agent delivery structure comprises a coating or agent-eluting structure. In such embodiments, the agent is delivered from the coating or agent-eluting structure on the delivery element 30.

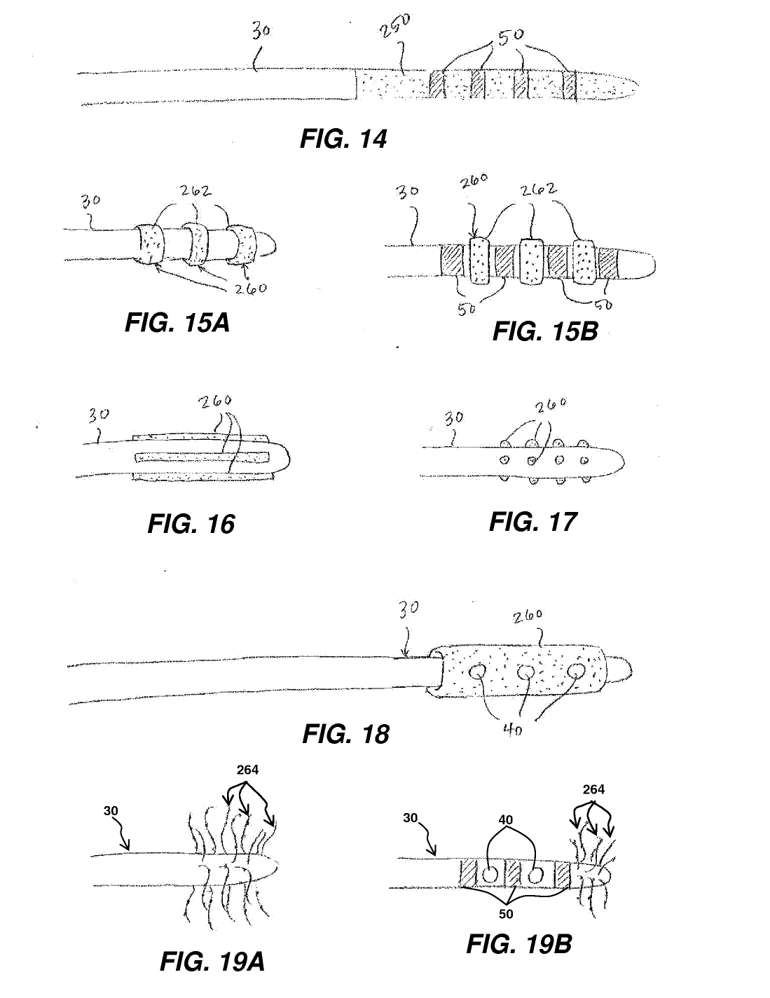

In some embodiments, the delivery element 30 is coated with an agent or an agent-eluting coating. FIG. 14 illustrates an embodiment of a delivery element 30 having electrodes 50 and an agent-eluting coating 250 covering its distal end. Typically the agent-eluting coating 250 is comprised of a polymer matrix that is thin and conformal so as to withstand significant deformations of the delivery element 30. Also, the polymer matrix is typically tailored to incorporate high concentrations of agent and to control the elution of the agent. In some embodiments, a polymer blend is used which offers advantages not found in single polymer coatings, such as the ability to tune elution rates and mechanical properties by varying the ratio of the two polymers.

The coating may be applied to the delivery element 30 by a variety of methods including dipping, spraying and deposition methods that can apply a coating solution or dry materials in very defined, precise patterns. In addition, the coatings may be covalently bonded to the surface of the delivery element 30 or simply adhered to the surface. Likewise, the coatings may be textured to provide various attributes.

The coating may be applied to specific portions of the delivery element 30, such as in longitudinal or circumferential (including partially circumferential) stripes, strips, dots, squares, and/or patches. The coating may be applied between particular electrodes and/or over particular electrodes. The coating may cover the entire distal end of the delivery element, the distal tip or specific portions of the delivery element. In some embodiments, the coating comprises multiple layers or multiple coatings may be used, each containing the same or different agents. The coatings may also be used in combination with other agent delivery techniques to deliver the same or different agents.

In some embodiments, the agent is delivered from a structure on the delivery element. In some embodiments, the structure is comprised of a polymer matrix incorporating the agent and controlling the elution of the agent. Typically, the structure extends from the surface of the delivery element, such as having the form of a raised surface or a protrusion. FIGS. 15A-15B illustrate embodiments having an agent-eluting structure 260 disposed on the surface of the distal end of a delivery element 30. In both embodiments, the structure 260 comprises circumferential stripes or strips 262 that extend around the shaft of the delivery element 30. In FIG. 15A, delivery element 30 comprises a catheter and the strips 262 are spaced apart along the distal end of the delivery element 30. In FIG. 15B, the delivery element 30 comprises a lead having electrodes 50. In this embodiment, the structure 260 comprises circumferential stripes or strips 262 that are disposed between the electrodes. Thus, the agent is eluted near the electrodes 50, such as for use in combination with electrical stimulation. The structure 260 may be disposed along specific portions of the delivery element 30, such as in longitudinal stripes or strips (such as FIG. 16), circumferential (including partially circumferential) stripes or strips, dots (such as FIG. 17), squares, and/or patches. FIG. 18 illustrates an embodiment wherein the delivery element 30 has an agent-eluting structure 260 extending along a portion of its distal end, wherein the structure 260 extends at least partially around the shaft of the delivery element 30 and includes an opening for at least one outlet port 40. Thus, the same or a different agent may be delivered from the delivery element 30 in addition to the agent delivered from the structure 260.

In some embodiments, the agent-eluting structure 260 comprises protrusions such as flexible hair-like protrusions 264 as illustrated in FIGS. 19A-19B. Such protrusions 264 may be comprised of any suitable material including polymers, fibers, microfibers, threads, filaments, or the like. The protrusions 264 may be coated with agent or infused with agent for controlled agent delivery. Typically the protrusions 264 have a first end fixed to the delivery element 30 and second end which is a free end, however it may be appreciated that the second end may also be fixed to the delivery element 30 forming a loop. In any case, the protrusions 264 may also assist in anchoring the delivery element 30 to tissue when implanted, such as near the target tissue, e.g. the DRG. FIG. 19A illustrates an embodiment of a delivery element 30 comprising a catheter having protrusions 264 extending radially outwardly from the shaft of the delivery element 30. FIG. 19B illustrates an embodiment of a delivery element 30 comprising a lead having at least one electrode 50, at least one outlet port 40 and at least one protrusion 264. Here the at least one protrusion 264 comprises a plurality of protrusions extending from the distal tip of the delivery element 30.

In some embodiments, the structure 260 is biodegradable. In such embodiments, the structure 260 may biodegrade in the body over time so that it is eventually eliminated from the implantation site.

4. Agent-Eluting Scaffold

In some embodiments, the agent is delivered from an implantable drug or agent-eluting scaffold which is positioned near the target tissue. The scaffold is comprised of a mesh-like framework having any suitable form, such as a sheet, tube or other shape. In some embodiments, the scaffold is comprised of an expandable metal alloy framework. In such embodiments, the framework typically has a mesh-like design to allow expansion and flexibility. In some instances the framework is comprised of a bare polymer or metal, such as stainless steel, 316L stainless steel, cobalt chrome alloy, L605 cobalt chrome alloy, or the like. When comprised of a bare polymer or metal, the scaffold is typically coated with a controlled release polymer which delivers the agent, such as by contact transfer. Coatings are typically spray coated or dip coated, however any suitable techniques may be used including those described above in relation to coatings. In some embodiments, the coating comprises three or more layers, e.g. a base layer for adhesion, a main layer for holding the agent, and sometimes a top coat to slow down the release of the agent and extend its effect. In other embodiments, the framework itself is formed from a material containing the agent, such as a controlled relased polymer. In such instances, the agent is eluted directly from the framework.

It may be appreciated that the scaffold may elute more than one agent or the same agent at different rates or concentrations. In some embodiments, the framework elutes an agent and a coating on the framework elutes a different agent. In other embodiments, the framework elutes an agent and a coating on the framework elutes the same agent at a different rate. In some embodiments, the scaffolding has a biodegradable coating which elutes an agent until the coating biodegrades away leaving behind the framework which then elutes a different agent upon its exposure.

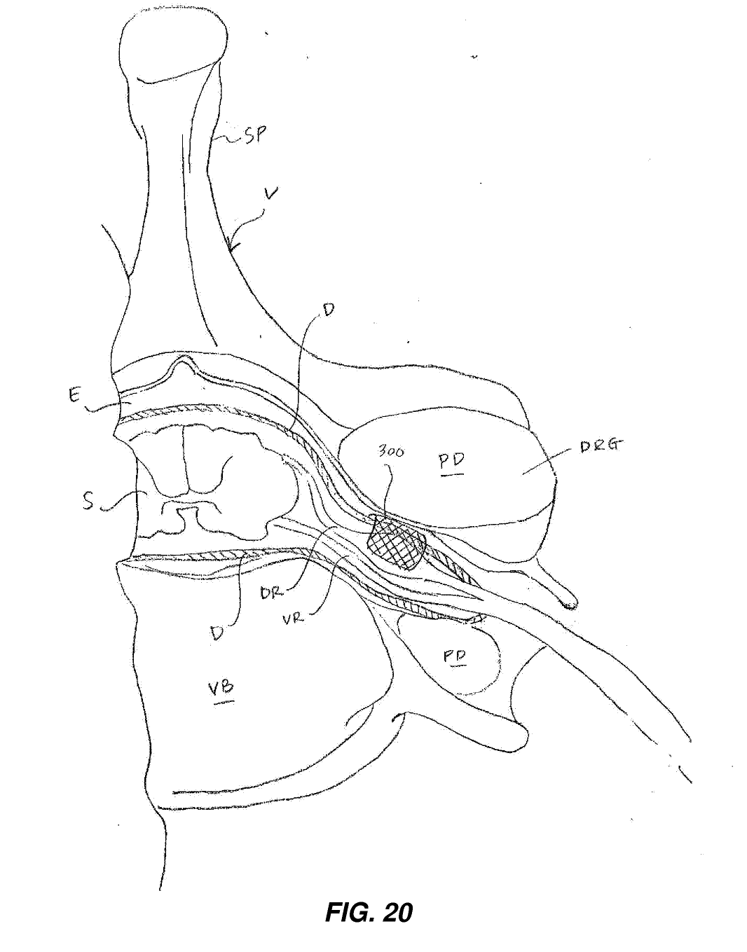

In some embodiments, the scaffold is positionable adjacent the DRG, including in contact with the DRG. When the scaffold has the form of a sheet, the sheet may be aligned with the DRG, such as extending along a surface of the DRG. In some embodiments, the sheet wraps partially or at least partially around the DRG. FIG. 20 illustrates example placement of a sheet 300 positioned adjacent the DRG, wrapping partially around the DRG. In this embodiment, the sheet 300 is positioned within the epidural space E at least partially within a foramen between the pedicles PD as shown. The sheet 300 may be delivered with the use of a delivery element 30 approaching the DRG via an epidural approach through the epidural space. It may also be appreciated that the delivery element 30 may approach the target DRG from outside of the spinal column, such as with an extraforminal approach, wherein the delivery element 30 is advanced into the foramen toward the spinal cord S. Alternatively, the sheet 300 may be delivered using an open procedure or using a variety of minimally invasive devices which directly access the foramen. In any case, the sheet 300 elutes the agent to the epidural space near the DRG.

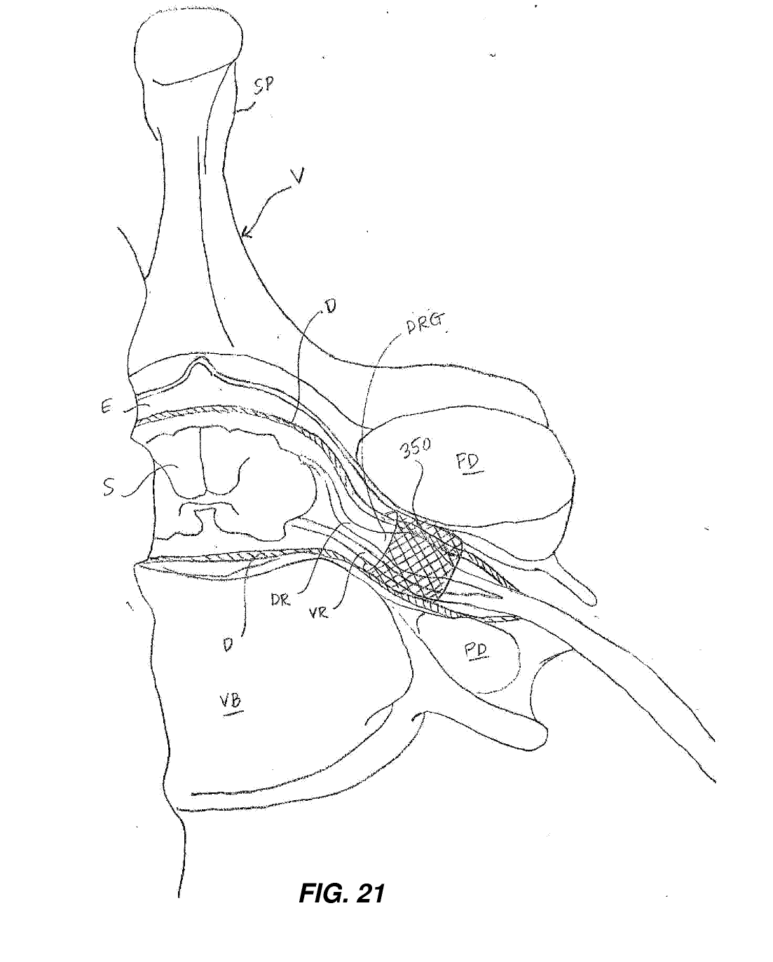

When the scaffold has the form of a tube, the tube may extend around the DRG so that the DRG resides at least partially within the tube. In some embodiments, the scaffold is positionable within a foramen. Such positioning may assist in anchoring the scaffolding in place, such as due to the restricted confines of the foramen, and/or may ensure predictable delivery to the target DRG due to a known anatomical relationship between the foramen and its associated DRG. FIG. 21 illustrates a tube 350 positioned within a foramen, between the pedicles PD, so that the tube 350 extends around the DRG. Since the tube 350 is positioned within the epidural space E, the tube 350 extends along the surface of the dura layer D which surrounds both the DRG and the nearby ventral root VR. However, the agent eluting from the tube 350 may be designed to target only the DRG, may only have an affect on the DRG, and/or may be used in conjunction with stimulation which only affects the DRG, to name a few scenarios. In other instances, delivery of the agent to the ventral root VR does not interfere with successful treatment of the patient.

In any case, the agent-eluting scaffolding delivers the agent according to the specifics of the agent and controlled release mechanism of the scaffolding. Such agent elution is tailored for the disease state or condition of the patient for which the patient is being treated. In some embodiments, when the scaffolding is positioned within a foramen, the target tissue for delivery is the vertebrae of the foramen itself or tissues lining the foramen. For example, such a scaffolding may be used to assist in keeping a foramen patent after a foraminotomy. A foraminotomy is a medical operation used to relieve pressure on nerves that are being compressed by a foramen, the passages through the bones of the vertebrae of the spine that pass nerve bundles to the body from the spinal cord. A foraminotomy is often performed to relieve the symptoms of spinal foraminal stenosis in cases where a nerve root is being compressed by bone, disc, scar tissue, or excessive ligament development resulting in a pinched nerve. The procedure is often performed as a minimally invasive procedure in which a small hole cut into the vertebra itself. Through this hole, using an arthroscope, the foramen can be visualized, and the impinging bone or material removed. By positioning the agent-eluting scaffolding at least partially within the foramen after the foraminotomy, the agent can be delivered within the foramen to assist in maintaining the patency of the foramen, such as by inhibiting tissue growth. Alternatively or in addition, the scaffolding may provide a structural support to assist in maintaining patency of the foramen.

5. Intrathecal Agent Delivery

In some embodiments, the agent is delivered through a delivery element 30 which is placed intrathecally or into the subarachnoid or intrathecal space, such as illustrated in FIG. 22. In such an embodiment, the delivery element 30 is positioned in a manner similar to that described in relation to placement in the epidural space. To begin, the intrathecal space is accessed via traditional methods. The delivery element 30 is then inserted into the intrathecal space and advanced in an antegrade direction within the intrathecal space along the spinal cord S. In this embodiment, the delivery element 30 comprises a catheter having at least one outlet port 40. The delivery element is advanced through the patient anatomy so that at least one of the outlet ports 40 is within a clinically effective distance to the target anatomy, such as the target DRG. Such advancement of the delivery element 30 toward the target DRG in this manner involves making a sharp turn along the nerve root sleeve angulation or angle θ, as illustrated in relation to FIG. 4. A turn of this severity is achieved with the use of a variety of delivery tools and design features of the delivery elements 30, such as were described in relation to FIGS. 8A-8D. In some embodiments, a flexible sheath may be used which is similar to that which is illustrated and described in relation to FIG. 8B. However, such a sheath used intrathecally will typically be more flexible than such a sheath used epidurally to reduce any risk of damaging the spinal cord or neural tissues. When used, the sheath has a distal end which is pre-curved to have an angle α. In some instances, the angle α is in the range of approximately 80 to 165 degrees. Passage of the sheath over the delivery element 30 causes the delivery element 30 to bend in accordance with the precurvature of the sheath. Thus, the sheath assists in steering the delivery element 30 along the spinal column S and toward a target DRG, such as in a lateral direction. However, steering of the delivery element 30 within the intrathecal space toward the target DRG is assisted by the spinal anatomy wherein the dura layer may assist in directing the delivery element 30 toward the target DRG. In such instances, a sheath is not needed and the delivery element 30 may be guided toward the target DRG with the use of an internal stylet, such as described and illustrated in relation to FIG. 8C. In some embodiments, the stylet has a distal end which is pre-curved, such as so that its radius of curvature is in the range of approximately 0.1 to 0.5 inches. The stylet is sized and configured to be advanced within a stylet lumen of the delivery element 30. Typically the stylet extends therethrough so that its distal end aligns with the distal end of the delivery element 30. Passage of the stylet through the delivery element 30 causes the element 30 to bend in accordance with the precurvature of the stylet. When approaching a target DRG, the curvature allows the delivery element 30 to curve toward the target DRG, such as along the nerve root angulation. This allows the delivery element 30 to be successfully positioned so that at least one of the outlet ports 40 is on, near, about or in the vicinity of the target DRG. In addition, the outlet ports 40 may be spaced to assist in making such a turn toward the DRG. Typically, the internal stylet is removed when the delivery element 30 is appropriately positioned at the target anatomy, e.g., DRG.

Once the delivery element 30 is positioned such as shown in FIG. 22, the agent may be delivered through the delivery element 30 to the target tissue, such as the DRG. The DRG is bathed in cerebrospinal fluid which efficiently delivers the agent to the DRG. And, since the dura layer D of the intrathecal space ends just outside or distally of the DRG, the cerebrospinal fluid does not flow outwardly or away through the foramen. Rather, the spinal nerves are enveloped in a pocket of cerebrospinal fluid. This allows the agent to be delivered and substantially remain in the area for a period of time. Example periods of time include at least about 1 minute, or at least about 5 minutes, or at least about 10 minutes or at least about 30 minutes or any integer between 1-minute and 30 minutes or more than 30 minutes. In some embodiments, depending on the concentration of the agent and the baricity of the vehicle used to deliver the agent, the agent can remain in the area it is delivered to for at least 1 or more hours. In some embodiments, where the agent is delivered with a particular delivery vehicle, e.g., nanoparticles, liposomes, gels and the like, the agent can remain in the area for extended periods of time, e.g., several hours, to 6 hours or between about 6-12 hours, or between about 12-24 hours or greater than 24 hours, e.g., at least 2 days or at least 3 days or more than 3 days. The agent will remain where it is delivered for extended periods of time when the delivery element is outside the main flow of CSF in the intrathecal space. For example, a DRG can be transfected with a gene at least 2 days after a single lumbar intrathecal injection of a vector. In some instances, this allows for smaller dosages of agent to be used and/or a reduced schedule of dosages than would be used elsewhere within the intrathecal space, or elsewhere within the epidural space or elsewhere within the body. In some embodiments, the flow of CSF is at least about 10-fold less in the DRG than in the epidural space, and therefore in some embodiments, an agent delivered to the DRG will remain in near proximity, e.g., in the lateral recess near the ganglia, after it was delivered for at least about 10-times longer as compared to if the agent was delivered to the epidural space. Accordingly, one can use a dose of the agent delivered to the DRG at least about 10-fold less concentrated than if the agent was delivered, for example, to the epidural space.

A variety of agents may be used. In particular, benzodiazepines, clonazepam (Klonopin, Rivotril, Ravotril, Rivatril, Clonex, Paxam, or Kriadex), morphine, baclofen and/or ziconotide may be used. It may be appreciated that a variety of other agents may be used, such as agents presented elsewhere herein.

In some embodiments, an agent is delivered to a target DRG through a delivery element 30 which is placed intrathecally and electrical stimulation is delivered to the target DRG through a separate delivery element 30 which is placed epidurally. In such embodiments, the intrathecal delivery element is a catheter and the epidural delivery element is a lead. In some instances, the combination therapy of intrathecal agent delivery and epidural stimulation delivery provides effects beyond those achievable by one type of therapy alone. Examples of such combination therapy are described below.

It may be appreciated that in some embodiments a first agent is delivered to a target DRG through a delivery element 30 which is placed intrathecally and a second agent and electrical stimulation is delivered to the target DRG through a separate delivery element 30 which is placed epidurally. In such embodiments, the intrathecal delivery element is a catheter and the epidural delivery element is a lead which also has an agent delivery structure such as outlet ports or a coating, etc. The first and second agents may be the same or different. In some instances, the combination therapy of intrathecal agent delivery and epidural agent delivery and stimulation delivery provides effects beyond those achievable by any one type of therapy alone or by any subcombination of these types of therapy. Examples of such combination therapy are described below.

### C. NEUROMODULATION METHODS

In some aspects of the present invention, the delivery device can be used for delivering an agent directly to a target anatomy, e.g., a DRG as well as in combination with electric stimulation of the target anatomy, e.g., the DRG. This combination allows for neuromodulation of the DRG, where neuromodulation comprises electrical stimulation as well as a variety of others forms of altering or modulating nerve activity by, for example, delivering agents to the DRG target areas.

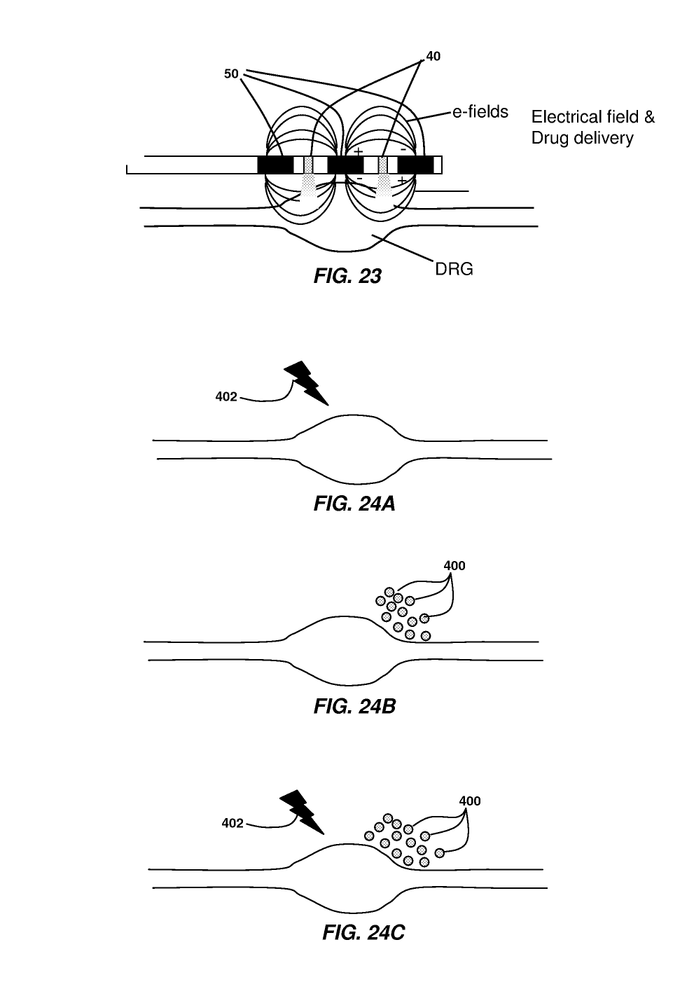

In regards to the combination neurostimulation and pharmacological agent delivery element, the distal tip of the delivery element 30 comprising the electrodes 50 and drug or agent outlet ports 40 can be placed in any location near the target spinal anatomy, e.g., the DRG to obtain the desired stimulation or modulation level. Additionally, the distal tip of the delivery element 30 comprising the electrodes 50 and agent outlet ports 40 can be placed so that modulation or stimulation energy patterns generated by the electrode will remain within or dissipate only within the targeted neural tissue, as shown in FIG. 23. In this embodiment, the targeted spinal anatomy neural tissue is a DRG.

One aspect of the present invention relates to a method of treating pain in a subject comprising positioning a delivery element 30 comprising a delivery lumen with an output port 40 in close proximity to a DRG, and delivering at least one agent from the distal end of the delivery element 30 to the DRG. In some embodiments, the delivery element 30 comprises a lead wherein the lead has at least one electrode at the distal end of the element, so that at least one electrode is positionable in proximity to the DRG, and providing stimulation energy to at least one electrode so as to stimulate the dorsal root ganglion. Together the positioning of the delivery element and the delivering of the agent and providing stimulation energy modulate, e.g., decrease pain sensations, without generating substantial sensations of paresthesia. In some embodiments, providing stimulation energy comprises providing stimulation energy at a level below a threshold for Aβ fiber recruitment. And, in some embodiments, providing stimulation energy comprises providing stimulation energy at a level below a threshold for Aβ fiber cell body recruitment.

In other embodiments, providing stimulation energy comprises: a) providing stimulation energy at a level above a threshold for Aδ fiber cell body recruitment, b) providing stimulation energy at a level above a threshold for C fiber cell body recruitment, c) providing stimulation energy at a level above a threshold for small myelenated fiber cell body recruitment, or d) providing stimulation energy at a level above a threshold for unmyelenated fiber cell body recruitment.

In still other embodiments, providing stimulation energy comprises providing stimulation energy at a level which is capable of modulating glial cell function within the dorsal root ganglion. For example, in some embodiments, providing stimulation energy comprises providing stimulation energy at a level which is capable of modulating satellite cell function within the dorsal root ganglion. In other embodiments, providing stimulation energy comprises providing stimulation energy at a level which is capable of modulating Schwann cell function within the dorsal root ganglion.

In yet other embodiments, providing stimulation energy comprises providing stimulation energy at a level which is capable of causing at least one blood vessel associated with the dorsal root ganglion to release an agent or send a cell signal which affects a neuron or glial cell within the dorsal root ganglion.

In some embodiments, electrical stimulation comprises selectively stimulating a small fiber cell body within a dorsal root ganglion of the subject while excluding an Aβ fiber cell body with the dorsal root ganglion of the subject. In some embodiments, the small fiber body comprises an Aδ fiber cell body. In other embodiments, the small fiber body comprises a C fiber cell body.

Some embodiments of the present invention include direct delivery of the agent to the DRG in combination with electrical stimulation of a nerve root ganglion, for example, electrical stimulation while delivering an agent to the DRG. In one embodiment, an agent is delivered before the electrode is activated. In other embodiments, the agent is delivered to the DRG after, or during the electrode activation.

In still other embodiments, an agent delivered to the DRG is pharmacologically active in the nerve root ganglion during stimulation of the nerve root ganglion. It is to be appreciated that embodiments of the present invention may be altered and modified to accommodate the specific requirements of the neural component being stimulated. For example, embodiments of the present invention may be used to directly stimulate a dorsal root ganglion or a nerve root ganglion of the sympathetic system using the appropriate pharmacological agents, agent release patterns and amounts as well as stimulation patterns and levels.

Some embodiments of the present invention include direct delivery of the agent to the DRG in combination with electrical stimulation of a nerve root ganglion, for example, electrical stimulation while delivering an agent to the DRG. In one embodiment, an agent is delivered before the electrode is activated. In other embodiments, the agent is delivered to the DRG after, or during the electrode activation.

In another embodiment, a method is provided for treating a subject with pain or a pain related disorder, comprising identifying a dorsal root ganglion associated with a sensation of pain by the patient, and delivering an agent to at least one DRG associated with the level of the pain, and optionally providing electrical stimulation to at the DRG so as to reduce the sensation of pain experienced by the subject. In some embodiments, the agent is also delivered to a non-neuronal cell, e.g., a glial cell, e.g., at least one glial cell which includes a satellite cell, or a Schwann cell, or astrocyte cell.

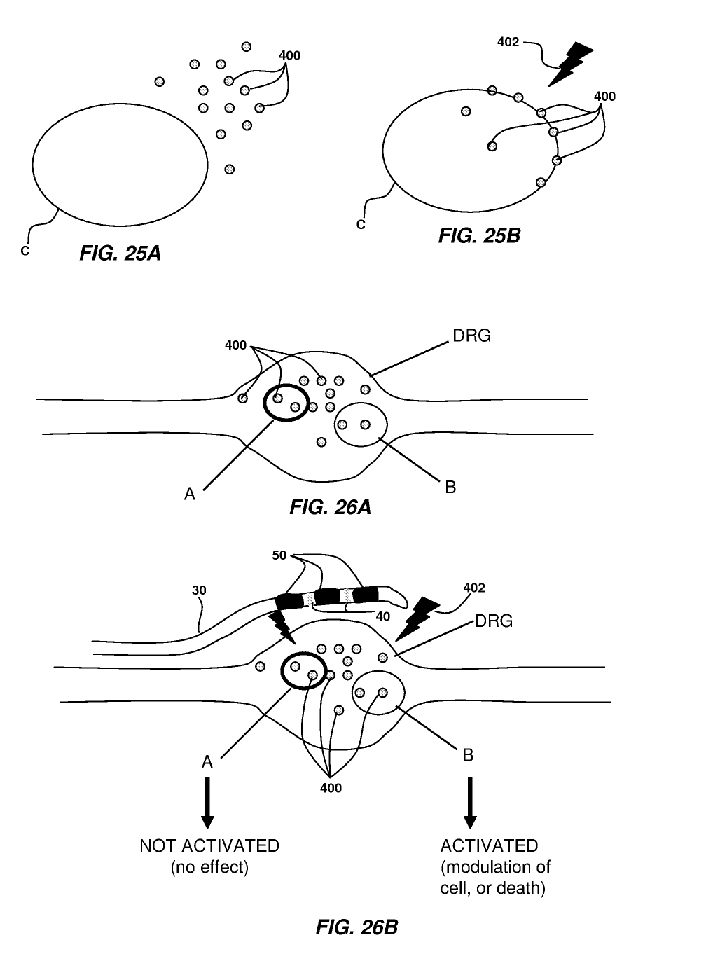

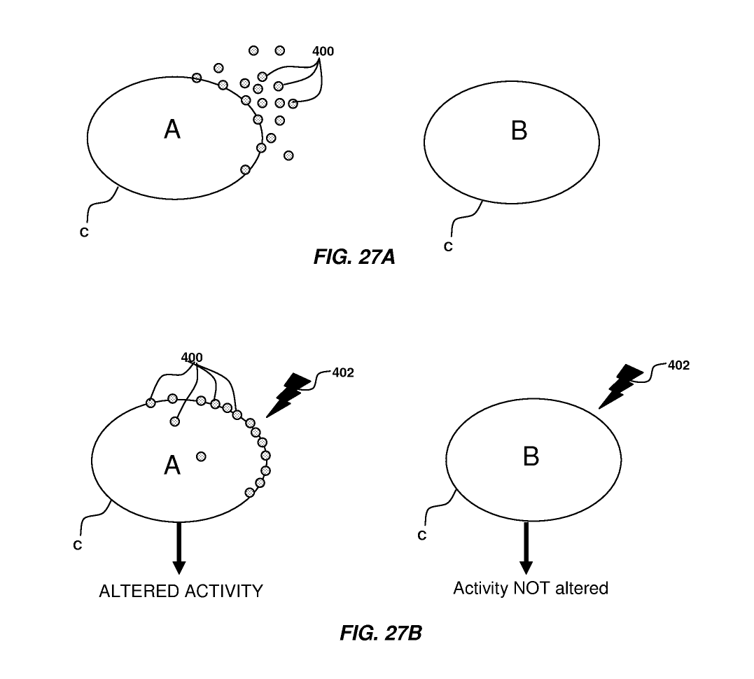

In particular, the neuromodulation system as disclosed herein can provide several advantages of combined agent delivery and electrical stimulation of the DRG. For example, the agent and electrical stimulation can function synergistically to decreased pain sensation in a subject, and to enhance the therapeutic effect of the agent (See FIG. 24A-24B). Alternatively, in some embodiments, the electrical stimulation increases the selectivity of an agent to target DRG cell bodies (see FIG. 25A-25B). Alternatively, in some embodiments, the electrical stimulation enables targeted activation of an agent delivered to the DRG (see FIG. 26A-26B). In another embodiment, the electrical stimulation causes differential enhancement of an agent to delivered target DRG cell bodies (see FIG. 27A-27B).

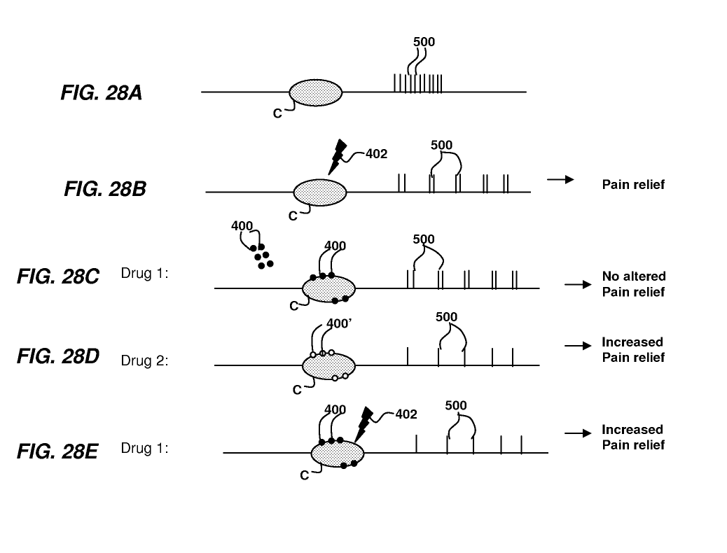

Turning now to FIGS. 28A-28E, various temporal patterns of agent delivery and electrical stimulation mechanisms can be used. While these various mechanisms potentate pain, each of them acts on the primary sensory neuron.

1. Synergistic Action of an Agent and Electrical Stimulation on the DRG

Referring to FIG. 24A-24C, in one embodiment, agent delivery to the DRG enhances the therapeutic effects of electrical stimulation of the DRG, and vice versa, the electrical stimulation enhances the therapeutic effect of the agent delivered to the DRG. For example, electrical stimulation 402 of the DRG without agent delivery can provide a desired level of treatment to a patient, such as evoking significant pain relief (FIG. 24A). Agent 400 delivery or pharmacologic neuromodulation of target tissue or cells (either neuronal or non-neuronal such as glial cells) can also provide a desired level of treatment to a patient, such as pain relief (FIG. 24B). The combination of both electrical stimulation 402 and agent 400 or chemical neuromodulation may be able to provide further relief, longer term relief or relief not achieved by either electrical stimulation or pharmacologic management alone (FIG. 24C). Accordingly, an agent 400 and electrical stimulation 402 function synergistically to increase their therapeutic effect as compared to their use alone.

2. Electrical Stimulation Increases the Selectivity of an Agent

Referring to FIG. 25A, in some embodiments, delivery of an agent 400, e.g., toxin or neurotoxin to a cell body C within a DRG (e.g., a soma cell) in its normal inactive state causes only mild attraction or uptake of the agent 400, e.g, toxin by the cell. In such embodiments, electrical stimulation 402 may be used to activate the cell body C making it preferentially targeted by the agent 400 as shown in FIG. 25B. Thus, electrical stimulation acts on the cell body C, not the agent. In some embodiments, toxins, e.g, neurotoxins, such as the d-conotoxins, are used to target neurons involved in the transduction of pain within the spinal cord. Toxins can be used to directly modulate cell function or destroy cells. Combination of electrical stimulation of cells in the DRG coupled with agent delivery, such as toxins, allows for selective ablation of certain cell types in the DRG, e.g., c-fibers and may be able to provide a therapeutic advantage. In some embodiments, agents which are toxins that affect other non-neuronal cells may also be preferentially targeted to specific tissues.

3. Electrical Stimulation Enables Targeted Activation of an Agent

Referring to FIG. 26A, in some embodiments, an agent 400 is delivered to the DRG using a delivery device targets all cell types, such as cell A and cell B, equally. In such embodiments, electrical stimulation 402 may be provided which selectively activates the agent 400 in at least one cell, such as cell B, but not at least another cell, such as cell A. FIG. 26B illustrates such activation. Here, a delivery element 30 is disposed near the DRG so that at least one of the electrodes 50 resides in proximity to the DRG. The agent 400 is delivered from the outlet ports 40 on the delivery element 30 and electrical stimulation 402 provided by at least one of the electrodes 50 selectively activates the agent 400 in at least one cell (cell B) but not another (cell A).

In some embodiments, an agent can be a pro-drug, e.g., a toxin or other large molecule which is voltage sensitive and becomes active when placed in an environment with a voltage differential. Exemplary agents which are voltage-sensitive include, but are not limited to certain dyes which are known to be activated by changing voltages. Use of such prodrugs, e.g., prodrug toxins which activate on voltage differential can be used for selectively neuromodulating and/or selectively destroying specific cell types in the DRG. For example, a prodrug toxin agent might be generalizable in which cells they target, adhere to, or infect, but the nature of the voltage sensitivity for activation of the toxicity, coupled with the fact that selective cell types will be preferentially modulated by the electrical stimulation e-field, allows for targeted activation of the agent to specific cell types in the DRG.

4. The Agent Enables Differential Enhancement of the Electrical Stimulation

Referring to FIG. 27A-27B, in some embodiments, the therapeutic effect of an agent 400 delivered to the DRG is enhanced using electrical stimulation 402. For example, an agent 400 delivered to a DRG using a delivery device will facilitate or enhance the effect of electrical stimulation of cell types within the DRG for analgesia. For example, chemical neuromodulation of a cell can increase, or makes the cell type more susceptible to electrical neuromodulation. For example, certain agents which function as an ion channel modulating agent can be used to change the membrane biophysics in a way to make a specific cell type more susceptible to the effects of an e-field and subsequent neuromodulation. This differential enhancement by the agent can provide enhanced pain relief.

In the case of FIG. 27A, an agent is delivered to a cell and can have a certain excitatory or inhibitory effect on function of the cell. In the case of 27B the drug is now inducing a specific sensitivity to stimulation such that the cells can be more efficiently targeted or have an especially specific and greater effect on cell function.

5. Temporal Patterns of Delivery of Agents to the DRG and Electrical Stimulation of the DRG

As shown in Table 1, the agent can be delivered to the DRG without electrical stimulation, at the same time (e.g. concurrently with), after or before electrical stimulation. In some embodiments, the electrical stimulation is temporally regulated to be coordinated with the delivery of an agent to the target spinal anatomy, e.g., DRG, for example, within about 1 second, or about 2 seconds or longer.

TABLE 1

| Agent Delivery to the | |
|---|---|
| spinal cord anatomy | Electrical stimulation of the target anatomy |
| Constant | Constant pulse |
| | Intermittent (pre-defined pulse, frequency |
| | parameters) |
| | On-demand (determined by patient) |
| Intermittent (pre-defined | Constant pulse |
| temporal regulation) | Intermittent (pre-defined pulse, frequency |
| | parameters) |
| | On-demand (determined by patient) |
| On demand (determined by | Constant pulse |
| the subject) | Intermittent (pre-defined pulse, frequency |
| | parameters) |
| | On-demand (determined by patient) |

In some embodiments, where both the delivery of the agent and the electrical stimulation is intermittent, they can be temporally regulated and coordinated together such that as an agent is in the delivery “on phase”, electrical stimulation does not occur, and when agent is in the delivery “off phase”, electrical stimulation pulse occurs. In alternative embodiments, both an agent delivery and electrical stimulation, together or individually, can be in “on phase” for a pre-defined period of time, followed by a period where agent delivery and electrical stimulation are both in the “off phase” for a pre-defined period of time.

Without wishing to be limited to theory, electrophysiological studies suggest that Prostaglandin E2 (PGE2), produced by COX enzymes, increases the excitability of DRG neurons in part by reducing the extent of membrane depolarization needed to activate TTX-R Na+ channels. This causes neurons to have more spontaneous firing and predisposed them to favor repetitive spiking (translates to more intense pain sensation). Also illustrated here is how other pro-inflammatory agents (Bradykinin, Capsaicin on the Vanilloid Receptor [VR1]) converge to effect the TTX-R Na+ channel. Opiate action is also upstream from the TTX-R Na+ channel modulation. Embodiments of the present invention advantageously utilize aspects of the pain pathway and neurochemistry to modify electrophysiological excitability of the DRG neurons where electrical stimulation is coupled with pharmacological agents (electrical stimulation alone or in combination with a pharmacological agent) to optimize the efficacy of the stimulation system.

Synergy of electrical and pharmacological modulation may also be obtained using a number of other available pharmacological blockers or other therapeutic agents using a variety of administration routes in combination with specific, directed stimulation of a nerve root ganglion, a dorsal root ganglia, the spinal cord or the peripheral nervous system. Pharmacological blockers include, for example, Na+ channel blockers, Ca++ channel blockers, NMDA receptor blockers and opioid analgesics. As illustrated in FIGS. 24-28 herein, encompassed herein are embodiments for a method for combined stimulation and agent delivery. As shown herein, the electrodes 40 and agent outlet ports 40 are in close proximity of the spinal anatomy, e.g., DRG and positioned to modify and/or influence c-fiber responsiveness. For example, one can deliver a sodium channel blocker (such as, for example, dilantin-[phenytoin], tegretol-[carbamazepine] or other Na+ channel blockers) to the target anatomy, e.g., DRG at the same time, or subsequent to electrical stimulation of the target anatomy, e.g., DRG. As an agent is delivered from the agent outlet port 40, the receptors on c-fibers are blocked thereby decreasing the responsiveness of c-fibers below the response threshold. Accordingly, as the activation potential of the c-fiber has been lowered, the larger diameter A-fiber neurons are preferentially stimulated or the response of the A-fiber remains above the threshold.

Referring to FIG. 28A, a cell body C within a DRG (e.g., a soma cell) is illustrated in an untreated state wherein action potentials 500 indicate sensations of pain by the subject. FIG. 28B illustrates pain relief (by reduced number of action potentials 500) with the application of electrical stimulation 402, such as with the use of a delivery element 30 as described herein. FIG. 28C illustrates the application of Drug 1 (agent 400) to the cell body C when electrical stimulation is not applied. In this instance, pain relief is the same as when electrical simulation is used alone (same pattern of action potentials 500 as illustrated in FIG. 28B). FIG. 28D illustrates the application of Drug 2 (agent 400′) to the cell body C when electrical stimulation is not applied. In this instance, pain relief is increased in comparison to electrical stimulation alone (FIG. 28B) and application of Drug 1 alone (FIG. 28C). FIG. 28E illustrates the application of Drug 1 (agent 400) to the cell body C along with the application of electrical stimulation 402 to the cell body C. In this instance, pain relief is increased to the level that was achieved with Drug 1 (FIG. 28D). Thus, electrical stimulation can alter the pain relief benefit derived from a particular drug, such as by increasing the effectiveness of the particular drug (for example to the level of another drug). This may be useful in a variety of circumstances, including when the other drug has other negative side effects.

Referring again to FIGS. 28A-28E, in the case of drug delivery, some agents will provide pain relief unto the direct actions of the drugs on the cells in the DRG (FIG. 28D, Drug 2). In other cases, drugs will be combined with electrical stimulation to induce a pain relieving effect. FIG. 28C, Drug 1 shows a case where the drug is delivered to the DRG and binds to the cells, but does not have a direct effect. In FIG. 28E, Drug 1 is then administered except this time a concomitant e-field is placed within the binding area of the drug to the cell thereby activating mechanisms by which pain relief can be induced and amplified. In this same way, on-demand systems can be developed to combine agent delivery and electrical stimulation together in a controlled manner (e.g, temporally regulated with respect to each other) to serve as a means by which pain relief can be induced without having to tonically deliver the agent and/or electricity. Furthermore, this avoids the risk of resistance or “tolerance” which can occur with tonic agent administration, or desensitivity on tonic electrical stimulation. Accordingly, the present invention provides a method for phased delivery of agents and/or electrical stimulation to prevent resistance or tolerance to the agent and/or electrical stimulation.

Embodiments of the present invention also provide numerous advantageous combinational therapies. For example, a pharmacological agent may be provided that acts within or influences reactions within the dorsal root ganglia in such a way that the amount of stimulation provided by electrode 50 may be reduced and yet still achieve a clinically significant effect. Alternatively, a pharmacological agent may be provided that acts within or influences reactions within the dorsal root ganglia in such a way that the efficacy of a stimulation provided is increased as compared to the same stimulation provided in the absence of the pharmacological agent. In one specific embodiment, the pharmacological agent is a channel blocker that, after introduction, the c-fiber receptors are effectively blocked such that a higher level of stimulation may be used that may be used in the presence of the channel blocking agent. In some embodiments, the agent may be released prior to stimulation. In other embodiments, the agent may be released during or after stimulation, or in combinations thereof. For example, there may be provided a treatment therapy where the agent is introduced alone, stimulation is provided alone, stimulation is provided in the presence of the agent, or provided at a time interval after the introduction of the agent in such a way that the agent has been given sufficient time to introduce a desired pharmacological effect in advance of the applied stimulation pattern. Embodiments of the stimulation systems and methods of the present invention enable fine tuning of C-fiber and Aβ-fiber thresholds using microelectrodes of the present invention having pharmacological agent coatings coupled with electrical stimulation.

### D. AGENTS FOR DELIVERY

In some embodiments, suitable agent and medicines for treating chronic nerve pain via the delivery devices (DD) 10, systems 1000 and methods as disclosed herein can be any agent, e.g., pharmaceutical agent useful to treat pain. In some embodiments, the agents can target the neuronal cell bodies, e.g., sensory neuron cell bodies or somas, the soma neuronal membranes, intracellular secondary messenger systems, gene expression systems (e.g., translational modifications, post-translational, transcriptional and post-transcriptional mechanisms), epigenetic modifications and the like. In some embodiments, the agents can act on the cell body membrane and integral membranes of the sensory neuron cell body, as well as the cell nucleus and intranuclear structures, ribosomes, mitochondria, t-junction, as well as peripheral and central axons emanating from the biplolar sensory neuronal cell. In some embodiments, the agent and/or electrical stimulation targets the t-junction such that t-junction reduces it's ability to act as a “low pass filter” in the conduction of action potentials from the periphery to the central nervous system.

Examples of such agents include, but are not limited to steroids, such as dexamethasone, and/or local anesthetics such as bupivicaine, lidocaine, and the like. In some embodiments, doxepin or opiate-class drugs can also be used with the delivery device (DD), systems and methods as disclosed.

In some embodiments, the delivery device 10 is adapted to comprise electrodes 50 positioned in close proximity to the DRG for electrical stimulation from the electrode in combination with delivery of the agent to the DRG. In the embodiment illustrated in FIG. 4C, output ports 40 for agent release are surrounded by electrodes 50. In other embodiments, any combination of agent delivery structures, e.g., agent outlet ports and electrode placement can be configured to achieve a desired clinical outcome.

Examples of desired clinical outcomes provided by delivery of an agent to the DRG before, during or after electrical simulation include but are not limited to reduction of inflammation or reduction in pain sensation or other neurological pathologies.

In some embodiments, an agent may include other compounds that, when placed within the body, allow the agent to be released at a certain level over time (i.e., a time released agent). In some embodiments, an agent is an analgesic, or an anti-inflammatory agent, and representative pharmacological agents include, but are not limited to: an opioid, a COX inhibitor, a PGE2 inhibitor, a Na+ channel inhibitor, and combinations thereof and/or another suitable agent to inhibit nociceptive or neuropathic or inflammatory pain, such as for example, Phenyloin, Carbamazepine, Lidocaine GDNF, Opiates, Vicodin, Ultram, and Morphine.

In some embodiments, the agent is a prodrug which is activated by electrical stimulation.

Exemplary agents which can be delivered to a target spinal anatomy, e.g., a DRG using the delivery device include, but are not limited to; receptor Agonists and Antagonists, including Alpha-Receptor Blockers I Agonists, Beta-Receptor Blockers I Agonists, CB-1 (cannaboid-1) receptor agonists and antagonists, Neurotrophic factor receptor (TrkA, TrkB, TrkC) agonists and antagonists, Opioid receptor (mu, delta and kappa subtypes) agonists and antagonists, Partial Opioid Receptor Agonists (e.g., buprenorphine, tramadol, etc), Serotonin (5HT) Receptor Agonists (e.g., amitriptyline, Amitriptyline) or Antagonists, including 5-HT1A agonists or antagonists, and 5-HT1A partial agonists, Norepinephrin Transporter Blockers, GABA receptor Agonists or Antagonists, Glutamate Receptor Agonists or Antagonists, Toll-like Receptors Agonists or Antagonists, NK-1 receptor agonists or antagonists, Neuropeptide Y receptor Agonists or Antagonists, Angiotensin receptor Agonists or Antagonists, Adenosin receptor Agonists or Antagonists, Neuropeptide Y receptor Agonists or Antagonists, Leptin Receptor Agonists or Antagonists, Glycinergic Receptor Agonists or Antagonists, Orphanin/Nociceptin receptor Agonists or Antagonists.

In some embodiments, agents which can be delivered to a target spinal anatomy, e.g., a DRG using the delivery device include, but are not limited to; agents which modulate ion and non-ionic conducting membrane channel proteins, such as but not limited to, Transient Receptor Potential (TRP) channel agonists or antagonists, Sodium channel Agonists or Antagonists, Potassium channel Agonists or Antagonists, Calcium Channel Agonists or Antagonists, Chloride Channel Agonists or Antagonists, Transporter Agonists or Antagonists, Aquaporin channel Agonists or Antagonists.

In some embodiments, agents which can be delivered to a target spinal anatomy, e.g., a DRG using the delivery device include, calcium channel antagonists, sodium channel antagonists, neurokinin receptor 1 (NK1) antagonists, selective serotonin reuptake inhibitors (SSRI) and/or selective serotonin and norepinephrine reuptake inhibitors (SSNRI), tricyclic antidepressant drugs, norepinephrine modulators, lithium, valproate, norepinephrine reuptake inhibitors, monoamine oxidase inhibitors (MAOIs), reversible inhibitors of monoamine oxidase (RIMAs), alpha-adrenoreceptor antagonists, atypical anti-depressants, benzodiazepines, corticotropin releasing factor (CRF) antagonists, gabapentin (e.g., NEURONTIN™), and pregabalin.

In some embodiments, an agent which can be delivered to a target spinal anatomy, e.g., a DRG using the delivery device includes neuroinflammatory modulators, for example, but are not limited to; Cytokine receptor agonists and antagonists (including but not limited to type I, II, TNF receptor family, chemokine receptor family, immunoglobulin receptor superfamily) Antibodies targeted for, but not limited to, IL-1 family, IL2 family, IL-6, TNT-α, IL-10, IFN-γ.

In other embodiments, an agent which can be delivered to a target spinal anatomy, e.g., a DRG using the delivery device includes intracellular signaling and enzyme modulators, for example, but are not limited to; antibodies raised against growth factors such as VEGF, BDNF, NGF, IGFs, e.g., IGF1, IGF2, NTs (16), GDNF, CNTF, etc., steroidal anti-inflammatory agents, free radical scavengers, such as Super oxide dismutase, NOS inhibitors, Calcineurin Inhibitors, Glutamic acid decarboxylase inhibitors, Fracktaline inhibitors, Matrix Metalloproteinase Inhibitors, Heme Oxygenase enhancers and inhibitors, NF-kappaB inhibitors, C-Jun N-terminal kinase (JNK) inhibitors and the like.

Other agents which can be delivered to the DRG using the delivery device as disclosed herein include N-methyl-D-aspartate (NMDA) receptor agonists or antagonists, for example, ketamine, which topically blocks the NMDA Ca2+ channels, and other inhibitors of NMDA (including inhibitors of NR2B and NR1 subunits). Gabapentin also is a glutamate antagonist. Carbamazepine is an AMPA (Na+ channel) receptor blocker, as is gabapentin. The 10-11 epoxide is the active molecule that modulates C fiber afferents at the Langerhans complex. Carbamazepine blocks peripheral sympathetic nerve receptors via the voltage-dependent sodium channels, in the same manner as it blocks these receptors in the dorsal root ganglion (DRG). Clonidine is an alpha 2 blocker that similarly blocks the alpha 2 receptor. Phenoxybenzamine is an alpha 1 agonist. It has much more power to block dorsal ganglionic afferents that synapse with the interneurons of the wide range neurons of areas V to IX of the dorsal horn, before ascending up Lissauer's spinothalamic tract, carrying afferent painful stimuli to the thalamus. Nifedipine is useful for non-NMDA, voltage-sensitive calcium-channel blockade, which down regulates nitric oxide (NO) synthesis. Accordingly, ketamine HCl USP, gabapentin, and phenoxybenzamine HCl can be delivered, and these can be delivered in combination, or alone.

In some embodiments, agents which can be delivered to a target spinal anatomy, e.g., a DRG using the delivery device include, but are not limited to a mitogen-activated protein kinase (MAPK) inhibitor, an α2-receptor agonist, a neuronal nicotinic acetylcholine receptor agonist, a soluble receptor and mixtures thereof, one or more agents selected from the following classes of receptor antagonists and agonists and enzyme activators and inhibitors, each class acting through a differing molecular mechanism of action for pain and inflammation inhibition: histamine receptor antagonists; bradykinin receptor antagonists; kallikrein inhibitors; tachykinin receptor antagonists, including neurokinin 1 and neurokinin 2 receptor subtype antagonists; calcitonin gene-related peptide (CGRP) receptor antagonists; interleukin receptor antagonists; inhibitors of enzymes active in the synthetic pathway for arachidonic acid metabolites, including phospholipase inhibitors, including PLA 2 isoform inhibitors and PLCγ isoform inhibitors, and lipooxygenase inhibitors; prostanoid receptor antagonists including eicosanoid EP-1 and EP-4 receptor subtype antagonists and thromboxane receptor subtype antagonists; leukotriene receptor antagonists including leukotriene B4 receptor subtype antagonists and leukotriene D4 receptor subtype antagonists; and adenosine triphosphate (ATP)-sensitive potassium channel openers. Each of the above agents functions either as an anti-inflammatory agent and/or as an anti-nociceptive, i.e., anti-pain or analgesic, agent. The selection of agents from these classes of compounds is tailored for the particular application.

In some embodiments, agents which can be delivered to a target spinal anatomy, e.g., a DRG using the delivery device include but are not limited to, inhibitor of TrkB, inhibitor of PGE2 EP receptor, inhibitor of MMP-2 and MMP-9, inhibitor of potassium channel Kri1.4, inhibitor of neurotensin receptor-2, and inhibitor of acid-sensing ion channel (ASIC-3). In some embodiments, an agent inhibitor can be using a RNA interferrencing (RNAi) agent, such as a siRNA, as discussed in Tan et al., “Therapeutic potential of RNA interference in Pain Medicine, 2009; Open Pain Journal, 2; 57-63, which is incorporated herein in its entirety by reference. In some embodiments, agents which can be delivered to a target spinal anatomy, e.g., a DRG using the delivery device include antagonists or inhibitors of channels on the central terminal of sensory neurons including, but not limited to, MOR (μ-opiod receptor), DOR (δ-opiod receptor), CB1 (cannaboid recetor 1), GABA_{A/B}, Caᵥ2.2, EP and B2 (bradykinin receptor), which is discussed in Woolf et al., Nociceptors-Noxious stimulus detectors, Neuron, 2007; 55; 353-364, which is incorporated herein in its entirety by reference.

In alternative embodiments, agents which can be delivered to a target spinal anatomy, e.g., a DRG using the delivery device include antagonists or inhibitors of transducer channels on the peripheral terminal of sensory neurons including, but not limited to TREK (heat-sensitive potassium channel), TASK, or antagonists or inhibitors of voltage-gated channels involved in generation of action potentials and/or action potential transduction, which include sodium channels Naᵥ1.6, Naᵥ1.7, Naᵥ1.8, and Naᵥ1.9.

In another embodiment, a sodium channel blocker such as QX-314 can be delivered to a spinal anatomy, e.g., DRG using the device as disclosed herein, as although QX-314 is ineffective at blocking sodium channels extracellularily (because it cannot gain access to the innerface of the channel), it has been demonstrated to inhibit sodium channels in TRPV1-expressing nociceptors. Further, electrical stimulation of the DRG can cause threshold activation of the sodium channel and therefore opening of the channel, and allow entry of QX-314 into the cell and effective inhibition of the sodium channel.

In some embodiments, agents which are delivered to a target spinal anatomy, e.g., DRG using the delivery device as disclosed herein include, for example, inhibitors or antagonists which inhibit TRP (transient receptor potentials channels) or sodium channels which are modulated during inflammation (which decrease the pain threshold at the site of inflammation), including agonists and antagonists of TRP1-4 (Transient receptor potential channels 1-4), TRPM8 (cold sensing TRP channel), inhibitor of TRPV1 (cold sensing channel), TRPA1 (cold sensing TRP channel), ASICs, P2X3, TREK (heat-sensitive potassium channel), TASK, TRPV1 agonists and antagonists are well known to persons of ordinary skill in the art, and include QX-314, Neuroges X, Anesiva, and TRPV3 antagonists include GRC 15133 and GRC 17173 (from Glenmark). Other TRPV 1 antagonists are known, including compounds in Phase I clinical trials: AMG628, AMG517, ABT102, compounds in Phase II clinical trials; GRC 6211, SB-705498, MK-2295, as well as TRPV1 agonists in Phase III clinical trials, NGX 4010 (capsascin), Zucapsaicin, and Capsaicin, sustained release, which are disclosed in Patapoutian et al., Nat. Rev. Drug Discovery, 2009; 8(1); 55-68. Other TRPV1 agonists include, WL-1001, WL-1002, capsazepine, quinazolone compound 26, AMG 0347, AMG 8163, A-784168, benzimidazole, GRC 6127. Antagonists of TPRV1 also include A-425619, BCTC, SB-705498, AMG 9810, A-425619, SB-705498, JNJ-17203212 (4-(3-trifluoromethyl-pyridin-2-yl)-piperazine-1-carboxylic acid (5-trifluoromethyl-pyridin-2-yl)-amide), a quinazolone termed compound 26, A-784168 (N-1H-indazol-4-yl-N′-[(1R)-5-piperidin-1-yl-2,3-dihydro-1H-inden-1-yl]urea) and JYL1421(N-(4-tert-butylbenzyl)-N′-[3-fluoro-4-(methylsulfonylamino)benzyl]thiourea), the structural formulas of which are disclosed in Jara-Oseguera et al., Curr Mol Pharmacol, 2008; 1(3); 255-269, which are effective in reversing the nociceptive behaviours associated with neuropathic pain, bone cancer pain, osteoarthritic pain.

In some embodiments, agents which can be delivered to a target spinal anatomy, e.g., a DRG using the delivery device includes immune modulators of neuropathic pain, for example including, but not limited to minocycline, phosphodiesterase inhibitors (propentofylline, AV-411, pentoxifylline), methotrexate, nucleotide receptor antagonists (activation of P2X and P2Y receptors modulate the activity of peripheral immune cells and microglia), p38 MAP kinase inhibitors, modulators of cytokine synthesis (e.g., neutralizing antibodies and receptor-trapping stratagies directed against IL 1, IL6, IL10, TNF and others) and activity, complement inhibitors, cannabinoids. (see Costigan et al., Annu Rev Neuroscience, 2009, 32; 1-32).

In some embodiments, agents which can be delivered to a target spinal anatomy, e.g., a DRG using the delivery device can be a purinoceptor agonists and antagonists including P2X receptor antagonists and P2Y receptor agonists, and a P2Y2 receptor agonist or a pharmaceutically acceptable salt thereof (also sometimes referred to as an “active agent” herein). Suitable P2Y2 receptor agonists are described in columns 9-10 of U.S. Pat. No. 6,264,975, U.S. Pat. No. 5,656,256, and U.S. Pat. No. 5,292,498.

In some embodiments, the delivery device as disclosed herein is used to deliver agents which typically do not have a therapeutically effective effect to reduce pain in the subject if deliveryed to non-somatic regions of a sensory neuron (e.g, if delivered to the distal axon or central axon in the dorsal column). For example, one advantage of the present delivery device as disclosed herein is direct delivery of agents to a target spinal anatomy, and where the target spinal anatomy is the DRG, the delivered agents can act directly on the sensory neuron cell body (e.g., soma).

In some embodiments, agents which can be delivered to a target spinal anatomy, e.g., a DRG using the delivery device include, anti-spasm agents, and include include serotonin receptor antagonists, tachykinin receptor antagonists, and ATP-sensitive potassium channel openers, calcium channel antagonists, endothelin receptor antagonists and the nitric oxide donors (enzyme activators).

Vanilloid Receptor Agonists

In some embodiments, agents which can be delivered to a target spinal anatomy, e.g., a DRG using the delivery device include a vanilloid agonist, which on sustained use desensitize the vanilloid receptor-1 (VR-1) which transduces heap pain during inflammation and the like. Without wishing to be bound by theory, the Vanilloid receptor-1 (VR1) is a multimeric cation channel prominently expressed in nociceptive primary afferent neurons (see, e.g., Caterina et al., Nature 389:8160824, 1997; Tominaga et al., Neuron 531-543, 1998). Activation of the receptor typically occurs at the nerve endings via application of painful heat (VR1 transduces heat pain) or during inflammation or exposure to vanilloids.

After an initial activation of VR1, VR1 agonists have been reported to desensitize VR1 to subsequent stimuli. This desensitization phenomenon has been exploited in order to produce analgesia to subsequent nociceptive challenge. For example, it has been shown that topical administration of resinferatoxin (RTX), which is a potent vanilloid receptor agonist, triggers a long-lasting insensitivity to chemical pain stimulation. It has recently been reported in U.S. patent application US2010/0222385, which is incorporated herein in its entirety by reference, that intraganglionic or intrathecal administration of vanilloid agonist, e.g., resinferatoxin (RTX) results in decreased pain sensation and decreased neuogenic inflammation and selective ablation of VR1-expressing neurons. Accordingly, in some embodiments, agents which can be delivered to a target spinal anatomy, e.g., a DRG using the delivery device include, a vanilloid agonist, such as, but not limited to resinferatoxin (RTX) or a capsaicin such as ovanil.

VR1 agonists are typically characterized by the presence of a vanilloid moiety that mediates binding and activation of the receptor. Any number of VR1 receptor agonists are useful for delivering to the target anatomy, e.g., spinal cord using the delivery device of the invention. Compounds that act as VR1 receptor agonists include resiniferatoxin and other resiniferatoxin-like complex polycyclic compounds such as tinyatoxin, capsaicin and other capsaicin analogs such as ovanil, and other compounds that include a vanilloid moiety that mediates binding and activation of VR1. Naturally occurring or native RTX is disclosed in U.S. patent application US2010/0222385, which is incorporated herein in its entirety by reference, as well as, RTX analog compounds such as tinyatoxin as well other compounds, e.g., 20-homovanillyl esters of diterpenes such as 12-deoxyphorbol 13-phenylacetate 20-homovanillate and mezerein 20-homovanillate, are described, for example, in U.S. Pat. Nos. 4,939,194; 5,021,450; and 5,232,684. Other resiniferatoxin-type phorboid vanilloids have also been identified (see, e.g., Szallasi et al., Brit. J. Phrmacol. 128:428-434, 1999). Often, the C₂₀-homovanillic moiety, the C₃-keto group and the ortho-ester phenyl group on ring C are important structural elements for activity of RTX and its analogs. As used herein, “a resiniferatoxin” or “an RTX” refers to naturally occurring RTX and analogs of RTX, including other phorbol vanilloids with VR1 agonist activity.

In some embodiments, a VR1 agonist which can be used includes capsaicin, which is a natural product in capsicum peppers that mediates the “hot” sensation characteristic of these peppers. As used herein, “a capsaicin” or “capsaicinoids” refers to capsaicin and capsaicin-related or analog compounds. Naturally occurring or native capsaicin has the structure as disclosed in U.S. patent application US2010/0222385, and can also occur as analogs of capsaicins are known in the art including vanillylacylamides, homovanillyl acylamides, carbamate derivatives, sulfonamide derivatives, urea derivatives, aralkylamides and thioamides, aralkyl aralkanamides, phenylacetamides and phenylacetic acid esters are known in the art. In one embodiment, the capsaicin analog olvanil (N-vanillyl-9-octadecenamide) is used in the methods of the invention. Examples of capsaicin and capsaicin analogs are described, for example, in the following patents and patent applications: U.S. Pat. No. 5,962,532; U.S. Pat. No. 5,762,963; U.S. Pat. No. 5,221,692; U.S. Pat. No. 4,313,958; U.S. Pat. No. 4,532,139; U.S. Pat. No. 4,544,668; U.S. Pat. No. 4,564,633; U.S. Pat. No. 4,544,669; and U.S. Pat. Nos. 4,493,848; 4,532,139; 4,564,633; and 4,544,668, which are all incorporated herein in their entirety by reference.

Other VR1 agonists are well known by persons of ordinary skill in the art, and can be readily identified by measuring binding to a compound to VR1 (VR1 binding assays are described in WO 00/50387, U.S. Pat. No. 5,232,684), or measuring the ability of the compound to stimulate Ca²⁺ influx, and/or the ability of the agent to kill cells that express the vanilloid receptor. VR1 agonists include, but are not limited to those disclosed in WO 00/50387, as well as and OLVANIL™, AM404, Anandamide, and 15-HPETE, which can be delivered to target spinal anatomies, e.g., DRG using the delivery devices as disclosed herein. In some embodiments, these agents can also be used to selectively ablate neuronal cell-types which express the VR1 receptor, e.g., C-fiber neurons. Preferred VR1 agonists, e.g., RTX, typically have a 10-fold, often a 100-fold, preferably a 1000-fold higher binding affinity for VR1 than native, i.e., the naturally occurring, capsaicin.

Serotonin Receptor Antagonists and Agonists

In some embodiments, an agent delivered by the devices and systems as disclosed herien is a serotonin receptor antagonist for the treatment of inflammatory pain and subjects with chronic pain. Serotonin (5-HT) produces pain by stimulating serotonin₂ (5-HT₂) and/or serotonin₃ (5-HT₃) receptors on nociceptive neurons in the periphery. 5-HT₃ receptors on peripheral nociceptors mediate the immediate pain sensation produced by 5-HT (Richardson et al., 1985). 5-HT₃ and 5-HT₂ receptor antagonists inhibit nociceptor activation and neurogenic inflammation.

Accordingly, in some embodiments, 5-HT₂ and 5-HT₃ receptor antagonists can be delivered, either individually or together. In some embodiments, a 5-HT₂ receptor antagonist is amitriptyline (ELAVIL™) which has beneficial effects in certain chronic pain patients. In some embodiments, a5-HT3 receptor antagonist is metoclopramide (REGLAN™) which is used clinically as an anti-emetic drug, can inhibit the pain due to inhibiting 5-HT release from platelets. Other suitable 5-HT₂ receptor antagonists include but are not limted to, imipramine, trazodone, desipramine and ketanserin. Ketanserin has been used clinically for its anti-hypertensive effects. Other suitable 5-HT₃ receptor antagonists include cisapride and ondansetron. SerotoninIB receptor antagonists can also be delivered to target spinal anatomies using the devices as disclosed herein, and include but are not limited to, yohimbine, N-[-methoxy-3-(4-methyl-1-piperanzinyl)phenyl]-2′-methyl-4′-(5-methyl-1,2,4-oxadiazol-3-yl)[1,1-b]phenyl]-4-carboxamide (“GR127935”) and methiothepin.

In some embodiments, agents which can be delivered to the DRG using the delivery device include agonists to 5-HT_{1A}, 5-HT_{1B} and 5-HT_{1D} receptors, which are known to inhibit adenylate cyclase activity. Thus including a low dose of these serotonin_{1A}, serotonin_{1B} and serotonin_{1D} receptor agonists in the solution should inhibit neurons mediating pain and inflammation. The same action is expected from serotonin_{1E} and serotonin_{1E} receptor agonists because these receptors also inhibit adenylate cyclase. Buspirone is a suitable 1A receptor agonist for use in the present invention. Sumatriptan is a suitable 1A, 1B, 1D and 1F receptor agonist. A suitable 1B and 1D receptor agonist is dihydroergotamine. A suitable 1E receptor agonist is ergonovine.

### Bradykinin Receptor Antagonists

In some embodiments, an agent delivered by the devices and systems as disclosed herien is a bradykinin receptor antagonist for the treatment of acute peripheral pain and inflammatory pain. Bradykinin receptors generally are divided into bradykinin₁(B₁) and bradykinin₂ (B₂) subtypes. Acute peripheral pain and inflammation produced by bradykinin is mediated by the B₂ subtype whereas bradykinin-induced pain in the setting of chronic inflammation is mediated via the B₁ subtype.

Bradykinin receptor antagonists can be peptides (small proteins). Antagonists to B₂ receptors block bradykinin-induced acute pain and inflammation. Therefore, depending on the application, an agent delivered by the devices as disclosed herein can be either, or both, bradykinin B₁ and B₂ receptor antagonists. Suitable bradykinin receptor antagonists include, but are not limited to, B₁ receptor antagonists: [des-Arg¹⁰] derivative of D-Arg-(Hyp³-Thi⁵-D-Tic⁷-Oic⁸)-BK (“the [des-Arg¹⁰] derivative of HOE 140″, available from Hoechst Pharmaceuticals); and [Leu⁸] des-Arg⁹-BK. Suitable bradykinin₂ receptor antagonists include: [D-Phe⁷]-BK; D-Arg-(Hyp³-Thi^{5,8}-D-Phe⁷)-BK (“NPC 349”); D-Arg-(Hyp³-D-Phe⁷)-BK (“NPC 567”); and D-Arg-(Hyp³-Thi⁵-D-Tic⁷-Oic⁸)-BK (“HOE 140”). These compounds are more fully described in the previously incorporated Perkins et al. 1993 and Dray et al. 1993 references.

Kallikrein Inhibitors

In some embodiments, an agent delivered by the devices and systems as disclosed herien is a kallikrenin inhibitors for the treatment of acute peripheral pan and inflammatory pain. Bradykinin is produced as a cleavage product by the action of kallikrein on high molecular weight kininogens in plasma. Therefore kallikrein inhibitors, such as aprotinin can be used as agents to inhibit bradykinin production and resultant pain and inflammation.

Tachykinin (TK) Receptor Antagonists.

In some embodiments, an agent delivered by the devices and systems as disclosed herien is a tachykinin receptor antagonist for the treatment of neurogenic inflammatory pain. Tachykinins (TKs) are a family of structurally related peptides that include substance P, neurokinin A (NKA) and neurokinin B (NKB), which induce neuronal stimulation, as well as endothelium-dependent vasodilation, plasma protein extravasation, mast cell recruitment and degranulation and stimulation of inflammatory cells. Due to the above combination of physiological actions mediated by activation of TK receptors, TK receptor inhibitors can be used as agents for the treatment of neurogenic inflammation.

Neurokinin 1 Receptor Subtype Antagonists

In some embodiments, an agent delivered by the devices and systems as disclosed herien is a NK1 receptor antagonist for the treatment of inflammatory pain. Substance P activates the neurokinin receptor subtype NK 1, to have multiple actions which produce inflammation and pain in the periphery after C-fiber activation, including vasodilation, plasma extravasation and degranulation of mast cells. Accordingly, agents delivered to the target spinal anatomies as disclosed herein for the treatment of inflammatory pain include substance P antagonist, such as but not limited to, ([D-Pro 9 [spiro-gamma-lactam]Leu 10,Trp 11]physalaemin-(1-11)) (“GR 82334”), and NK 1 receptor antagonists such as 1-imino-2-(2-methoxy-phenyl)-ethyl)-7,7-diphenyl-4-perhydro-isoindolone(3aR,7aR) (“RP 67580”); and 2S,3S-cis-3-(2-methoxybenzylamino)-2-benzhydrylquinuclidine (“CP 96,345”).

Neurokinin₂ Receptor Subtype Antagonists

In some embodiments, an agent delivered by the devices and systems as disclosed herien is a NK2 receptor antagonist for the treatment of inflammatory pain. Neurokinin A is a peptide which is co-localized in sensory neurons with substance P and which also promotes inflammation and pain. Neurokinin A activates the specific neurokinin receptor, NK₂. NK₂ antagonists which can be delivered to the target spinal cord antomies using the devices as disclosed herein include, without limitation, ((S)—N-methyl-N-[4-(4-acetylamino-4-phenylpiperidino)-2-(3,4-dichlorophenyl)butyl]benzamide (“(±)-SR 48968”); Met-Asp-Trp-Phe-Dap-Leu (“MEN 10,627”); and cyc(Gln-Trp-Phe-Gly-Leu-Met) (“L 659,877”).

CGRP Receptor Antagonists

In some embodiments, an agent delivered by the devices and systems as disclosed herein is a CGRP receptor antagonist for the treatment of pain and inflammatory pain. Calcitonin gene-related peptide (CGRP) is a peptide which is also co-localized in sensory neurons with substance P, and which acts as a vasodilator and potentiates the actions of substance. An example of a suitable CGRP receptor antagonist is α-CGRP-(8-37), a truncated version of CGRP. This polypeptide inhibits the activation of CGRP receptors.

Cyclooxygenase Inhibitors

In some embodiments, non-steroidal anti-inflammatory drugs (NSAIDS) can be delivered to the target spinal anatomy, e.g., a DRG using the delivery device as disclosed herein for the treatment of inflammatory pain. Such NSAID's include, but are not limited to, Aspirin, COX 2 inhibitors such as CELECOXIB™, CELEBREX™, DICLOFENAC™, IBUPROFEN™, KETOPROFEN™, NAPROXEN™ Other agents which can be delivered to the DRG using the delivery device as disclosed herein include, for example, but is not limited to aspirin, acetaminophen (TYLENOL™), ibuprofen (MOTRIN™, ADVIL™) naproxen (ALEVE™, NAPROSYN™), and narcotic drugs including morphine, oxycodone, and hydrocodone (VICODIN™). In some embodiments, any one or a combination of COX-2 inhibitors disclosed in U.S. Application US2003/02039956, which is incorported herein in its entirety by reference, can be delivered using the devices, systems and methods as disclosed herein.

NSAID also include, but without limitation, diclofenac, naproxen, indomethacin, ibuprofen, etc. and are generally nonselective inhibitors of both isoforms of COX, but may show greater selectively for COX-1 over COX-2, although this ratio varies for the different compounds. Antagonists of the eicosanoid receptors (EP-1, EP-2, EP-3, EP-4, DP, FP and TP) can also be delivered as well as antagonists of the thromboxane A2 for the treatment of inflammatory pain. In some embodiments, ketorolac (TORADOL™) can be delivered using the devices for the treatment of inflammatory pain.

In some embodiments, COX-2 inhibitor agents delivered by the devices as disclosed herein have increased selectivity for COX-2 vs. COX-I, for example, agents in the rank order of potency include, but are not limited to DuP 697>SC-58451, celecoxib>nimesulide=meloxicam=piroxicam=NS-398=RS-57067& gt;SC-57666>SC-58125>flosulide>etodolac>L-745,337>DFU-T-614. Suitable COX-2 inhibitors which can be delivered using the device as disclosed herein include, without limitation: CELECOXIB, MELOXICAM, NIMESULIDE, NIMESULIDE, DICLOFENAC, FLOSULIDE, N-[2-(cyclohexyloxy)-4-nitrophenyl]-methanesulfonamide (NS-398), 1-[(4-methylsulfonyl)phenyl]-3-trifluoromethyl-5-[(4-fluoro) phenyl]pyrazole (SC58125), and the following compounds as described in Riendeau, D. et al., (1997) Can. J. Physiol. Pharmacol. 75: 1088-95: DuP 697.

Lipooxygenase Inhibitors

In some embodiments, an agent delivered by the devices and systems as disclosed herein is a lipooxygenase inhibitor for the treatment of inflammatory pain in the subject. Inhibition of the enzyme lipooxygenase inhibits the production of leukotrienes, such as leukotriene B₄, which is known to be an important mediator of inflammation and pain. An example of a 5-lipooxygenase antagonist is 2,3,5-trimethyl-6-(12-hydroxy-5,10-dodecadiynyl)-1,4-benzoquinone (“AA 861”).

Prostanoid Receptor Antagonists

In some embodiments, an agent delivered by the devices and systems as disclosed herein is a prostanoid receptor antagonist for the treatment of inflammatory pain in the subject. Specific prostanoids produced as metabolites of arachidonic acid mediate their inflammatory effects through activation of prostanoid receptors. Examples of classes of specific prostanoid antagonists are the eicosanoid EP-1 and EP-4 receptor subtype antagonists and the thromboxane receptor subtype antagonists. A suitable prostaglandin E₂ receptor antagonist is 8-chlorodibenz[b,f][1,4]oxazepine-10(11H)-carboxylic acid, 2-acetylhydrazide (“SC 19220”). A suitable thromboxane receptor subtype antagonist is [15-[1α,2β(5Z), 3β,4α]-7-[3-[2-(phenylamino)-carbonyl]hydrazino]methyl]-7-oxobicyclo[2,2,1]-hept-2-yl]-5-heptanoic acid (“SQ 29548”).

Opioid Receptor Agonists

In some embodiments, an agent delivered by the devices and systems as disclosed herein is an opioid for the treatment of chronic pain and/or inflammatory pain in the subject. In particular, opioid receptor agonists, including μ-opioid, δ-opioid, and κ-opioid receptor subtype agonists which can be delivered to a target spinal anatomy, e.g., a DRG using the delivery device, as small cells in the DRG expresses MOR1 (mu-opioid receptor) and the doral horn (DH) expresses DOR (delta-opioid receptor). Suitable opiods which can be delivered include, but are not limited to, Alfentanil, Buprenorphine, Carfentanil, Codeine, Dextropropoxyphene, Dihydrocodeine, Diamorphine, Endorphin, Fentanyl, Heroin, Hydrocodone, Hydromorphone, Methadone, Morphine, Oxycodone, Pethidine/meperidine, Remifentanil, Sufentanil, Tramadol and derivatives and analogues thereof.

The μ-receptors are located on sensory neuron terminals in the periphery and activation of these receptors inhibits sensory neuron activity. δ- and κ-receptors are located on sympathetic efferent terminals and inhibit the release of prostaglandins, thereby inhibiting pain and inflammation. Examples of suitable μ-opioid receptor agonists are fentanyl and Try-D-Ala-Gly4N-MePheFNH(CH₂)—OH (“DAMGO”). An example of a suitable δ-opioid receptor agonist is [D-Pen²,D-Pen⁵]enkephalin (“DPDPE”). An example of a suitable κ-opioid receptor agonist is (trans)-3,4-dichloro-N-methyl-N-[2-(1-pyrrolidnyl)cyclohexyl]-benzene acetamide (“U50,488”).

Other opioids which can be delivered by the devices as disclosed herein for the treatment of chronic and inflammatory pain in the subject include, fentanyl, sufentanil and fentanyl congeners are well known in the art, see, e.g., sufentanil (e.g., U.S. Pat. No. 3,998,834; chemical name: ((N-[4-(methyoxymethyl)-1-[2-(2-thienyl)ethyl]-4-piperidinyl]-N-phenylpropanamide 2-hydroxy-1,2,3,-propanetricarboxylate (1:1); C₂₂H₃₀N₂O₂S), fentanyl (e.g., U.S. Pat. No. 3,141,823; chemical name: N-phenyl-N-[1-(2-phenylethyl)-4-piperidinyl]propanamide), alfentanil (e.g., U.S. Pat. No. 4,167,574; chemical name: N-[1-[2-(4-ethyl-4,5-dihydro-5-oxo-1H-tetrazol-1-yl)ethyl]-4-(methoxymethyl)-4-piperidinyl]-N-phenylpropanamide (C₂₁H₃₂N₆O₃)), lofenatnil (e.g., U.S. Pat. No. 3,998,834; chemical name: 3-methyl-4-[(1-oxopropyl)phenylamino]-1-(2-phenylethyl)-4-piperidinecarboxy lic acid methyl ester), carfentanil (chemical name: methyl-4-[(1-oxopropyl)phenylamino]-1-(2-phenylethyl)-4-piperidinecarboxylate (C₂₄H₃₀N₂O₃)), remifentanil (chemical name: 3-[4-methoxycarbonyl-4-[(1-oxopropyl) phenylamino]1-piperidine]propanoic acid), trefentanil (chemical name: N-(1-(2-(4-ethyl-4,5-dihydro-5-oxo-1H-tetrazol-1-yl)ethyl)-4-phenyl-4-piperidinyl)-N-(2-fluorophenyl)-propanamide, and mirfentanil (chemical name: [N-(2-pyrazinyl)-N-(1-phenethyl4-piperidinyl)-2-furamide).

Fentanyl and fentanyl congeners and other opioids which can be delivered by the devices as disclosed herein for the treatment of chronic and inflammatory pain in the subject are discussed in Goodman and Gilman's The Pharmacological Basis of Therapeutics, Chapter 23, “Opioid Analgesics and Antagonists”, pp. 521-555 (9th Ed. 1996); Baly et al. 1991 Med. Res. Rev. 11:403-36 (evolution of the 4-anilidopiperidine opioids); and Feldman et al. 1991 J. Med. Chem. 34:2202-8 (design, synthesis, and pharmacological evaluation of opioid analgesics). For additional information on fentanyl and fentanyl congeners, see, e.g., Scholz et al. 1996 Clin. Pharmacokinet. 31:275-92 (clinical pharmacokinetics of alfentanil, fentanyl, and sufentanil); Meert 1996 Pharmacy World Sci. 18:1-15 (describing pharmacotherapy of morphine, fentanyl, and fentanyl congeners); Lemmens et al. 1995 Anesth. Analg. 80:1206-11 (pharmacokinetics of mirfentanil); Minto et al., 1997 Int. Anesthesiol. Clin. 35:49-65 (review of recently developed opioid analgesics); James 1994 Expert Opin. Invest. Drugs 3:331-40 (discussion of remifentanil); Rosow 1993 Anesthesiology 79:875-6 (discussion of remifentanil); Glass 1995 Eur. J. Anaesthesiol. Suppl. 10:73-4. (pharmacology of remifentanil); and Lemmens et al. 1994 Clin. Pharmacol. Ther.56:261-71 (pharmacokinetics of trefentanil).

Agent delivered by the delivery devices as disclosed herein, such as fentanyl or a fentanyl congener can be provided in the formulation as the opioid base and/or the opioid pharmaceutically acceptable salt. A pharmaceutically acceptable salt embraces the inorganic and the organic salt. Representative salts include a member selected from the group consisting of hydrobromide, hydrochloride, mutate, citrate, succinate, n-oxide, sulfate, malonate, acetate, phosphate dibasic, phosphate monobasic, acetate trihydrate, bi(heplafluorobutyrate), maleate, bi(methylcarbamate), bi(pentafluoropropionate), mesylate; bi(pyridine-3-carboxylate), bi(trifluoroacetate), bitartrate, chlorhydrate, fumarate and sulfate pentahydrate.

Purinoceptor Antagonists and Agonists

In some embodiments, an agent delivered by the devices and systems as disclosed herein is a purinoceptor antagonist or agonist for the treatment of inflammatory pain or nociceptive in the subject. Extracellular ATP acts as a signaling molecule through interactions with P₂ purinoceptors. In particular, ATP depolarizes sensory neurons and plays a role in nociceptor activation since ATP released from damaged cells stimulates P₂X receptors leading to depolarization of nociceptive nerve-fiber terminals. P₂X purinoceptors which are ligand-gated ion channels possessing intrinsic ion channels permeable to Na⁺, K⁺, and Ca²⁺. P₂X receptors are important for primary afferent neurotransmission and nociception. The P2X₃ receptor has a highly restricted distribution and is selectively expressed in sensory C-fiber sensory neurons. Accordingly, antagonists of P2X₃ which an be delivered using the devices as disclosed herein for the treatment of inflammatory pain, include, by way of example, suramin and pyridoxylphosphate-6-azophenyl-2,4-disulfonic acid (“PPADS”).

Adenosine Triphosphate (ATP)-Sensitive Potassium Channel Openers (KCOs)

In some embodiments, an agent delivered by the devices and systems as disclosed herein is an ATP-Sensitive Potassium Channel Openers (KCOs) for the treatment of inflammatory pain in the subject. ATP-sensitive potassium channels are expressed in numerous tissues, including vascular and non-vascular smooth muscle and brain, Opening of these channels causes potassium (K⁺) efflux and hyperpolarizes the cell membrane, causing a reduction in intracellular free calcium through inhibition of voltage-dependent calcium (Ca²⁺) channels and receptor operated Ca²⁺ channels. K⁺ channel openers (KCOs) thus inhibit the effects of ATP-sensitive K+ channels which are typically activated during to nerve stimulation and release of inflammatory mediators. Quast, U., et al., Cardiovasc. Res., Vol. 28, pp. 805-810 (1994).

ATP-sensitive potassium channel openers (KCOs) exhibit synergistic action. Potassium channels that are ATP-sensitive (K_{ATP}) couple the membrane potential of a cell to the cell's metabolic state via sensitivity to adenosine nucleotides. K_{ATP} channels are inhibited by intracellular ATP but are stimulated by intracellular nucleotide diphosphates. The activity of these channels is controlled by the electrochemical driving force to potassium and intracellular signals (e.g., ATP or a G-protein), but are not gated by the membrane potential per se. K_{ATP} channels hyperpolarize the membrane and thus allow them to control the resting potential of the cell. ATP-sensitive potassium currents have been discovered in skeletal muscle, brain, and vascular and nonvascular smooth muscle. Opening of these channels causes potassium efflux and hyperpolarizes the cell membrane. This hyperpolarization (1) induces a reduction in intracellular free calcium through inhibition of voltage-dependent Ca²⁺ channels by reducing the probability of opening L-type or T-type calcium channels, (2) restrains agonist induced (at receptor operated channels) Ca²⁺ release from intracellular sources through inhibition of inositol triphosphate (IP₃) formation, and (3) lowers the efficiency of calcium as an activator of contractile proteins. The combined actions of these two classes of drugs (ATP-sensitive potassium channel openers and calcium channel antagonists) will clamp the target cells into a relaxed state or one which is more resistant to activation.

Potassium-channel opener drugs, such as pinacidil, will open these channels causing K⁺ efflux and hyperpolarization of the cell membrane. Suitable ATP-sensitive K⁺ channel openers for the practice of the present invention include: (−)pinacidil; cromakalim; nicorandil; minoxidil; N-cyano-N′-[1,1-dimethyl-[2,2,3,3-³H]propyl]-N″-(3-pyridinyl)guanidine (“P 1075”); and N-cyano-N′-(2-nitroxyethyl)-3-pyridinecarboximidamide monomethansulphonate (“KRN 2391”)

MAPK Inhibitors

In some embodiments, an agent delivered by the devices and systems as disclosed herein is a MAPK inhibitor for the treatment of inflammatory pain in the subject. Representative examples of MAPK inhibitor compounds suitable for the invention include, for example, 4-(4-fluorophenyl)-2-(4-methylsulfinylphenyl)-5-(4-pyridyl)-1H-imidazole (SB203580), 4-(3-Iodophenyl)-2-(4-methylsulfinylphenyl)-5-(4-pyridyl)-1H-imidazole (SB203580-iodo), 4-(4-fluorophenyl)-2-(4-hydroxyphenyl)-5-(4-pyridyl)-1H-imid azole (SB202190), 5-(2-amino-4-pyrimidyl)-4-(4-fluorophenyl)-1-(4-piperidinyl) imidazole (SB220025), 4-(4-fluorophenyl)-2-(4-nitrophenyl)-5-(4-pyridyl)-1H-imidaz ole (PD 169316), and 2′-amino-3′-methoxyflavone (PD98059).

Tumor Necrosis Factor (TNF) Receptor Family

In some embodiments, an agent delivered by the devices and systems as disclosed herein is an inhibitor of TNFα or a TNF-receptor for the treatment of inflammatory pain in the subject. TNF-α is a cytokine mainly produced by activated macrophages, and plays a central role in the sequence of cellular and molecular events underlying the inflammatory response. Among the proinflammatory actions of TNF, it stimulates the release of other proinflammatory cytokines including IL-1, IL-6, and IL-8. TNFα also induces the release of matrix metalloproteinases from neutrophils, fibroblasts and chondrocytes, and has many other biological actions including cytotoxicity, anti-viral activity, immunoregulatory activities, and transcriptional regulation of several genes that are mediated by specific TNF receptors. 12 different TNF-related receptors have been identified (TNFR-1, TNFR-2, TNFR-RP, CD27, CD30, CD40, NGF receptor, PV-T2, PV-A53R, 4-1BB, OX-40, and Fas) with which eight different TNF-related cytokines associate.

The chimeric TNF soluble receptor (also termed the “chimeric TNF inhibitor” in U.S. Pat. No. 5,447,851) has been shown to bind TNFα with high affinity and is an effective inhibitor of the biological activity of TNFα. In addition, a second example is a chimeric fusion construct comprised of the ligand-binding domain of a TNF receptor with portions of the Fc antibody (also termed Fc fusion soluble receptors) which have been created for TNFα receptors. In another embodiment, a soluble TNF receptor: Fc fusion protein, or modified forms thereof (e.g., the monomeric or dimeric forms), as disclosed in U.S. Pat. No. 5,605,690 can be delivered to the target anatomy using the devices as disclosed herein. In some embodiments, agents which inhibit TNFα for delivery using the devices as disclosed herein include, but are not limited to, a soluble TNF receptor: Fc fusion protein (ENBREL™), SC-58451, RS-57067, SC-57666 and L-745,337.

Ion Channel Blockers

Ion channel blockers can be delivered using the devices as disclosed herein for the treatment of inflammatory pain, chronic pain, nociceptive pain and/or inflammatory pain. Without wishing to be limited to theory, ion-channels can be either anion-channels or cation-channels. Anion-channels are channels that facilitate the transport of anions (e.g., chloride, bicarbonate, and organic ions such as bile acid) across cell membranes. Cation-channels are channels that facilitate the transport of cations (e.g., divalent cations such as Ca⁺² or Ba⁺² or monovalent cations such as Na⁺, K⁺, or H⁺) across cell membranes. In some embodiments of the aspects described herein, the ion-channel which are inhibited are a Na⁺, or a Ca²⁺ or a K⁺ ion-channel.

Sodium Channels

In some embodiments, the delivery devices as disclosed herein deliver a sodium channel blocker agent to the target spinal anatomy, e.g., DRG, for the treatment of inflammatory pain, nociceptive pain or neuropathic pain after nerve injury.

In some embodiments of the aspects described herein, the ion-channel modulator is a sodium pump blocker. As used herein, the terms “sodium pump blocker,” “sodium pump inhibitor,” and “sodium pump antagonist” refer to compounds that inhibit or block the flow of sodium and/or potassium ions across a cell membrane.

As used herein, a “Na⁺ ion-channel” is an ion-channel which displays selective permeability to Na⁺ ions. The term “Sodium channel blockers” or “sodium channel blocking compounds” encompass any chemicals that bind selectively to a sodium channel and thereby deactivate the sodium channel. Agents which function as sodium channel blockers can bind to the SS1 or SS2 subunit of a sodium channel and include without limitation, tetrodotoxin (TTX) and saxitoxin, as disclosed in U.S. Pat. No. 6,407,088, (hereby incorporated in its entirety by reference).

Without wishing to be bound by theory, Naᵥ1.1 is largely expressed by large neurons, while Naᵥ1.6 and Naₓ is expressed in medium to large neurons. In small, c-fiber or noceptive neurons, Naᵥ1.7, Naᵥ1.8 and Naᵥ1.9 are preferentially expressed, and are responsible for rapid depolarization in action potential. Naᵥ1.3 and Naₓ are increased after spinal cord injury, and TTX-resistant sodium channel Naᵥ1.8 is decreased in injured neurons, but upregulated in surrounded uninjured, but sensitized neurons.

Accordingly, in some embodments, sodium channel blockers which selectively block Naᵥ1.7, Naᵥ1.8 and Naᵥ1.9 are useful in the methods and systems for the treatment of pain, as well as sodium channel blockers which inhibit any one of Naᵥ1.3, Naᵥ1.8 and Naₓ are useful in the methods of the present invention to treat neuropathic pain or pain folloing nerve injury.

In some embodiments, a sodium channel blocker agent delivered to the target spinal anatomy, e.g., DRG, can be selected from the group comprising, dilantin-[phenyloin], tegretol-[carbamazepine] Phenyloin, Carbamazepine, Lidocaine, morphine, mexiletine or other Na+ channel blockers.

Intravenous application of the sodium channel blocker lidocaine can suppress the ectopic activity and reverse the tactile allodynia at concentrations that do not affect general behavior and motor function. [Mao, J. and L. L. Chen, Systemic lidocaine for neuropathic pain relief. Pain, 2000. 87: p. 7-17]. In a placebo-controlled study, continuous infusion of lidocaine caused reduced pain scores in patients with peripheral nerve injury, and in a separate study, intravenous lidocaine reduced pain intensity associated with postherpetic neuralgia (PHN). [Mao, J. and L. L. Chen, Systemic lidocaine for neuropathic pain relief. Pain, 2000. 87: p. 7-17. Anger, T., et al., Medicinal chemistry of neuronal voltage-gated sodium channel blockers. Journal of Medicinal Chemistry, 2001. 44 (2): p. 115-137]. LIDODERM®, lidocaine applied in the form of a dermal patch, is currently the only FDA approved treatment for PHN.

A variety of sodium channel blockers can be delivered by the delivery device, for example as disclosed in U.S. Patent Application US2010/0144661 and U.S. Pat. No. 6,030,974, and can include, but is not limited to, substituted benzodiazepinones, benzoxazepinones and benzothiazepinones compounds as disclosed in U.S. Patent Application US2010/0144715. In some embodiments, a sodium channel is a tetrodotoxin or saxitoxin, or their analogues/derivatives can be delivered at a concentration of between about 0.001-10 mM as disclosed in U.S. Patent Application US2010/0215771. In some embodiments, a sodium channel is a compound that binds to the SS1 or SS2 extracellular mouth of the α-subunit thereof, which include saxitoxin and its derivatives and analogues and tetrodotoxin and their derivatives and analogues as disclosed in U.S. Patent Application US2010/0144767 and U.S. Pat. Nos. 6,407,088, 6,030,974 (hereby incorporated in their entirety by reference). Adams, et al., U.S. Pat. Nos. 4,022,899 and 4,029,793 pertain to a local anesthetic composition of tetrodotoxin or desoxytetrodotoxin, and another compound, generally a conventional local anesthetic compound or a similar compound having nerve-blocking properties.

Tetrodotoxin can be used as a local anesthetic and is ten thousand times more powerful than commonly used local non-narcotics. Tetrodotoxin preparations in combination with other widely used anesthetics have been noted in U.S. Pat. No. 4,022,899 and U.S. Pat. No. 4,029,793. Use of tetrodotoxin as a local anaesthetic and analgesic and its topical administration is described in U.S. Pat. No. 6,599,906 Ku. The systemic use of Tetrodotoxin as an analgesic is described in U.S. Pat. No. 6,407,088. Tetrodotoxin (“TTX”), also known as Puffer Fish toxin, maculotoxin, spheroidine, tarichatoxin, tetrodontoxin, and fugu poison, is a biological toxin found in puffer fish (Tetradontiae). The chemical name is octahydro-12-(hydroxymethyl)-2-imino-5, 9:7, 10aH-[1,3]dioxocino[6,5-d]pyrimidine-4,7,10,11,12-pentol with a molecular formula C₁₁H₁₇N₃O₈ and a molecular weight of 319.27. TTX can be extracted from marine organisms (e.g. JP 270719) or synthesized by methods well known to those skilled in the art, e.g. in U.S. Pat. No. 6,552,191, U.S. Pat. No. 6,478,966, U.S. Pat. No. 6,562,968 and US 2002/0086997.

Tetrodoxin's “derivatives and analogues” are disclosed in U.S. Pat. Nos. 6,030,974 and 6,846,475 and includes, but is not limited to, amino perhydroquinazoline compounds having the molecular formula C₁₁H₁₇N₃O₈, anhydro-tetrodotoxin, tetrodaminotoxin, methoxytetrodotoxin, ethoxytetrodotoxin, deoxytetrodotoxin and tetrodonic acid, 6 epi-tetrodotoxin, 11-deoxytetrodotoxin as well as the hemilactal type TTX analogues (e.g. 4-epi-TTX, 6-epi-TTX, 11-deoxy-TTX, 4-epi-11-deoxy-TTX, TTX-8-O-hemisuccinate, chiriquitoxin, 11-nor-TTX-6(S)-ol, 11-nor-TTX-6(R)-ol, 11-nor-TTX-6,6-diol, 11-oxo-TTX and TTX-11-carboxylic acid), the lactone type TTX analogues (e.g. 6-epi-TTX (lactone), 11-deoxy-TTX (lactone), 11-nor-TTX-6(S)-ol (lactone), 11-nor-TTX-6(R)-ol (lactone), 11-nor-TTX-6,6-diol (lactone), 5-deoxy-TTX, 5,11-dideoxy-TTX, 4-epi-5,11-didroxy-TTX, 1-hydroxy-5,11-dideoxy-TTX, 5,6,11-trideoxy-TTX and 4-epi-5,6,11-trideoxy-TTX) and the 4,9-anhydro type TTX analogues (e.g. 4,9-anhydro-TTX, 4,9-anhydro-6-epi-TTX, 4,9-anhydro-11-deoxy-TTX, 4,9-anhydro-TTX-8-O-hemisuccinate, 4,9-anhydro-TTX-11-O-hemisuccinate). The typical analogs of TTX possess only ⅛ to 1/40 of the toxicity of endogenous TTX in mice.

In some embodiments of the aspects described herein, a sodium channel inhibitor or blocker does not significantly modulate an amiloride-sensitive sodium channel. An amiloride-sensitive sodium channel is a membrane-bound ion-channel that is highly sodium-selective (e.g., does not allow the entry or exit of any potassium ions) and is a constitutively active ion-channel. Amiloride-sensitive sodium channels are also referred to as epithelial sodium channel (“ENaC”) and sodium channel non-neuronal 1 (“SCNN1”) in the art.

Ca²⁺ Channel Antagonists.

In some embodiments, an agent delivered by the devices and systems as disclosed herein is a calcium channel antagonist for the treatment of inflammatory pain in the subject. As used herein, a “Ca²⁺ ion-channel” is an ion-channel which displays selective permeabiltiy to Ca²⁺ ions. It is sometimes synonymous as voltage-dependent calcium channel, although there are also ligand-gated calcium channels. See for example, F. Striggow and B. E. Ehrlich, “Ligand-gated calcium channels inside and out”, Curr. Opin. Cell Biol. 8 (4): 490-5 (1996). Exemplary Ca²⁺ ion-channels include, but are not limited to, L-type, P-type/Q-type, N-type, R-type, and T-type. In some embodiments of the aspects described herein, the Ca²⁺ ion-channel is a L-type Ca²⁺ ion-channel.

Accordingly, in some embodiments of the aspects described herein, the ion-channel modulator delivered by the delivery device as disclosed herein is a calcium channel blocker. As used herein, the terms “calcium channel blocker,” “calcium channel inhibitor,” and “calcium channel antagonist” refer to compounds that inhibit or block the flow of calcium ions across a cell membrane. Calcium channel blockers are also known as calcium ion influx inhibitors, slow channel blockers, calcium ion antagonists, calcium channel antagonist drugs and as class IV antiarrhythmics.

Calcium channel antagonists can interfere, e.g., block the transmembrane flux of calcium ions required for activation of cellular responses mediating neuroinflammation. Exemplary calcium channel blocker include, but are not limited to, amiloride, amlodipine, bepridil, diltiazem, felodipine, isradipine, mibefradil, nicardipine, nifedipine (dihydropyridines), nickel, nimodinpine, nisoldipine, nitric oxide (NO), norverapamil, verapamil, and analogs, derivatives, pharmaceutically acceptable salts, and/or prodrugs thereof. Nifedipine can reduce the release of arachidonic acid, prostaglandins, and leukotrienes that are evoked by various stimuli.

In some embodiments of the aspects described herein, the calcium channel blocker is a beta-blocker. Exemplary beta-blockers include, but are not limited to, Alprenolol, Bucindolol, Carteolol, Carvedilol (has additional α-blocking activity), Labetalol, Nadolol, Penbutolol, Pindolol, Propranolol, Timolol, Acebutolol, Atenolol, Betaxolol, Bisoprolol, Celiprolol, Esmolol, Metoprolol, Nebivolol, Butaxamine, and ICI-118,551 (3-(isopropylamino)-1-[(7-methyl-4-indanyl)oxy]butan-2-ol), and analogs, derivatives, pharmaceutically acceptable salts, and/or prodrugs thereof.

The dihydropyridines, including nisoldipine, act as specific inhibitors (antagonists) of the voltage-dependent gating of the L-type subtype of calcium channels. Systemic administration of the calcium channel antagonist nifedipine during cardiac surgery previously has been utilized to prevent or minimize coronary artery vasospasm. Seitelberger, R., et al., Circulation, Vol. 83, pp. 460-468 (1991).

Calcium channel antagonists and ATP-sensitive potassium channel openers likewise exhibit synergistic action. Opening of ATP-sensitive potassium channel causes potassium efflux and hyperpolarizes the cell membrane. This hyperpolarization (1) induces a reduction in intracellular free calcium through inhibition of voltage-dependent Ca²⁺ channels by reducing the probability of opening L-type or T-type calcium channels, (2) restrains agonist induced (at receptor operated channels) Ca²⁺ release from intracellular sources through inhibition of inositol triphosphate (IP₃) formation, and (3) lowers the efficiency of calcium as an activator of contractile proteins. The combined actions of these two classes of drugs (ATP-sensitive potassium channel openers and calcium channel antagonists) will clamp the target cells into a relaxed state or one which is more resistant to activation. Accordingly, in some embodiments, both a calcium channel antagonist and a KCO are delivered indivdually or together using the devices as disclosed herein.

In some embodiments, calcium channel antagonists can be combined with tachykinin and/or bradykinin antagonist to provide synergistic effects in mediating neuroinflammation. Calcium channel antagonists interfere with a common mechanism involving elevation of intracellular calcium, part of which enters through L-type channels. Suitable calcium channel antagonists for delivery for the treatment of pain include, but are not limited to, nisoldipine, nifedipine, nimodipine, lacidipine, isradipine and amlodipine

Potassium Channels

In some embodiments, an agent delivered by the devices and systems as disclosed herein is a potassium (K⁺) channel antagonist for the treatment of inflammatory pain in the subject. As used herein, a “K⁺ ion-channel” is an ion-channel which displays selective permeabiltiy to K⁺ ions. There are four major classes of potassium channels: calcium-activated potassium channel, which opens in response to presence of calcium ions or other signaling molecules; inwardly rectifying potassium channel, which passes current (positive charge) more easily in the inward direction (into the cell); tandem pore domain potassium channels, which are constitutively open or possess high basal activation; and voltage-gated potassium channels, which open or close in response to changes in the transmembrane voltage.

Exemplary K⁺ ion-channel include, but are not limited to, BK channel, SK channel, ROMK (Kᵢᵣ1.1), GPCR regulated (Kᵢᵣ3.x), ATP-sensitive (Kᵢᵣ6.x), TWIK, TRAAK, TREK, TASK, hERG (Kᵥ11.1), and KvLQT1 (Kᵥ7.1). In some embodiments the K⁺ ion-channel is a ATP-sensitive K⁺ channel which is a K⁺ ion-channel that is that is gated by ATP. ATP-sensitive potassium channels are composed of Kᵢᵣ6.x-type subunits and sulfonylurea receptor (SUR) subunits, along with additional components. See for example, Stephan, et al., “Selectivity of repaglinide and glibenclamide for the pancreatic over the cardiovascular K(ATP) channels”, Diabetologia 49 (9): 2039-48 (2006). ATP-sensitive K⁺ channels can be further identified by their position within the cell as being either sarcolemmal (“sarcK_{ATP}”), mitochondrial (“mitoK_{ATP}”), or nuclear (“nucK_{ATP}”).

In some embodiments, a potassium channel agonist, which is a K⁺ ion-channel modulator which facilitates ion transmission through K⁺ ion-channels can be delivered to target spinal cord anatomies using the device as disclosed herein. Exemplary potassium channel agonists include, but are not limited to diazoxide, minoxidil, nicorandil, pinacidil, retigabine, flupirtine, lemakalim, L-735534, and analogs, derivatives, pharmaceutically acceptable salts, and/or prodrugs thereof.

Additional exemplary K⁺ ion-channel modulators which can be delivered using the devices as disclosed herein for the treatment of pain, e.g, inflammatory pain, include, but are not limited to, 2,3-Butanedione monoxime; 3-Benzidino-6-(4-chlorophenyl)pyridazine; 4-Aminopyridine; 5-(4-Phenoxybutoxy)psoralen; 5-Hydroxydecanoic acid sodium salt; L-α-Phosphatidyl-D-myo-inositol; 4,5-diphosphate, dioctanoyl; Aal; Adenosine 5′-(β,γ-imido)triphosphate tetralithium salt hydrate; Agitoxin-1; Agitoxin-2; Agitoxin-3; Alinidine; Apamin; Aprindine hydrochloride; BDS-I; BDS-II; BL-1249; BeKm-1; CP-339818; Charybdotoxin; Charybdotoxin; Chlorzoxazone; Chromanol 293B; Cibenzoline succinate; Clofilium tosylate; Clotrimazole; Cromakalim; CyPPA; DK-AH 269; Dendrotoxin-I; Dendrotoxin-K; Dequalinium chloride hydrate; DPO-1 needles; Diazoxide; Dofetilide; E-4031; Ergtoxin; Glimepiride; Glipizide; Glybenclamide; Heteropodatoxin-2; Hongotoxin-1; ICA-105574; IMID-4F hydrochloride; Iberiotoxin; Ibutilide hemifumarate salt; Isopimaric Acid; Kaliotoxin-1; Levcromakalim; Lq2; Margatoxin; Mast Cell Degranulating Peptide; Maurotoxin; Mephetyl tetrazole; Mepivacaine hydrochloride; Minoxidil; Minoxidil sulfate salt; N-Acetylprocainamide hydrochloride; N-Salicyloyltryptamine; NS1619; NS1643; NS309; NS8593 hydrochloride; Nicorandil; Noxiustoxin; Omeprazole; PD-118057; PNU-37883A; Pandinotoxin-Kα; Paxilline; Penitrem A; Phrixotoxin-2; Pinacidil monohydrate; Psora-4; Quinine; Quinine hemisulfate salt monohydrate; Quinine hydrobromide; Quinine hydrochloride dehydrate; Repaglinide; Rutaecarpine; S(+)-Niguldipine hydrochloride; SG-209; Scyllatoxin; Sematilide monohydrochloride monohydrate; Slotoxin; Stromatoxin-1; TRAM-34; Tamapin; Tertiapin; Tertiapin-Q trifluoroacetate salt; Tetracaine; Tetracaine hydrochloride; Tetraethylammonium chloride; Tityustoxin-Kα; Tolazamide; UCL 1684; UCL-1848 trifluoroacetate salt; UK-78282 monohydrochloride; VU 590 dihydrochloride hydrate; XE-991; ZD7288 hydrate; Zatebradine hydrochloride; α-Dendrotoxin; β-Dendrotoxin; δ-Dendrotoxin; γ-Dendrotoxin; β-Bungarotoxin; and analogs, derivatives, pharmaceutically acceptable salts, and/or prodrugs thereof.

In some embodiments of the aspects described herein, the ion-channel is a Na⁺/K⁺ pump. The Na⁺/K⁺ pump is also referred to as simply as the sodium pump in the art. The Na⁺/K⁺ pump is an electrogeneic transmembrane ATPase. It is a highly-conserved integral membrane protein that is expressed in virtually all cells of higher organisms. The sodium pump is responsible for the maintenance of ionic concentration gradients across the cell membrane by pumping three Na⁺ out of the cell and two K⁺ into the cell. Since this channel requires the expenditure of energy by hydrolysis of ATP for this action, it is, therefore, called as Na⁺/K⁺-ATPase. It has been estimated that roughly 25% of all cytoplasmic ATP is hydrolyzed by sodium pumps in resting humans. In nerve cells, approximately 70% of the ATP is consumed to drive Na⁺/K⁺-ATPase. The Na⁺/K⁺-ATPase helps maintain resting potential, avail transport, and regulate cellular volume. It also functions as signal transducer/integrator to regulate MAPK pathway, ROS, as well as intracellular calcium.

In some embodiments, an agent delivered by the devices and systems as disclosed herein is a Na⁺/K⁺ pump antagonist for the treatment of inflammatory pain in the subject. Na⁺/K⁺ pump maintain the volume of the cell. The pump transports 3 Na⁺ ions out of the cell and in exchange takes 2 K⁺ ions into the cell. As the membrane is far less permeable to Na⁺ ions than K⁺ ions the sodium ions have a tendency to stay there. This represents a continual net loss of ions out of the cell. The opposing osmotic tendency that results operates to drive the water molecules out of the cells. Furthermore, when the cell begins to swell, this automatically activates the Na⁺—K⁺ pump, which moves still more ions to the exterior.

The catalytic subunit of the Na⁺/K⁺-ATPase is expressed in various isoforms (α1, α2, α3).

In some embodiments of the aspects described herein, an agent which can be delivered to a DRG using the delivery device as disclosed herein is a modulator or an inhibitor or an antagonist of the ion-channel. As used herein, the term “inhibitor” with respect to “an inhibitor of an ion channel” refers to an agent or compounds which inhibit or decrease the flow of ions through an ion-channel.

In some embodiments of the aspects described herein, the modulator is an agonist of the ion-channel, for example, where it is desirable to increase an ion channel activation which is present specifically on an inhibitory neuron. As used herein, the term “agonist” as used in reference to an “ion channel agonist” refers to agent and compounds which increase the flow of ions through an ion-channel.

In some embodiments of the aspects described herein, an ion channel modulator modulates at least one activity of the ion-channel by at least 5%, at least 10%, at least 15%, at least 20%, at least 25%, at least 30%, at least 40%, at least 50%, at least 60%, at least 70%, at least 80%, at least 90%, or at least 95%, at least 98% or more relative to a control with no modulation.

In some embodiments of the aspects described herein, at least one activity of the ion-channel is inhibited or lowered by at least 5%, at least 10%, at least 15%, at least 20%, at least 25%, at least 30%, at least 40%, at least 50%, at least 60%, at least 70%, at least 80%, at least 90%, at least 95%, at least 98%, or 100% (e.g. complete loss of activity) relative to control with no modulator.

In some embodiments of the aspects described herein, the ion-channel modulator has an IC₅₀, for inhibiting the activity of an ion channel, of less than or equal to 500 nM, 250 nM, 100 nM, 50 nM, 10 nM, 1 nM, 0.1 nM, 0.01 nM or 0.001 nM.

In some embodiments of the aspects described herein, the ion-channel modulator inhibits the flow of ions through the ion-channel by at least 5%, at least 10%, at least 15%, at least 20%, at least 25%, at least 30%, at least 40%, at least 50%, at least 60%, at least 70%, at least 80%, at least 90%, at least 95%, at least 98%, or 100% (e.g. complete stop of ion flow through the channel) relative to a control with no modulator.

In some embodiments of the aspects described herein, the ion-channel modulator increases the flow of ions through the ion-channel by at least 5%, at least 10%, at least 15%, at least 20%, at least 25%, at least 30%, at least 40%, at least 50%, at least 60%, at least 70%, at least 80%, at least 90%, at least 100%, at least 1.5 fold, at least by 2-fold, at least 3-fold, at least 4-fold, or at least 5-fold or more relative to a control with no modulator.

In some embodiments of the aspects described herein, the ion-channel modulator increases concentration of ions, e.g. sodium, in a cell by at least 5%, at least 10%, at least 15%, at least 20%, at least 25%, at least 30%, at least 40%, at least 50%, at least 60%, at least 70%, at least 80%, at least 90%, at least 100%, at least 1.5 fold, at least by 2-fold, at least 3-fold, at least 4-fold, or at least 5-fold or more relative to a control with no modulator.

Without wishing to be bound by a theory, an ion-channel modulator can modulate the activity of an ion-channel through a number of different mechanisms. For example, a modulator can bind with the ion-channel and physically block the ions from going through the channel. An ion-channel modulator can bring about conformational changes in the ion-channel upon binding, which may increase or decrease the interaction between the ions and the channel or may increase or decrease channel opening.

A modulator can modulate the energy utilizing activity, e.g. ATPase activity, of the ion-channel. In some embodiments of the aspects described herein, the ion-channel modulator inhibits the ATPAse activity of the ion-channel.

In some embodiments of the aspects described herein, an ion-channel modulator can inhibit the ATPase activity of an ATP-dependent channel, e.g., a Na⁺/K⁺-ATPase by at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, at least 98%, or 100% (complete inhibition) relative to a control without the modulator. Without wishing to be bound by theory, ATPase activity can be measured by measuring the dephosphorylation of adenosine-triphosphate by utilizing methods well known to the skilled artisan for measuring such dephosphorylation reactions.

In some embodiments of the aspects described herein, an ion-channel modulator inhibits a sodium channel activation by at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, at least 98%, or 100% (complete inhibition) relative to a control without the modulator.

Without limitation, the ion-channel modulator can be a small organic molecule, small inorganic molecule, a polysaccharide, a peptide, a protein, a nucleic acid, an extract made from biological materials such as bacteria, plants, fungi, animal cells, animal tissue, and any combinations thereof.

In some embodiments, an ion-channel modulator can be an antiarrhytmic agent. As used herein, the term “antiarrhythmic agent” refers to compounds that are used to treat, or control, cardiac arrhythmias, such as atrial fibrillation, atrial flutter, ventricular tachycardia, and ventricular fibrillation. Generally an antiarrhythmic agent's mechanism of action conforms to one or more of the four Vaughan-Williams classifications. The four main classes in the Vaughan Williams classification of antiarrhythmic agents are as follow: Class I agents interfere with the Na⁺ channel; Class II agents are anti-sympathetic nervous system agents, most agents in this class are beta blockers; Class III agents affect K⁺ efflux; and Class IV agents affect Ca²⁺ channels and the AV node. Since the development of the original Vaughan-Williams classification system, additional agents have been used that don't fit cleanly into categories I through IV. These agents are also included in the term “antiarrhythmic agent.” Exemplary antiarrhytmic agents include, but are not limited to, Quinidine, Procainamide, Disopyramide, Lidocaine, Phenyloin, Flecamide, Propafenone, Moricizine, Propranolol, Esmolol, Timolol, Metoprolol, Atenolol, Bisoprolol, Amiodarone, Sotalol, Ibutilide, Dofetilide, E-4031, Diltiazem, Adenosine, Digoxin, adenosine, magnesium sulfate, and analogs, derivatives, pharmaceutically acceptable salts, and/or prodrugs thereof.

In some embodiments of the aspects described herein, the ion-channel modulator is selected from the group consisting of bufalin; digoxin; ouabain; nimodipine; diazoxide; digitoxigenin; ranolazine; lanatoside C; Strophantin K; uzarigenin; desacetyllanatoside A; actyl digitoxin; desacetyllanatoside C; strophanthoside; scillaren A; proscillaridin A; digitoxose; gitoxin; strophanthidiol; oleandrin; acovenoside A; strophanthidine digilanobioside; strophanthidin-d-cymaroside; digitoxigenin-L-rhamnoside; digitoxigenin theretoside; strophanthidin; digoxigenin-3,12-diacetate; gitoxigenin; gitoxigenin 3-acetate; gitoxigenin-3,16-diacetate; 16-acetyl gitoxigenin; acetyl strophanthidin; ouabagenin; 3-epigoxigenin; neriifolin; acetyhieriifolin cerberin; theventin; somalin; odoroside; honghelin; desacetyl digilanide; calotropin; calotoxin; convallatoxin; oleandrigenin; periplocyrnarin; strophanthidin oxime; strophanthidin semicarbazone; strophanthidinic acid lactone acetate; ernicyrnarin; sannentoside D; sarverogenin; sarmentoside A; sarmentogenin; proscillariditi; marinobufagenin; Amiodarone; Dofetilide; Sotalol; Ibutilide; Azimilide; Bretylium; Clofilium; N-[4-[[1-[2-(6-Methyl-2-pyridinyl)ethyl]-4-piperidinyl]carbonyl]phenyl]methanesulfonamide (E-4031); Nifekalant; Tedisamil; Sematilide; Ampyra; apamin; charybdotoxin; 1-Ethyl-2-benzimidazolinone (1-EBIO); 3-Oxime-6,7-dichloro-1H-indole-2,3-dione (NS309); Cyclohexyl-[2-(3,5-dimethyl-pyrazol-1-yl)-6-methyl-pyrimidin-4-yl]-amine (CyPPA); GPCR antagonists; ifenprodil; glibenclamide; tolbutamide; diazoxide; pinacidil; halothane; tetraethylammonium; 4-aminopyridine; dendrotoxins; retigabine; 4-aminopyridine; 3,4-diaminopyridine; diazoxide; Minoxidil; Nicorandi; Retigabine; Flupirtine; Quinidine; Procainamide; Disopyramide; Lidocaine; Phenyloin; Mexiletine; Flecamide; Propafenone; Moricizine; atenolol; ropranolol; Esmolol; Timolol; Metoprolol; Atenolol; Bisoprolol; Amiodarone; Sotalol; Ibutilide; Dofetilide; Adenosine; Nifedipine; δ-conotoxin; κ-conotoxin; μ-conotoxin; ω-conotoxin; ω-conotoxin GVIA; ω-conotoxin ω-conotoxin CNVIIA; ω-conotoxin CVIID; ω-conotoxin AM336; cilnidipine; L-cysteine derivative 2A; ω-agatoxin IVA; N,N-dialkyl-dipeptidyl-amines; SNX-111 (Ziconotide); caffeine; lamotrigine; 202W92 (a structural analog of lamotrigine); phenyloin; carbamazepine; 1,4-dihydro-2,6-dimethyl-5-nitro-4-[thieno[3,2-c]-pyridin-3-yl]-3-pyridinecarboxylic acid, 1-phenylethyl ester; 1,4-dihydro-2,6-dimethyl-5-nitro-4-[thieno[3,2-c]-pyridin-3-yl]-3-pyridinecarboxylic acid, 1-methyl-2-propynyl ester; 1,4-dihydro-2,6-dimethyl-5-nitro-4-[3,2-c]pyridin-3-yl]-3-pyridinecarboxylic acid, cyclopropylmethyl ester; 1,4-dihydro-2,6-dimethyl-5-nitro-4-[thieno(3,2-c)pyridin-3-yl]-3-pyridinecarboxylic acid, butyl ester; (S)-1,4-Dihydro-2,6-dimethyl-5-nitro-4-[thieno[3,2c]pyridin-3-yl]-3-pyridinecarboxylic acid, 1-methylpropyl ester; 1,4-Dihydro-2,6-dimethyl-5-nitro-4-thieno[3,2-c]pyridin-3-yl]-3-pyridinecarboxylic acid, methyl ester; 1,4-Dihydro-2,6-dimethyl-5-nitro-4-[thieno[3,2-c]pyridin-3-yl]-3-pyridinecarboxylic acid, 1-methylethyl ester; 1,4-Dihydro-2,6-dimethyl-5-nitro-4-thieno[3,2-c]pyridin-3-yl]-3-pyridinecarboxylic acid, 2-propynyl ester; 1,4-Dihydro-2,6-dimethyl-5-nitro-4-[thieno[3,2-c]pyridin-3-yl]-3-pyridinecarboxylic acid, 1-methyl-2propynyl ester; 1,4-Dihydro-2,6-dimethyl-5-nitro-4-[thieno[3,2-c]pyridin-3-yl]-3-pyridinecarboxylic acid, 2-butynyl este; 1,4-Dihydro-2,6-dimethyl-5-nitro-4-[thieno[3,2-c]pyridin-3-yl]-3-pyridinecarboxylic acid, 1-methyl-2butynyl este; 1,4-Dihydro-2,6-dimethyl-5-nitro-4-[thieno[3,2-c]pyridin-3-yl]-3-pyridinecarboxylic acid, 2,2-dimethylpropyl ester; 1,4-Dihydro-2,6-dimethyl-5-nitro-4-thieno[3,2-c]pyridin-3-yl]-3-pyridinecarboxylic acid, 3-butynyl ester; 1,4-Dihydro-2,6-dimethyl-5-nitro-4-[thieno3,2-c]pyridin-3-yl]-3-pyridinecarboxylic acid, 1,1-dimethyl-2propynyl ester; 1,4-Dihydro-2,6-dimethyl-5-nitro-4-[thieno3,2-c]pyridin-3-yl-3-pyridinecarboxylic acid, 1,2,2-trimethylpropyl ester; R(+)-1,4-Dihydro-2,6-dimethyl-5-nitro-4[thieno[3,2-c]pyridin-3-yl]-3-pyridinecarboxylic (2Amethyl-1-phenylpropyl)ester; S-(−)-1,4-Dihydro-2,6-dimerhyl-5-nitro-4[thieno[3,2-c]pyridin-3-yl]-3-pyridinecarboxylic acid, 2-methyl-1-phenylpropyl ester; 1,4-Dihydro-2,6-dimethyl-5-nitro-4-[thieno[3,2c]-pyridin-3-yl]-3-pyridinecarboxylic acid, 1-methylphenylethyl ester; 1,4-Dihydro-2,6-dimethyl-5-nitro-4-[thieno[3,2-c]pyridin-3-yl]-3-pyridinecarboxylic acid, 1-phenylethyl ester; 1,4-Dihydro-2,6-dimethyl-5-nitro-4-[thieno[3,2c]-pyridin-3-yl]-3-pyridinecarboxylic acid, (1-phenylpropyl)ester; 1,4-Dihydro-2,6-dimethyl-5-nitro-4-[thieno[3,2c]-pyridin-3-yl]-3-pyridinecarboxylic acid, (4-methoxyphenyl)methyl ester; 1,4-Dihydro-2,6-dimethyl-5-nitro-4-[thieno[3,2c]-pyridin-3-yl]-3-pyridinecarboxylic acid, 1-methyl-2-phenylethyl ester; 1,4-Dihydro-2,6-dimethyl-5-nitro-4-[thieno[3,2c]-pyridin-3-yl]-3-pyridinecarboxylic acid, 2-phenylpropyl ester; 1,4-Dihydro-2,6-dimethyl-5-nitro-4-[thieno[3,2c]-pyridin-3-yl]-3-pyridinecarboxylic acid, phenylmethyl ester; 1,4-Dihydro-2,6-dimethyl-5-nitro-4-[thieno[3,2c]-pyridin-3-yl]-3-pyridinecarboxylic acid, 2-phenoxyethyl ester; 1,4-Dihydro-2,6-dimethyl-5-nitro-4-thieno-3,2-c]pyridin-3-yl]-3-pyridinecarboxylic acid, 3-phenyl-2propynyl este; 1,4-Dihydro-2,6-dimethyl-5-nitro-4-[thieno[3,2c]-pyridin-3-yl]-3-pyridinecarboxylic acid, 2-methoxy-2-phenylethyl ester; (S)-1,4-Dihydro-2,6-dimethyl-5-nitro-4-[thieno[3,2-c]pyridin-3-yl]-3-pyridinecarboxylic acid, 1-phenylethyl este; (R)-1,4-Dihydro-2,6-dimethyl-5-nitro-4-[thieno[3,2-c]pyridin-3-yl]-3-pyridinecarboxylic acid, 1-phenylethyl este; 1,4-Dihydro-2,6-dimethyl-5-nitro-4-[thieno[3,2c]-pyridin-3-yl]-3-pyridinecarboxylic acid, cyclopropylmethyl ester; 1,4-Dihydro-2,6-dimethyl-5-nitro-4-thieno[3,2-c]pyridin-3-yl]-3-pyridinecarboxylic acid, 1-cyclopropylethyl este; 1,4-Dihydro-2,6-dimethyl-5-nitro-4-[thieno[3,2c]-pyridin-3-yl]-3-pyridinecarboxylic acid, 2-cyanoethyl ester; 1,4-Dihydro-4-(2-{5-[4-(2-methoxyphenyl)-1-1piperazinyl]pentyl}-3-furanyl)-2,6-dimethyl-5-nitro-3-pyridinecarboxylic acid, methyl ester; 4-(4-Benzofurazanyl)-1,4-dihydro-2,6-dimethyl-5-nitro-3-pyridinecarboxylic acid, {4-[4-(2-methoxyphenyl)-1-piperazinyl]butyl}ester; 1,4-Dihydro-2,6-dimethyl-5-nitro-4-(3-pyridinyl)-3-pyridinecarboxylic acid, {4-[4-(2-pyrimidinyl)-1-piperazinyl]butyl}ester; 4-(3-Furanyl)-1,4-dihydro-2,6-dimethyl-5-nitro-3pyridinecarboxylic acid, {2-[4-(2-methoxyphenyl)-1piperazinyl]ethyl}ester; 4-(3-Furanyl)-1,4-dihydro-2,6-dimethyl-5-nitro-3pyridinecarboxylic acid, {2-[4-(2-pyrimidinyl)-1piperazinyl]ethyl}ester; 1,4-Dihydro-2,6-dimethyl-4-(1-methyl-1H-pyrrol-2-yl)-5-nitro-3-pyridinecarboxylic acid, {4-[4-(2-methoxyphenyl) 1-piperazinyl]butyl}ester; 1,4-Dihydro-2,6-dimethyl-4-(1-methyl-1H-pyrrol-2-yl)-5-nitro-3-pyridinecarboxylic acid, {4-[4-(2pyrimidinyl)-1-piperazinyl]butyl}ester; 1,4-Dihydro-2,6-dimethyl-5-nitro-4-(3-thienyl)-3-pyridinecarboxylic acid, {2-[4-(2-methoxyphenyl)-1-piperazinyl]ethyl}ester; 1,4-Dihydro-2,6-dimethyl-5-nitro-4-(3-thienyl)-3-pyridinecarboxylic acid, {2-[4-(2-pyrimidinyl)-1-piperazinyl]ethyl}ester; 4-(3-Furanyl)-1,4-dihydro-2,6-dimethyl-5-nitro-3-pyridinecarboxylic acid, {4-[4-(2-pyrimidinyl)-1-piperazinyl]butyl}ester; (4-(2-Furanyl)-1,4-dihydro-2,6-dimethyl-5-nitro-3-pyridinecarboxylic acid, {4-[4-(2-pyrimidinyl)-1-piperazinyl]butyl}ester; 1,4-Dihydro-2,6-dimethyl-5-nitro-4-(2-thienyl)-3-pyridinecarboxylic acid, {2-[4-(2-methoxyphenyl)-1-piperazinyl]ethyl}ester; 1,4-Dihydro-2,6-dimethyl-4-(1-methyl-1H-pyrrol-2-yl)-5-nitro-3-pyridinecarboxylic acid, {2-[4-(2-methoxyphenyl)-1-piperazinyl]ethyl}ester; 1,4-Dihydro-2,6-dimethyl-4-(1-methyl-1H-pyrrol-2-yl)-5-nitro-3-pyridinecarboxylic acid, {2-[4-(2pyrimidinyl) 1-piperazinyl]ethyl}ester; 5-(4-Chlorophenyl)-N-(3,5-dimethoxyphenyl)-2-furancarboxamide (A-803467); and analogs, derivatives, pharmaceutically acceptable salts, and/or prodrugs thereof.

In some embodiments of the aspects described herein, the ion-channel modulator is bufalin or analogs, derivatives, pharmaceutically acceptable salts, and/or prodrugs thereof. Exempalry bufalin analogs and derivatives include, but are not limited to, 7β-Hydroxyl bufalin; 3-epi-7β-Hydroxyl bufalin; 1β-Hydroxyl bufalin; 15α-Hydroxyl bufalin; 15β-Hydroxyl bufalin; Telocinobufagin (5-hydroxyl bufalin); 3-epi-Telocinobufagin; 3-epi-Bufalin-3-O-β-d-glucoside; 11β-Hydroxyl bufalin; 12β-Hydroxyl bufalin; 1β,7β-Dihydroxyl bufalin; 16α-Hydroxyl bufalin; 7β,16α-Dihydroxyl bufalin; 1β,12β-Dihydroxyl bufalin; resibufogenin; norbufalin; 3-hydroxy-14(15)-en-19-norbufalin-20,22-dienolide; 14-dehydrobufalin; bufotalin; arenobufagin; cinobufagin; marinobufagenin; proscillaridin; scillroside; scillarenin; and 14,15-epoxy-bufalin. Without limitation, analogs and derivatives of bufalin include those that can cross the blood-brain barrier. Herein, bufadienolides and analogs and derivatives thereof are also considered bufalin analaogs or derivatives thereof. Further bufalin or bufadienolide analogs and derivatives amenable to the present invention include those described in U.S. Pat. No. 3,080,362; U.S. Pat. No. 3,136,753; U.S. Pat. No. 3,470,240; U.S. Pat. No. 3,560,487; U.S. Pat. No. 3,585,187; U.S. Pat. No. 3,639,392; U.S. Pat. No. 3,642,770; U.S. Pat. No. 3,661,941; U.S. Pat. No. 3,682,891; U.S. Pat. No. 3,682,895; U.S. Pat. No. 3,687,944; U.S. Pat. No. 3,706,727; U.S. Pat. No. 3,726,857; U.S. Pat. No. 3,732,203; U.S. Pat. No. 3,806,502; U.S. Pat. No. 3,812,106; U.S. Pat. No. 3,838,146; U.S. Pat. No. 4,001,401; U.S. Pat. No. 4,102,884; U.S. Pat. No. 4,175,078; U.S. Pat. No. 4,242,33; U.S. Pat. No. 4,380,624; U.S. Pat. No. 5,314,932; U.S. Pat. No. 5,874,423; and U.S. Pat. No. 7,087,590 and those described in Min, et al., J. Steroid. Biochem. Mol. Biol., 91(1-2): 87-98 (2004); Kamano, Y. & Pettit, G. R. J. Org. Chem., 38 (12): 2202-2204 (1973); Watabe, et al., Cell Growth Differ, 8(8): 871 (1997); and Mahringer et al., Cancer Genomics and Proteomics, 7(4): 191-205 (2010).

In some embodiments, an agent as disclosed herein can be coupled to a ligand. Ligands can provide enhanced affinity for a selected target are also termed targeting ligands. Ligands in general can include therapeutic modifiers, e.g., for enhancing uptake; diagnostic compounds; or reporter groups e.g., for monitoring distribution. General examples include lipids, steroids, vitamins, sugars, proteins, peptides, polyamines, peptide mimics, and oligonucleotides.

Ligands can include a naturally occurring substance, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a specific sensory neuron cell type, e.g., a c-fiber. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic, an antibody or an aptamer.

Other examples of ligands include dyes, porphyrins (TPPC4, texaphyrin, Sapphyrin), polycyclic aromatic hydrocarbons (e.g., phenazine, dihydrophenazine), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O 3-(oleoyl)lithocholic acid, O3-(oleoyl)cholenic acid, dimethoxytrityl, or phenoxazine) and peptide conjugates (e.g., antennapedia peptide, Tat peptide), PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidyl species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The ligand can be, for example, a lipopolysaccharide, an activator of p38 MAP kinase, or an activator of NF-κB.

In another aspect, the ligand is a moiety, e.g., a vitamin, which is taken up by a target DRG cell body. Exemplary vitamins include vitamin A, E, and K. Other exemplary vitamins include are B vitamin, e.g., folic acid, B12, riboflavin, biotin, pyridoxal. Also included are HAS, low density lipoprotein (LDL) and high-density lipoprotein (HDL).

In some preferred embodiments, the ligand is a carbohydrate, e.g., monosaccharide, disaccharide, trisaccharide, oligosaccharide, and polysaccharide. Exemplary carbohydrate ligands include, but are not limited to, ribose, arabinose, xylose, lyxose, ribulose, xylulose, allose, altrose, glucose, mannose, gulose, idose, galactose, N—Ac-galatose, talose, psicose, fructose, sorbose, tagatose, fucose, fuculose, rhamonse, sedoheptulose, octose, nonose (neuraminic acid), sucrose, lactose, maltose, trehalose, turanose, cellobiose, raffinose, melezitose, maltotriose, acarbose, stachyose, fructooligosaccharide, galactooligosaccharides, mannanoligosaccharides, glycogen, starch (amylase, amylopectin), cellulose, beta-glucan (zymosan, lentinan, sizofuran), maltodextrin, inulin, levan beta (2->6), chitin, wherein the carbohydrate may be optionally substituted.

When the carbohydrate ligand comprises two or more sugars, each sugar can be independently selected from the group consisting of ribose, arabinose, xylose, lyxose, ribulose, xylulose, allose, altrose, glucose, mannose, gulose, idose, galactose, N—Ac-galatose, talose, psicose, fructose, sorbose, tagatose, fucose, fuculose, rhamonse, sedoheptulose, octose, and nonose (neuraminic acid), wherein the sugar may be optionally substituted. Without limitation each sugar can independently have the L- or the D-conformation. Furthermore, the linkage between two sugars can be independently α or β.

In alternative embodiments, an agent which is delivered to a DRG using the delivery device and system as disclosed herein is a functional genomic agent, including, but not limited to, RNA interference (RNAi) Technology (short interfering RNA molecules), Recombinant DNA, nucleic acid homologues and analogues, including protein-nucleic acid (PNA), pseudo-complementary-PNA, locked nucleic acid (LNA); Viral Vector based gene delivery, Bacterial Vector based gene delivery and histone modulating agents.

In alternative embodiments, an agent which is delivered to a DRG using the delivery device and system as disclosed is a biologic, for example, a toxin, for example, for selective ablation or death of a target cell in the DRG. In some embodiments, a toxin can be any toxin know to persons of ordinary skill in the art, and include, for example, botulinum toxin, conotoxins. In some embodiments, a toxin is an immunotoxin. An immunotoxin is typically composed of a targeting moiety, such as a ligand, growth factor or antibody that has cell type selectivity linked to a protein toxin or an antibody with extraordinary potency (Hall et al, 2001; Cancer Res; 81; 93-124). A suitable targeting moiety for use in an immunotoxin as disclosed herein would recognize and deliver the whole molecule to a specific receptor on the surface of selected sensory neuron in the DRG which is targeted for ablation or selective killing Thus, a toxin can trigger cell death by reaching the cytosol and catalytically inactivating vital cell process, or by modifying the sensory neuron cell membrane. Toxins used in immunotoxins are typically conjugated to a targeting moiety which can be an antibody that recognizes and binds to a surface receptor specifically expressed on the sensory neuron in the DRG, or be a ligand to a receptor which is specifically expressed on the surface of sensory neuron selected to be killed. Commonly used immunotoxins employs ribonucleases conjugated to monoclonal antibodies (MAb) (Hurset et al, 2002; 43; 953-959), often targeting the surface receptors of sensory neurons and carrying toxins capable of killing the cell with a single molecule (Yamaizumi et al, 1978; 15:245-250; Eiklid et al, 1980; 126:321-326).

In some embodiments, a toxin molecule or fragment thereof, or alternatively, an immunotoxin or fragment thereof can be delivered to a DRG using the delivery device as disclosed herien. In some embodiments, a toxins (or immunotoxins) includes, but are not limited to; protein toxin, bacterial toxin and plant toxin. Examples plant toxins include, but are not limited to, plant halotoxins, class II ribosome inactivating protein, plant hemitoxins, class I ribosome inactivating protein, saporin (SAP); pokeweed antiviral protein (PAP); bryodin 1; bouganin and gelonin, anthrax toxin; diphtheria toxin (DT); pseudomonal endotoxin (PE); streptolysin O; or naturally occurring variants, or genetically engineered variants or fragments thereof. Further examples of plant toxins useful as effector molecules in the methods as disclosed herein include, but are not limited to, ricin A chain (RTA); ricin B (RTB); abrin; mistletoe, lectin and modeccin or naturally occurring variants, or genetically engineered variants or fragments thereof. In some embodiments, a plant toxin is a ribotoxin, for example but not limited to ricin A chain (RTA). In further embodiments, a plant toxin can be a nuclease, for example but not limited to sarcin; restrictocin. In some embodiments, a cytotoxic molecule is delivered to the DRG using the delivery devices as disclosed herein, which include, for example a cytokine, such as, but not limited to, IL-1; IL-2; IL-3; IL-4; IL-13; interferon-□; tumor necrosis factor-alpha (TNF□); IL-6; granulosa colony stimulating factor (G-CSF); GM-CSF or natural variants or genetically engineered variants thereof. In some embodiments, a toxin is a nuclease or has endonucleolytic activity, for example a DNA nuclease or DNA endonuclease, for example DNA endonuclease I or natural variants or genetically engineered variant thereof. In alternative embodiments, a nuclease can be a RNA nuclease or RNA endonuclease, for example but not limited to RNA endonuclease I; RNA endonuclease II; RNA endonuclease III. In some embodiments, a RNA nuclease can be for example, but not limited to angliogenin, Dicer, RNase A or variants or fragments thereof.

In alternative embodiments, a toxin agent delivered to the DRG using the delivery devices as disclosed herein, include proteolytic enzymes, such as, but not limited to caspase enzymes; calpain enzymes; cathepsin enzymes; endoprotease enzymes; granzymes; matrix metalloproteases; pepsins; pronases; proteases; proteinases; rennin; trypsin or variants or fragments thereof.

In alternative embodiments, a toxin agent delivered to the DRG using the delivery devices as disclosed herein, includes a molecule that is capable of inducing a cell death pathway in the cell. In such embodiments, a toxin molecule which is capable of inducing cell death includes, a pro-apoptotic molecules, such as but not limited to Hsp90; TNF□; DIABLO; BAX; inhibitors of Bcl-2; Bad; poly ADP ribose polymerase-1 (PARP-1): Second Mitochondrial-derived Activator or Caspases (SMAC); apoptosis inducing factor (AIF); Fas (also known as Apo-1 or CD95); Fas Ligand (FasL) or variants or fragments thereof. In alternative embodiments, a toxin agent delivered to the DRG using the delivery devices as disclosed herein, tags a target polypeptide for protein degradation, e.g., can tag a ion channel such as a voltage gated sodium channel or a receptor expressed on the surface of a DRG for degradation. In such embodiments, such toxins that tag a target protein for degredation include, but are not limited to, ubiquitin; Small Ubiquitin-related Modifier (SUMO); DNA methyltransferase (DNA MTase); Histone acetylation enzyme (HAT) and variants or fragments thereof.

In some embodiments, an agent is delivered to the target spinal anatomy, e.g., DRG is a RNA interfering (RNAi) agent. As used herein, the term “RNA interference molecule” or “RNAi molecule” or “RNAi agent” are used interchangeably herein to refer to an RNA molecule, such as a double stranded RNA, which functions to inhibit gene expression of a target gene through RNA-mediated target transcript cleavage or RNA interference. Stated another way, the RNA interference inducing molecule induces gene silencing of the target gene. The overall effect of an RNA interference inducing molecule is gene silencing of the target gene. A double-stranded RNA, such as that used in siRNA, has different properties than single-stranded RNA, double-stranded DNA or single-stranded DNA. Each of the species of nucleic acids is bound by mostly non-overlapping sets of binding proteins in the cell and degraded by mostly non-overlapping sets of nucleases. The nuclear genome of all cells is DNA-based and as such is unlikely to produce immune responses except in autoimmune disease (Pisetsky. Clin Diagn Lab Immunol. 1998 January; 51:1-6). Single-stranded RNA (ssRNA) is the form endogenously found in eukaryotic cells as the product of DNA transcription. Cellular ssRNA molecules include messenger RNAs (and the progenitor pre-messenger RNAs), small nuclear RNAs, small nucleolar RNAs, transfer RNAs and ribosomal RNAs. Single-stranded RNA can induce interferon and inflammatory immune response via TLR7 and TLR8 receptors (Proc Natl Acad Sci. 2004. 101:5598-603; Science. 2004. 303:1526-9; Science. 2004. 303:1529-3). Double-stranded RNA induces a size-dependent immune response such that dsRNA larger than 30 bp activates the interferon response, while shorter dsRNAs feed into the cell's endogenous RNA interference machinery downstream of the Dicer enzyme. MicroRNAs (miRNAs), including short temporal RNAs and small modulatory RNAs, are the only known cellular dsRNA molecules in mammals and were not discovered until 2001 (Kim. 2005. Mol Cells. 19:1-15). Response to extracellular RNA in the bloodstream, double- or single-stranded of any length, is rapid excretion by the kidneys and degradation by enzymes (PLOS Biol. 2004. 2:18-20).

Accordingly, the RNA interference-inducing molecule referred to in the specification includes, but is not limited to, unmodified and modified double stranded (ds) RNA molecules including, short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also may contain 3′ overhangs, preferably 3′UU or 3′TT overhangs. In one embodiment, the siRNA molecules of the present invention do not include RNA molecules that comprise ssRNA greater than about 30-40 bases, about 40-50 bases, about 50 bases or more. In one embodiment, the siRNA molecules of the present invention have a double stranded structure. In one embodiment, the siRNA molecules of the present invention are double stranded for more than about 25%, more than about 50%, more than about 60%, more than about 70%, more than about 80%, more than about 90% of their length.

As used herein, “gene silencing” induced by RNA interference refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without introduction of RNA interference. In one preferred embodiment, the mRNA levels are decreased by at least about 70%, about 80%, about 90%, about 95%, about 99%, about 100%.

In another embodiment, siRNAs useful according the methods of the present invention are found in WO 05/042719, WO 05/013886, WO 04/039957, and U.S. Pat. App. No. 20040248296 which are incorporated in their entirety herein by reference. Other useful siRNAs useful in the methods of the present invention include, but are not limited to, those found in U.S. Pat. App. Nos. 20050176666, 20050176665, 20050176664, 20050176663, 20050176025, 20050176024, 20050171040, 20050171039, 20050164970, 20050164968, 20050164967, 20050164966, 20050164224, 20050159382, 20050159381, 20050159380, 20050159379, 20050159378, 20050159376, 20050158735, 20050153916, 20050153915, 20050153914, 20050148530, 20050143333, 20050137155, 20050137153, 20050137151, 20050136436, 20050130181, 20050124569, 20050124568, 20050124567, 20050124566, 20050119212, 20050106726, 20050096284, 20050080031, 20050079610, 20050075306, 20050075304, 20050070497, 20050054598, 20050054596, 20050053583, 20050048529, 20040248174, 20050043266, 20050043257, 20050042646, 20040242518, 20040241854, 20040235775, 20040220129, 20040220128, 20040219671, 20040209832, 20040209831, 20040198682, 20040191905, 20040180357, 20040152651, 20040138163, 20040121353, 20040102389, 20040077574, 20040019001, 20040018176, 20040009946, 20040006035, 20030206887, 20030190635, 20030175950, 20030170891, 20030148507, 20030143732, and WO 05/060721, WO 05/060721, WO 05/045039, WO 05/059134, WO 05/045041, WO 05/045040, WO 05/045039, WO 05/027980, WO 05/014837, WO 05/002594, WO 04/085645, WO 04/078181, WO 04/076623, and WO 04/04635, which are all incorporated herein in their entirety by reference.

In some embodiments, an agent delivered as disclosed herein increases gene expression of a gene, and is a synthetic modified RNAs (herein referred to as “MOD-RNA”) to induce protein expression in tissues, e.g., in the target spinal anatomy, e.g., DRG. In some embodiments, the cardiomyocytes are mammalian cardiomyocytes, for example human cardiomyocytes.

Admininstration of MOD-RNA results in a very rapid onset of protein expression, with protein expression levels significantly higher, e.g., at least about 2-fold higher, as compared to cells transfected than non-MOD RNA. In some embodiments, the optimal dose range for transfection with MOD-RNA is between 10-30 ng per 1000 cells, and that such a dose is non-toxic to cells.

Synthetic modified RNA's for delivery using the devices and methods as disclosed herein are are described in U.S. Provisional Application 61/387,220, filed Sep. 28, 2010, and U.S. Provisional Application 61/325,003, filed on Apr. 16, 2010, both of which are incorporated herein in their entirety by reference. In some embodiments, the synthetic, modified RNA molecule is not expressed in a vector, and the synthetic, modified RNA molecule can be a naked synthetic, modified RNA molecule. In some embodiments, a composition can comprises at least one synthetic, modified RNA molecule present in a lipid complex.

In a further embodiment, an agent delivered using the device as disclosed herein can be a small activating RNA, which is are disclosed in WO06/013559, US2005/0226848A1, WO2009/086428A2, U.S. Pat. No. 6,022,863, which are incorporated herein in their entirety by reference.

### E. DOSAGES OF AN AGENT DELIVERED BY THE DELIVERY DEVICE

In some embodiments, the agent release module is adapted for delivery of an agent or drug formulation over extended periods of time. Such agent release modules may be adapted for administration of an agent over several hours (e.g., 2 hours, 12 hours, or 24 hours to 48 hours or more), to several days (e.g., 2 to 5 days or more, from about 100 days or more), to several months or years. In some of these embodiments, an agent release module is adapted for delivery for a period ranging from about 1 month to about 12 months or more. An agent release module may be one that is adapted to administer an agent or drug formulation to a subject for a period of, for example, from about 2 hours to about 72 hours, from about 4 hours to about 36 hours, from about 12 hours to about 24 hours, from about 2 days to about 30 days, from about 5 days to about 20 days, from about 7 days or more, from about 10 days or more, from about 100 days or more; from about 1 week to about 4 weeks, from about 1 month to about 24 months, from about 2 months to about 12 months, from about 3 months to about 9 months, from about 1 month or more, from about 2 months or more, or from about 6 months or more; or other ranges of time, including incremental ranges, within these ranges, as needed, e.g., for the treatment or management of pain of the subject. In these embodiments, high-concentration formulations of an agent as described herein are of particular interest for use in the invention.

In one embodiment, the volume/time delivery rate of the agent is substantially constant (e.g., delivery is generally at a rate±about 5% to 10% of the cited volume over the cited time period, e.g., a volume rate of about a range of rates of from about 0.01 μg/hr to about 200 μg/hr, and which can be delivered at a volume rate of from about 0.01 μl/day to about 100 μl/day (i.e., from about 0.0004 μl/hr to about 4 μl/hr), preferably from about 0.04 μl/day to about 10 μl/day, generally from about 0.2 μl/day to about 5 μl/day, typically from about 0.5 μl/day to about 1 μl/day.

In one embodiment, the volume/time delivery rate of the agent is patterned or a temporal delivery, for example, delivery of a specific volume can be delivered followed by a specific time period of no delivery of an agent, followed by delivery of a specific volume, and repeating of the cycle. The amount of an agent delivered to the delivery site in the “on phase” (e.g., drug delivery phase) can be determined by the volume of drug delivered or alternatively, by a specific time period for delivery. For example, without limitation, an agent can be delivered in an “on” phase for a defined period of time of about 1 minute, or about 2 minutes, or about 5 minutes, or about 30 mins or about 1 hr, or longer than one hour, or any predetermined timeperiod in between, followed by a delivery “off phase” (e.g., no drug delivery phase) for a defined period of time of about 1 minute, or about 2 minutes, or about 5 minutes, or about 30 mins or about 1 hr, or about 2 hrs or about 3 hrs or about 6 hours or about 12 hours, or longer than 12 hours or any predetermined timeperiod in between. In alternative embodiments, an agent can be delivered in an “on” phase for a defined volume of delivery, for example, of about 0.01 μl, or about 0.05 μl, or about 0.1 μl, or about 0.2 μl, or about 0.5 μl or about 1.0 μl or about 2.0 μl more than about 2.0 μl, or any integer between 0.01 μl and 2.0 μl, or any predetermined volume of agene delivery, followed by a delivery “off” period (e.g., no drug delivery phase) for a defined period of time of about 1 minute, or about 2 minutes, or about 5 minutes, or about 30 mins or about 1 hr, or about 2 hrs or about 3 hrs or about 6 hours or about 12 hours, or longer than 12 hours or any predetermined timeperiod in between.

In some embodiments where the delivery elements 30 are leads, the drug delivery “on” and “off” phases may be coordinated with the electicical stimulation, e.g., electrical simulation can occur when the drug delivery is “off” but alternatively, depending on the agent delivered, can also occur when the drug delivery is in the “on” phase. In some embodiments, even with intermittent or patterned delivery of an agent, the rate of delivery of an agent can be delivered at rate from about 0.01 μg/hr to about 200 μg/hr, and which can be delivered at a volume rate of from about 0.01 μl/day to about 100 μl/day (i.e., from about 0.0004 μl/hr to about 4 μl/hr), preferably from about 0.04 μl/day to about 10 μl/day, generally from about 0.2 μl/day to about 5 μl/day, typically from about 0.5 μl/day to about 1 μl/day.

In general, an agent release module useful in the agent delivery device as disclosed herein can deliver agent at a low dose, e.g., from about 0.01 μg/hr to about 200 μg/hr, and preferably at a low volume rate e.g., on the order of nanoliters to microliters per day. In one embodiment, a volume rate of from about 0.01 μl/day to about 2 ml/day is accomplished by delivery of about 80 μl/hour over a period of 24 hours, with the delivery rate over that 24 hours period fluctuating over that period by about ±5% to 10%.

In some embodiments, the concentration of the agent can be and can be administered at a flow rate of at least about least 0.001 mg/mL, or at least about 0.01 mg/mL, or at least about 0.05 mg/mL or at least about 0.1 mg/mL, or at least about 0.5 mg/mL, 1 mg/mL, 10 mg/mL, 25 mg/mL, 50 mg/mL, 75 mg/mL, 100 mg/mL, 150 mg/mL, 200 mg/mL, 225 mg/mL, 250 mg/mL, 300 mg/mL, 350 mg/mL, 400 mg/mL, 450 mg/mL, 500 mg/mL, or greater. An agent delivered by a delivery device can be in solution, e.g., are dissolved in a liquid.

An agent delivered by a delivery device can be in a concentration which is lower than the dose of the agent delivered systemically or by another normal routine administration commonly used for administration of that agent in the art. In some embodiments, an agent is deliverered to the target spinal cord location at at least 5-fold, or at least about 10-fold or at least about 20-fold lower dose than the conventional dose for that agent when administered to the subject systemically or by another routine administration commonly used for administration of that agent in the art.

In some embodiments, the dose of the agent delivered to the target anatomy by the device as disclosed herein is determined by the concentration of the agent and the flow rate of the delivery of the agent to the target anatomy. In some embodiments, the concentration of the agent is lower than the conventionally used concentrations for that agent administered systemically or by the conventional administration route by at least about 5-fold, or at least about 10-fold, or at least about 50-fold, or at least about 100-fold, or at least about 200-fold, or at least about 500-fold or at least about 1000-fold, or any concentration interger between 5-fold and 1000-fold as compared to the concentration used when the agent is administered systemically or by the conventional administration route.

In some embodiments, the release of the agent from the reservoir (or agent holding chamber) of the agent release module is controlled by the subject, and the agent release module comprises a controllable pump.

Suitable amounts of an agent, e.g., a pharmaceutical agent useful to treat pain, e.g., chronic pain can range from about 0.5 cc up to a continuous drip for an initial therapeutic treatment. In some embodiments, agents can be delivered in concentrations ranging from about 1 nanogram per cc to about 10 g per cc, where the concentration of an agent depends on the type of agent (e.g. siRNA, small molecule, toxin, protein or antibody, etc), the potency of the specific agent used and the severity of the pain experienced by the subject. In some embodiments, the reservoir may be charged on a regularly scheduled basis, or it may be recharged as needed, as determined by the physician monitoring the patient's pain.

Abnormal regulation can be a result of excitation of the pathways or loss of inhibition of the pathways, a net result being an increase in perception or response. Agents suitable for use in the systems, methods and devices as disclosed herein can be directed to either blocking the transmission of signals or stimulating the inhibitory feedback. In some embodiments, electrical stimulation permits such stimulation of the target neurons. The electrical stimulation parameters can be adjusted and optimized for maximal benefit and coordinated effects with the delivered agent at the DRG, and to minimize side effects.

In general, an agent delivered to the DRG by the delivery device as disclosed herein is delivered at a volume rate that is compatible with delivery of an agent to the DRG, and at a dose that is therapeutically effective in reduction of pain (e.g., sufficient to accomplish substantial management of pain) while reducing the presence or risk of side effects that can be associated with administration of such agents, e.g., for example, where the agent has know side-effects, such as an opioid drug.

Subjects suffering from or susceptible to pain can receive alleviation of pain according to the method of the invention for any desired period of time. In general, administration of an agent to the target anatomy, e.g., DRG according to the methods of the present invention can be sustained for several hours (e.g., 2 hours, 12 hours, or 24 hours to 48 hours or more), to several days (e.g., 2 to 5 days or more), to several months or years. Typically, delivery can be continued for a period ranging from about 1 month to about 12 months or more. An agent delivered to the target anatomy, e.g., a DRG by the delivery device as disclosed herein can be administered to an individual for a period of, for example, from about 2 hours to about 72 hours, from about 4 hours to about 36 hours, from about 12 hours to about 24 hours, from about 2 days to about 30 days, from about 5 days to about 20 days, from about 7 days or more, from about 10 days or more, from about 100 days or more, from about 1 week to about 4 weeks, from about 1 month to about 24 months, from about 2 months to about 12 months, from about 3 months to about 9 months, from about 1 month or more, from about 2 months or more, or from about 6 months or more; or other ranges of time, including incremental ranges, within these ranges, as needed. This extended period of agent delivery is made possible by the ability of the invention to provide both adequate pain relief, while minimizing the severity of agent side effects (e.g., some agents such as opioids have side effects of nausea, vomiting, sedation, confusion, respiratory depression, addition etc.). In particular embodiments, an agent delivered to the DRG by the delivery device as disclosed herein can be delivered to the subject's DRG without the need for re-accessing the device and/or without the need for re-filling the device. In these embodiments, high-concentration formulations of an agent delivered to the DRG are of particular interest.

Preferably, an agent delivered to the DRG by the delivery device as disclosed herein can is delivered in a patterned fashion, more preferably in a substantially continuous fashion, e.g., substantially uninterrupted for a pre-selected period of drug delivery, and more preferably at a substantially constant, pre-selected rate or range of rates (e.g., amount of drug per unit time, or volume of drug formulation for a unit time). The drug is preferably delivered at a low volume rate of from about 0.01 μl/day to about 2 ml/day, preferably about 0.04 μl/day to about 1 ml/day, generally about 0.2 μl/day to about 0.5 ml/day, typically from about 2.0 μl/day to about 0.25 ml/day.

Specific delivery of an agent to the DRG at a low volume rate is a preferred embodiment of the invention. In general, low volume rate agent delivery to the DRG avoids accumulation of agent at the delivery site (e.g., depot or pooling effect) by providing for a rate of administration that is less than, the same as, or only very slightly greater than the rate of removal of agent from the delivery site (e.g., by absorption of agent in tissues and surrounding cells at the delivery site, movement of agent away from the delivery site by flow of blood or other bodily fluids, etc.). Thus, in addition to providing a delivery system for direct delivery of an agent to the DRG, the system and devices provide for delivery of highly potent agents, including, but not limited to, opiates, sodium channel blockers in a method for treating pain by balancing the rates of agent absorption and agent delivery to accomplish administration of a therapeutically effective amount of agent, while avoiding accumulation of agent at the delivery site.

In some embodiments, a DRG agent delivery device as disclosed herein can release the agent at the delivery target site in a substantially continuous preselected rate. For example, in some embodiments, an agent can be delivered at a rate of from about 0.01 μg/hr to about 200μ.g/hr, usually from about 0.01 μg/hr, 0.25 μg/hr, or 3 μg/hr to about 85 μg/hr, and typically between about 5 μg/hr to about 100 μg/hr. In some embodiments, an agent is delivered to the DRG at a rate of from about 0.01 μg/hr, 0.1 μg/hr, 0.25 μg/hr, 1 μg/hr, generally up to about 200 μg/hr. Appropriate amounts of the agent and the rate of delivery can be readily determined by the ordinarily skilled artisan based upon, for example, the relative potency of the agent or the drug formulation. The actual dose of agent delivered will vary with a variety of factors such as the potency and other properties of the selected agent used (e.g., lipophilicity, etc.).

In one embodiment, an agent delivered by a delivery device can be present in a formulation in a concentration substantially higher than conventional formulations, e.g., current commercially available formulations. By “substantially higher,” it is intended that the agent is present in the formulation in a concentration of at least about 2, at least about 5, at least about 10, at least about 20, at least about 50, at least about 100, at least about 250, at least about 500, at least about 1000, at least about 1500, at least about 2000, at least about 2500, at least about 3000, at least about 3500, at least about 4000, at least about 5000, at least about 6000, at least about 7000, at least about 8000, at least about 9000, at least about 10,000 times, or greater, than the solubility of agent in normal aqueous solution or conventional formulations for intrathecal or intravenous administration.

An agent delivered by a delivery device can be in a concentration of at least about 0.5 mg/mL, 1 mg/mL, 10 mg/mL, 25 mg/mL, 50 mg/mL, 75 mg/mL, 100 mg/mL, 150 mg/mL, 200 mg/mL, 225 mg/mL, 250 mg/mL, 300 mg/mL, 350 mg/mL, 400 mg/mL, 450 mg/mL, 500 mg/mL, or greater. An agent delivered by a delivery device can be in solution, e.g., are dissolved in a liquid.

In some embodiments, delivery of an agent directly to the DRG using the delivery device as disclosed herein is advantageous in instances where delivery of the agent by other routes has become undesirable, e.g., the subject has experienced intractable adverse side effects with oral, intravenous, or conventional intrathecal delivery of such an agent, or conventionally administered subcutaneous infusions (e.g., using a syringe driver system or other delivery system that requires relatively high volume delivery). Delivery using a delivery device as disclosed herein is convenient for the subject, as the implantation is permanent and also delivers the agent specifically to the DRG, thus minimizing non-specific side-effects. Additionally, agent delivery to the delivery device as disclosed herein can also increase patient compliance, prevent agent diversion and abuse, reduce the risk of infection associated with external pumps or other methods that require repeated breaking of the skin and/or maintenance of a port for administration.

Pharmaceutical grade organic or inorganic carriers and/or diluents suitable for delivery of an agent delivered by a delivery device can include any physiologically acceptable carriers. Exemplary liquid carriers for use in accordance with the present invention can be sterile non-aqueous or aqueous solutions which contain no materials other than the active ingredient. In general, hydrophobic solvents are generally preferred due to the lipophilicity of an agent. The formulations can optionally further comprise buffers, such as sodium phosphate at physiological pH value, physiological saline or both (i.e., phosphate-buffered saline). Suitable aqueous carriers may optionally further comprise more than one buffer salt, as well as other salts (such as sodium and potassium chlorides) and/or other solutes.

In some exemplary embodiments, an agent delivered by a delivery device can comprise a low molecular weight (e.g., MW less than about 300 g/mol) alcohol. In these embodiments, an agent delivered by a delivery device can be present in a formulation in a concentration of from about 0.5 mg/mL to about 500 mg/mL, from about 1 mg/mL to about 450 mg/mL, from about 50 mg/mL to about 400 mg/mL, from about 75 mg/mL to about 300 mg/mL, or from about 100 mg/mL to about 250 mg/mL. Suitable low molecular weight alcohols include those which are pharmaceutically acceptable, and which preferably comprise an aromatic moiety, and which are relatively immiscible in water (e.g., less than about 5, less than about 4, less than about 3, less than about 2, less than about 1 gram can dissolve in 25 ml H₂O), including, but not limited to, benzyl alcohol, and derivatives thereof. Small amounts of other pharmaceutically acceptable substances such as other pharmaceutically acceptable alcohols, e.g., ethanol, or water, may also be present, and, if present, are present in an amount of less than about 10%, less than about 5%, or less than about 1%.

In additional exemplary embodiments, an agent delivered by a delivery device can comprise a nonionic surfactant, in an alcohol ester, e.g., an ester of a low molecular weight alcohol as described above. In these embodiments, an agent delivered by a delivery device can be present in a formulation in a concentration of from about 0.5 mg/ml or 1 mg/mL to about 500 mg/mL, from about 50 mg/mL to about 300 mg/mL, from about 75 mg/mL to about 275 mg/mL, or from about 100 mg/mL to about 250 mg/mL. Suitable alcohol esters include those which are pharmaceutically acceptable, which preferably comprise an aromatic moiety, and which are insoluble in water, including, but not limited to, benzyl benzoate, and derivatives thereof. Small amounts of pharmaceutically acceptable substances such as pharmaceutically acceptable alcohols or other pharmaceutically acceptable alcohol esters, or water, may also be present, and, if present, are present in an amount of less than about 10%, less than about 5%, or less than about 1%. In a particular embodiment, the alcohol ester is 100% benzyl benzoate, with the agent to be delivered to the target spinal anatomy of the subject.

Suitable nonionic surfactants include those which are pharmaceutically acceptable, including but not limited to, polysorbate, e.g., polysorbate 20, polysorbate 40, polysorbate 60; sorbitan trioleate; polyoxyethylene polyoxypropyleneglycol, e.g., polyoxyethylene(160)glycol, and polyoxypropylene(30)glycol. Other nonionic surfactants which are suitable for use in the formulations include nonionic surfactants of the fatty acid polyhydroxy alcohol ester type such as sorbitan monolaurate, monooleate, monostearate or monopalmitate, sorbitan tristearate or trioleate, adducts of polyoxyethylene and fatty acid polyhydroxy alcohol esters such as polyoxyethylene sorbitan monolaurate, monooleate, monostearate, monopalmitate, tristearate or trioleate, polyethylene glycol fatty acid esters such as polyoxyethyl stearate, polyethylene glycol 400 stearate, polyethylene glycol 2000 stearate, in particular ethylene oxide-propylene oxide block copolymers of the Pluronics (Wyandotte) or Synperonic (ICI). In particular embodiments, the nonionic surfactant is polysorbate 20, polysorbate 40, polysorbate 60, or sorbitan trioleate, or mixtures of one or more of the foregoing.

In general, a nonionic surfactant is present in the formulation in a concentration of from about 50 mg/mL to about 200 mg/mL, from about 75 mg/mL to about 175 mg/mL, or from about 100 mg/mL to about 150 mg/mL.

Delivery of an agent to the DRG by the delivery device as disclosed herein is useful where delivery by other routes has become undesirable, e.g., the subject has experienced intractable adverse side effects with oral, intravenous, or conventional intrathecal administration of an agent, or ineffective treatment of pain. Delivery of an agent using the delivery devices as disclosed herein is convenient for the subject, as the delivery device implantation and removal procedures are a one time intervention. Also the DRG agent delivery device also allows for increased patient compliance, prevent agent diversion and abuse, reduce the risk of infection associated with external pumps or other methods that require repeated breaking of the skin and/or maintenance of a port for administration.

An agent delivered to the DRG by the delivery devices as disclosed herein at a low volume rate is a particularly preferred embodiment of the invention. In general, low volume rate agent delivery avoids accumulation of agent at the delivery site (e.g., depot or pooling effect) by providing for a rate of administration that is less than, the same as, or only very slightly greater than the rate of removal of agent from the delivery site (e.g., by absorption of agent in tissues at the site, movement of agent away from the site by flow of blood or other bodily fluids, etc.). Thus, in addition to enabling delivery of an agent to the target anatomy, e.g., the DRG, it allows delivery of highly potent agents, such as opioid antagonist, e.g., morphine, fentanyl and fentanyl congeners (e.g., sufentanil), and provides a method for treating pain by elegantly balancing the rates of agent absorption and agent delivery to accomplish administration of a therapeutically effective amount of agent, while avoiding accumulation of agent at the delivery site.

Formulations of particular interest for delivery are characterized in an agent to be delivered by the delivery device as disclosed herein can be present in a high concentration, as described above. An agent delivered by a delivery device can be soluble in the formulation, i.e., little or no agent precipitates form when the formulation comes in contact with an aqueous environment such as a body fluid.

The formulations comprising an agent to be delivered by the delivery device as disclosed herein can comprise additional active or inert components that are pharmaceutically acceptable and compatible with the active ingredient. Suitable excipients can comprise dextrose, glycerol, alcohol (e.g., ethanol), and the like, and combinations of one or more thereof with vegetable oils, propylene glycol, polyethylene glycol, benzyl alcohol, benzyl benzoate, dimethyl sulfoxide (DMSO), organics, and the like to provide a suitable composition. In addition, if desired, the composition can comprise hydrophobic or aqueous surfactants, dispersing agents, wetting or emulsifying agents, isotonic agents, pH buffering agents, dissolution promoting agents, stabilizers, antiseptic agents and other typical auxiliary additives employed in the formulation of pharmaceutical preparations.

Exemplary additional active ingredients that can be present in the formulations useful with the invention can include an opioid antagonist (e.g., to further decrease the possibility of addiction, or dependence, see, e.g., an exemplary osmotic dosage formulation comprising an opioid agonist and an opioid antagonist is described in U.S. Pat. No. 5,866,164, incorporated herein by reference.

### F. METHODS OF IMPLANTING THE DELIVERY DEVICE

The agent release module of the DRG agent delivery device can be implanted at any suitable implantation site. As noted infra, an implantation site is a site within the body of a subject at which an agent release module is introduced and positioned. Implantation sites include, but are not necessarily limited to a subdermal, subcutaneous, intramuscular, or other suitable site within a subject's body. Subcutaneous implantation sites are preferred because of convenience in implantation and removal of the agent delivery device. In some embodiments, the implantation site is at or near the DRG delivery site (e.g., the delivery site is not distant from the implantation site), and thus should be a site compatible with DRG delivery of agent (e.g., a subcutaneous site). Where the implantation site of the agent release module and the DRG delivery site are distant, then the agent release module can be implanted at a subcutaneous site, and the delivery of agent or drug formulation from a agent release module to the target DRG delivery site can be accomplished by transporting the agent or drug formulation via a catheter or lead, as described herein.

The DRG delivery site is an anatomical area of the body to which the agent or drug formulation is delivered.

In the examples herein, the device may be implanted using a variety of surgical methods. Methods to implant such devices wherein the distal end of the delivery element is located in proximity with the DRG is disclosed in U.S. patent applications 2010/0137938, 2010/0249875, US2008/0167698 and International Application, WO2010/083308, WO2008/070807, WO2006/029257, each of which are incorporated herein in their entirety by reference.

The method may further include monitoring pain experienced by the mammal and determining when the pain may be sufficient to indicate the need for additional medicine to be delivered to the determined nerve tissue. An agent, e.g., pain agent or analgesic can be repeatedly introduced into the reservoir in response to monitored pain experienced by the subject as desired.

Accordingly, the present disclosure advantageously provides implantable agent delivery systems that may be periodically and repeatedly charged with medicine for treating chronic nerve pain over an extended period of time, as well as methods suitable for the treatment of chronic nerve pain. It has been observed that the systems and methods disclosed herein advantageously enable treatment of chronic pain and overcome disadvantages with prior treatment devices and methods. For example, several advantages of the systems and devices herein include, without limitation, direct delivery of an agent to the DRG thus circumventing any side effect from non-specific or systemic administration or delivery to the CSF, also enabling lower doses of an agent to be delivered, thus reducing risk of unpleasant side effects, a combination of delivery of an agent to the DRG with electrical stimulation of the DRG, either simultaneously or temporally synchronized, for optimal agent delivery and therapeutic efficient for the treatment of pain. Additionally, the present systems, devices and methods of treatment enable an integrated system which enable user or patient controlled pain management which is substantially shielded from visual observation by a casual observer, as well as a completely internal system decreasing the risk of infection.

In some embodiments, the device and system may remain functional in the body of a subject for extended periods of time, such as for at least a year or at least 2 years, or between 2-5 years, or for 5-10 years or more than 10 years without removal of the system from the subject.

### G. DISORDERS SUSCEPTIBLE TO MANAGEMENT WITH THE DELIVERY DEVICES, SYSTEMS AND METHODS

Pain is amenable to alleviation using the methods, systems and delivery device as disclosed herein and includes, but is not necessarily limited to, various types of acute or chronic pain, including cancer pain, inflammatory disease pain, neuropathic pain, nociceptive pain, postoperative pain, iatrogenic pain, complex regional pain syndrome, failed-back pain, soft tissue pain, joint pain, bone pain, central pain, injury pain, arthritic pain, hereditary disease, infectious disease, headache, causalgia, hyperesthesia, sympathetic dystrophy, phantom limb syndrome, and denervation. This invention is particularly useful in the treatment of pain of long duration or chronic pain.

In general, administration of an agent, e.g. drug formulation using the delivery devices systems and methods according to the invention can be used to facilitate management of pain (e.g., palliative care through, e.g., systemic or centrally mediated analgesia) that is associated with any of a wide variety of disorders, conditions, or diseases. “Pain” as used herein, unless specifically noted otherwise, is meant to encompass pain of any duration and frequency, including, but not limited to, acute pain, chronic pain, intermittent pain, and the like. Causes of pain may be identifiable or unidentifiable. Where identifiable, the origin of pain may be, for example, of malignant, non-malignant, infectious, non-infectious, or autoimmune origin.

Of particular interest is the management of pain associated with disorders, diseases, or conditions that require long-term therapy, e.g., chronic and/or persistent diseases or conditions for which therapy involves treatment over a period of several days (e.g., about 3 days to 10 days), to several weeks (e.g., about 2 weeks or 4 weeks to 6 weeks), to several months or years, up to including the remaining lifetime of the subject. Subjects who are not presently suffering from a disease or condition, but who are susceptible to such may also benefit from prophylactic pain management using the devices and methods of the invention, e.g., prior to traumatic surgery. Pain amenable to therapy according to the invention may involve prolonged episodes of pain alternating with pain-free intervals, or substantially unremitting pain that varies in severity.

In general, pain can be nociceptive, somatogenic, neurogenic, or psychogenic. Somatogenic pain can be muscular or skeletal (i.e., osteoarthritis, lumbosacral back pain, posttraumatic, myofascial), visceral (i.e., pancreatitis, ulcer, irritable bowel), ischemic (i.e., arteriosclerosis obliterans), or related to the progression of cancer (e.g., malignant or non-malignant). Neurogenic pain can be due to posttraumatic and postoperative neuralgia, can be related to neuropathies (i.e., diabetes., toxicity, etc.), and can be related to nerve entrapment, facial neuralgia, perineal neuralgia, postamputation, thalamic, causalgia, and reflex sympathetic dystrophy.

Specific examples of pain-related disorders, conditions, diseases, and origins of pain amenable to management according to the present invention include, but are not necessarily limited to, cancer pain (e.g., metastatic or non-metastatic cancer), inflammatory disease pain, neuropathic pain, postoperative pain, iatrogenic pain (e.g., pain following invasive procedures or high dose radiation therapy, e.g., involving scar tissue formation resulting in a debilitating compromise of freedom of motion and substantial pain), complex regional pain syndromes, failed-back pain (e.g., acute or chronic back pain), soft tissue pain, joints and bone pain, central pain, injury (e.g., debilitating injuries, e.g., paraplegia, quadriplegia, etc., as well as non-debilitating injury (e.g., to back, neck, spine, joints, legs, arms, hands, feet, etc.)), arthritic pain (e.g., rheumatoid arthritis, osteoarthritis, arthritic symptoms of unknown etiology, etc.), hereditary disease (e.g., sickle cell anemia), infectious disease and resulting syndromes (e.g., Lyme disease, AIDS, etc.), headaches (e.g., migranes), causalgia, hyperesthesia, sympathetic dystrophy, phantom limb syndrome, denervation, and the like. Pain can be associated with any portion(s) of the body, e.g., the musculoskeletal system, visceral organs, skin, nervous system, etc.

Cancer pain is an example of one broad category of pain that can be alleviated according to the methods of the invention. One of the underlying causes of cancer pain is the severe local stretching of tissues by the neoplastic lesion. For example, as the cancer cells proliferate in an unrestricted manner, the tissues in the local region of cancer cell proliferation are subjected to mechanical stress required to displace tissue and accommodate the increased volume occupied by the tumor mass. When the tumor burden is confined to a small enclosed compartment, such as the marrow of a bone, the resulting pressure can result in severe pain. Another cause of cancer pain can result from the aggressive therapies used to combat the patient's cancer, e.g., radiation therapy, chemotherapy, etc. Such cancer therapies can involve localized or widespread tissue damage, resulting in pain.

Pain associated with any type of cancer is amenable to alleviation according to the invention. Specific examples of cancers that can be associated with pain (due to the nature of the cancer itself or therapy to treat the cancer) include, but are not necessarily limited to lung cancer, bladder cancer, melanoma, bone cancer, multiple myeloma, brain cancer, non-Hodgkins lymphoma, breast cancer, oral cancers, cervical cancer, ovarian cancer, colon cancer, rectal cancer, pancreatic cancer, dysplastic nevi, endocrine cancer, prostate cancer, head and neck cancers, sarcoma, Hodgkins disease, skin cancer, kidney cancer, stomach cancer, leukemia, testicular cancer, liver cancer, uterine cancer, and aplastic anemia. Certain types of neuropathic pain can also be amenable to treatment according to the invention.

Back pain, which is also amenable to management using the methods of the invention, is another broad category of pain that can be alleviated by application of the methods of the invention. Back pain is generally due to one or more of the following six causes: (i) stress on intervertebral facet joints, caused by slippage, arthritis, wedging, or scoliosis; (ii) radiculopathy, the mechanical compression of the nerve root due to bulging discs or tumors; (iii) tendonitis or tendon sprain; (iv) muscle spasm or muscle sprain; (v) ischemia, a local insufficiency in circulatory flow; and (vi) neuropathy, damage to nervous tissue of metabolic etiology or arising from cord tumors or central nervous system disease.

In some embodiments, the delivery devices, systems and methods as disclosed herein can be used to manage pain in patients who are opioid naive or who are no longer opioid naive, although due to the potency of the agents administered, patients are preferably not opioid naive. Exemplary opioid naive patients are those who have not received long-term opioid therapy for pain management. Exemplary non-opioid naive patients are those who have received short-term or long-term opioid therapy and have developed tolerance, dependence, or other undesirable side effect. For example, subjects who have intractable adverse side effects with oral, intravenous, or intrathecal morphine, or morphine analogues and derivatives, e.g., transdermal fentanyl patches, or conventionally administered subcutaneous infusions of fentanyl, morphine or other opioid can achieve good analgesia and maintain favorable side-effects profiles with delivery of agents and drug formulations when administered using the methods, delivery devices and systems as disclosed herein, for example, in the dose ranges and/or low volume rates described above.

In some embodiments, a physician can locate the source of the pain before installing the delivery device into a subject. It is desirable that the source of pain be accurately located in order for the patient to receive the most pain-relief benefit from the implant. A patient experiencing chronic nerve pain may verbally identify the location of the pain to the physician. The physician may also utilize the patient's prior medical history, imaging diagnostic tests, such as MRI or CT scans, or any other suitable diagnostic tests, in order to ascertain the location of the nerve tissue causing the chronic pain. In some embodiments, the physician identifies the spinal level associated with the chronic pain, including a peripheral nerve bundle including, but not limited to, the brachial plexus.

In further embodiments, the devices, systems and methods as disclosed herein may be routinely used for treatment of post-thoracotomy syndrome and non-entrapped dermatomal peripheral neuropathy, as well as any syndrome for chronic pain at the axial skeleton, excluding intrathecal locations.

Movement disorders are amenable to alleviation using the methods, systems and delivery device as disclosed herein and includes, but is not necessarily limited to, Akathisia, Akinesia (lack of movement), Associated Movements (Mirror Movements or Homolateral Synkinesis), Athetosis (contorted torsion or twisting), Ataxia, Ballismus (violent involuntary rapid and irregular movements) and Hemiballismus (affecting only one side of the body), Bradykinesia (slow movement), Cerebral palsy, Chorea (rapid, involuntary movement), including Sydenham's chorea, Rheumatic chorea and Huntington's disease, Dystonia (sustained torsion), including Dystonia muscularum, Blepharospasm, Writer's cramp, Spasmodic torticollis (twisting of head and neck), and Dopamine-responsive dystonia (hereditary progressive dystonia with diurnal fluctuation or Segawa's disease), Geniospasm (episodic involuntary up and down movements of the chin and lower lip), Myoclonus (brief, involuntary twitching of a muscle or a group of muscles), Metabolic General Unwellness Movement Syndrome (MGUMS), Multiple Sclerosis, Parkinson's disease, Restless Legs Syndrome RLS (WittMaack-Ekboms disease), Spasms (contractions), Stereotypic movement disorder, Stereotypy (repetition), Tardive dyskinesia, Tic disorders (involuntary, compulsive, repetitive, stereotyped), including Tourette's syndrome, Tremor (oscillations), Rest tremor (approximately 4-8 Hz), Postural tremor, Kinetic tremor, Essential tremor (approximately 6-8 Hz variable amplitude), Cerebellar tremor (approximately 6-8 Hz variable amplitude), Parkinsonian tremors (approximately 4-8 Hz variable amplitude), Physiological tremor (approximately 10-12 Hz low amplitude), and Wilson's disease.

The methods, systems and devices as disclosed herein may be used to treat movement disorders as described in U.S. Provisional Patent No. 61/438,895 entitled, “Devices, Systems and Methods for the Targeted Treatment of Movement Disorders”, incorporated herein by reference. The targeted treatment of such conditions is provided with minimal deleterious side effects, such as undesired motor responses or undesired stimulation of unaffected body regions. This is achieved by directly neuromodulating a target anatomy associated with the condition while minimizing or excluding undesired neuromodulation of other anatomies. Tt may be appreciated that neuromodulation may include a variety of forms of altering or modulating nerve activity by delivering electrical and/or pharmaceutical agents directly to a target area, such as the DRG.

The present invention may be defined in any of the following numbered paragraphs:

1. A neuromodulation system comprising:
a delivery element having a distal end and at least one outlet port disposed near the distal end, wherein the distal end is configured for positioning at least one of the at least one outlet ports near a dorsal root ganglion;
an agent release module connectible with the delivery element, the agent release module having an agent release mechanism; and
an agent releaseable from the agent release mechanism so as to be delivered from the at least one outlet port according to a controlled release pattern to at least assist in neuromodulating the dorsal root ganglion.
   2. The neuromodulation system as in paragraph 1, wherein the agent is chargeable and the agent release mechanism includes a mechanism for charging the agent so that the agent is delivered by iontophoretic flux according to the controlled release pattern.
   3. The neuromodulation system as in paragraphs 1 or 2, wherein the agent is selected from one or more of the group consisting of: lidocaine, epinephrine, fentanyl, fentanyl hydrochloride, ketamine, dexamethasone, hydrocortisone, peptides, proteins, Angiotension II antagonist, Antriopeptins, Bradykinin, Tissue Plasminogen activator, Neuropeptide Y, Nerve growth factor (NGF), Neurotension, Somatostatin, octreotide, Immunomodulating peptides and proteins, Bursin, Colony stimulating factor, Cyclosporine, Enkephalins, Interferon, Muramyl dipeptide, Thymopoietin, TNF, growth factors, Epidermal growth factor (EGF), Insulin-like growth factors I & II (IGF-I & II), Inter-leukin-2 (T-cell growth factor) (Il-2), Nerve growth factor (NGF), Platelet-derived growth factor (PDGF), Transforming growth factor (TGF) (Type I or δ) (TGF), Cartilage-derived growth factor, Colony-stimulating factors (CSFs), Endothelial-cell growth factors (ECGFs), Erythropoietin, Eye-derived growth factors (EDGF), Fibroblast-derived growth factor (FDGF), Fibroblast growth factors (FGFs), Glial growth factor (GGF), Osteosarcoma-derived growth factor (ODGF), Thymosin, Transforming growth factor (Type II or β)(TGF).
   4. The neuromodulation system as in any of paragraphs 1-3, wherein the agent is selected from one or more of the group consisting of: opioids, COX inhibitors, PGE2 inhibitors, Na+ channel inhibitors.
   5. The neuromodulation system as in any of paragraphs 1-4, wherein the agent is an agonist or antagonist of a receptor or ion channel expressed by a dorsal root ganglion.
   6. The neuromodulation system as in as in any of paragraphs 1-5, wherein the agent is an agonist or antagonist of a receptor or ion channel which is upregulated in a dorsal root ganglion in response to nerve injury, inflammation, neuropathic pain, and/or nociceptive pain.
   7. The neuromodulation system as in any of paragraphs 1-6, wherein the ion channel expressed by the dorsal root ganglion is selected from the group consisting of: voltage gated sodium channels (VGSC), voltage gated Calcium Channels (VGCC), voltage gated potassium channel (VGPC), acid-sensing ion channels (ASICs).
   8. The neuromodulation system as in any of paragraphs 1-7, wherein the voltage-gated sodium channel includes TTX-resistant voltage gated sodium channels.
   9. The neuromodulation system as in any of paragraphs 1-8, wherein the TTX-resistant voltage gated sodium channels include Naᵥ1.8 and Naᵥ1.9.
   10. The neuromodulation system as in any of paragraphs 1-9, wherein the voltage-gated sodium channel includes TTX-sensitive voltage gated sodium channels.
   11. The neuromodulation system as in any of paragraphs 1-10, wherein the TTX-sensitive voltage gated sodium channels is Brain III (Naᵥ1.3).
   12. The neuromodulation system as in any of paragraphs 1-11, wherein the receptor is selected from ATP receptor, NMDA receptors, EP4 recetors, metrix metalloproteins (MMPs), TRP receptors, neurtensin receptors.
   13. The neuromodulation system as in any of paragraphs 1-12, wherein the delivery element further comprises at least one electrode which is capable of delivering electrical energy.
   14. The neuromodulation system as in any of paragraphs 1-13, wherein the electrical energy at least assists in creating the iontophoretic flux of the agent.
   15. The neuromodulation system as in any of paragraphs 1-14, wherein the at least one electrode in close proximity to the at least one outlet port.
   16. The neuromodulation system as in any of paragraphs 1-5, wherein the agent release module further comprises a pulse generator which provides the electrical energy in a manner which impacts the effect of the agent on at least a portion of the dorsal root ganglion.
   17. The neuromodulation system as in any of paragraphs 1-16, wherein the electrical energy is provided once the agent has targeted at least a portion of the dorsal root ganglion.
   18. The neuromodulation system as in any of paragraphs 1-17, wherein the electrical energy is provided in a manner that targets at least one particular type of cell within the dorsal root ganglion.
   19. The neuromodulation system as in any of paragraphs 1-18, wherein the controlled release pattern is determined to impact an effect of the electrical energy on at least a portion of the dorsal root ganglion.
   20. The neuromodulation system as in any of paragraphs 1-19, wherein the agent and/or the controlled release pattern is determined to enhance the ability of the electrical energy to excite or inhibit a primary sensory neuron in the dorsal root ganglion.
   21. The neuromodulation system as in any of paragraphs 1-20, wherein the agent and/or the controlled release pattern is determined to cause a change in the open probability of at least one sodium channel.
   22. The neuromodulation system as in any of paragraphs 1-21, wherein the agent release mechanism delivers the agent to assist in neuromodulating the dorsal root ganglion over time.
   23. The neuromodulation system as in any of paragraphs 1-22, wherein the agent release mechanism comprises a matrix impregnated with the agent so that the matrix releases the agent over time according to the controlled release pattern.
   24. The neuromodulation system as in any of paragraphs 1-23, wherein the matrix comprises an erodible material.
   25. The neuromodulation system as in any of paragraphs 1-24, wherein the agent comprises a carrier particle.
   26. The neuromodulation system as in any of paragraphs 1-25, wherein the carrier particle is selected from one or more from the group consisting of: a macromolecule complex, nanocapsule, microsphere, bead or lipid-based system, micelle, mixed micelle, liposome or lipid:oligonucleotide complex of uncharacterized structure, dendrimer, virosome, nanocrystal, quantum dot, nanoshell, nanorod.
   27. The neuromodulation system as in any of paragraphs 1-26, wherein the agent comprises a targeting molecule which targets the dorsal root ganglion.
   28. The neuromodulation system as in any of paragraphs 1-27, wherein the targeting molecule has a specific affinity for a cell surface marker expressed on at least one cell within the dorsal root ganglion.
   29. The neuromodulation system as in any of paragraphs 1-28, wherein the at least one cell comprises at least one cell body of a c-fiber.
   30. The neuromodulation system as in any of paragraphs 1-29, wherein the agent comprises a gellable material which retains the agent near the dorsal root ganglion after delivery.
   31. The neuromodulation system as in any of paragraphs 1-30, wherein the gellable material is gellable upon delivery.
   32. The neuromodulation system as in any of paragraphs 1-31, wherein positioning the distal end of the delivery element comprises positioning at least one of the at least one outlet port on or in contact with the dorsal root ganglion epinurium.
   33. The neuromodulation system as in any of paragraphs 1-33, wherein the delivery element is not implanted into the dorsal root ganglion.
   34. An intrathecal agent delivery system comprising:
a delivery element having a distal end and at least one outlet port disposed near the distal end, wherein the delivery element is configured for advancement within an intrathecal space along a spinal cord and then along a dorsal root to position at least one of the at least one outlet ports near an associated dorsal root ganglion;
an agent release module connectible with the delivery element, the agent release module having an agent release mechanism; and
an agent releaseable from the agent release mechanism so as to be delivered from the at least one outlet port to at least assist in neuromodulating the dorsal root ganglion.
   35. The intrathecal delivery system as in paragraph 34, wherein the delivery element includes a stylet, wherein the stylet has a curved distal end configured to assist in guiding the delivery element along a root sleeve angulation of the dorsal root during advancement.
   36. The intrathecal delivery system as in paragraph 34 or 35, wherein the agent comprises a targeting molecule which targets the agent to the dorsal root ganglion.
   37. The intrathecal delivery system as in any of paragraphs 34-36, wherein the targeting molecule has a specific affinity for a cell surface marker expressed on at least one cell within the dorsal root ganglion.
   38. The intrathecal delivery system as in any of paragraphs 34-37, wherein the agent comprises a benzodiazepine, clonazepam, morphine, baclofen and/or ziconotide.
   39. The intrathecal delivery system as in any of paragraphs 34-39, wherein the agent comprises a genomic agent or biologic.
   40. The intrathecal delivery system as in any of paragraphs 34-39, wherein the agent is activatable by electrical stimulation.
   41. The intrathecal delivery system as in any of paragraphs 34-40, wherein the agent enhances the ability of electrical stimulation to excite or inhibit a primary sensory neuron in the dorsal root ganglion.
   42. The intrathecal delivery system as in any of paragraphs 34-41, wherein the agent enhances the ability of electrical stimulation to target at least one specific cell within the dorsal root ganglion.
   43. The intrathecal delivery system as in any of paragraphs 34-42, wherein the agent release module includes electronic circuitry capable of generating stimulation energy for delivery to a delivery element having an electrode.
   44. The intrathecal delivery system as in any of paragraphs 34-43, wherein the electronic circuitry includes memory programmable with an electrical stimulation parameter set and an agent delivery parameter set.
   45. The intrathecal delivery system as in any of paragraphs 34-44, wherein the parameter sets cause the agent and the stimulation energy to be delivered in a predetermined coordinated manner.
   46. An agent delivery system comprising:
a delivery element having a distal end, at least one agent delivery structure disposed near the distal end and at least one electrode disposed near the distal end, wherein the distal end is configured for positioning at least one of at least one agent delivery structures and at least one of the at least one electrodes near a dorsal root ganglion; and
a pulse generator connectable with the delivery element, wherein the pulse generator includes memory programmable with an electrical stimulation parameter set that controls delivery of electrical energy from the at least one electrode in a predetermined manner dependent on the delivery of an agent from the at least one of the at least one agent delivery structures.
   47. The agent delivery system as in paragraph 46, wherein the agent delivery structure comprises an agent-eluting coating.
   48. An agent delivery system as in paragraph 46 or 47, wherein the agent delivery structure comprises an agent-eluting structure.
   49. An agent delivery system as in any of paragraphs 46-48, wherein the agent delivery structure comprises an agent outlet port.
   50. An agent delivery system as in any of paragraphs 46-49, wherein the pulse generator further comprises an agent release mechanism which releases agent to the at least one agent outlet port.
   51. An agent delivery system as in any of paragraphs 46-50, wherein the pulse generator includes memory programmable with an agent delivery parameter set that controls delivery of the agent from the agent release mechanism.
   52. An agent delivery system as in any of paragraphs 46-51, wherein the delivery of the electrical energy is controlled to impact the effect of the agent on at least a portion of the dorsal root ganglion.
   53. An agent delivery system as in any of paragraphs 46-52, wherein the delivery of the electrical energy is timed to maximize the effect of the agent on the at least a portion of the dorsal root ganglion.
   54. An agent delivery system as in any of paragraphs 46-53, wherein the delivery of the electrical energy is controlled based on an impact the delivery agent has on the effect of the electrical energy on at least a portion of the dorsal root ganglion.
   55. An agent delivery system as in any of paragraphs 46-54, wherein the delivery of the electrical energy is reduced during delivery of the agent.
   56. A neuromodulation system comprising:
an agent delivery system including a delivery element having a distal end, at least one agent delivery structure disposed near the distal end and at least one electrode disposed near the distal end, wherein the distal end is configured for positioning at least one of the at least one agent delivery structure and at least one of the at least one electrodes near a dorsal root ganglion; and
an agent releaseable from the at least one agent delivery structure, wherein electrical energy provided by the at least one electrode assists in neuromodulating the dorsal root ganglion by activating a cell body within the dorsal root ganglion so that the cell body is preferentially targeted by the agent.
   57. The neuromodulation system as in paragraph 56, wherein activating the cell body comprises depolarizing the cell body.
   58. The neuromodulation system as in paragraph 56 or 57, wherein the cell body is preferentially activated based on its size and/or membrane properties.
   59. The neuromodulation system as in any of paragraphs 56-58, wherein the agent comprises a toxin.
   60. A neuromodulation system comprising:
an agent delivery system including a delivery element having a distal end, at least one agent delivery structure disposed near the distal end and at least one electrode disposed near the distal end, wherein the distal end is configured for positioning at least one of the agent delivery structures and at least one of the one electrodes near a dorsal root ganglion; and
an agent releaseable from the at least one agent delivery structure, wherein electrical energy provided by the at least one electrode selectively activates the agent in a first cell type within the dorsal root ganglion while not activating the agent in a second cell type within the dorsal root ganglion.
   61. The neuromodulation system as in paragraph 60, wherein the agent comprises a pro-drug.
   62. The neuromodulation system as in paragraph 60 or 61, wherein the agent is selected from one or any combination selected from the group consisting of: opioids, COX inhibitors, PGE2 inhibitors, Na+ channel inhibitors.
   63. The neuromodulation system as in any of paragraphs 60-62, wherein the agent is an agonist or antagonist of a receptor or ion channel which is upregulated in a dorsal root ganglion in response to nerve injury, inflammation, neuropathic pain, and/or nociceptive pain.
   64. The neuromodulation system as in any of paragraphs 60-63, wherein the ion channel expressed by the dorsal root ganglion is selected from the group consisting of: voltage gated sodium channels (VGSC), voltage gated Calcium Channels (VGCC), voltage gated potassium channel (VGPC), acid-sensing ion channels (ASICs).
   65. The neuromodulation system as in any of paragraphs 60-64, wherein the voltage-gated sodium channel includes TTX-resistant voltage gated sodium channels.
   66. The neuromodulation system as in any of paragraphs 60-65, wherein the TTX-resistant voltage gated sodium channels include Naᵥ1.8 and Naᵥ1.9.
   67. The neuromodulation system as in any of paragraphs 60-66, wherein the voltage-gated sodium channel includes TTX-sensitive voltage gated sodium channels.
   68. The neuromodulation system as in any of paragraphs 60-67, wherein the TTX-sensitive voltage gated sodium channels is Brain III (Naᵥ1.3).
   69. The neuromodulation system as in any of paragraphs 60-68, wherein the receptor is selected from ATP receptor, NMDA receptors, EP4 receptors, matrix metalloproteins (MMPs), TRP receptors, neurtensin receptors.

### REFERENCES

All references cited in the specification and throughout the application are incorporated herein in their entirety.

1. A neuromodulation system comprising:
a delivery element having a distal end and at least one outlet port disposed near the distal end, wherein the distal end is configured for positioning at least one of the at least one outlet ports near a dorsal root ganglion;
an agent release module connectible with the delivery element, the agent release module having an agent release mechanism; and
an agent releaseable from the agent release mechanism so as to be delivered from the at least one outlet port according to a controlled release pattern to at least assist in neuromodulating the dorsal root ganglion.

2. The neuromodulation system as in claim 1, wherein the agent is chargeable and the agent release mechanism includes a mechanism for charging the agent so that the agent is delivered by iontophoretic flux according to the controlled release pattern.

3. The neuromodulation system as in claim 2, wherein the agent is selected from one or more of the group consisting of: lidocaine, epinephrine, fentanyl, fentanyl hydrochloride, ketamine, dexamethasone, hydrocortisone, peptides, proteins, Angiotension II antagonist, Antriopeptins, Bradykinin, Tissue Plasminogen activator, Neuropeptide Y, Nerve growth factor (NGF), Neurotension, Somatostatin, octreotide, Immunomodulating peptides and proteins, Bursin, Colony stimulating factor, Cyclosporine, Enkephalins, Interferon, Muramyl dipeptide, Thymopoietin, TNF, growth factors, Epidermal growth factor (EGF), Insulin-like growth factors I & II (IGF-I & II), Inter-leukin-2 (T-cell growth factor) (Il-2), Nerve growth factor (NGF), Platelet-derived growth factor (PDGF), Transforming growth factor (TGF) (Type I or δ) (TGF), Cartilage-derived growth factor, Colony-stimulating factors (CSFs), Endothelial-cell growth factors (ECGFs), Erythropoietin, Eye-derived growth factors (EDGF), Fibroblast-derived growth factor (FDGF), Fibroblast growth factors (FGFs), Glial growth factor (GGF), Osteosarcoma-derived growth factor (ODGF), Thymosin, Transforming growth factor (Type II or β)(TGF).

4. The neuromodulation system as in claim 1, wherein the agent is selected from one or more of the group consisting of: opioids, COX inhibitors, PGE2 inhibitors, Na+ channel inhibitors.

5. The neuromodulation system as in claim 1, wherein the agent is an agonist or antagonist of a receptor or ion channel expressed by a dorsal root ganglion.

6. The neuromodulation system as in claim 1, wherein the agent is an agonist or antagonist of a receptor or ion channel which is upregulated in a dorsal root ganglion in response to nerve injury, inflammation, neuropathic pain, and/or nociceptive pain.

7. The neuromodulation system as in claim 6, wherein the ion channel expressed by the dorsal root ganglion is selected from the group consisting of: voltage gated sodium channels (VGSC), voltage gated Calcium Channels (VGCC), voltage gated potassium channel (VGPC), acid-sensing ion channels (ASICs).

8. The neuromodulation system as in claim 7, wherein the voltage-gated sodium channel includes TTX-resistant voltage gated sodium channels.

9. The neuromodulation system as in claim 8, wherein the TTX-resistant voltage gated sodium channels include Naᵥ1.8 and Naᵥ1.9.

10. The neuromodulation system as in claim 7, wherein the voltage-gated sodium channel includes TTX-sensitive voltage gated sodium channels.

11. The neuromodulation system as in claim 10, wherein the TTX-sensitive voltage gated sodium channels is Brain III (Naᵥ1.3).

12. The neuromodulation system as in claim 6, wherein the receptor is selected from ATP receptor, NMDA receptors, EP4 receptors, metrix metalloproteins (MMPs), TRP receptors, neurtensin receptors.

13. The neuromodulation system as in claim 1, wherein the delivery element further comprises at least one electrode which is capable of delivering electrical energy.

14. The neuromodulation system as in claim 13, wherein the electrical energy at least assists in creating the iontophoretic flux of the agent.

15. The neuromodulation system as in claim 13, wherein the at least one electrode in close proximity to the at least one outlet port.

16. The neuromodulation system as in claim 13, wherein the agent release module further comprises a pulse generator which provides the electrical energy in a manner which impacts the effect of the agent on at least a portion of the dorsal root ganglion.

17. The neuromodulation system as in claim 16, wherein the electrical energy is provided once the agent has targeted at least a portion of the dorsal root ganglion.

18. The neuromodulation system as in claim 16, wherein the electrical energy is provided in a manner that targets at least one particular type of cell within the dorsal root ganglion.

19. The neuromodulation system as in claim 13, wherein the controlled release pattern is determined to impact an effect of the electrical energy on at least a portion of the dorsal root ganglion.

20. The neuromodulation system as in claim 19, wherein the agent and/or the controlled release pattern is determined to enhance the ability of the electrical energy to excite or inhibit a primary sensory neuron in the dorsal root ganglion.

21. The neuromodulation system as in claim 20, wherein the agent and/or the controlled release pattern is determined to cause a change in the open probability of at least one sodium channel.

22. The neuromodulation system as in claim 1, wherein the agent release mechanism delivers the agent to assist in neuromodulating the dorsal root ganglion over time.

23. The neuromodulation system as in claim 1, wherein the agent release mechanism comprises a matrix impregnated with the agent so that the matrix releases the agent over time according to the controlled release pattern.

24. The neuromodulation system as in claim 23, wherein the matrix comprises an erodible material.

25. The neuromodulation system as in claim 1, wherein the agent comprises a carrier particle.

26. The neuromodulation system as in claim 25, wherein the carrier particle is selected from one or more from the group consisting of: a macromolecule complex, nanocapsule, microsphere, bead or lipid-based system, micelle, mixed micelle, liposome or lipid:oligonucleotide complex of uncharacterized structure, dendrimer, virosome, nanocrystal, quantum dot, nanoshell, nanorod.

27. The neuromodulation system as in claim 1, wherein the agent comprises a targeting molecule which targets the dorsal root ganglion.

28. The neuromodulation system as in claim 27, wherein the targeting molecule has a specific affinity for a cell surface marker expressed on at least one cell within the dorsal root ganglion.

29. The neuromodulation system as in claim 27, wherein the at least one cell comprises at least one cell body of a c-fiber.

30. The neuromodulation system as in claim 1, wherein the agent comprises a gellable material which retains the agent near the dorsal root ganglion after delivery.

31. The neuromodulation system as in claim 30, wherein the gellable material is gellable upon delivery.

32. The neuromodulation system as in claim 1, wherein positioning the distal end of the delivery element comprises positioning at least one of the at least one outlet port on or in contact with the dorsal root ganglion epinurium.

33. The neuromodulation system as in claim 1, wherein the delivery element is not implanted into the dorsal root ganglion.

34. An intrathecal agent delivery system comprising:
a delivery element having a distal end and at least one outlet port disposed near the distal end, wherein the delivery element is configured for advancement within an intrathecal space along a spinal cord and then along a dorsal root to position at least one of the at least one outlet ports near an associated dorsal root ganglion;
an agent release module connectible with the delivery element, the agent release module having an agent release mechanism; and
an agent releaseable from the agent release mechanism so as to be delivered from the at least one outlet port to at least assist in neuromodulating the dorsal root ganglion.

35. The intrathecal delivery system as in claim 34, wherein the delivery element includes a stylet, wherein the stylet has a curved distal end configured to assist in guiding the delivery element along a root sleeve angulation of the dorsal root during advancement.

36. The intrathecal delivery system as in claim 34, wherein the agent comprises a targeting molecule which targets the agent to the dorsal root ganglion.

37. The intrathecal delivery system as in claim 36, wherein the targeting molecule has a specific affinity for a cell surface marker expressed on at least one cell within the dorsal root ganglion.

38. The intrathecal delivery system as in claim 34, wherein the agent comprises a benzodiazepine, clonazepam, morphine, baclofen and/or ziconotide.

39. The intrathecal delivery system as in claim 34, wherein the agent comprises a genomic agent or biologic.

40. The intrathecal delivery system as in claim 34, wherein the agent is activatable by electrical stimulation.

41. The intrathecal delivery system as in claim 34, wherein the agent enhances the ability of electrical stimulation to excite or inhibit a primary sensory neuron in the dorsal root ganglion.

42. The intrathecal delivery system as in claim 34, wherein the agent enhances the ability of electrical stimulation to target at least one specific cell within the dorsal root ganglion.

43. The intrathecal delivery system as in claim 34, wherein the agent release module includes electronic circuitry capable of generating stimulation energy for delivery to a delivery element having an electrode.

44. The intrathecal delivery system as in claim 43, wherein the electronic circuitry includes memory programmable with an electrical stimulation parameter set and an agent delivery parameter set.

45. The intrathecal delivery system as in claim 44, wherein the parameter sets cause the agent and the stimulation energy to be delivered in a predetermined coordinated manner.

46. An agent delivery system comprising:
a delivery element having a distal end, at least one agent delivery structure disposed near the distal end and at least one electrode disposed near the distal end, wherein the distal end is configured for positioning at least one of at least one agent delivery structures and at least one of the at least one electrodes near a dorsal root ganglion; and
a pulse generator connectable with the delivery element, wherein the pulse generator includes memory programmable with an electrical stimulation parameter set that controls delivery of electrical energy from the at least one electrode in a predetermined manner dependent on the delivery of an agent from the at least one of the at least one agent delivery structures.

47. The agent delivery system as in claim 46, wherein the agent delivery structure comprises an agent-eluting coating.

48. An agent delivery system as in claim 46, wherein the agent delivery structure comprises an agent-eluting structure.

49. An agent delivery system as in claim 46, wherein the agent delivery structure comprises an agent outlet port.

50. An agent delivery system as in claim 49, wherein the pulse generator further comprises an agent release mechanism which releases agent to the at least one agent outlet port.

51. An agent delivery system as in claim 50, wherein the pulse generator includes memory programmable with an agent delivery parameter set that controls delivery of the agent from the agent release mechanism.

52. An agent delivery system as in claim 46, wherein the delivery of the electrical energy is controlled to impact the effect of the agent on at least a portion of the dorsal root ganglion.

53. An agent delivery system as in claim 52, wherein the delivery of the electrical energy is timed to maximize the effect of the agent on the at least a portion of the dorsal root ganglion.

54. An agent delivery system as in claim 46, wherein the delivery of the electrical energy is controlled based on an impact the delivery agent has on the effect of the electrical energy on at least a portion of the dorsal root ganglion.

55. An agent delivery system as in claim 46, wherein the delivery of the electrical energy is reduced during delivery of the agent.

56. A neuromodulation system comprising:
an agent delivery system including a delivery element having a distal end, at least one agent delivery structure disposed near the distal end and at least one electrode disposed near the distal end, wherein the distal end is configured for positioning at least one of the at least one agent delivery structure and at least one of the at least one electrodes near a dorsal root ganglion; and
an agent releaseable from the at least one agent delivery structure, wherein electrical energy provided by the at least one electrode assists in neuromodulating the dorsal root ganglion by activating a cell body within the dorsal root ganglion so that the cell body is preferentially targeted by the agent.

57. The neuromodulation system as in claim 56, wherein activating the cell body comprises depolarizing the cell body.

58. The neuromodulation system as in claim 56, wherein the cell body is preferentially activated based on its size and/or membrane properties.

59. The neuromodulation system as in claim 56, wherein the agent comprises a toxin.

60. A neuromodulation system comprising:
an agent delivery system including a delivery element having a distal end, at least one agent delivery structure disposed near the distal end and at least one electrode disposed near the distal end, wherein the distal end is configured for positioning at least one of the agent delivery structures and at least one of the one electrodes near a dorsal root ganglion; and
an agent releaseable from the at least one agent delivery structure, wherein electrical energy provided by the at least one electrode selectively activates the agent in a first cell type within the dorsal root ganglion while not activating the agent in a second cell type within the dorsal root ganglion.

61. The neuromodulation system as in claim 60, wherein the agent comprises a pro-drug.

62. The neuromodulation system as in claim 60, wherein the agent is selected from one or any combination selected from the group consisting of: opioids, COX inhibitors, PGE2 inhibitors, Na+ channel inhibitors.

63. The neuromodulation system as in claim 60, wherein the agent is an agonist or antagonist of a receptor or ion channel which is upregulated in a dorsal root ganglion in response to nerve injury, inflammation, neuropathic pain, and/or nociceptive pain.

64. The neuromodulation system as in claim 63, wherein the ion channel expressed by the dorsal root ganglion is selected from the group consisting of: voltage gated sodium channels (VGSC), voltage gated Calcium Channels (VGCC), voltage gated potassium channel (VGPC), acid-sensing ion channels (ASICs).

65. The neuromodulation system as in claim 64, wherein the voltage-gated sodium channel includes TTX-resistant voltage gated sodium channels.

66. The neuromodulation system as in claim 65, wherein the TTX-resistant voltage gated sodium channels include Naᵥ1.8 and Naᵥ1.9.

67. The neuromodulation system as in claim 64, wherein the voltage-gated sodium channel includes TTX-sensitive voltage gated sodium channels.

68. The neuromodulation system as in claim 67, wherein the TTX-sensitive voltage gated sodium channels is Brain III (Naᵥ1.3).

69. The neuromodulation system as in claim 63, wherein the receptor is selected from ATP receptor, NMDA receptors, EP4 receptors, matrix metalloproteins (MMPs), TRP receptors, neurtensin receptors.
